(19) United States
(12) Patent Application Publication
(10) Pub. No.: US 2023/0058312 A1
(43) Pub. Date: Feb. 23, 2023

(54) ARYL AND HETEROARYL COMPOUNDS, AND THERAPEUTIC USES THEREOF IN CONDITIONS ASSOCIATED WITH THE ALTERATION OF THE ACTIVITY OF GALACTOCEREBROSIDASE

(71) Applicant: GAIN THERAPEUTICS SA, Lugano (CH)

(72) Inventors: Ana Maria GARCÍA COLLAZO, Esplugues de Llobregat, Barcelona (ES); Elena CUBERO JORDÀ, Barcelona (ES); Xavier BARRIL ALONSO, Montferri (ES); Manolo BELLOTTO, Lugano (CH)

(73) Assignee: GAIN THERAPEUTICS SA, Lugano (CH)

(21) Appl. No.: 17/779,485

(22) PCT Filed: Nov. 25, 2020
(86) PCT No.: PCT/IB2020/061156

§ 371 Date: May 24, 2022

(30) Foreign Application Priority Data
Nov. 25, 2019 (EP) 19383036.1

(51) Int. Cl. A61K 31/341; C07D 513/04; C07D 471/04; C07D 307/52; C07D 405/12; C07D 405/14; C07D 417/12; C07C 237/52; C07D 487/04; C07C 317/40; C07D 403/14; C07D 307/54; C07D 417/14; C07D 413/14; C07D 295/02; C07D 213/53; C07D 213/40; C07C 275/40; C07D 307/14; C07D 213/75; C07D 295/13; C07D 307/68; A61K 31/519; A61K 31/4525; A61K 31/4025; A61K 31/4725; A61K 31/501; A61K 31/4184; A61K 31/428; A61K 31/167; A61K 31/5025; A61K 31/18; A61K 31/443; A61K 31/416; A61K 31/4155; A61K 31/4178; A61K 31/4418; A61K 31/4406; A61K 31/17; A61K 31/5375; A61K 38/46; A61K 38/00; A61K 38/45

(52) U.S. Cl. CPC A61K 31/341; C07D 513/04; C07D 471/04; C07D 307/52; C07D 405/12; C07D 405/14; C07D 417/12; C07C 237/52; C07D 487/04; C07C 317/40; C07D 403/14; C07D 307/54; C07D 417/14; C07D 413/14; C07D 295/02; C07D 213/53; C07D 213/40; C07C 275/40; C07D 307/14; C07D 213/75; C07D 295/13; C07D 307/68; A61K 31/519; A61K 31/4525; A61K 31/4025; A61K 31/4725; A61K 31/501; A61K 31/4184; A61K 31/428; A61K 31/167; A61K 31/5025; A61K 31/18; A61K 31/443; A61K 31/416; A61K 31/4155; A61K 31/4178; A61K 31/4418; A61K 31/4406; A61K 31/17; A61K 31/5375; A61K 38/465; A61K 38/005; A61K 38/45; C07C 2601/02

(57) ABSTRACT

The application is directed to compounds of formulae (IA) and (IB): (IA) and (IB), and their salts and solvates, wherein R^{1a}, R^{2a}, ^{A1}, A², A³, A⁴, R^{1b}, R^{2b}, B¹, B², B³, and G are as set forth in the specification, as well as to methods for their preparation, N pharmaceutical compositions comprising the same, and use thereof for the treatment and/or prevention of, e.g., lysosomal storage diseases, such as Krabbe's disease, and α-synucleinopathies, such as Parkinson's disease.

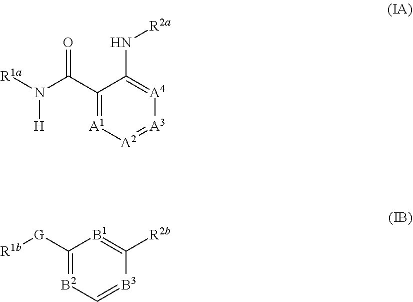

### CROSS-REFERENCE TO RELATED APPLICATION

This application claims priority to European Patent Application No. EP19383031.1, filed on Nov. 25, 2019, the entirety of which is incorporated by reference herein.

### FIELD OF THE DISCLOSURE

The present disclosure is related to aryl and heteroaryl compounds, and the use of the aryl and heteroaryl compounds in the treatment and/or prevention of conditions associated with the alteration of the activity of galactocerebrosidase in a patient, such as, for example, lysosomal storage diseases and α-synucleinopathies. The present disclosure is also related to the use of the aryl and heteroaryl compounds described herein in the treatment and/or prevention of medical disorders in a patient, such as, for example, Krabbe's disease, demyelinating disorders, galactosylsphingosine related disorders, globoid cell leukodystrophy, multiple sclerosis (MS), Parkinson's disease, peripheral neuropathy, progressive multiple sclerosis, pulmonary artery enlargement in COPD, open angle glaucoma, Lewy body dementia, and multiple system atrophy (MSA).

### BACKGROUND OF THE DISCLOSURE

Krabbe's disease, suggested to arise from galactocerebrosidase enzyme deficiency, is very rare lysosomal storage disease. The condition associated with galactocerebrosidase is known to be caused by a deficiency of the enzyme galactocerebrosidase due to mutations in the gene.

Galactocerebrosidase is an enzyme that in humans is encoded by the GALC gene and it removes galactose from ceramide derivatives (galactocerebrosides). Mutations in the GALC gene have been associated with many lysosomal disorders, like Krabbe's disease. Loss of function of the galactocerebrosidase enzyme results in the accumulation of its undigested substrates, most toxically, the sphingolipid psychosine and a progressive demyelination of the central and peripheral nervous systems. Such mutations in the GALC gene have also been suggested associated with α-synucleinopathies, such as Parkinson's disease and Lewy body dementia. See, e.g., Marshall and Bongarzone, J. Neurosci. Res. 94(11):1328-1332 (2016); Scott-Hewitt et al., Neural Regeneration Research 13(3):393-401 (2018); Abdelkarim et al., Scientific Reports 8:12462 (2018); and Smith et al., ASN Neuro 3(4):213-222 (2011).

Krabbe's (or Krabbe) disease is (also known as globoid cell leukodystrophy or galactosylceramide lipidosis) is a rare and often fatal lysosomal storage disease that results in progressive damage to the nervous system. Krabbe's disease involves dysfunctional metabolism of sphingolipids and is inherited in an autosomal recessive pattern. Infants with Krabbe's disease are normal at birth. Symptoms begin between the ages of 3 and 6 months with irritability, fevers, limb stiffness, seizures, feeding difficulties, vomiting, and slowing of mental and motor development. Other symptoms include muscle weakness, spasticity, deafness, optic atrophy, optic nerve enlargement, blindness, paralysis, and difficulty when swallowing. Prolonged weight loss may also occur. Juvenile and adult-onset cases of Krabbe's disease also occur, which have similar symptoms but slower progression. Krabbe's disease is caused by mutations in the GALC gene located on chromosome 14 (14q31), which is inherited in an autosomal recessive manner. Mutations in the GALC gene cause a deficiency of an enzyme called galactosylceramidase. In rare cases, it may be caused by a lack of active saposin A (a derivative of prosaposin). The buildup of unmetabolized lipids adversely affects the growth of the nerve's protective myelin sheath (the covering that insulates many nerves) resulting in demyelination and severe progressive degeneration of motor skills.

Mutations in the gene encoding galactocerebrosidase are also a risk factor for synucleinopathies, such as Parkinson's disease and diffuse Lewy Body disease. Parkinson's disease is a degenerative disorder of the central nervous system associated with death of dopamine-containing cells in a region of the midbrain. Diffuse Lewy Body disease is a dementia that is sometimes confused with Alzheimer's disease.

Small molecules capable of binding allosterically or competitively to mutated galactocerebrosidase enzyme, thereby stabilizing the enzyme against degradation (chaperones), constitute an important therapeutic target in conditions associated with the alteration of the activity of galactocerebrosidase. By binding and stabilizing mutant proteins, these chemical chaperones facilitate protein folding and eventually increase their transport to the lysosome. Improved trafficking of the mutant protein from the ER to the lysosome results in the reduction of lysosome size and correction of the storage. These chaperones may also increase the stability of mutant enzymes toward degradation in the lysosome. See, e.g., Patniak et al., Journal of Medicinal Chemistry 55(12):5734-5748 (2012).

It has been surprisingly found that compounds of formulae (IA) and (IB) are capable of binding to galactocerebrosidase thereby stabilizing the enzyme against denaturation.

### BRIEF SUMMARY OF THE DISCLOSURE

The present disclosure is related to the discovery that aryl and heteroaryl compounds represented by formulae (IA), (IIA), (IB), (IIB), and (IIIB) are capable of binding to galactocerebrosidase (mutated or not) and are thus useful in the treatment or prevention of, e.g., a lysosomal storage disease, such as Krabbe's disease, or α-synucleinopathies, such as Parkinson's disease, or other conditions associated with the alteration of the activity of galactocerebrosidase.

In one aspect, the present disclosure provides a method of treating or preventing a condition associated with the alteration of the activity of galactocerebrosidase in a patient in need thereof, comprising administering an effective amount of a compound of formula (IA) or formula (IB), or a salt or solvate thereof, as described herein. Compounds represented by formulae (IA) and (IIA), and formulae (IB), (IIB) and (IIIB), and the salts and solvates thereof, are herein collectively referred to as “Compounds of the Disclosure” (each individually referred to as a “Compound of the Disclosure”).

In another aspect, the present disclosure provides a method of treating or preventing a lysosomal storage disease, such as Krabbe's disease, in a patient in need thereof by administering an effective amount of a Compound of the Disclosure.

In another aspect, the present disclosure provides a method of treating or preventing an α-synucleinopathy, such as Parkinson's disease, in a patient in need thereof by administering an effective amount of a Compound of the Disclosure.

In another aspect, the present disclosure is directed to method of treating or preventing a disease or disorder selected from the group consisting of: Krabbe's disease, demyelinating disorders, galactosylsphingosine related disorders, globoid cell leukodystrophy, multiple sclerosis (MS), Parkinson's disease, peripheral neuropathy, progressive multiple sclerosis, pulmonary artery enlargement in COPD, open angle glaucoma, Lewy body dementia, and multiple system atrophy (MSA), comprising administering to a patient in need thereof an effective amount of a Compound of the Disclosure.

In another aspect, the methods described herein further comprise administering to the patient at least one other therapeutic agent. In another aspect, the therapeutic agent is an effective amount of an enzyme for enzyme replacement therapy. In another aspect, the enzyme is galactocerebrosidase or an analog thereof. In another aspect, the therapeutic agent is an effective amount of a small molecule chaperone. In another aspect, the small molecule chaperone binds competitively to an enzyme. In another aspect, the small molecule chaperone is selected from the group consisting of iminoalditols, iminosugars, aminosugars, thiophenylglycosides, glycosidase, sulfatase, glycosyl transferase, phosphatase, and peptidase inhibitors.

In another aspect, the therapeutic agent is an effective amount of substrate reduction agent for substrate reduction therapy.

A number of compounds useful in the treatment or prevention of the present disclosure have not been heretofor reported. Thus, one aspect of the present disclosure is directed to the novel compounds of formulae (IA), (IIA), (IB), (IIB), and (IIIB), and the salts and solvates thereof. Another aspect of the present disclosure is directed to pharmaceutical compositions comprising these novel compounds of formulae (IA), (IIA), (IB), (IIB), and (IIIB), and the salts and solvates thereof, and at least one pharmaceutically acceptable excipient.

In another aspect, the present disclosure provides compounds of formula (IA), and the salts and solvates thereof, with the proviso that no more than one of A¹, A², A³, or A⁴ is N.

In another aspect, the present disclosure provides compounds of formula (IIA), and the salts and solvates thereof.

In another aspect, the present disclosure provides compounds of formula (IIA), and the salts and solvates thereof, with the following provisos: 1) when A¹ is N and R^{2′} is —C₁₋₄ alkyl-C(═O)NHRa^{a′}, then Ra^{a′} is other than -(5- to 10-membered)-C₂₋₉ heterocyclyl; or 2) when A⁴ is N, then R^{2a′} is other than —C(═O)Ra^{a′}.

In another aspect, the present disclosure provides compounds of formula (IB), and the salts and solvates thereof, with the proviso that no more than one of B¹, B², or B³ is N.

In another aspect, the present disclosure provides compounds of formulae (JIB) and (IIIB), and the salts and solvates thereof, with the proviso that no more than one of B¹, B², or B³ is N.

In another aspect, the present disclosure provides a Compound of the Disclosure, as described herein, for use in the prevention or treatment of a condition associated with the alteration of the activity of galactocerebrosidase in a patient in need thereof.

In another aspect, the present disclosure provides a Compound of the Disclosure, as described herein, for use in the prevention or treatment of a lysosomal storage disease, such as Krabbe's disease.

In another aspect, the present disclosure provides a Compound of the Disclosure, as described herein, for use in the prevention or treatment of an α-synucleinopathy, such as Parkinson's disease.

In another aspect, the present disclosure provides a Compound of the Disclosure, as described herein, for use in the prevention or treatment of a disease or disorder selected from the group consisting of: Krabbe's disease, demyelinating disorders, galactosylsphingosine related disorders, globoid cell leukodystrophy, multiple sclerosis (MS), Parkinson's disease, peripheral neuropathy, progressive multiple sclerosis, pulmonary artery enlargement in COPD, open angle glaucoma, Lewy body dementia, and multiple system atrophy (MSA).

In another aspect, the present disclosure is also directed to the use of a Compound of the Disclosure, as described herein, for the treatment or prevention of a condition associated with the alteration of the activity of galactocerebrosidase in a patient in need thereof, such as lysosomal storage diseases and α-synucleinopathies described herein.

In another aspect, the present disclosure provides a pharmaceutical composition comprising a Compound of the Disclosure, as described herein, and at least one pharmaceutically acceptable excipient.

In another aspect, the present disclosure provides a Compound of the Disclosure, as described herein, for use as a medicament.

In another aspect, the present disclosure provides use of a Compound of the Disclosure, as described herein, in the preparation of a medicament for the prevention or treatment of a condition associated with the alteration of the activity of galactocerebrosidase in a patient in need thereof, such as lysosomal storage diseases and α-synucleinopathies described herein.

In another aspect, the present disclosure provides a pharmaceutical composition comprising a Compound of the Disclosure, as described herein, and at least one pharmaceutically acceptable excipient, for use in the treatment or prevention of a condition associated with the alteration of the activity of galactocerebrosidase in a patient in need thereof, such as lysosomal storage diseases and α-synucleinopathies described herein.

Other aspects and advantages of the disclosure will be readily apparent from the following detailed description of the disclosure. The embodiments and advantages of the disclosure will be realized and attained by means of the elements and combinations particularly pointed out in the appended claims.

It is to be understood that both the foregoing summary and the following detailed description are exemplary and explanatory only and are not restrictive of the disclosure as claimed.

### DETAILED DESCRIPTION OF THE DISCLOSURE

One aspect of the disclosure is based on the use of Compounds of the Disclosure for binding to mutated galactocerebrosidase. In view of this property, Compounds of the Disclosure are expected to be useful for treating or preventing, e.g., Krabbe's disease and other diseases or conditions described herein.

Compounds of the Disclosure useful in this aspect of the disclosure are compounds of formula (IA) and formula (IB):

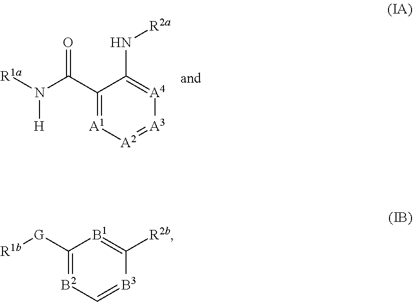

and the pharmaceutically acceptable salts and solvates thereof, wherein A¹, A², A³, A⁴, R^{1a}, R^{2a}, B¹, B², B³, G, R^{1b}, and R^{2b} are as defined below.

In another aspect, Compounds of the Disclosure are compounds of formula (IA):

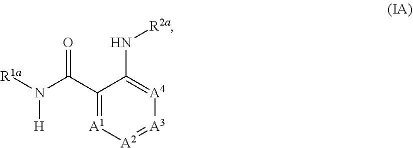

and the pharmaceutically acceptable salts and solvates thereof, wherein

A¹, A², A³, and A⁴ are each independently selected from the group consisting of N, CH and C(R^{3a});

each R^{3a} is independently selected from the group consisting of halogen, —OH, —C₁₋₄ alkyl, halo(C₁₋₄ alkyl), —C₁₋₄ alkoxy, halo(C₁₋₄ alkoxy), and —CN;

R^{1a} is selected from the group consisting of —C₁₋₄ alkyl, —C₃₋₁₀ cycloalkyl, —C₁₋₄ alkyl-C₃₋₁₀ cycloalkyl, —C₆₋₁₀ aryl, —C₁₋₄ alkyl-C₆₋₁₀ aryl, -(5- to 10-membered)-C₁₋₉ heteroaryl, —C₁₋₄ alkyl-(5- to 10-membered)-C₁₋₉ heteroaryl, -(5- to 10-membered)-C₂₋₉ heterocyclyl, and —C₁₋₄ alkyl-(5- to 10-membered)-C₂₋₉ heterocyclyl, wherein said alkyl, cycloalkyl, alkylcycloalkyl, aryl, alkylaryl, heteroaryl, alkylheteroaryl, heterocyclyl and alkylheterocyclyl groups are optionally substituted with 1, 2 or 3 substituents each independently selected from the group consisting of halogen, hydroxy, CN, —ORb^{a}, —SRb^{a}, —N(Rb^{a})₂, —C₁₋₄ alkyl optionally substituted with 1, 2, or 3 halogen atoms, optionally substituted —C₆₋₁₀ aryl, optionally substituted -(5- to 10-membered)-C₁₋₉ heteroaryl, and -(5- to 10-membered)-C₂₋₉ heterocyclyl; and wherein said cycloalkyl, alkylcycloalkyl, aryl, alkylaryl, heteroaryl, alkylheteroaryl, heterocyclyl and alkylheterocyclyl is optionally fused to a further (second) ring; and

R^{2a} is selected from the group consisting of —C₁₋₄ alkyl, —C(═O)Ra^{a}, —C(═O)NHRa^{a}, —S(═O)₂Ra^{a}, —C₁₋₄ alkyl-C(═O)Ra^{a}, —C₁₋₄ alkyl-C(═O)NHRa^{a}, —C₁₋₄ alkyl-C(═O)N(Ra^{a})₂, —C₁₋₄ alkyl-S(═O)₂Ra^{a}, —C₁₋₄ alkyl-S(═O)₂—N(Ra^{a})₂, —C₁₋₄ alkyl-C₃₋₁₀ cycloalkyl, —C₁₋₄ alkyl-C₆₋₁₀ aryl, -(5- to 10-membered)-C₁₋₉ heteroaryl, —C₁₋₄ alkyl-(5- to 10-membered)-C₁₋₉ heteroaryl, (5- to 10-membered)-C₂₋₉ heterocyclyl, and —C₁₋₄ alkyl-(5- to 10-membered)-C₂₋₉ heterocyclyl, wherein said alkyl, cycloalkyl, alkylcycloalkyl, aryl, alkylaryl, heteroaryl, alkylheteroaryl, heterocyclyl and alkylheterocyclyl groups are optionally substituted with 1, 2 or 3 substituents each independently selected from the group consisting of halogen, hydroxy, —CN, —C(═O)Ra^{a}, —ORb^{a}, —SRb^{a}, —N(Rb^{a})₂, (═O), —C₁₋₄ alkyl optionally substituted with 1, 2, or 3 halogen atoms, optionally substituted C₆₋₁₀ aryl, optionally substituted -(5- to 10-membered)-C₁₋₉ heteroaryl, and -(5- to 10-membered)-C₂₋₉ heterocyclyl; and wherein said cycloalkyl, alkylcycloalkyl, aryl, alkylaryl, heteroaryl, alkylheteroaryl, heterocyclyl and alkylheterocyclyl is optionally fused to a further (second) ring; and Ra^{a} is selected from the group consisting of —C₁₋₄ alkyl, —C₃₋₁₀ cycloalkyl, —C₁₋₄ alkyl-C₃₋₁₀ cycloalkyl, —C₆₋₁₀ aryl, —C₁₋₄ alkyl-C₆₋₁₀ aryl, -(5- to 10-membered)-C₁₋₉ heteroaryl, —C₁₋₄ alkyl-(5- to 10-membered)-C₁₋₉ heteroaryl, -(5- to 10-membered)-C₂₋₉ heterocyclyl, and —C₁₋₄ alkyl-(5- to 10-membered)-C₂₋₉ heterocyclyl, wherein said alkyl, cycloalkyl, alkylcycloalkyl, aryl, alkylaryl, heteroaryl, alkylheteroaryl, heterocyclyl and alkylheterocyclyl groups are optionally substituted with 1, 2 or 3 substituents each independently selected from the group consisting of halogen, hydroxy, —CN, —ORb^{a}, —SRb^{a}, —N(Rb^{a})₂, —C₁₋₄ alkyl optionally substituted with 1, 2, or 3 halogen atoms, optionally substituted —C₆₋₁₀ aryl, optionally substituted -(5- to 10-membered)-C₁₋₉ heteroaryl, and -(5- to 10-membered)-C₂₋₉ heterocyclyl; and wherein said cycloalkyl, alkylcycloalkyl, aryl, alkylaryl, heteroaryl, alkylheteroaryl, heterocyclyl and alkylheterocyclyl is optionally fused to a further (second) ring;

each Rb^{a} is independently hydrogen, —C₁₋₄ alkyl, —C₃₋₁₀ cycloalkyl, or -(5- to 10-membered)-C₂₋₉ heterocyclyl, wherein said alkyl, cycloalkyl or heterocyclyl group is optionally substituted by 1, 2 or 3 fluorine atoms.

In another embodiment of this aspect of the disclosure, Compounds of the Disclosure are compounds of formula (IA), and the pharmaceutically acceptable salts and solvates thereof, as defined above, wherein R^{2a} is selected from the group consisting of —C₁₋₄ alkyl, —C(═O)Ra^{a}, —S(═O)₂Ra^{a}, —C₁₋₄ alkyl-C(═O)Ra^{a}, —C₁₋₄ alkyl-C(═O)NHRa^{a}, —C₁₋₄ alkyl-C(═O)N(Ra^{a})₂, —C₁₋₄ alkyl-S(═O)₂Ra^{a}, —C₁₋₄ alkyl-S(═O)₂—N(Ra^{a})₂, —C₁₋₄ alkyl-C₃₋₁₀ cycloalkyl, —C₁₋₄ alkyl-C₆₋₁₀ aryl, -(5- to 10-membered)-C₁₋₉ heteroaryl, —C₁₋₄ alkyl-(5- to 10-membered)-C₁₋₉ heteroaryl, (5- to 10-membered)-C₂₋₉ heterocyclyl, and —C₁₋₄ alkyl-(5- to 10-membered)-C₂₋₉ heterocyclyl, wherein said alkyl, cycloalkyl, alkylcycloalkyl, aryl, alkylaryl, heteroaryl, alkylheteroaryl, heterocyclyl and alkylheterocyclyl groups are optionally substituted with 1, 2 or 3 substituents each independently selected from the group consisting of halogen, hydroxy, —CN, —C(═O)Ra^{a}, —ORb^{a}, —SRb^{a}, —N(Rb^{a})₂, (═O), —C₁₋₄ alkyl optionally substituted with 1, 2, or 3 halogen atoms, optionally substituted C₆₋₁₀ aryl, optionally substituted -(5- to 10-membered)-C₁₋₉ heteroaryl, and -(5- to 10-membered)-C₂₋₉ heterocyclyl; and wherein said cycloalkyl, alkylcycloalkyl, aryl, alkylaryl, heteroaryl, alkylheteroaryl, heterocyclyl and alkylheterocyclyl is optionally fused to a further (second) ring.

In another embodiment of this aspect of the disclosure, Compounds of the Disclosure are compounds of formula (IA), and the pharmaceutically acceptable salts and solvates thereof, as defined above, wherein each R^{3a} is independently selected from the group consisting of halogen, —C₁₋₄ alkyl, —C₁₋₄ alkoxy, and —CN.

In another embodiment of this aspect of the disclosure, Compounds of the Disclosure are compounds of formula (IA), and the pharmaceutically acceptable salts and solvates thereof, wherein A¹, A², A³, and A⁴ are CH.

In another embodiment of this aspect of the disclosure, Compounds of the Disclosure are compounds of formula (IA), and the pharmaceutically acceptable salts and solvates thereof, wherein one of A¹, A², A³, and A⁴ is C(R^{3a}) and the ones not C(R^{3a}) are CH. In some embodiments, R^{3a} is —OH. In some embodiments, R^{3a} is halo(C₁₋₄ alkyl), such as trifluoromethyl. In some embodiments, R^{3a} is halogen, such as F or Cl. In some embodiments, R^{3a} is halo(C₁₋₄ alkoxy), such as —OCF₃.

In another embodiment of this aspect of the disclosure, Compounds of the Disclosure are compounds of formula (IA), and the pharmaceutically acceptable salts and solvates thereof, wherein two of A¹, A², A³, and A⁴ is C(R^{3a}) and the ones not C(R^{3a}) are CH. In some embodiments, R^{3a} is —OH. In some embodiments, R^{3a} is halo(C₁₋₄ alkyl), such as trifluoromethyl. In some embodiments, R^{3a} is halogen, such as F or Cl. In some embodiments, R^{3a} is halo(C₁₋₄ alkoxy), such as —OCF₃.

In another embodiment of this aspect of the disclosure, Compounds of the Disclosure are compounds of formula (IA), and the pharmaceutically acceptable salts and solvates thereof, wherein A¹ is N and A², A³, and A⁴ are each independently selected from the group consisting of CH and C(R^{3a}). In some embodiments, R^{3a} is —OH. In some embodiments, R^{3a} is halo(C₁₋₄ alkyl), such as trifluoromethyl. In some embodiments, R^{3a} is halogen, such as F or Cl. In some embodiments, R^{3a} is halo(C₁₋₄ alkoxy), such as —OCF₃. In another embodiment, A², A³, and A⁴ are each CH.

In another embodiment, Compounds of the Disclosure are compounds of formula (IA), and their pharmaceutically acceptable salts and solvates thereof, wherein A² is N and A¹, A³, and A⁴ are each independently selected from the group consisting of CH and C(R^{3a}) In some embodiments, R^{3a} is —OH. In some embodiments, R^{3a} is halo(C₁₋₄ alkyl), such as trifluoromethyl. In some embodiments, R^{3a} is halogen, such as F or Cl. In some embodiments, R^{3a} is halo(C₁₋₄ alkoxy), such as —OCF₃. In another embodiment, A¹, A³, and A⁴ are each CH.

In another embodiment, Compounds of the Disclosure are compounds of formula (IA), and the pharmaceutically acceptable salts and solvates thereof, wherein A³ is N and A¹, A², and A⁴ are each independently selected from the group consisting of CH and C(R^{3a}) In some embodiments, R^{3a} is —OH. In some embodiments, R^{3a} is halo(C₁₋₄ alkyl), such as trifluoromethyl. In some embodiments, R^{3a} is halogen, such as F or Cl. In some embodiments, R^{3a} is halo(C₁₋₄ alkoxy), such as —OCF₃. In another embodiment, A¹, A², and A⁴ are each CH.

In another embodiment, Compounds of the Disclosure are compounds of formula (IA), and the pharmaceutically acceptable salts and solvates thereof, wherein A⁴ is N and A¹, A², and A³ are each independently selected from the group consisting of CH and C(R^{3a}) In some embodiments, R^{3a} is —OH. In some embodiments, R^{3a} is halo(C₁₋₄ alkyl), such as trifluoromethyl. In some embodiments, R^{3a} is halogen, such as F or Cl. In some embodiments, R^{3a} is halo(C₁₋₄ alkoxy), such as —OCF₃. In another embodiment, A¹, A², and A³ are each CH.

In another embodiment, Compounds of the Disclosure are compounds of formula (IA), and the pharmaceutically acceptable salts and solvates thereof, wherein two of A¹, A², A³, and A⁴ are N, and those that are not N are each independently selected from the group consisting of CH and C(R^{3a}). In some embodiments, R^{3a} is —OH. In some embodiments, R^{3a} is halo(C₁₋₄ alkyl), such as trifluoromethyl. In some embodiments, R^{3a} is halogen, such as F or Cl. In some embodiments, R^{3a} is halo(C₁₋₄ alkoxy), such as —OCF₃.

In another embodiment, Compounds of the Disclosure are compounds of formula (IA), and the pharmaceutically acceptable salts and solvates thereof, wherein three of A¹, A², A³, and A⁴ are N, and the one not N is selected from the group consisting of CH and C(R^{3a}). In some embodiments, R^{3a} is —OH. In some embodiments, R^{3a} is halo(C₁₋₄ alkyl), such as trifluoromethyl. In some embodiments, R^{3a} is halogen, such as F or Cl. In some embodiments, R^{3a} is halo(C₁₋₄ alkoxy), such as —OCF₃.

In another embodiment, Compounds of the Disclosure are compounds of formula (IA), having the structure of formula (IIA):

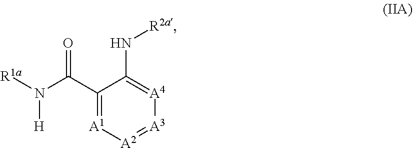

and the pharmaceutically acceptable salts and solvates thereof, wherein A¹, A², A³, and A⁴ are each independently selected from the group consisting of N, CH and C(R^{3a}) provided that no more than one of A¹, A², A³, or A⁴ is N;

each R^{3a} is independently selected from the group consisting of halogen, —OH, —C₁₋₄ alkyl, halo(C₁₋₄ alkyl), —C₁₋₄ alkoxy, halo(C₁₋₄ alkoxy), and CN;

R^{1a} is selected from the group consisting of —C₁₋₄ alkyl, —C₃₋₁₀ cycloalkyl, —C₁₋₄ alkyl-C₃₋₁₀ cycloalkyl, —C₆₋₁₀ aryl, —C₁₋₄ alkyl-C₆₋₁₀ aryl, (5- to 10-membered)-C₁₋₉ heteroaryl, —C₁₋₄ alkyl-(5- to 10-membered)-C₁₋₉ heteroaryl, (5- to 10-membered)-C₂₋₉ heterocyclyl, and —C₁₋₄ alkyl-(5- to 10-membered)-C₂₋₉ heterocyclyl, wherein said alkyl, cycloalkyl, alkylcycloalkyl, aryl, alkylaryl, heteroaryl, alkylheteroaryl, heterocyclyl and alkylheterocyclyl groups are optionally substituted with 1, 2 or 3 substituents each independently selected from the group consisting of halogen, hydroxy, —CN, —ORb^{a}, —SRb^{a}, —N(Rb^{a})₂, —C₁₋₄ alkyl optionally substituted with 1, 2, or 3 halogen atoms, optionally substituted C₆₋₁₀ aryl, optionally substituted (5- to 10-membered)-C₁₋₉ heteroaryl, and (5- to 10-membered)-C₂₋₉ heterocyclyl; and wherein said cycloalkyl, alkylcycloalkyl, aryl, alkylaryl, heteroaryl, alkylheteroaryl, heterocyclyl and alkylheterocyclyl is optionally fused to a further (second) ring;

R^{2a′} is selected from the group consisting of —C(═O)Ra^{a}, —S(═O)₂Ra^{a}, —C₁₋₄ alkyl-C(═O)NHRa^{a′}, —C₁₋₄ alkyl-C(═O)N(Ra^{a′})₂, —C₁₋₄ alkyl-S(═O)₂—N(Ra^{a′})₂, wherein said alkyl group is optionally substituted with 1, 2 or 3 substituents each independently selected from the group consisting of halogen, hydroxy, —CN, —C₁₋₄ alkyl optionally substituted with 1, 2, or 3 halogen atoms;

Ra^{a′} is selected from the group consisting of —C₆₋₁₀ aryl, —C₁₋₄ alkyl-C₆₋₁₀ aryl, (5- to 10-membered)-C₁₋₉ heteroaryl, —C₁₋₄ alkyl-(5- to 10-membered)-C₁₋₉ heteroaryl, -(5- to 10-membered)-C₂₋₉ heterocyclyl, and —C₁₋₄ alkyl-(5- to 10-membered)-C₂₋₉ heterocyclyl, wherein said aryl, alkylaryl, heteroaryl, alkylheteroaryl, heterocyclyl and alkylheterocyclyl groups are optionally substituted with 1, 2 or 3 substituents each independently selected from the group consisting of halogen, hydroxy, —CN, —ORb^{a}, —SRb^{a}, —N(Rb^{a})₂, —C₁₋₄ alkyl optionally substituted with 1, 2, or 3 halogen atoms, optionally substituted C₆₋₁₀ aryl, optionally substituted (5- to 10-membered)-C₁₋₉ heteroaryl, and (5- to 10-membered)-C₂₋₉ heterocyclyl; and wherein said cycloalkyl, alkylcycloalkyl, aryl, alkylaryl, heteroaryl, alkylheteroaryl, heterocyclyl and alkylheterocyclyl is optionally fused to a further (second) ring; and

each Rb^{a} is independently hydrogen, —C₁₋₄ alkyl, —C₃₋₁₀ cycloalkyl, or -(5- to 10-membered)-C₂₋₉ heterocyclyl, wherein said alkyl, cycloalkyl or heterocyclyl group is optionally substituted by 1, 2 or 3 fluorine atoms.

In another embodiment, Compounds of the Disclosure are compounds of formula (IIA), as defined above, and the pharmaceutically acceptable salts and solvates thereof, with the following provisos: 1) when A¹ is N and R^{2a} is —C₁₋₄ alkyl-C(═O)NHRa^{a′}, then Ra^{a′} is other than -(5- to 10-membered)-C₂₋₉ heterocyclyl; or 2) when A⁴ is N, then R^{2a′} is other than —C(═O)Ra^{a′}.

In some aspects, Compounds of the Disclosure are compounds of formula (IA), having the structure of formula (IIA):

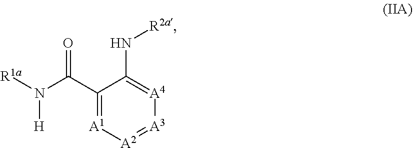

and the pharmaceutically acceptable salts and solvates thereof, wherein A¹, A², A³, and A⁴ are each independently selected from the group consisting of N, CH and C(R^{3a}), provided that at least one of A¹, A², A³, or A⁴ is N;

each R^{3a} is independently selected from the group consisting of halogen, —OH, C₁₋₄ alkyl, halo(C₁₋₄)alkyl, C₁₋₄ alkoxy, halo(C₁₋₄ alkoxy), and CN;

R^{1a} is selected from the group consisting of —C₁₋₄ alkyl, —C₃₋₁₀ cycloalkyl, —C₁₋₄ alkyl-C₃₋₁₀ cycloalkyl, —C₆₋₁₀ aryl, —C₁₋₄ alkyl-C₆₋₁₀ aryl, (5- to 10-membered)-C₁₋₉ heteroaryl, —C₁₋₄ alkyl-(5- to 10-membered)-C₁₋₉ heteroaryl, (5- to 10-membered)-C₂₋₉ heterocyclyl, and —C₁₋₄ alkyl-(5- to 10-membered)-C₂₋₉ heterocyclyl, wherein said alkyl, cycloalkyl, alkylcycloalkyl, aryl, alkylaryl, heteroaryl, alkylheteroaryl, heterocyclyl and alkylheterocyclyl groups are optionally substituted with 1, 2 or 3 substituents each independently selected from the group consisting of halogen, hydroxy, —CN, —ORb^{a}, —SRb^{a}, —N(Rb^{a})₂, —C₁₋₄ alkyl optionally substituted with 1, 2, or 3 halogen atoms, optionally substituted C₆₋₁₀ aryl, optionally substituted (5- to 10-membered)-C₁₋₉ heteroaryl, and (5- to 10-membered)-C₂₋₉ heterocyclyl; and wherein said cycloalkyl, alkylcycloalkyl, aryl, alkylaryl, heteroaryl, alkylheteroaryl, heterocyclyl and alkylheterocyclyl is optionally fused to a further (second) ring;

R^{2a′} is selected from the group consisting of —C(═O)Ra^{a′}, —S(═O)₂Ra^{a′}, —C₁₋₄ alkyl-C(═O)NHRa^{a′}, —C₁₋₄ alkyl-C(═O)N(Ra^{a′})₂, —C₁₋₄ alkyl-S(═O)₂—N(Ra^{a′})₂, wherein said alkyl group is optionally substituted with 1, 2 or 3 substituents each independently selected from the group consisting of halogen, hydroxy, —CN, —C₁₋₄ alkyl optionally substituted with 1, 2, or 3 halogen atoms;

Ra^{a′} is selected from the group consisting of —C₆₋₁₀ aryl, —C₁₋₄ alkyl-C₆₋₁₀ aryl, (5- to 10-membered)-C₁₋₉ heteroaryl, —C₁₋₄ alkyl-(5- to 10-membered)-C₁₋₉ heteroaryl, -(5- to 10-membered)-C₂₋₉ heterocyclyl, and —C₁₋₄ alkyl-(5- to 10-membered)-C₂₋₉ heterocyclyl, wherein said aryl, alkylaryl, heteroaryl, alkylheteroaryl, heterocyclyl and alkylheterocyclyl groups are optionally substituted with 1, 2 or 3 substituents each independently selected from the group consisting of halogen, hydroxy, —CN, —ORb^{a}, —SRb^{a}, —N(Rb^{a})₂, —C₁₋₄ alkyl optionally substituted with 1, 2, or 3 halogen atoms, optionally substituted C₆₋₁₀ aryl, optionally substituted (5- to 10-membered)-C₁₋₉ heteroaryl, and (5- to 10-membered)-C₂₋₉ heterocyclyl; and wherein said cycloalkyl, alkylcycloalkyl, aryl, alkylaryl, heteroaryl, alkylheteroaryl, heterocyclyl and alkylheterocyclyl is optionally fused to a further (second) ring; and

each Rb^{a} is independently hydrogen, —C₁₋₄ alkyl, —C₃₋₁₀ cycloalkyl, or -(5- to 10-membered)-C₂₋₉ heterocyclyl, wherein said alkyl, cycloalkyl or heterocyclyl group is optionally substituted by 1, 2 or 3 fluorine atoms.

In another embodiment, Compounds of the Disclosure are compounds of formula (IA), and the pharmaceutically acceptable salts and solvates thereof, wherein R^{1a} is —C₆₋₁₀ aryl or —C₁₋₄ alkyl-C₆₋₁₀ aryl, wherein said aryl or alkylaryl is optionally substituted with 1, 2 or 3 substituents each independently selected from the group consisting of halogen, hydroxy, —CN, —ORb^{a}, —SRb^{a}, —N(Rb^{a})₂, —C₁₋₄ alkyl optionally substituted with 1, 2, or 3 halogen atoms, optionally substituted —C₆₋₁₀ aryl, optionally substituted -(5- to 10-membered)-C₁₋₉ heteroaryl, and -(5- to 10-membered)-C₂₋₉ heterocyclyl, wherein Rb^{a} is as defined above.

In another embodiment, R^{1a} is unsubstituted C₆₋₁₀ aryl or C₆₋₁₀ aryl substituted with 1, 2 or 3 substituents each independently selected from the group consisting of halogen, hydroxy, —CN, —ORb^{a}, —SRb^{a}, —N(Rb^{a})₂, —C₁₋₄alkyl optionally substituted with 1, 2, or 3 halogen atoms, optionally substituted —C₆₋₁₀ aryl, optionally substituted -(5- to 10-membered)-C₁₋₉ heteroaryl and -(5- to 10-membered)-C₂₋₉ heterocyclyl. In another aspect, R^{1a} is unsubstituted —C₆₋₁₀ aryl or —C₆₋₁₀ aryl substituted with 1 or 2 substituents each independently selected from the group consisting of halogen, hydroxy, —CN, —O(C₁₋₄)alkyl, —S(C₁₋₄)alkyl, —N(C₁₋₄ alkyl)₂, —NH(C₁₋₄ alkyl), and —C₁₋₄ alkyl optionally substituted with 1, 2, or 3 halogen atoms. In another embodiment, R^{1a} is unsubstituted —C₆₋₁₀ aryl. In another embodiment, R^{1a} is unsubstituted phenyl. In another embodiment, R^{1a} is —C₆₋₁₀ aryl substituted with 1 or 2 substituents each independently selected from the group consisting of halogen, hydroxy, —CN, —O(C₁₋₄)alkyl, —S(C₁₋₄)alkyl, —N(C₁₋₄ alkyl)₂, —NH(C₁₋₄ alkyl), and —C₁₋₄ alkyl optionally substituted with 1, 2, or 3 halogen atoms. In another embodiment, R^{1a} is phenyl substituted with 1 or 2 substituents each independently selected from the group consisting of halogen, hydroxy, —CN, —O(C₁₋₄)alkyl, —S(C₁₋₄)alkyl, —N(C₁₋₄ alkyl)₂, —NH(C₁₋₄ alkyl), and —C₁₋₄ alkyl. In another embodiment, R^{1a} is phenyl substituted with methyl or ethyl. In another embodiment, R^{1a} is phenyl substituted at the ortho-position. In another embodiment, R^{1a} is phenyl substituted at the meta-position. In another embodiment, R^{1a} is phenyl substituted at the para-position.

In another embodiment, R^{1a} is unsubstituted —C₁₋₄ alkyl-C₆₋₁₀ aryl or —C₁₋₄ alkyl-C₆₋₁₀ aryl optionally substituted with 1, 2 or 3 substituents each independently selected from the group consisting of halogen, hydroxy, —CN, —ORb^{a}, —SRb^{a}, —N(Rb^{a})₂, —C₁₋₄alkyl optionally substituted with 1, 2, or 3 halogen atoms, optionally substituted —C₆₋₁₀ aryl, optionally substituted -(5- to 10-membered)-C₁₋₉ heteroaryl and -(5- to 10-membered)-C₂₋₉ heterocyclyl. In another embodiment, R^{1a} is unsubstituted —C₁₋₄ alkyl-C₆₋₁₀ aryl or —C₁₋₄ alkyl-C₆₋₁₀ aryl substituted with 1 or 2 substituents each independently selected from the group consisting of halogen, hydroxy, —CN, —O(C₁₋₄)alkyl, —S(C₁₋₄)alkyl, —N(C₁₋₄ alkyl)₂, —NH(C₁₋₄ alkyl), and —C₁₋₄ alkyl optionally substituted with 1, 2, or 3 halogen atoms. In another embodiment, R^{1a} is unsubstituted —C₁₋₄ alkyl-C₆₋₁₀ aryl. In another embodiment, R^{1a} is unsubstituted benzyl or unsubstituted phenethyl. In another aspect, R^{1a} is —C₁₋₄ alkyl-C₆₋₁₀ aryl substituted with 1 or 2 substituents each independently selected from the group consisting of halogen, hydroxy, —CN, —O(C₁₋₄)alkyl, —S(C₁₋₄)alkyl, —N(C₁₋₄ alkyl)₂, —NH(C₁₋₄ alkyl), and —C₁₋₄ alkyl optionally substituted with 1, 2, or 3 halogen atoms. In another embodiment, R^{1a} is benzyl or phenethyl substituted with 1 or 2 substituents each independently selected from the group consisting of halogen, hydroxy, —CN, —O(C₁₋₄)alkyl, —S(C₁₋₄)alkyl, —N(C₁₋₄ alkyl)₂, —NH(C₁₋₄ alkyl), and —C₁₋₄ alkyl optionally substituted with 1, 2, or 3 halogen atoms.

In another embodiment, Compounds of the Disclosure are compounds of formula (IA), and the pharmaceutically acceptable salts and solvates thereof, wherein R^{1a} is —C₃₋₁₀ cycloalkyl or —C₁₋₄ alkyl-C₃₋₁₀ cycloalkyl, wherein said cycloalkyl or alkylcycloalkyl is optionally substituted with 1, 2 or 3 substituents each independently selected from the group consisting of halogen, hydroxy, —CN, —ORb^{a}, —SRb^{a}, —N(Rb^{a})₂, —C₁₋₄ alkyl optionally substituted with 1, 2, or 3 halogen atoms, optionally substituted —C₆₋₁₀ aryl, optionally substituted -(5- to 10-membered)-C₁₋₉ heteroaryl, and -(5- to 10-membered)-C₂₋₉ heterocyclyl, wherein Rb^{a} is as defined above; and wherein said cycloalkyl is optionally fused to a further (second) ring. In another embodiment, R^{1a} is an unsubstituted —C₃₋₁₀ cycloalkyl fused to a phenyl ring. In another embodiment, R^{1a} is an unsubstituted pentyl or hexyl ring fused to a phenyl ring.

In another embodiment, Compounds of the Disclosure are compounds of formula (IA), and the pharmaceutically acceptable salts and solvates thereof, wherein R^{1a} is -(5- to 10-membered)-C₁₋₉ heteroaryl or —C₁₋₄ alkyl-(5- to 10-membered)-C₁₋₉ heteroaryl, wherein said heteroaryl or alkylheteroaryl is optionally substituted with 1, 2 or 3 substituents each independently selected from the group consisting of halogen, hydroxy, —CN, —ORb^{a}, —SRb^{a}, —N(Rb^{a})₂, —C₁₋₄ alkyl optionally substituted with 1, 2, or 3 halogen atoms, optionally substituted —C₆₋₁₀ aryl, optionally substituted -(5- to 10-membered)-C₁₋₉ heteroaryl, and -(5- to 10-membered)-C₂₋₉ heterocyclyl, wherein Rb^{a} is as defined above. In another embodiment, R^{1a} is unsubstituted -(5- to 10-membered)-C₁₋₉ heteroaryl or -(5- to 10-membered)-C₁₋₉ heteroaryl substituted with 1, 2 or 3 substituents each independently selected from the group consisting of halogen, hydroxy, —CN, —ORb^{a}, —SRb^{a}, —N(Rb^{a})₂, —C₁₋₄alkyl optionally substituted with 1, 2, or 3 halogen atoms, optionally substituted —C₆₋₁₀ aryl, optionally substituted -(5- to 10-membered)-C₁₋₉ heteroaryl and -(5- to 10-membered)-C₂₋₉ heterocyclyl. In another embodiment, R^{1a} is unsubstituted -(5- to 10-membered)-C₁₋₉ heteroaryl or -(5- to 10-membered)-C₁₋₉ heteroaryl substituted with 1 or 2 substituents each independently selected from the group consisting of halogen, hydroxy, —CN, —O(C₁₋₄)alkyl, —S(C₁₋₄)alkyl, —N(C₁₋₄alkyl)₂, —NH(C₁₋₄ alkyl), and —C₁₋₄ alkyl optionally substituted with 1, 2, or 3 halogen atoms. In another embodiment, R^{1a} is unsubstituted -(5- to 10-membered)-C₁₋₉ heteroaryl. In another embodiment, R^{1a} is unsubstituted -(5- or 6-membered)-C₁₋₃ heteroaryl. In another embodiment, R^{1a} is unsubstituted furanyl. In another embodiment, R^{1a} is unsubstituted furan-2-yl. In another embodiment, R^{1a} is -(5- or 6-membered)-C₁₋₃ heteroaryl substituted with 1 or 2 substituents each independently selected from the group consisting of halogen, hydroxy, —CN, —O(C₁₋₄)alkyl, —S(C₁₋₄)alkyl, —N(C₁₋₄ alkyl)₂, —NH(C₁₋₄ alkyl), and —C₁₋₄ alkyl optionally substituted with 1, 2, or 3 halogen atoms.

In another embodiment, R^{1a} is unsubstituted —C₁₋₄ alkyl-(5- to 10-membered)-C₁₋₉ heteroaryl or —C₁₋₄ alkyl-(5- to 10-membered)-C₁₋₉ heteroaryl optionally substituted with 1, 2 or 3 substituents each independently selected from the group consisting of halogen, hydroxy, —CN, —ORb^{a}, —SRb^{a}, —N(Rb^{a})₂, —C₁₋₄alkyl optionally substituted with 1, 2, or 3 halogen atoms, optionally substituted —C₆₋₁₀ aryl, optionally substituted -(5- to 10-membered)-C₁₋₉ heteroaryl and -(5- to 10-membered)-C₂₋₉ heterocyclyl. In another embodiment, R^{1a} is unsubstituted —C₁₋₄ alkyl-(5- to 10-membered)-C₁₋₉ heteroaryl or —C₁₋₄ alkyl-(5- to 10-membered)-C₁₋₉ heteroaryl substituted with 1 or 2 substituents each independently selected from the group consisting of halogen, hydroxy, —CN, —O(C₁₋₄)alkyl, —S(C₁₋₄)alkyl, —N(C₁₋₄ alkyl)₂, —NH(C₁₋₄ alkyl), and —C₁₋₄ alkyl optionally substituted with 1, 2, or 3 halogen atoms. In another embodiment, R^{1a} is unsubstituted —C₁₋₄ alkyl-(5- to 10-membered)-C₁₋₉ heteroaryl. In another embodiment, R^{1a} is unsubstituted —C₁₋₄ alkyl-(5- or 6-membered)-C₁₋₃ heteroaryl. In another embodiment, R^{1a} is unsubstituted furan-2-ylmethyl. In another embodiment, R^{1a} is —C₁₋₄ alkyl-(5- or 6-membered)-C₁₋₃ heteroaryl substituted with 1 or 2 substituents each independently selected from the group consisting of halogen, hydroxy, —CN, —O(C₁₋₄)alkyl, —S(C₁₋₄)alkyl, —N(C₁₋₄ alkyl)₂, —NH(C₁₋₄ alkyl), and —C₁₋₄ alkyl optionally substituted with 1, 2, or 3 halogen atoms.

In another embodiment, Compounds of the Disclosure are compounds of formula (IA), and the pharmaceutically acceptable salts and solvates thereof, wherein Rb^{a} is hydrogen or —C₁₋₄ alkyl.

In another embodiment, Compounds of the Disclosure are compounds of formula (IA), and the pharmaceutically acceptable salts and solvates thereof, wherein R^{2a} is —C₁₋₄ alkyl-(5- to 10-membered)-C₁₋₉ heteroaryl, wherein said alkylheteroaryl group is optionally substituted with 1, 2 or 3 substituents each independently selected from the group consisting of halogen, hydroxy, —CN, —C(═O)Ra^{a}, —ORb^{a}, —SRb^{a}, —N(Rb^{a})₂, (═O), —C₁₋₄ alkyl optionally substituted with 1, 2, or 3 halogen atoms, optionally substituted C₆₋₁₀ aryl, optionally substituted -(5- to 10-membered)-C₁₋₉ heteroaryl, and -(5- to 10-membered)-C₂₋₉ heterocyclyl; and wherein said cycloalkyl, alkylcycloalkyl, aryl, alkylaryl, heteroaryl, alkylheteroaryl, heterocyclyl and alkylheterocyclyl is optionally fused to a further (second) ring, wherein Ra^{a} and Rb^{a} are as defined above.

In another embodiment, Compounds of the Disclosure are compounds of formula (IA), and the pharmaceutically acceptable salts thereof, wherein R^{2a} is —C(═O)NHRa^{a}, wherein Ra^{a} is as defined above.

In another embodiment, Compounds of the Disclosure are compounds of formula (IA), and the pharmaceutically acceptable salts and solvates thereof, wherein R^{2a} is —C₁₋₄ alkyl-C(═O)NHRa^{a} or —C₁₋₄ alkyl-C(═O)N(Ra^{a})₂, wherein Ra^{a} is as defined above.

In another embodiment, Compounds of the Disclosure are compounds of formula (IA), and the pharmaceutically acceptable salts and solvates thereof, wherein R^{2a} is —S(═O)₂Ra^{a}, wherein Ra^{a} is as defined above. In another embodiment, Ra^{a} is selected from the group consisting of —C₆₋₁₀ aryl, -(5- to 10-membered)-C₁₋₉ heteroaryl, —C₃₋₁₀ cycloalkyl, and -(5- to 10-membered)-C₂₋₉ heterocyclyl, wherein said aryl, heteroaryl, cycloalkyl, and heterocyclyl groups are optionally substituted with 1, 2 or 3 substituents each independently selected from the group consisting of halogen, hydroxy, —CN, —ORb^{a}, —SRb^{a}, —N(Rb^{a})₂, —C₁₋₄alkyl optionally substituted with 1, 2, or 3 halogen atoms, optionally substituted —C₆₋₁₀ aryl, optionally substituted -(5- to 10-membered)-C₁₋₉ heteroaryl and -(5- to 10-membered)-C₂₋₉ heterocyclyl, and wherein said aryl, heteroaryl, cycloalkyl, and heterocyclyl is optionally fused to a further (second) ring. In another embodiment, Ra^{a} is phenyl fused to cycloalkyl or heterocyclyl to give a bicyclic ring system, e.g.,

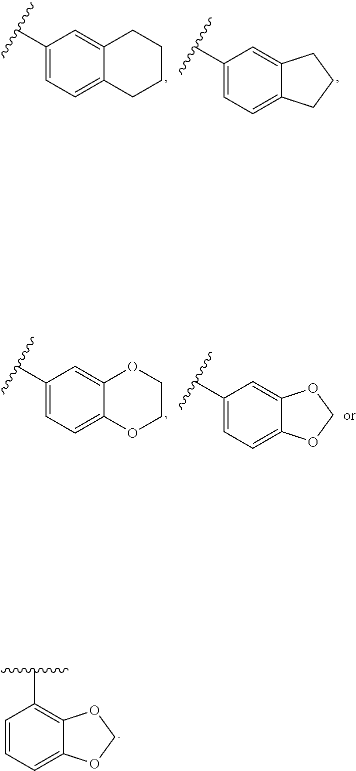

In some aspects, Compounds of the Disclosure are compounds of any one of formulae (IA) or (IIA) selected form the group consisting of

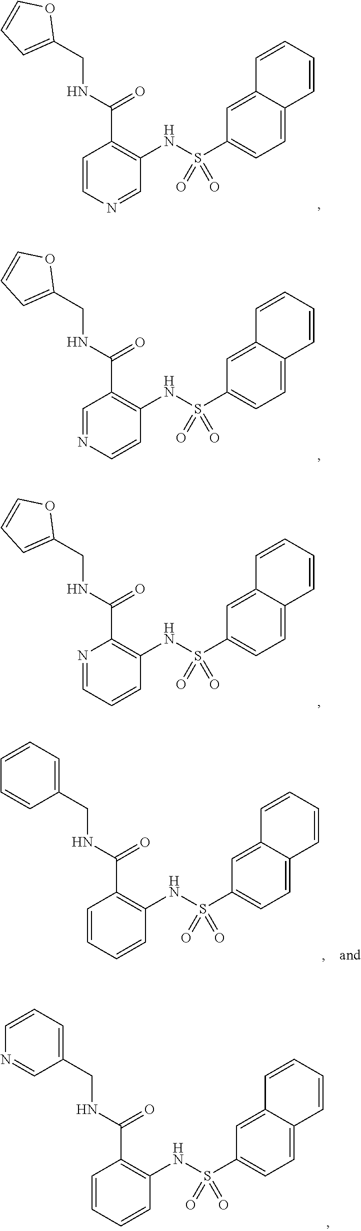 [SMILES: O=C(NCc1ccco1)c1ccncc1NS(=O)(=O)c1ccc2ccccc2c1 ; O=C(NCc1ccco1)c1cnccc1NS(=O)(=O)c1ccc2ccccc2c1 ; O=C(NCc1ccco1)c1ncccc1NS(=O)(=O)c1ccc2ccccc2c1 ; O=C(NCc1ccccc1)c1ccccc1NS(=O)(=O)c1ccc2ccccc2c1 ; O=C(NCc1cccnc1)c1ccccc1NS(=O)(=O)c1ccc2ccccc2c1]

and the pharmaceutically acceptable salts and solvates thereof.

In another aspect, Compounds of the Disclosure that can be employed in the methods of the present disclosure include compounds of formula (IA) selected from the group consisting of

[chemical structure]

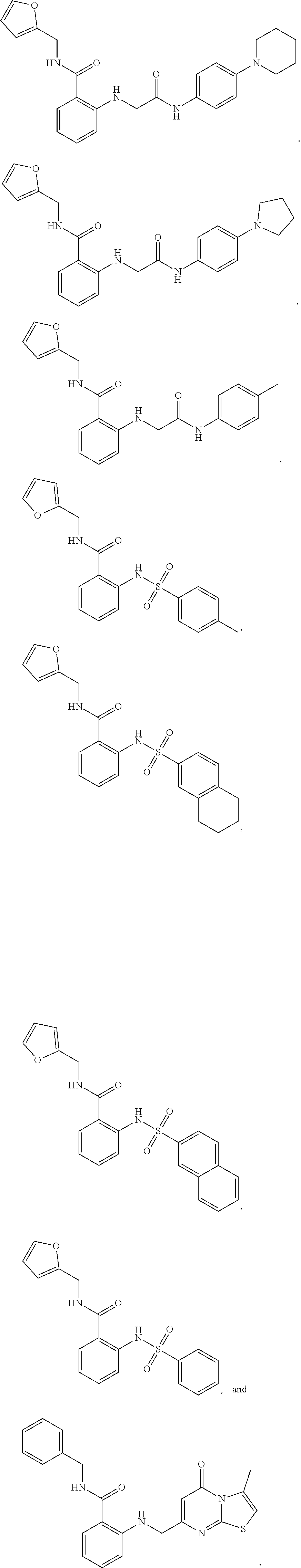

and, and the pharmaceutically acceptable salts and solvates thereof.

In another aspect, Compounds of the Disclosure that can be employed in the methods of the present disclosure include compounds of formula (IA) selected from the group consisting of

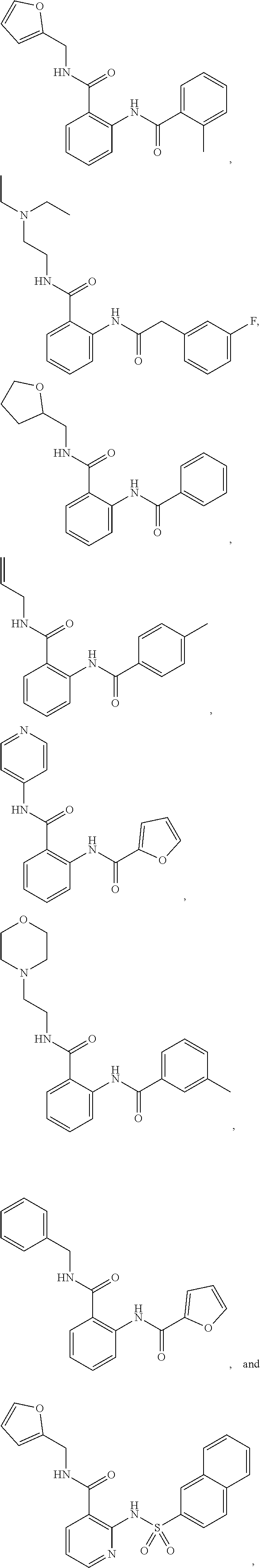

and the pharmaceutically acceptable salts and solvates thereof.

In another aspect, Compounds of the Disclosure useful in the methods described herein are compounds of formula (IB):

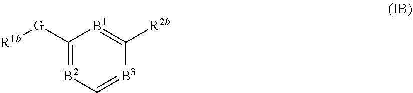

and the pharmaceutically acceptable salts and solvates thereof, wherein

G is —C(═O)—NH— or —NH—C(═O)—;

B¹, B², and B³ are each independently selected from the group consisting of N, CH and C(R^{3b});

each R^{3b} is independently selected from the group consisting of halogen, C₁₋₄ alkyl, halo(C₁₋₄ alkyl), —OH, C₁₋₄ alkoxy, halo(C₁₋₄ alkoxy), and CN;

R^{1b} is selected from the group consisting of —C₁₋₄ alkyl, —C₃₋₁₀ cycloalkyl, —C₁₋₄ alkyl-C₃₋₁₀ cycloalkyl, —C₆₋₁₀ aryl, —C₁₋₄ alkyl-C₆₋₁₀ aryl, —C₂₋₄ alkylene-C₆₋₁₀ aryl, (5- to 10-membered)-C₁₋₉ heteroaryl, —C₁₋₄ alkyl-(5- to 10-membered)-C₁₋₉ heteroaryl, —C₂₋₄ alkylene-(5- to 10-membered)-C₁₋₉ heteroaryl, (5- to 10-membered)-C₂₋₉ heterocyclyl, —C₁₋₄ alkyl-(5- to 10-membered)-C₂₋₉ heterocyclyl, and —C₂₋₄ alkenyl-(5- to 10-membered)-C₂₋₉ heterocyclyl, wherein said alkyl, cycloalkyl, alkylcycloalkyl, aryl, alkylaryl, heteroaryl, alkylheteroaryl, alkenylheteroaryl, heterocyclyl and alkylheterocyclyl groups are optionally substituted with 1, 2 or 3 substituents each independently selected from the group consisting of halogen, hydroxy, —CN, —ORb^{b}, —SRb^{b}, —N(Rb^{b})₂, —C₁₋₄ alkyl optionally substituted with 1, 2, or 3 halogen atoms, optionally substituted C₆₋₁₀ aryl, optionally substituted (5- to 10-membered)-C₁₋₉ heteroaryl, and (5- to 10-membered)-C₂₋₉ heterocyclyl; and wherein said cycloalkyl, alkylcycloalkyl, aryl, alkylaryl, heteroaryl, alkylheteroaryl, alkenylheteroaryl, heterocyclyl and alkylheterocyclyl is optionally fused to a further (second) ring;

R^{2b} is —C₆₋₁₀ aryl, -(5- to 10-membered)-C₁₋₉ heteroaryl, —C(═O)Ra^{b}, —S(═O)₂Ra^{b}, —C(═O)—NH—Ra^{b}, —S(═O)₂—NH—Ra^{b}, —C₁₋₄ alkyl-C(═O)Ra^{b}, —C₁₋₄ alkyl-S(═O)₂Ra^{b}, or —N(Rb^{b})₂, wherein said aryl and heteroaryl groups are optionally substituted with 1, 2 or 3 substituents each independently selected from the group consisting of halogen, hydroxy, CN, —ORb^{b}, —SRb^{b}, —N(Rb^{b})₂, (═O), —C₁₋₄alkyl optionally substituted with 1, 2, or 3 substituents each independently selected from the group consisting of halogen, CN, —ORb^{b}, and —N(Rb^{b})₂, optionally substituted —C₆₋₁₀ aryl, optionally substituted -(5- to 10-membered)-C₁₋₉ heteroaryl, -(5- to 10-membered)-C₂₋₉ heterocyclyl, and —C₃₋₁₀ cycloalkyl; and wherein said aryl, heteroaryl, and heterocyclyl are optionally fused to a further (second) ring; or

R^{2b} and R^{3b} attached to an adjacent carbon atom together form a 5- or 6-membered heterocyclic ring containing one N-atom substituted with —S(═O)₂Ra^{b};

Ra^{b} is selected from the group consisting of —C₁₋₄ alkyl, —C₃₋₁₀ cycloalkyl, —C₁₋₄ alkyl-C₃₋₁₀ cycloalkyl, —C₆₋₁₀ aryl, —C₁₋₄ alkyl-C₆₋₁₀ aryl, (5- to 10-membered)-C₁₋₉ heteroaryl, —C₁₋₄ alkyl-(5- to 10-membered)-C₁₋₉ heteroaryl, (5- to 10-membered)-C₂₋₉ heterocyclyl, and —C₁₋₄ alkyl-(5- to 10-membered)-C₂₋₉ heterocyclyl, wherein said alkyl, cycloalkyl, alkylcycloalkyl, aryl, alkylaryl, heteroaryl, alkylheteroaryl, heterocyclyl and alkylheterocyclyl groups are optionally substituted with 1, 2 or 3 substituents each independently selected from the group consisting of halogen, hydroxy, —CN, —ORb^{b}, —SRb^{b}, —N(Rb^{b})₂, —C₁₋₄ alkyl optionally substituted with 1, 2, or 3 halogen atoms, optionally substituted C₆₋₁₀ aryl, optionally substituted (5- to 10-membered)-C₁₋₉ heteroaryl, and (5- to 10-membered)-C₂₋₉ heterocyclyl; and wherein said cycloalkyl, alkylcycloalkyl, aryl, alkylaryl, heteroaryl, alkylheteroaryl, heterocyclyl and alkylheterocyclyl is optionally fused to a further (second) ring; and

each Rb^{b} is independently hydrogen, —C(═O)Ra^{b}, —S(═O)₂Ra^{b}, —C₁₋₄ alkyl, —C₃₋₁₀ cycloalkyl, -(5- to 10-membered)-C₂₋₉ heterocyclyl, or optionally substituted —C₆₋₁₀ aryl, wherein said alkyl, cycloalkyl or heterocyclyl group is optionally substituted by 1, 2 or 3 fluorine atoms.

In another embodiment, Compounds of the Disclosure are compounds of formula (IB), and the pharmaceutically acceptable salts and solvates thereof, as defined above, wherein each R^{3b} is independently selected from the group consisting of halogen, C₁₋₄ alkyl, —OH, C₁₋₄ alkoxy, and CN.

In another embodiment, Compounds of the Disclosure are compounds of formula (IB), wherein G is —C(═O)—NH— having formula (IIB):

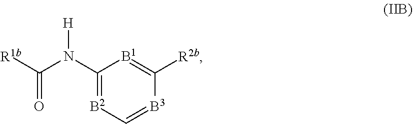

and the pharmaceutically acceptable salts and solvates thereof, wherein B¹, B², B³, R^{1b}, and R^{2b} are as defined for formula (IB).

In another embodiment, Compounds of the Disclosure are compounds of formula (IB), wherein G is —NH—C(═O)— having formula (IIIB):

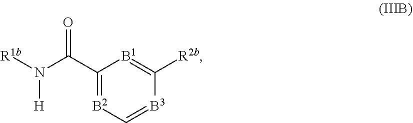

and the pharmaceutically acceptable salts and solvates thereof, wherein B¹, B², B³, R^{1b}, and R^{2b} are as defined for formula (IB).

In another embodiment of this aspect of the disclosure, Compounds of the Disclosure are compounds of any one of formulae (IB), (IIB), and (IIIB), and the pharmaceutically acceptable salts and solvates thereof, wherein B¹, B², and B³ are CH.

In another embodiment of this aspect of the disclosure, Compounds of the Disclosure are compounds of any one of formulae (IB), (IIB), and (IIIB), and the pharmaceutically acceptable salts and solvates thereof, wherein one of B¹, B², and B³ is C(R^{3b}) and the ones not C(R^{3b}) are CH. In some embodiments, R^{3b} is —OH. In some embodiments, R^{3b} is halo(C₁₋₄ alkyl), such as trifluoromethyl. In some embodiments, R^{3b} is halogen, such as F or Cl. In some embodiments, R^{3b} is halo(C₁₋₄ alkoxy), such as —OCF₃.

In another embodiment of this aspect of the disclosure, Compounds of the Disclosure are compounds of any one of formulae (IB), (IIB), and (IIIB), and the pharmaceutically acceptable salts and solvates thereof, wherein two of B¹, B², and B³ is C(R^{3b}) and the one not C(R^{3b}) is CH. In some embodiments, R^{3b} is —OH. In some embodiments, R^{3b} is halo(C₁₋₄ alkyl), such as trifluoromethyl. In some embodiments, R^{3b} is halogen, such as F or Cl. In some embodiments, R^{3b} is halo(C₁₋₄ alkoxy), such as —OCF₃.

In another embodiment, Compounds of the Disclosure are compounds of any one of formulae (IB), (IIB), and (IIIB), and the pharmaceutically acceptable salts and solvates thereof, wherein one of B¹, B² and B³ is N.

In another embodiment, Compounds of the Disclosure are compounds of any one of formulae (IB), (IIB), and (IIIB), and the pharmaceutically acceptable salts and solvates thereof, wherein two of B¹, B² and B³ are N.

In another embodiment, Compounds of the Disclosure are compounds of any one of formulae (IB), (IIB), and (IIIB), and the pharmaceutically acceptable salts and solvates thereof, wherein B¹, B² and B³ are N.

In another embodiment, Compounds of the Disclosure are compounds of any one of formulae (IB), (IIB), and (IIIB), and the pharmaceutically acceptable salts and solvates thereof, wherein B¹ is N and B² and B³ are each independently selected from the group consisting of CH and C(R^{3b}). In some embodiments, R^{3b} is —OH. In some embodiments, R^{3b} is halo(C₁₋₄ alkyl), such as trifluoromethyl. In some embodiments, R^{3b} is halogen, such as F or Cl. In some embodiments, R^{3b} is halo(C₁₋₄ alkoxy), such as —OCF₃. In another embodiment, B² and B³ are both CH.

In another embodiment, Compounds of the Disclosure are compounds of any one of formulae (IB), (IIB), and (IIIB), and their pharmaceutically acceptable salts and solvates thereof, wherein B² is N and B¹ and B³ are each independently selected from the group consisting of CH and C(R^{3b}). In some embodiments, R^{3b} is —OH. In some embodiments, R^{3b} is halo(C₁₋₄ alkyl), such as trifluoromethyl. In some embodiments, R^{3b} is halogen, such as F or Cl. In some embodiments, R^{3b} is halo(C₁₋₄ alkoxy), such as —OCF₃. In another embodiment, B¹ and B³ are both CH.

In another embodiment, Compounds of the Disclosure are compounds of any one of formulae (IB), (IIB), and (IIIB), and the pharmaceutically acceptable salts and solvates thereof, wherein B³ is N and B¹ and B² are each independently selected from the group consisting of CH and C(R^{3b}). In some embodiments, R^{3b} is —OH. In some embodiments, R^{3b} is halo(C₁₋₄ alkyl), such as trifluoromethyl. In some embodiments, R^{3b} is halogen, such as F or Cl. In some embodiments, R^{3b} is halo(C₁₋₄ alkoxy), such as —OCF₃. In another embodiment, B¹ and B² are both CH.

In another embodiment, Compounds of the Disclosure are compounds of any one of formulae (IB), (IIB), and (IIIB), and the pharmaceutically acceptable salts and solvates thereof, wherein B¹ and B² are both N and B³ is CH or C(R^{3b}). In some embodiments, R^{3b} is —OH. In some embodiments, R^{3b} is halo(C₁₋₄ alkyl), such as trifluoromethyl. In some embodiments, R^{3b} is halogen, such as F or Cl. In some embodiments, R^{3b} is halo(C₁₋₄ alkoxy), such as —OCF₃. In another embodiment, B³ is CH.

In another embodiment, Compounds of the Disclosure are compounds of any one of formulae (IB), (IIB), and (IIIB), and the pharmaceutically acceptable salts and solvates thereof, wherein B¹ and B³ are both N and B² is CH or C(R^{3b}). In some embodiments, R^{3b} is —OH. In some embodiments, R^{3b} is halo(C₁₋₄ alkyl), such as trifluoromethyl. In some embodiments, R^{3b} is halogen, such as F or Cl. In some embodiments, R^{3b} is halo(C₁₋₄ alkoxy), such as —OCF₃. In another embodiment, B² is CH.

In another embodiment, Compounds of the Disclosure are compounds of any one of formulae (IB), (IIB), and (IIIB), and the pharmaceutically acceptable salts and solvates thereof, wherein B² and B³ are both N and B¹ is CH or C(R^{3b}). In some embodiments, R^{3b} is —OH. In some embodiments, R^{3b} is halo(C₁₋₄ alkyl), such as trifluoromethyl. In some embodiments, R^{3b} is halogen, such as F or Cl. In some embodiments, R^{3b} is halo(C₁₋₄ alkoxy), such as —OCF₃. In another embodiment, B¹ is CH.

In another embodiment, Compounds of the Disclosure are compounds of any one of formulae (IB), (IIB), and (IIIB), and the pharmaceutically acceptable salts and solvates thereof, wherein R^{1b} is —C₆₋₁₀ aryl or —C₁₋₄ alkyl-C₆₋₁₀ aryl, wherein said aryl or alkylaryl is optionally substituted with 1, 2 or 3 substituents each independently selected from the group consisting of halogen, hydroxy, —CN, —ORb^{b}, —SRb^{b}, —N(Rb^{b})₂, —C₁₋₄ alkyl optionally substituted with 1, 2, or 3 halogen atoms, optionally substituted —C₆₋₁₀ aryl, optionally substituted -(5- to 10-membered)-C₁₋₉ heteroaryl, and -(5- to 10-membered)-C₂₋₉ heterocyclyl, wherein Rb^{b} is as defined above.

In another embodiment, Compounds of the Disclosure are compounds of any one of formulae (IB), (IIB), and (IIIB), and the pharmaceutically acceptable salts and solvates thereof, wherein R^{1b} is unsubstituted —C₁₋₄ alkyl-C₆₋₁₀ aryl or —C₁₋₄ alkyl-C₆₋₁₀ aryl optionally substituted with 1, 2 or 3 substituents each independently selected from the group consisting of halogen, hydroxy, —CN, —ORb^{a}, —SRb^{a}, —N(Rb^{b})₂, —C₁₋₄alkyl optionally substituted with 1, 2, or 3 halogen atoms, optionally substituted —C₆₋₁₀ aryl, optionally substituted -(5- to 10-membered)-C₁₋₉ heteroaryl and -(5- to 10-membered)-C₂₋₉ heterocyclyl. In another embodiment, R^{1b} is unsubstituted —C₁₋₄ alkyl-C₆₋₁₀ aryl or —C₁₋₄ alkyl-C₆₋₁₀ aryl substituted with 1 or 2 substituents each independently selected from the group consisting of halogen, hydroxy, —CN, —O(C₁₋₄)alkyl, —S(C₁₋₄)alkyl, —N(C₁₋₄ alkyl)₂, —NH(C₁₋₄ alkyl), and —C₁₋₄ alkyl optionally substituted with 1, 2, or 3 halogen atoms.

In another embodiment, Compounds of the Disclosure are compounds of any one of formulae (IB), (IIB), and (IIIB), and the pharmaceutically acceptable salts and solvates thereof, wherein R^{1b} is —C₃₋₁₀ cycloalkyl or —C₁₋₄ alkyl-C₃₋₁₀ cycloalkyl, wherein said cycloalkyl or alkylcycloalkyl is optionally substituted with 1, 2 or 3 substituents each independently selected from the group consisting of halogen, hydroxy, —CN, —ORb^{b}, —SRb^{b}, —N(Rb^{b})₂, —C₁₋₄ alkyl optionally substituted with 1, 2, or 3 halogen atoms, optionally substituted —C₆₋₁₀ aryl, optionally substituted -(5- to 10-membered)-C₁₋₉ heteroaryl, and -(5- to 10-membered)-C₂₋₉ heterocyclyl, wherein Rb^{b} is as defined above; and wherein said cycloalkyl is optionally fused to a further (second) ring.

In another embodiment, Compounds of the Disclosure are compounds of any one of formulae (IB), (IIB), and (IIIB), and the pharmaceutically acceptable salts and solvates thereof, wherein R^{1b} is -(5- to 10-membered)-C₁₋₉ heteroaryl, —C₁₋₄ alkyl-(5- to 10-membered)-C₁₋₉ heteroaryl, or —C₂₋₄ alkenyl-(5- to 10-membered)-C₁₋₉ heteroaryl, wherein said heteroaryl, alkylheteroaryl, or alkenylheteroaryl is optionally substituted with 1, 2 or 3 substituents each independently selected from the group consisting of halogen, hydroxy, —CN, —ORb^{b}, —SRb^{a}, —N(Rb^{b})₂, —C₁₋₄ alkyl optionally substituted with 1, 2, or 3 halogen atoms, optionally substituted —C₆₋₁₀ aryl, optionally substituted -(5- to 10-membered)-C₁₋₉ heteroaryl, and -(5- to 10-membered)-C₂₋₉ heterocyclyl, wherein Rb^{b} is as defined above.

In another embodiment, Compounds of the Disclosure are compounds of any one of formulae (IB), (IIB), and (IIIB), and the pharmaceutically acceptable salts and solvates thereof, wherein R^{1b} is unsubstituted -(5- to 10-membered)-C₁₋₉ heteroaryl or -(5- to 10-membered)-C₁₋₉ heteroaryl substituted with 1, 2 or 3 substituents each independently selected from the group consisting of halogen, hydroxy, —CN, —ORb^{b}, —SRb^{b}, —N(Rb^{b})₂, —C₁₋₄alkyl optionally substituted with 1, 2, or 3 halogen atoms, optionally substituted —C₆₋₁₀ aryl, optionally substituted -(5- to 10-membered)-C₁₋₉ heteroaryl and -(5- to 10-membered)-C₂₋₉ heterocyclyl. In another embodiment, R^{1b} is unsubstituted -(5- to 10-membered)-C₁₋₉ heteroaryl or -(5- to 10-membered)-C₁₋₉ heteroaryl substituted with 1 or 2 substituents each independently selected from the group consisting of halogen, hydroxy, —CN, —O(C₁₋₄)alkyl, —S(C₁₋₄)alkyl, —N(C₁₋₄alkyl)₂, —NH(C₁₋₄ alkyl), and —C₁₋₄ alkyl optionally substituted with 1, 2, or 3 halogen atoms. In another aspect, R^{1b} is unsubstituted -(5- to 10-membered)-C₁₋₉ heteroaryl. In another embodiment, R^{1b} is unsubstituted furanyl. In another aspect, R^{1b} is unsubstituted furan-2-yl. In another embodiment, R^{1b} is -(5- to 10-membered)-C₁₋₉ heteroaryl substituted with 1 or 2 substituents each independently selected from the group consisting of halogen, hydroxy, —CN, —O(C₁₋₄)alkyl, —S(C₁₋₄)alkyl, —N(C₁₋₄ alkyl)₂, —NH(C₁₋₄ alkyl), and —C₁₋₄ alkyl optionally substituted with 1, 2, or 3 halogen atoms.

In another embodiment, Compounds of the Disclosure are compounds of any one of formulae (IB), (IIB), and (IIIB), and the pharmaceutically acceptable salts and solvates thereof, wherein R^{1b} is unsubstituted —C₁₋₄ alkyl-(5- to 10-membered)-C₁₋₉ heteroaryl or —C₁₋₄ alkyl-(5- to 10-membered)-C₁₋₉ heteroaryl optionally substituted with 1, 2 or 3 substituents each independently selected from the group consisting of halogen, hydroxy, —CN, —ORb^{b}, —SRb^{b}, —N(Rb^{b})₂, —C₁₋₄alkyl optionally substituted with 1, 2, or 3 halogen atoms, optionally substituted —C₆₋₁₀ aryl, optionally substituted -(5- to 10-membered)-C₁₋₉ heteroaryl and -(5- to 10-membered)-C₂₋₉ heterocyclyl. In another embodiment, R^{1b} is unsubstituted —C₁₋₄ alkyl-(5- to 10-membered)-C₁₋₉ heteroaryl or —C₁₋₄ alkyl-(5- to 10-membered)-C₁₋₉ heteroaryl substituted with 1 or 2 substituents each independently selected from the group consisting of halogen, hydroxy, —CN, —O(C₁₋₄)alkyl, —S(C₁₋₄)alkyl, —N(C₁₋₄ alkyl)₂, —NH(C₁₋₄ alkyl), and —C₁₋₄ alkyl optionally substituted with 1, 2, or 3 halogen atoms. In another embodiment, R^{1b} is unsubstituted —C₁₋₄ alkyl-(5- to 10-membered)-C₁₋₉ heteroaryl. In another embodiment, R^{1b} is unsubstituted furan-2-yl-(C₁₋₄ alkyl)-.

In another embodiment, Compounds of the Disclosure are compounds of any one of formulae (IB), (IIB), and (IIIB), and the pharmaceutically acceptable salts and solvates thereof, wherein R^{1b} is unsubstituted —C₂₋₄ alkenyl-(5- to 10-membered)-C₁₋₉ heteroaryl or —C₂₋₄ alkenyl-(5- to 10-membered)-C₁₋₉ heteroaryl optionally substituted with 1, 2 or 3 substituents each independently selected from the group consisting of halogen, hydroxy, —CN, —ORb^{b}, —SRb^{b}, —N(Rb^{b})₂, —C₁₋₄alkyl optionally substituted with 1, 2, or 3 halogen atoms, optionally substituted —C₆₋₁₀ aryl, optionally substituted -(5- to 10-membered)-C₁₋₉ heteroaryl and -(5- to 10-membered)-C₂₋₉ heterocyclyl. In another embodiment, R^{1b} is unsubstituted —C₂₋₄ alkenyl-(5- to 10-membered)-C₁₋₉ heteroaryl or —C₂₋₄ alkenyl-(5- to 10-membered)-C₁₋₉ heteroaryl substituted with 1 or 2 substituents each independently selected from the group consisting of halogen, hydroxy, —CN, —O(C₁₋₄)alkyl, —S(C₁₋₄)alkyl, —N(C₁₋₄ alkyl)₂, —NH(C₁₋₄ alkyl), and —C₁₋₄ alkyl optionally substituted with 1, 2, or 3 halogen atoms. In another embodiment, R^{1b} is unsubstituted —C₂₋₄ alkenyl-(5- to 10-membered)-C₁₋₉ heteroaryl. In another embodiment, R^{1b} is unsubstituted furan-2-yl-ethenyl.

In another embodiment, Compounds of the Disclosure are compounds of any one of formulae (IB), (IIB), and (IIIB), and the pharmaceutically acceptable salts and solvates thereof, wherein R^{1b} is —C₁₋₄ alkyl optionally substituted with 1, 2 or 3 substituents each independently selected from the group consisting of halogen, hydroxy, —CN, —ORb^{b}, —SRb^{b}, —N(Rb^{b})₂, —C₁₋₄ alkyl optionally substituted with 1, 2, or 3 halogen atoms, optionally substituted C₆₋₁₀ aryl, optionally substituted (5- to 10-membered)-C₁₋₉ heteroaryl, and (5- to 10-membered)-C₂₋₉ heterocyclyl; and wherein said cycloalkyl, alkylcycloalkyl, aryl, alkylaryl, heteroaryl, alkylheteroaryl, alkenylheteroaryl, heterocyclyl and alkylheterocyclyl is optionally fused to a further (second) ring, wherein Rb^{b} is as defined herein. In another embodiment, R^{1b} is unsubstituted —C₁₋₄ alkyl. In another embodiment, R^{1b} is —C₁₋₄ alkyl substituted with —ORb^{b}, —SRb^{b}, or —N(Rb^{b})₂, wherein Rb^{b} is as described herein. In another embodiment, R^{1b} is —C₁₋₄ alkyl substituted with —ORb^{b}, —SRb^{b}, or —N(Rb^{b})₂, wherein each Rb^{b} is independently hydrogen, —C(═O)Ra^{b}, —S(═O)₂Ra^{b}, —C₁₋₄ alkyl, —C₃₋₁₀ cycloalkyl, -(5- to 10-membered)-C₂₋₉ heterocyclyl, or optionally substituted —C₆₋₁₀ aryl, wherein said alkyl, cycloalkyl or heterocyclyl group is optionally substituted by 1, 2 or 3 fluorine atoms.

In another embodiment, Compounds of the Disclosure are compounds of any one of formulae (IB), (IIB), and (IIIB), and the pharmaceutically acceptable salts and solvates thereof, wherein R^{2b} is —C₆₋₁₀ aryl, -(5- to 10-membered)-C₁₋₉ heteroaryl, —C(═O)Ra^{b}, —S(═O)₂Ra^{b}, —C(═O)—NH—Ra^{b}, —S(═O)₂—NH—Ra^{b}, —C₁₋₄ alkyl-C(═O)Ra^{b}, —C₁₋₄ alkyl-S(═O)₂Ra^{b}, or —N(Rb^{b})₂, wherein said aryl and heteroaryl groups are optionally substituted with 1, 2 or 3 substituents each independently selected from the group consisting of halogen, hydroxy, CN, —ORb^{b}, —SRb^{b}, —N(Rb^{b})₂, (═O), —C₁₋₄alkyl optionally substituted with 1, 2, or 3 substituents each independently selected from the group consisting of halogen, CN, —ORb^{b}, and —N(Rb^{b})₂, optionally substituted —C₆₋₁₀ aryl, optionally substituted -(5- to 10-membered)-C₁₋₉ heteroaryl, -(5- to 10-membered)-C₂₋₉ heterocyclyl, and —C₃₋₁₀ cycloalkyl; and wherein said aryl, heteroaryl, and heterocyclyl is optionally fused to a further (second) ring; wherein Ra^{b} and Rb^{b} are as described herein.

In another embodiment, Compounds of the Disclosure are compounds of any one of formulae (IB), (IIB), and (IIIB), and the pharmaceutically acceptable salts and solvates thereof, wherein R^{2b} is —C₆₋₁₀ aryl or -(5- to 10-membered)-C₁₋₉ heteroaryl, wherein said aryl and heteroaryl groups are optionally substituted with 1, 2 or 3 substituents each independently selected from the group consisting of halogen, hydroxy, CN, —ORb^{b}, —SRb^{b}, —N(Rb^{b})₂, (═O), —C₁₋₄alkyl optionally substituted with 1, 2, or 3 substituents each independently selected from the group consisting of halogen, CN, —ORb^{b}, and —N(Rb^{b})₂, optionally substituted —C₆₋₁₀ aryl, optionally substituted -(5- to 10-membered)-C₁₋₉ heteroaryl, -(5- to 10-membered)-C₂₋₉ heterocyclyl, and —C₃₋₁₀ cycloalkyl; and wherein said aryl, heteroaryl, and heterocyclyl is optionally fused to a further (second) ring; wherein Rb^{b} is as described herein.

In another embodiment, Compounds of the Disclosure are compounds of any one of formulae (IB), (IIB), and (IIIB), and the pharmaceutically acceptable salts and solvates thereof, wherein R^{2b} is —S(═O)₂Ra^{b}, —C(═O)—NH—Ra^{b}, —S(═O)₂—NH—Ra^{b}, —C₁₋₄ alkyl-C(═O)Ra^{b}, —C₁₋₄ alkyl-S(═O)₂Ra^{b}, or —N(Rb^{b})₂, wherein Ra^{b} and Rb^{b} are as described herein. In another aspect, R^{2b} is —C(═O)—NH—Ra^{b} or —S(═O)₂—NH—Ra^{b}, wherein Ra^{b} is —C₆₋₁₀ aryl optionally substituted with 1, 2 or 3 substituents each independently selected from the group consisting of halogen, hydroxy, —CN, —ORb^{b}, —SRb^{b}, —N(Rb^{b})₂, and —C₁₋₄ alkyl optionally substituted with 1, 2, or 3 halogen atoms.

In another embodiment, Compounds of the Disclosure are compounds of any one of formulae (IB), (IIB), and (IIIB), and the pharmaceutically acceptable salts and solvates thereof, wherein R^{2b} and R^{3b} attached to an adjacent carbon atom together form a 5- or 6-membered N-containing heterocyclic ring substituted at the N-atom with —S(═O)₂Ra^{b}; wherein Ra^{b} is as described herein.

In another embodiment, Compounds of the Disclosure are compounds of any one of formulae (IB), (IIB), and (IIIB), and the pharmaceutically acceptable salts and solvates thereof, wherein Rb^{b} is hydrogen or —C₁₋₄ alkyl.

In another embodiment, Compounds of the Disclosure are compounds of any one of formulae (IB), (IIB), and (IIIB), and the pharmaceutically acceptable salts and solvates thereof, wherein Rb^{b} is hydrogen, —C(═O)Ra^{b}, —S(═O)₂Ra^{b}, —C₁₋₄ alkyl, —C₃₋₆ cycloalkyl, -(5- to 6-membered)-C₂₋₉ heterocyclyl, or —C₆₋₁₀ aryl optionally substituted with 1, 2 or 3 substituents each independently selected from the group consisting of halogen, hydroxy, CN, —O(C₁₋₄ alkyl), —S(C₁₋₄ alkyl), —NH(C₁₋₄ alkyl), —N(C₁₋₄ alkyl)₂, and —C₁₋₄alkyl optionally substituted by 1, 2 or 3 fluorine atoms.

In another embodiment, Compounds of the Disclosure that can be employed in the methods of the present disclosure include compounds of formula (IB), where G is —C(═O)—NH—, having formula (JIB) selected from the group consisting of

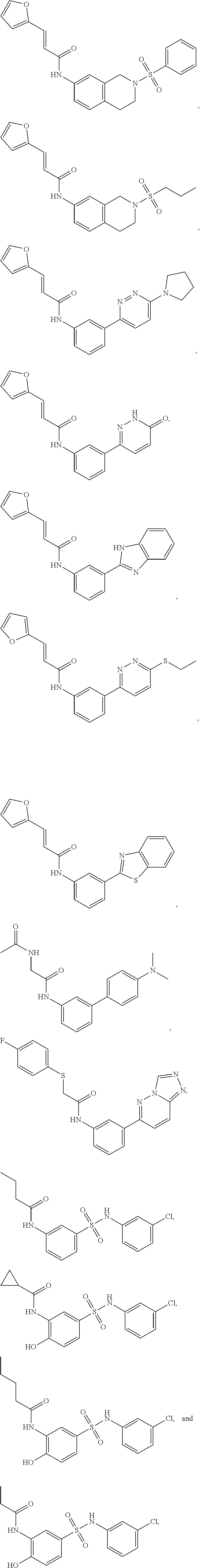

and the pharmaceutically acceptable salts and solvates thereof.

In another aspect, Compounds of the Disclosure include compounds of formula (IB), where G is —C(═O)—NH—, having formula (IIB) selected from the group consisting of

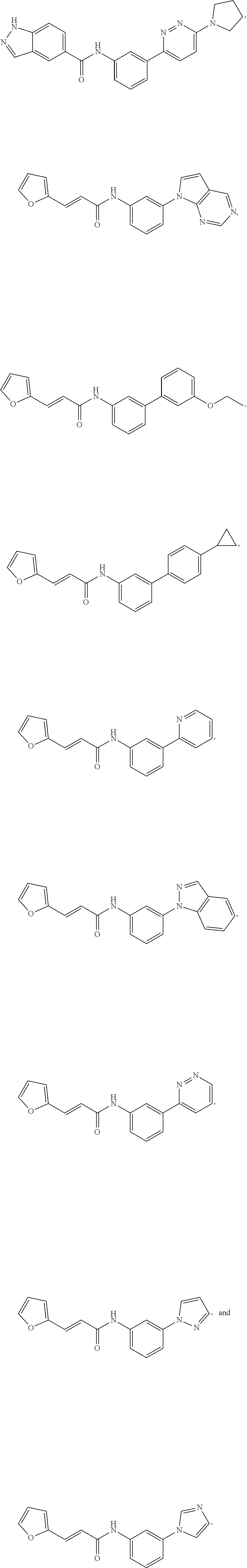

and the pharmaceutically acceptable salts and solvates thereof.

In another aspect, Compounds of the Disclosure include compounds of formula (IB), where G is C(═O)—NH—, having formula IIB selected from the group consisting of

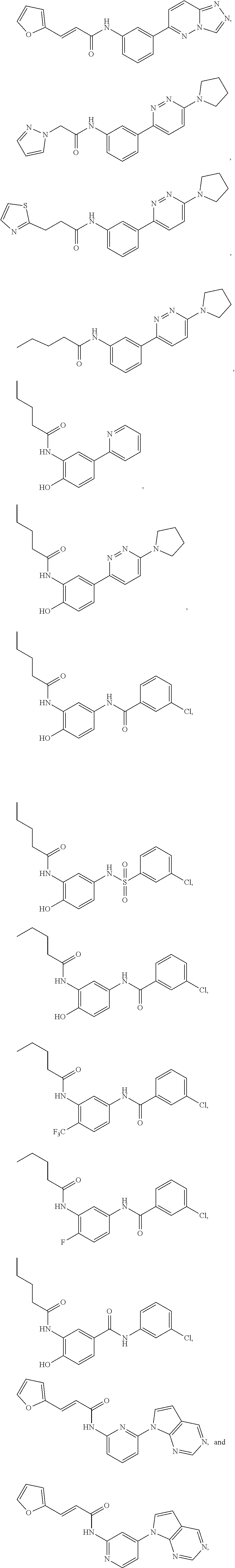

and the pharmaceutically acceptable salts and solvates thereof. In another aspect, Compounds of the Disclosure include compounds of formula (AB), where G is —NH—C(═O)—, having formula (IIIB) selected from the group consisting of

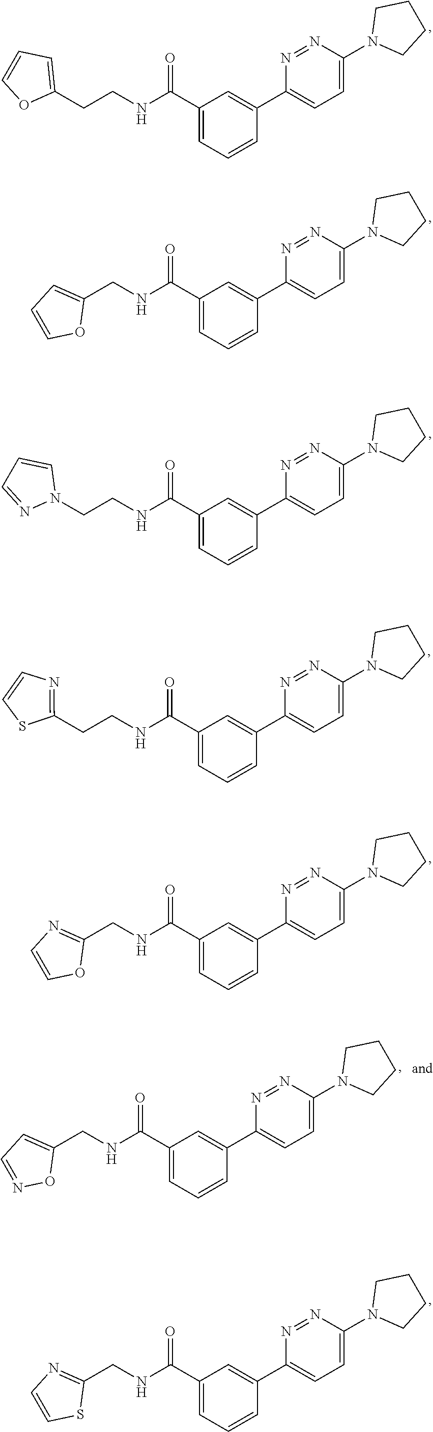

and the pharmaceutically acceptable salts and solvates thereof.

As used herein, the terms “halogen” or “halo” refer to —F, —Cl, —Br, or —I.

As used herein, the term “hydroxyl” or “hydroxy” refers to the group —OH.

As used herein, the term “alkyl” refers to a linear or branched hydrocarbon chain radical consisting of carbon and hydrogen atoms, containing no unsaturation, which is attached to the rest of the molecule by a single bond and, unless otherwise specified, an alkyl radical typically has from 1 to 4 carbon atoms, i.e., C₁₋₄ alkyl. Exemplary C₁₋₄ alkyl groups can be methyl, ethyl, n-propyl, i-propyl, n-butyl, tert-butyl, i-butyl and sec-butyl. In another embodiment, the alkyl is C₁₋₂ alkyl (methyl or ethyl).

As used herein, the term “alkenyl” refers to a linear or branched hydrocarbon chain radical consisting of carbon and hydrogen atoms, containing one or more double bonds, which is attached to the rest of the molecule by a single bond. Useful alkenyl groups are selected from straight-chain and branched-chain C₂₋₄ alkenyl groups. As used herein, the term “C₂₋₄ alkenyl” as used by itself or as part of another group refers to straight chain and branched non-cyclic hydrocarbons having from 2 to 4 carbon atoms and including at least one carbon-carbon double bond. Representative C₂₋₄ alkenyl groups include ethenyl (i.e., vinyl), propenyl, isopropenyl, butenyl, and sec-butenyl.

As used herein, the term “C₁₋₄ alkoxy” refers to oxygen substituted by one of the C₁₋₄ alkyl groups mentioned above (e.g., methoxy, ethoxy, propoxy, iso-propoxy, butoxy, tert-butoxy, iso-butoxy, and sec-butoxy), for example by one of the C₁₋₂ alkyl groups.

Useful “halo(C₁₋₄ alkyl)” groups include any of the above-mentioned C₁₋₄ alkyl groups, preferably any of the above-mentioned C₁₋₂ alkyl groups, substituted by one or more fluorine, chlorine, bromine or iodine atoms (e.g., fluoromethyl, difluoromethyl, difluorochloromethyl, trifluoromethyl, pentafluoroethyl, 1,1-difluoroethyl, 2,2-difluoroethyl, 2,2,2-trifluoroethyl, 3,3,3-trifluoropropyl, 4,4,4-trifluorobutyl, and trichloromethyl groups).

Useful “halo(C₁₋₄ alkoxy)” groups include any of the above-mentioned C₁₋₄ alkoxy groups, preferably any of the above-mentioned C₁₋₂ alkoxy groups, substituted by one or more fluorine, chlorine, bromine or iodine atoms (e.g., fluoromethoxy, difluoromethoxy, difluorochloromethoxy, trifluoromethoxy, pentafluoroethoxy, 1,1-difluoroethoxy, 2,2-difluoroethoxy, 2,2,2-trifluoroethoxy, 3,3,3-trifluoropropoxy, 4,4,4-trifluorobutoxy, and trichloromethoxy groups).

As used herein, the term “cycloalkyl” embraces saturated carbocyclic radicals and, unless otherwise specified, a cycloalkyl radical typically has from 3 to 6 carbon atoms. Examples of cycloalkyl groups include cyclopropyl, cyclobutyl, cyclopentyl, cyclohexyl and cycloheptyl. It is, for example, cyclopropyl, cyclopentyl and cyclohexyl. In another embodiment, the cycloalkyl group is C₃₋₁₀ cycloalkyl.

As used herein, the term “alkylcycloalkyl” when employed in the definition of a substituent refers to a cycloalkyl group as defined above which is linked through an alkylene radical, such as C₁₋₄ alkylene, with the core structure which it substitutes. As an example, a cyclopentylethyl substituent is a substituent consisting of a cyclopentyl group linked through an ethylene group to the core structure which it substitutes.

As used herein, the terms “heterocyclyl” or “heterocyclic group” embrace typically a monocyclic or polycyclic, non-aromatic, saturated or unsaturated C₂₋₁₀ carbocyclic ring, such as a 5- to 10-membered radical, in which one or more, for example 1, 2, 3 or 4 of the carbon atoms, for example, 1 or 2 of the carbon atoms are replaced by a heteroatom selected from N, O and S. In one embodiment, the heterocyclyl is a C₃₋₇ heterocyclyl, i.e., a heterocycle having 3-7 carbon atoms and at least one heteroatom. In another embodiment, a heterocyclyl is a (5- to 10-membered)-C₂₋₉ heterocyclyl, i.e., a heterocycle having 5- to 10-members, of which 2-9 members are carbon. In another embodiment, the heteroatom is N. In another embodiment, the heteroatom is O.

In another embodiment, the heterocyclyl radicals are saturated. A heterocyclic radical can be a single ring or two or more fused rings wherein at least one ring contains a heteroatom. When a heterocyclyl radical carries one or more substituents, the substituents can be the same or different.

A said optionally substituted heterocyclyl is typically unsubstituted or substituted with 1, 2 or 3 substituents which can be the same or different. Examples of heterocyclic radicals include piperidyl, pyrrolidyl, pyrrolinyl, piperazinyl, morpholinyl, thiomorpholinyl, pyrazolinyl, pyrazolidinyl, quinuclidinyl, tetrazolyl, cromanyl, isocromanyl, imidazolidinyl, oxiranyl, azaridinyl, 4,5-dihydro-oxazolyl and 3-aza-tetrahydrofuranyl. The substituents are, for example, selected from halogen atoms, for example, fluorine or chlorine atoms, hydroxy groups, alkoxycarbonyl groups in which the alkyl moiety has from 1 to 4 carbon atoms, hydroxycarbonyl groups, carbamoyl groups, nitro groups, cyano groups, C₁₋₄ alkyl groups optionally substituted by one or more halogen atoms, C₁₋₄ alkoxy groups, optionally substituted by one or more halogen atoms and C₁₋₄ hydroxyalkyl groups.

As used herein, the term “alkylheterocyclyl” when employed in the definition of a substituent refers to a heterocyclyl group as defined above which is linked through an alkylene radical with the core structure which it substitutes. In one embodiment, the alkylheterocyclyl is a —C₁₋₄ alkyl-(5- to 10-membered)-C₂₋₉ heterocyclyl.

As used herein, the term “aryl” designates typically a C₆₋₁₀ monocyclic or polycyclic aryl radical such as phenyl and naphthyl. In another embodiment, the aryl is phenyl. A said optionally substituted aryl radical is typically unsubstituted or substituted with 1, 2 or 3 substituents which can be the same or different. The substituents are, for example, selected from halogen atoms, for example, fluorine or chlorine atoms, hydroxy groups, alkoxycarbonyl groups in which the alkyl moiety has from 1 to 4 carbon atoms, hydroxycarbonyl groups, carbamoyl groups, nitro groups, cyano groups, C₁₋₄ alkyl groups optionally substituted by one or more halogen atoms, C₁₋₄ alkoxy groups, optionally substituted by one or more halogen atoms and C₁₋₄ hydroxyalkyl groups. When an aryl radical carries 2 or more substituents, the substituents can be the same or different. Unless otherwise specified, the substituents on an aryl group are typically themselves unsubstituted.

As used herein, the term “alkylaryl” when employed in the definition of a substituent refers to an aryl group as defined above which is linked through an alkylene radical, such as C₁₋₄ alkylene, with the core structure which it substitutes.

As used herein, the term “heteroaryl” designates typically a 5- to 10-membered ring system, comprising at least one heteroaromatic ring and containing at least one heteroatom selected from O, S and N, typically 1, 2, 3, or 4 heteroatoms.

A heteroaryl group can comprise a single ring or two or more fused rings wherein at least one ring contains a heteroatom. A said optionally substituted heteroaryl group is typically unsubstituted or substituted with 1, 2 or 3 substituents which can be the same or different. The substituents are, for example, selected from halogen atoms, for example, fluorine, chlorine or bromine atoms, alkoxycarbonyl groups in which the alkyl moiety has from 1 to 4 carbon atoms, carbamoyl groups, nitro groups, hydroxy groups, C₁₋₄ alkyl groups, optionally substituted by one or more halogen atoms and C₁₋₄ alkoxy groups, optionally substituted by one or more halogen atoms. When a heteroaryl radical carries 2 or more substituents, the substituents can be the same or different. Unless otherwise specified, the substituents on a heteroaryl radical are typically themselves unsubstituted.

Examples of heteroaryl groups include pyridyl, pyrazinyl, pyrimidinyl, pyridazinyl, furyl, tetrazolyl, benzofuranyl, oxadiazolyl, oxazolyl, isoxazolyl, benzoxazolyl, imidazolyl, benzimidazolyl, thiazolyl, thiadiazolyl, thienyl, pyrrolyl, pyridinyl, benzothiazolyl, indolyl, indazolyl, purinyl, quinolyl, isoquinolyl, phthalazinyl, naphthyridinyl, quinoxalinyl, quinazolinyl, quinolizinyl, cinnolinyl, triazolyl, indolizinyl, indolinyl, isoindolinyl, isoindolyl, imidazolidinyl, pteridinyl, thianthrenyl, pyrazolyl, 2H-pyrazolo[3,4-d]pyrimidinyl, 1H-pyrazolo[3,4-d]pyrimidinyl, thieno[2,3-d]pyrimidinyl, and the various pyrrolopyridyl radicals.

In another embodiment, the heteroaryl is a (5- to 10-membered)-C₂₋₉ heteroaryl. In another embodiment, the heteroaryl is optionally substituted with 1, 2, or 3 groups independently selected from the group consisting of halogen, hydroxy, —CN, —ORb, —SRb, —N(Rb)₂, —C₁₋₄alkyl optionally substituted with 1, 2, or 3 halogen atoms, optionally substituted C₆₋₁₀ aryl, optionally substituted (5- to 10-membered)-C₁₋₉ heteroaryl, and (5- to 10-membered)-C₂₋₉ heterocyclyl; said cycloalkyl, alkylcycloalkyl, aryl, alkylaryl, heteroaryl, alkylheteroaryl, heterocyclyl, and alkylheterocyclyl is optionally fused to a further (second) ring.

The mention of optionally substituted heteroaryl radicals or rests within the present disclosure is intended to cover the N-oxides obtainable from these radicals when they comprise N-atoms.

As used herein, the term “alkylheteroaryl” when employed in the definition of a substituent refers to an heteroaryl group as defined above which is linked through an alkylene radical with the core structure which it substitutes. In another embodiment, the alkylheteroaryl is a —C₁₋₄ alkyl-(5- to 10-membered)-C₁₋₉ heteroaryl.

As used herein, the term “alkenylheteroaryl” when employed in the definition of a substituent refers to an heteroaryl group as defined above which is linked through an alkenylene radical with the core structure which it substitutes. In another embodiment, the alkenylheteroaryl is a —C₂₋₄ alkenyl-(5- to 10-membered)-C₁₋₉ heteroaryl. The term “no more than” prior to a number or series of numbers is understood to include the number adjacent to the term “no more than,” and all preceding numbers or integers that could logically be included, as clear from context. When “no more than” is present before a series of numbers or a range, it is understood that “no more than” can modify each of the numbers in the series or range.

The term “at least” prior to a number or series of numbers is understood to include the number adjacent to the term “at least,” and all subsequent numbers or integers that could logically be included, as clear from context. When at least is present before a series of numbers or a range, it is understood that “at least” can modify each of the numbers in the series or range.

The term “pharmaceutically acceptable” refers to compositions and molecular entities that are physiologically tolerable and do not typically produce an allergic reaction or a similar unfavorable reaction, such as gastric disorders, dizziness and suchlike, when administered to a human or animal. For example, the term “pharmaceutically acceptable” means it is approved by a regulatory agency of a state or federal government or is included in the U.S. Pharmacopoeia or other generally recognized pharmacopoeia for use in animals, and more particularly in humans.

The term “treatment” or “treating” refers to administering a therapy in an amount, manner or mode effective to improve a condition, symptom, or parameter associated with a condition or to prevent progression of a condition, to either a statistically significant degree or to a degree detectable to one skilled in the art. An effective amount, manner, or mode can vary depending on the subject and can be tailored to the patient.

By an “effective” amount or a “therapeutically effective amount” of a drug or pharmacologically active agent is meant a nontoxic but sufficient amount of the drug or agent to provide the desired effect. The amount that is “effective” will vary from subject to subject, depending on the age and general condition of the individual, the particular active agent or agents, and the like. Thus, it is not always possible to specify an exact “effective amount.” However, an appropriate “effective” amount in any individual case may be determined by one of ordinary skill in the art using routine experimentation.

The term “prevention” or “to prevent” refers to the reduction in the risk of acquiring or developing a given disease or disorder, or the reduction or inhibition of the recurrence or a disease or disorder.

The term “about”, as used herein in connection with a measured quantity, refers to the normal variations in that measured quantity, as expected by the skilled artisan making the measurement and exercising a level of care commensurate with the objective of measurement and precision of the measuring equipment. Typically, the term “about” includes the recited number±10%. Thus, “about 10” means 9 to 11.

As used herein, the term “optionally substituted” refers to a group that can be unsubstituted or substituted.

The term “patient” as used herein refers to a human. In some embodiments, the patient is an adult. In some embodiments, the patient is a geriatric patient. In some embodiments, the patient is a child. In some embodiments, the patient is an infant. In some embodiments, the patient is a toddler. In some embodiments, the patient is a preadolescent. In some embodiments, the patient is an adolescent.

As used herein, the term “child” is a human being between the stages of birth and puberty.

The term “puberty” is the process of physical changes through which a child's body matures into an adult body capable of sexual reproduction. On average, girls begin puberty around ages 10-11 and end puberty around 15-17; boys begin around ages 11-12 and end around 16-17.

As used herein, the term “infant” is the synonym for “baby,” the very young offspring of a human. The term “infant” is typically applied to young children under one year of age.

As used herein, the term “toddler” refers to a child of 12 to 36 months old.

As used herein, the term “preadolescent” refers to a person of 10-13 years old.

As used herein, the term “adolescent” refers to a person between ages 10 and 19.

The term “solvate” means any form of the active compound of the disclosure which has another molecule (for example a polar solvent such as water or ethanol, a cyclodextrin or a dendrimer) attached to it through noncovalent bonds. Methods of solvation are known within the art.

The disclosure also provides salts of the Compounds of the Disclosure. Non-limiting examples are sulphates; hydrohalide salts; phosphates; lower alkane sulphonates; arylsulphonates; salts of C₁₋₂₀ aliphatic mono-, di- or tribasic acids which can contain one or more double bonds, an aryl nucleus or other functional groups such as hydroxy, amino, or keto; salts of aromatic acids in which the aromatic nuclei may or may not be substituted with groups such as hydroxyl, lower alkoxyl, amino, mono- or di-lower alkylamino sulphonamido. Also included within the scope of the disclosure are quaternary salts of the tertiary nitrogen atom with lower alkyl halides or sulphates, and oxygenated derivatives of the tertiary nitrogen atom, such as the N-oxides. In preparing dosage formulations, those skilled in the art will select the pharmaceutically acceptable salts.

Solvates and salts can be prepared by methods known in the state of the art. Note that the non-pharmaceutically acceptable solvates also fall within the scope of the disclosure because they can be useful in preparing pharmaceutically acceptable salts and solvates.

The Compounds of the Disclosure also seek to include compounds that differ only in the presence of one or more isotopically enriched atoms. For example, compounds having the present structures except for the replacement of a hydrogen by a deuterium or tritium, or the replacement of a carbon by a carbon enriched in ¹¹C, ¹³C or ¹⁴C or the replacement of a nitrogen by a ¹⁵N enriched nitrogen are within the scope of this disclosure.

Some of the compounds disclosed herein can contain one or more asymmetric centers and can thus give rise to enantiomers, diastereomers, and other stereoisomeric forms, such as epimers. The present disclosure is meant to encompass the uses of all such possible forms, as well as their racemic and resolved forms and mixtures thereof. The individual enantiomers can be separated according to methods known to those of ordinary skill in the art in view of the present disclosure. When the compounds described herein contain olefinic double bonds or other centers of geometric asymmetry, and unless specified otherwise, it is intended that they include both E and Z geometric isomers. All tautomers are intended to be encompassed by the present disclosure as well.

As used herein, the term “stereoisomers” is a general term for all isomers of individual molecules that differ only in the orientation of their atoms in space. It includes enantiomers and isomers of compounds with more than one chiral center that are not mirror images of one another (diastereomers).

The term “chiral center” refers to a carbon atom to which four different groups are attached.

The term “epimer” refers to diastereomers that have opposite configuration at only one of two or more tetrahedral streogenic centers present in the respective molecular entities.

The term “stereogenic center” is an atom, bearing groups such that an interchanging of any two groups leads to a stereoisomer.

The terms “enantiomer” and “enantiomeric” refer to a molecule that cannot be superimposed on its mirror image and hence is optically active wherein the enantiomer rotates the plane of polarized light in one direction and its mirror image compound rotates the plane of polarized light in the opposite direction.

The term “racemic” refers to a mixture of equal parts of enantiomers and which mixture is optically inactive.

The term “resolution” refers to the separation or concentration or depletion of one of the two enantiomeric forms of a molecule.

The terms “a” and “an” refer to one or more.

Some reactions for preparing Compounds of the Disclosure involve employing amino protecting groups. As used herein, an “amine protecting group” or “amino protecting group” refers to a group that blocks (i.e., protects) the amine functionality while reactions are carried out on other functional groups or parts of the molecule. Those skilled in the art will be familiar with the selection, attachment, and cleavage of amine protecting groups and will appreciate that many different protective groups are know in the art, the suitability of one protective group or another being dependent on the particular synthetic scheme planned. Treatises on the subject are available for consultation, such as Wuts, P. G. M. & Greene, T. W., Greene's Protective Groups in Organic Synthesis, 4rd Ed. (J. Wiley & Sons, 2007), herein incorporated by reference in its entirety. Suitable amine protecting groups include methyl carbamate, tert-butyloxycarbonyl (tert-butyl carbamate; BOC), 9-fluorenylmethyl carbamate, benzyl carbamate, 2-(trimethylsilyl)ethyl carbamate, trifluoroacetamide, benzylamine, allylamine, tritylamine, trichloroacetyl, trifluoroacetyl, p-toluenesulfonyl, and allyl carbamate. In another embodiment, the protected amino group can be a phthalimide-protected amino group (NPhth).

As used herein, the term “enzyme replacement therapy” or “ERT” refers to administering an exogenously-produced natural or recombinant enzyme or analog thereof to a patient in need thereof. In the case of a lyosomal storage disease, for example, the patient accumulates harmful levels of a substrate (i.e., material stored) in lysosomes due to a deficiency or defect in an enzyme responsible for metabolizing the substrate, or due to a deficiency in an enzymatic activator required for proper enzymatic function. Enzyme replacement therapy is provided to the patient to reduce the levels of (i.e., debulk) accumulated substrate in affected tissues. Enzyme replacement therapies for treating lysosomal storage diseases are known in the art. In accordance with a combination therapy of the disclosure, a lysosomal enzyme, e.g., galactocerebrosidase, can be used for enzyme replacement therapy to reduce the levels of corresponding substrate, e.g., galactocerebroside, in a patient having a lysosomal storage disease such as Krabbe's disease.

As used herein, the term “substrate reduction therapy” or “SRT” is a therapeutic approach used to treat certain metabolic disorders, e.g., lysosomal storage disorders, in which substrate, e.g., glycolipid, accumulation is counteracted not by replacing the deficient enzyme but by reducing the substrate level to better balance residual activity of the deficient enzyme. See, e.g., Coutinho et al., Int. J. Mol. Sci. 17:1065 (2016). Substrate reduction therapy and enzyme replacement therapy (see above) can have unique, independent, and potentially complementary mechanisms of action in the treatment of lyosomal storage disease and other diseases.

The general principle of SRT is that a substrate reduction agent is administered to a patient to partially inhibit the biosynthesis of the substrate, which accumulates in the absence of a specific lysosomal enzyme. As used herein, the term “substrate reduction agent” is a small molecule that reduces the number of substrate molecules requiring catabolism within the lysosome, thus contributing to balance the rate of synthesis with the impaired rate of catabolism. Substrate reduction agents are known in the art.

As used herein, an “effective amount” of an enzyme, when administered to a subject in a combination therapy of the disclosure, is an amount sufficient to improve the clinical course of a lysosomal storage disease, where clinical improvement is measured by any of the variety of defined parameters well known to the skilled artisan.

As used herein the term “small molecule chaperone” refers to a compound, other than a Compound of the Disclosure, that is capable of binding allosterically or competitively to a mutated enzyme, e.g., 0-galactosidase, thereby stabilizing the enzyme against degradation. In some embodiments, the small molecule chaperone facilitates proper folding and transport of an enzyme to its site of action. Small molecule chaperones for the treatment of lysosomal storage diseases are known in the art. See, e.g., US 2016/0207933 A1 and WO 2011/049737 A1.

α-Synucleinopathies are neurodegenerative diseases characterized by the abnormal accumulation of aggregates of α-synuclein protein in neurons, nerve fibres, or glial cells. There is a well-established clinical association between mutations in the glucocerebrosidase gene and the development of more prevalent multifactorial disorders including Parkinson's disease and other synucleinopathies. See, Siebert, M., et al., Brain 137:1304-1322 (2014). According to Siebert et al., there is a reciprocal relationship between glucocerebrosidase activity (wild-type and mutant) and α-synuclein in synucleinopathies, such as Parkinson's disease and dementia with Lewy bodies. This reciprocal relationship suggests that therapies for Gaucher's disease, which are targeted towards augmenting glucocerebrosidase activity or decreasing glucocerebrosides storage could prove to be provising strategies for modulating α-synuclein proteostasis and its subsequent aggregation and oligomerization.

### Synthesis of Compounds of the Disclosure

Compounds of the Disclosure can be prepared using methods known to those skilled in the art in view of this disclosure, or by illustrative methods shown in the schemes below. Additional methods of synthesis are described and illustrated in the working examples set forth below.

Scheme 1, 2, 3, 11, and 12 illustrate exemplary synthetic paths to obtain compounds of formula (IA) wherein A¹, A², A³ and A⁴ can be nitrogen atoms in different combinations.

Scheme 4 illustrates the synthetic path to obtain compounds of formula (IB) wherein B¹=B²=B³=N. These compounds have formula (IVB).

Schemes 5 and 8-10 illustrate exemplary synthetic paths to obtain compounds of formula (IB) wherein only one of B¹, B² and B³ can be a nitrogen atom. These compounds have formulae (VB), (XVIIB), (XXB), and (XXIIIB), respectively.

Schemes 6 and 7 illustrate exemplary synthetic paths to obtain the reverse amide compounds of formula (IIIB), wherein B¹, B² and B³ are as defined for formula (IB).

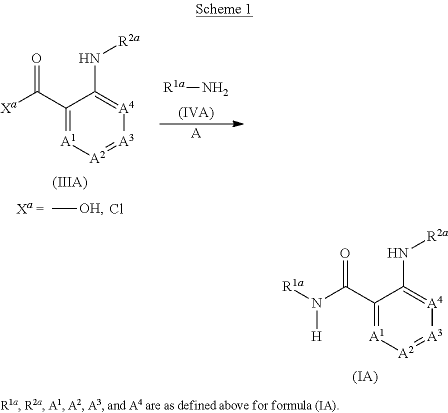

### Reaction A

In a method, according to the disclosure, a compound of formula (IIIA), wherein A¹, A², A³, and A⁴ are as defined above can be reacted with an amine compound of formula (IVA) to yield compounds of formula (IA) according to the disclosure as illustrated in reaction A of the scheme above (Scheme 1) following standard conditions.

The carboxylic acid or acid chloride of the compound of formula (IIIA) is subsequently converted to a substituted amide group by reaction with a compound of formula (IVA) to yield the compound of formula (IA) according to the invention as illustrated in Scheme 1.

Reaction A is carried out under standard amide coupling conditions, for example in the presence of a suitable coupling agent (e.g. 1,1′-carbonyldiimidazole, N,N′-cyclohexylcarbodiimide, 1-(3-dimethylaminopropyl)-3-ethylcarbodiimide (or hydrochloride thereof), N,N′-disuccinimidyl carbonate, benzotriazol-1-yloxytris(dimethylamino)phosphonium hexafluoro-phosphate, 2-(1H-benzotriazol-1-yl)-1,1,3,3-tetramethyluronium hexafluorophosphate (i.e. O-(1H-benzotriazol-1-yl)-N,N,N′,N′-tetramethyluronium hexafluorophosphate), benzotriazol-1-yloxytris-pyrrolidinophosphonium hexafluorophosphate, bromo-tris-pyrrolidinophosphonium hexafluorophosphate, propylphosphonic anhydride, 2-(1H-benzotriazol-1-yl)-1,1,3,3-tetramethyluronium tetra-fluorocarbonate, 1-cyclohexylcarbodiimide-3-propyloxymethyl polystyrene, 0-(7-azabenzotriazol-1-yl)-N,N,N′,N′-tetramethyluronium hexafluorophosphate, O-benzotriazol-1-yl-N,N,N′,N′-tetramethyluronium hexfluoroborate), optionally in the presence of a suitable base (e.g. sodium hydride, sodium bicarbonate, potassium carbonate, pyridine, triethylamine, dimethylaminopyridine, diisopropylamine, diisopropylethylamine, sodium hydroxide, potassium tert-butoxide and/or lithium diisopropylamide (or variants thereof) and an appropriate solvent (e.g. tetrahydrofurane, pyridine, toluene, dichloromethane, chloroform, acetonitrile, dimethylformamide, trifluoromethylbenzene, dioxane or triethylamine). Such reactions may be performed in the presence of a further additive such as 1-hydroxybenzotriazole hydrate.

The reaction mixture is stirred at low temperature or room temperature or heated until the starting materials have been consumed. The reaction may be carried out with protecting groups present and those protecting groups may be removed after the reaction. Suitable protecting groups are known to the person skilled in the art (see T. W. Greene, “Protective Groups in Organic Synthesis”, 3rd Edition, New York, 1999).

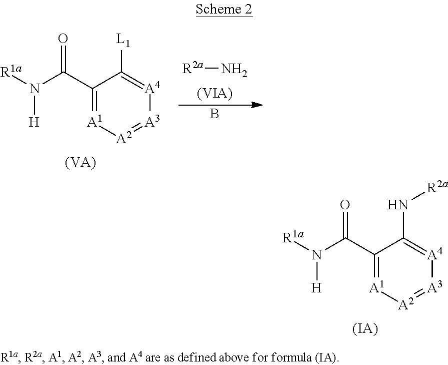

### Reaction B

In another method, according to the disclosure, a compound of formula (VA), wherein A¹, A², A³, and A⁴ are defined above and L¹ is a leaving group, such as halogen, triflate, tosylate or a mesylate group which can be transformed into the —NHR^{2a} group to yield (IA) according to the disclosure as illustrated in reaction B of the scheme above (Scheme 2) following standard conditions.

The leaving group of the compound of formula (VA) is converted by reaction with an amine (VIA) to a corresponding amine group to yield the compound of formula (IA) according to the disclosure as illustrated in reaction B of the schemes above (Scheme 2). Reaction B is carried out under standard nucleophilic substitution conditions, for example in the presence a suitable base (e.g., N,N-diisopropylethylamine, 4-dimethylaminopyridine, 2,6-lutidine, triethylamine, pyridine, ammonium chloride, sodium hydride, potassium carbonate, sodium carbonate, sodium hydrogen carbonate, sodium hydroxide, sodium acetate or sodium nitrite) and an appropriate solvent (e.g., acetonitrile, dichloromethane, tetrahydrofuran, benzene, diethyl ether, toluene, dimethylformamide, water, ethanol or mixture thereof). Such reactions can be used a base or acid in a further step such as, acetic acid, hydrogen chloride or sodium hydroxide.

The reaction mixture is stirred at a low temperature or room temperature or heated until the starting materials have been consumed. The reaction can be carried out with protecting groups present and those protecting groups can be removed after reaction. Suitable protecting groups are known to the person skilled in the art (see T. W. Greene, “Protective Groups in Organic Synthesis”, 3rd Edition, New York, 1999).

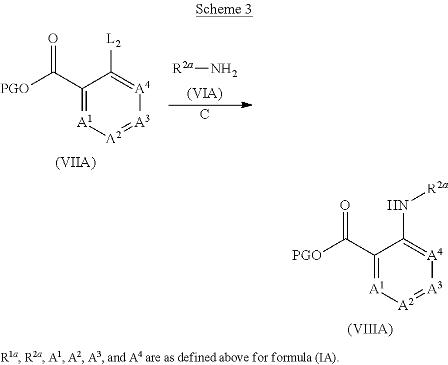

### Reaction C

In another method, according to the disclosure, a compound of formula (VIIA), wherein A¹, A², A³, and A⁴ are defined above, PG is a protecting group, and L² is a leaving group, such as halogen, triflate, tosylate or a mesylate group which can be transformed into the —NHR^{2a} group to yield (VIIIA) according to the disclosure as illustrated in reaction C of the scheme above (Scheme 2) by reaction with an amine compound of formula (VIA) following standard reaction conditions.

The leaving group of the compound of formula (VIIA) is converted by reaction with an amine (VIA) to a corresponding amine group to yield the compound of formula (VIIIA) according to the disclosure as illustrated in reaction C of the schemes above (Scheme 3). Reaction C is carried out under standard nucleophilic substitution conditions, for example in the presence a suitable base (e.g., N,N-diisopropylethylamine, 4-dimethylaminopyridine, 2,6-lutidine, triethylamine, pyridine, ammonium chloride, sodium hydride, potassium carbonate, sodium carbonate, sodium hydrogen carbonate, sodium hydroxide, sodium acetate or sodium nitrite) and an appropriate solvent (e.g., acetonitrile, dichloromethane, tetrahydrofuran, benzene, diethyl ether, toluene, dimethylformamide, water, ethanol or mixture thereof). Such reactions can be used a base or acid in a further step such as, acetic acid, hydrogen chloride or sodium hydroxide.

The reaction mixture is stirred at a low temperature or room temperature or heated until the starting materials have been consumed. The reaction can be carried out with protecting groups present and those protecting groups can be removed after reaction. Suitable protecting groups are known to the person skilled in the art (see T. W. Greene, “Protective Groups in Organic Synthesis”, 3rd Edition, New York, 1999).

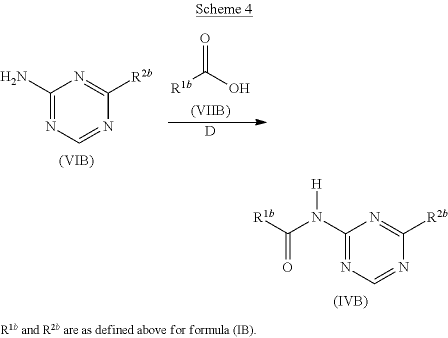

### Reaction D

The carboxylic acid or acid chloride of the compound of formula (VIIB) is converted to a substituted amide group to yield the compound of formula (IVB) according to the invention as illustrated in Scheme 4.

Reaction D is carried out under standard amide coupling conditions, for example in the presence of a suitable coupling agent (e.g. 1,1′-carbonyldiimidazole, N,N′-cyclohexylcarbodiimide, 1-(3-dimethylaminopropyl)-3-ethylcarbodiimide (or hydrochloride thereof), N,N′-disuccinimidyl carbonate, benzotriazol-1-yloxytris(dimethylamino)phosphonium hexafluoro-phosphate, 2-(1H-benzotriazol-1-yl)-1,1,3,3-tetramethyluronium hexafluorophosphate (i.e. O-(1H-benzotriazol-1-yl)-N,N,N′,N′-tetramethyluronium hexafluorophosphate), benzotriazol-1-yloxytris-pyrrolidinophosphonium hexafluorophosphate, bromo-tris-pyrrolidinophosphonium hexafluorophosphate, propylphosphonic anhydride, 2-(1H-benzotriazol-1-yl)-1,1,3,3-tetramethyluronium tetra-fluorocarbonate, 1-cyclohexylcarbodiimide-3-propyloxymethyl polystyrene, 0-(7-azabenzotriazol-1-yl)-N,N,N′,N′-tetramethyluronium hexafluorophosphate, O-benzotriazol-1-yl-N,N,N′,N′-tetramethyluronium hexfluoroborate), optionally in the presence of a suitable base (e.g. sodium hydride, sodium bicarbonate, sodium carbonate, potassium carbonate, pyridine, triethylamine, dimethylaminopyridine, diisopropylamine, diisopropylethylamine, sodium hydroxide, potassium tert-butoxide and/or lithium diisopropylamide (or variants thereof) and an appropriate solvent (e.g. N-methyl-2-pyrrolidone, tetrahydrofurane, pyridine, toluene, ethanol, dichloromethane, chloroform, acetonitrile, dimethylformamide, trifluoromethylbenzene, dioxane or trimethylamine, or mixtures thereof). Such reactions may be performed in the presence of a further additive such as 1-hydroxybenzotriazole hydrate.

The reaction mixture is stirred at low temperature or room temperature or heated until the starting materials have been consumed. The reaction may be carried out with protecting groups present and those protecting groups may be removed after the reaction. Suitable protecting groups are known to the person skilled in the art (see T. W. Greene, “Protective Groups in Organic Synthesis”, 3rd Edition, New York, 1999).

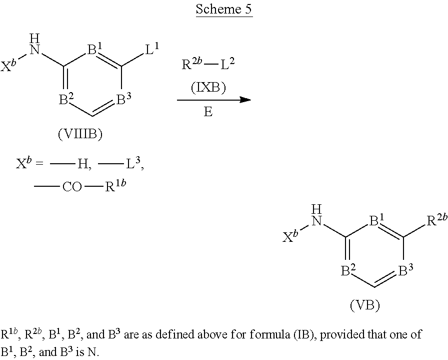

### Reaction E

In another method, according to the disclosure, a compound of formula (VIIIB), wherein B¹, B², and B³ are defined above and L¹ represents a suitable leaving group, such as iodo, bromo, chloro or a sulphonate group (e.g. —OS(O)₂CF₃, —OS(O)₂CH₃ or —OS(O)₂PhMe), reacts with a compound of formula (IXB) to yield (VB) according to the disclosure as illustrated in reaction E of the scheme above (Scheme 5).

Reaction E is carried out in standard coupling conditions by reaction of compound (VIIIB) with a compound (IXB) of formula:

L²-R^{2b}

wherein R^{2b} is as defined above and L² represents a suitable group such as halogen, alkali metal group (e. g. lithium), a Grignand reagent (e.g. MgX), —B(OH)₂, —B(OR)₂ or —Sn(R)₃, in which each R independently represents an alkyl group, or, in the case of —B(OR)₂, the respective R groups may be linked together to form a 4- to 6-membered cyclic group. The reaction may be performed, for example in the presence of a suitable catalyst system, e.g. a metal (or a salt or complex thereof) such as Pd, Cu, Pd/C, PdCl₂, Pd(OAc)₂, Pd(Ph₃P)₄, Pd(Ph₃P)₂Cl₂ (i.e. palladium tetrakistriphenylphosphine), Pd₂(dba)₃ or NiCl₂ and a ligand such as t-Bu3P, (C₆H₁₁)₃P, Ph₃P, AsPh₃, P(o-Tol)₃, 1,2-bis(diphenylphosphino)ethane, 2,2′-bis(di-tert-butylphosphino)-1,1′-biphenyl, xantphos, or a mixture thereof, together with a suitable base such as, sodium carbonate, potassium phosphate, cesium carbonate, sodium hydroxide, potassium hydroxide, potassium carbonate, cesium fluoride, triethylamine, diisopropylethylamine, sodium tert-butoxide, or potassium tert-butoxide (or mixtures thereof) in a suitable solvent such as dioxane, toluene, ethanol, dimethylformamide, ethylene glycol dimethyl ether, water, dimethylsulfoxide, acetonitrile, dimethylacetamide, N-methylpyrrolidinone, tetrahydrofuran or mixtures thereof. The reaction may also be carried out for example at room temperature or above. Alternative reactions conditions include microwave irradiation conditions.

The reaction can be carried out with protecting groups present and those protecting groups can be removed after the reaction. Suitable protecting groups are known to the person skilled in the art (see T. W. Greene, “Protective Groups in Organic Synthesis,” 3rd Edition, New York, 1999).

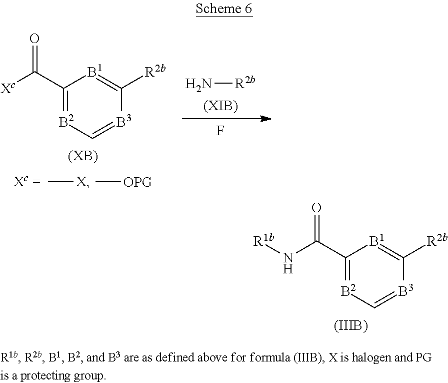

### Reaction F

In another method, according to the disclosure, a compound of formula (XB), wherein B¹, B², and B³ are as defined above and X^{c} represents halogen or a group —OPG, wherein PG os a protecting group, reacts with a compound of formula (XIB) to yield the compound of formula (IIIB) according to the disclosure as illustrated in reaction F of the scheme above (Scheme 6).

The carboxylic acid or acid chloride of the compound (XB) is subsequently converted to a substituted amide group to yield the compound of formula (IIIB) according to the invention as illustrated in Scheme 6. Reaction F is carried out under standard amide coupling conditions, for example in the presence of a suitable coupling agent (e.g. 1,1′-carbonyldiimidazole, N,N′-cyclohexylcarbodiimide, 1-(3-dimethylaminopropyl)-3-ethylcarbodiimide (or hydrochloride thereof), N,N′-disuccinimidyl carbonate, benzotriazol-1-yloxytris(dimethylamino)phosphonium hexafluorophosphate, 2-(1H-benzotriazol-1-yl)-1,1,3,3-tetramethyluronium hexafluorophosphate (i.e. O-(1H-benzotriazol-1-yl)-N,N,N′,N′-tetramethyluronium hexafluorophosphate), benzotriazol-1-yloxytris-pyrrolidinophosphonium hexafluorophosphate, bromo-tris-pyrrolidinophosphonium hexafluorophosphate, propylphosphonic anhydride, 2-(1H-benzotriazol-1-yl)-1,1,3,3-tetramethyluronium tetra-fluorocarbonate, 1-cyclohexylcarbodiimide-3-propyloxymethyl polystyrene, 0-(7-azabenzotriazol-1-yl)-N,N,N′,N′-tetramethyluronium hexafluorophosphate, O-benzotriazol-1-yl-N,N,N′,N′-tetramethyluronium hexfluoroborate), optionally in the presence of a suitable base (e.g. sodium hydride, sodium bicarbonate, potassium carbonate, pyridine, triethylamine, dimethylaminopyridine, diisopropylamine, diisopropylethylamine, sodium hydroxide, potassium tert-butoxide and/or lithium diisopropylamide or variants thereof) and an appropriate solvent (e.g. tetrahydrofurane, pyridine, toluene, dichloromethane, chloroform, acetonitrile, dimethylformamide, trifluoromethylbenzene, dioxane or triethylamine). Such reactions may be performed in the presence of a further additive such as 1-hydroxybenzotriazole hydrate.

The reaction mixture is stirred at low temperature or room temperature or heated until the starting materials have been consumed. The reaction may be carried out with protecting groups present and those protecting groups may be removed after the reaction. Suitable protecting groups are known to the person skilled in the art (see T. W. Greene, “Protective Groups in Organic Synthesis”, 3rd Edition, New York, 1999).

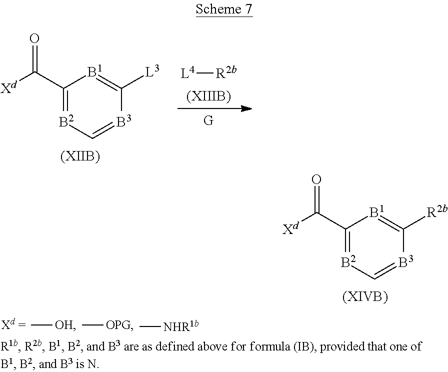

### Reaction G

In another method, according to the disclosure, a compound of formula (XIIB), wherein L³ represents a suitable leaving group, X^{d} can be —OH, —NH—R^{1b}, or —OPG, where PG is a protecting group and each of R^{1b}, B¹, B², and B³ are as defined above, is reacted with a compound of formula (XIIIB), wherein L⁴ is a group suitable for a coupling reaction and R^{2b} is as defined above, to yield compounds of formula (XIVB), as illustrated in reaction G of the scheme above (Scheme 7).

Reaction G is carried out in standard coupling conditions by reaction of compound (XIIB) with a compound (XIIIB) of formula:

L⁴-R^{2b}

wherein R^{2b} is as defined above and L⁴ represents a suitable group such as halogen, alkali metal group (e. g. lithium), a Grignand reagent (e.g. MgX), —B(OH)₂, B(OR)₂ or —Sn(R)₃, or a precursor of any of them in which each R independently represents an alkyl group, or, in the case of —B(OR)₂, the respective R groups may be linked together to form a 4- to 6-membered cyclic group. The reaction may be performed, for example in the presence of a suitable catalyst system, e.g. a metal (or a salt or complex thereof) such as Pd, Cu, Pd/C, PdCl₂, Pd(OAc)₂, Pd(Ph₃P)₄, Pd(Ph₃P)₂Cl₂ (i.e. palladium tetrakistriphenylphosphine), Pd₂(dba)₃ or NiCl₂ and a ligand such as t-Bu3P, (C₆H₁₁)₃P, Ph₃P, AsPh₃, P(o-Tol)₃, 1,2-bis(diphenylphosphino)ethane, 2,2′-bis(di-tert-butylphosphino)-1,1′-biphenyl, xantphos, or a mixture thereof, together with a suitable base such as, sodium carbonate, potassium phosphate, cesium carbonate, sodium hydroxide, potassium hydroxide, potassium carbonate, cesium fluoride, triethylamine, diisopropylethylamine, sodium tert-butoxide, or potassium tert-butoxide (or mixtures thereof) in a suitable solvent such as dioxane, toluene, ethanol, dimethylformamide, ethylene glycol deimethyl ether, water, dimethylsulfoxide, acetonitrile, dimethylacetamide, N-methylpyrrolidinone, tetrahydrofuran or mixtures thereof. The reaction may also be carried out for example at room temperature or above. Alternative reactions conditions include microwave irradiation conditions.

The reaction can be carried out with protecting groups present and those protecting groups can be removed after the reaction. Suitable protecting groups are known to the person skilled in the art (see T. W. Greene, “Protective Groups in Organic Synthesis,” 3rd Edition, New York, 1999).

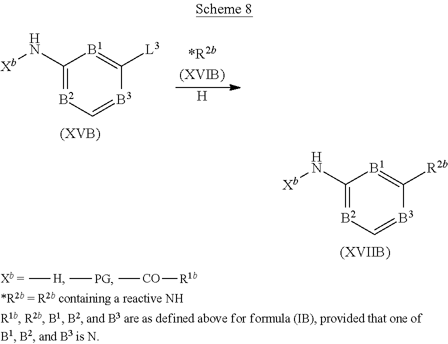

### Reaction H

In another method, according to the disclosure, a compound of formula (XVB), wherein X^{b} can be —H, —CO—R^{1b}, -PG, where PG is a protecting group and L³ is a leaving group. and each of R^{1b}, B¹, B², and B³ are as defined above, is reacted with a compound of formula (XVIB), wherein *R^{2b} is a R^{2b} precursor containing a NH suitable for the reaction, to yield compounds of formula (XVIIB), as illustrated in reaction H of the scheme above (Scheme 8).

Reaction H is used to prepare compounds of formula (XVIIB) by reaction of a compound of formula (XVB) with a compound of formula (XVIB) wherein L³ represents a leaving group such as iodo, bromo, chloro or a sulphonate group (e.g. —OS(O)₂CF₃, —OS(O)₂CH₃ or —OS(O)₂PhMe). Said reaction may be performed under standard conditions in the presence of a suitable base such as pyridine, triethylamine, dimethylaminopyridine, diisopropylamine, sodium hydroxide, or mixtures thereof), and an appropriate solvent such as pyridine, dichloromethane, chloroform, tetrahydrofuran, dimethylformamide, dimethylsulphoxide, water or mixtures thereof and, for example, at around room temperature or above, or under microwave irradiation reaction conditions.

The reaction may also be carried out in the presence of an appropriate metal catalyst (or a salt or complex thereof) such as Cu, Cu(OAc)₂, CuI (or CuI/diamine complex) copper tris(triphenyl-phosphine)bromide, Pd(OAc)₂, tris(dibenzylideneacetone) dipalladium(O) (Pd₂(dba)₃) or NiCl₂ and also optionally in the presence of an additive such as Ph₃P, 2,2′-bis(diphenylphosphino)-1,1′-binaphthyl, xantphos, (1R,2R)—N1,N2-dimethylcyclohexane-1,2-diamine, NaI or an appropriate crown ether such as 18-crown-6-benzene, in the presence of an appropriate base such as sodium hydride, triethylamine, pyridine, N,N′-dimethylethylenediamine, imidazole, sodium carbonate, potassium carbonate, tripotassium phosphate, potassium phosphate, cesium carbonate, sodium tert-butoxide or potassium tert-butoxide (or a mixture thereof, optionally in the presence of 4 A molecular sieves), in a suitable solvent (e.g. dichloromethane, dioxane, toluene, ethanol, isopropanol, dimethylformamide, ethylene glycol, ethylene glycol dimethyl ether, water, dimethylsulfoxide, acetonitrile, dimethylacetamide, N-methylpyrrolidinone, tetrahydrofuran) or a mixture thereof. This reaction may be carried out under microwave irradiation reaction conditions.

The reaction mixture may be stirred at room temperature or heated until the starting materials have been consumed. The reaction may be carried out with protecting groups present and those protecting groups may be removed after the reaction. Suitable protecting groups are known to the person skilled in the art (see T. W. Greene, “Protective Groups in Organic Synthesis”, 3rd Edition, New York, 1999).

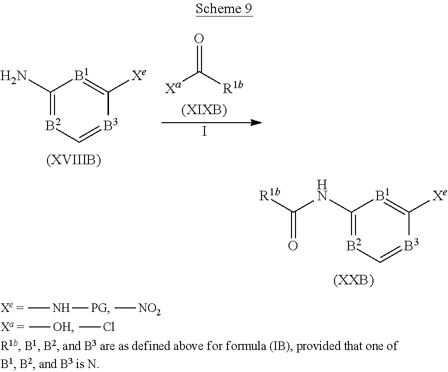

### Reaction I

In another method, according to the disclosure, a compound of formula (XVIIIB), wherein X^{e} can be —NO₂, or —NH-PG, where PG is a protecting group, and each of B¹, B², and B³ are as defined above, is reacted with a compound of formula (XIXB), wherein X^{a} can be —OH, or —Cl and R^{1b} is as defined above, to yield compounds of formula (XXB), as illustrated in reaction I of the scheme above (Scheme 9)

When X^{e} is a —NO₂, the nitro group can be reduced under standard reduction conditions to the corresponding primary amine and used in further reactions to obtain compounds of formula (IB).

When X^{e} is a —NH-PG, the protected primary amine can be deprotected using standard procedures and be used in further reactions to obtain compounds of formula (IB).

The amine of the compound of formula (XVIIIB) is converted to a substituted amide group by reaction with a compound of formula (XIXB) to yield the compound of formula (XXB) according to the invention as illustrated in Scheme 9.

Reaction I is carried out under standard condensation conditions, for example in the presence of a suitable coupling agent (e.g. 1,1′-carbonyldiimidazole, N,N′-cyclohexylcarbodiimide, 1-(3-dimethylaminopropyl)-3-ethylcarbodiimide (or hydrochloride thereof), N,N′-disuccinimidyl carbonate, benzotriazol-1-yloxytris(dimethylamino)phosphonium hexafluoro-phosphate, 2-(1H-benzotriazol-1-yl)-1,1,3,3-tetramethyluronium hexafluorophosphate (i.e. O-(1H-benzotriazol-1-yl)-N,N,N′,N′-tetramethyluronium hexafluorophosphate), benzotriazol-1-yloxytris-pyrrolidinophosphonium hexafluorophosphate, bromo-tris-pyrrolidinophosphonium hexafluorophosphate, propylphosphonic anhydride, 2-(1H-benzotriazol-1-yl)-1,1,3,3-tetramethyluronium tetra-fluorocarbonate, 1-cyclohexylcarbodiimide-3-propyloxymethyl polystyrene, O-(7-azabenzotriazol-1-yl)-N,N,N′,N′-tetramethyluronium hexafluorophosphate, O-benzotriazol-1-yl-N,N,N′,N′-tetramethyluronium hexfluoroborate), optionally in the presence of a suitable base (e.g. sodium hydride, sodium bicarbonate, potassium carbonate, pyridine, triethylamine, dimethylaminopyridine, diisopropylamine, diisopropylethylamine, sodium hydroxide, potassium tert-butoxide and/or lithium diisopropylamide (or variants thereof) and an appropriate solvent (e.g. tetrahydrofurane, pyridine, toluene, dichloromethane, chloroform, acetonitrile, dimethylformamide, trifluoromethylbenzene, dioxane or triethylamine). Such reactions may be performed in the presence of a further additive such as 1-hydroxybenzotriazole hydrate. The reaction mixture is stirred at low temperature or room temperature or heated until the starting materials have been consumed.

Alternatively, the reaction can be performed by applying microwave radiation in a suitable microwave oven, for example at a temperature of 100° C. for 4 h or at 85° C. for 3 h.

The reaction may be carried out with protecting groups present and those protecting groups may be removed after the reaction. Suitable protecting groups are known to the person skilled in the art (see T. W. Greene, “Protective Groups in Organic Synthesis”, 3rd Edition, New York, 1999).

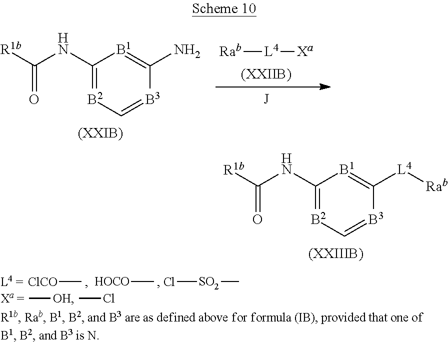

### Reaction J

In another method, according to the disclosure, a compound of formula (XXIB), wherein X^{a} can be —OH or —Cl and each of B¹, B², and B³ are as defined above, is reacted with a compound of formula (XXIIB), wherein L⁴ can be ClCO—, HOCO—, Cl—SO₂— and Ra^{b} is as defined above, to yield compounds of formula (XXIIIB), as illustrated in reaction J of the scheme above (Scheme 10)

The amine of the compound of formula (XXIB) is converted for example to a substituted amide or sulphonamide group by reaction with a compound of formula (XXIIB) to yield the compound of formula (XXIIIB) according to the invention as illustrated in Scheme 10.

Reaction J is carried out under standard condensation conditions, for example in the presence of a suitable coupling agent (e.g. 1,1′-carbonyldiimidazole, N,N′-cyclohexylcarbodiimide, 1-(3-dimethylaminopropyl)-3-ethylcarbodiimide (or hydrochloride thereof), N,N′-disuccinimidyl carbonate, benzotriazol-1-yloxytris(dimethylamino)phosphonium hexafluoro-phosphate, 2-(1H-benzotriazol-1-yl)-1,1,3,3-tetramethyluronium hexafluorophosphate (i.e. O-(1H-benzotriazol-1-yl)-N,N,N′,N′-tetramethyluronium hexafluorophosphate), benzotriazol-1-yloxytris-pyrrolidinophosphonium hexafluorophosphate, bromo-tris-pyrrolidinophosphonium hexafluorophosphate, propylphosphonic anhydride, 2-(1H-benzotriazol-1-yl)-1,1,3,3-tetramethyluronium tetra-fluorocarbonate, 1-cyclohexylcarbodiimide-3-propyloxymethyl polystyrene, O-(7-azabenzotriazol-1-yl)-N,N,N′,N′-tetramethyluronium hexafluorophosphate, O-benzotriazol-1-yl-N,N,N′,N′-tetramethyluronium hexfluoroborate), optionally in the presence of a suitable base (e.g. sodium hydride, sodium bicarbonate, potassium carbonate, pyridine, triethylamine, dimethylaminopyridine, diisopropylamine, diisopropylethylamine, sodium hydroxide, potassium tert-butoxide and/or lithium diisopropylamide (or variants thereof) and an appropriate solvent (e.g. tetrahydrofurane, pyridine, toluene, dichloromethane, chloroform, acetonitrile, dimethylformamide, trifluoromethylbenzene, dioxane or triethylamine). Such reactions may be performed in the presence of a further additive such as 1-hydroxybenzotriazole hydrate. The reaction mixture is stirred at low temperature or room temperature or heated until the starting materials have been consumed.

Alternatively, the reaction can be performed by applying microwave radiation in a suitable microwave oven, for example at a temperature of 100° C. for 4 h or at 85° C. for 3 h.

The reaction may be carried out with protecting groups present and those protecting groups may be removed after the reaction. Suitable protecting groups are known to the person skilled in the art (see T. W. Greene, “Protective Groups in Organic Synthesis”, 3rd Edition, New York, 1999).

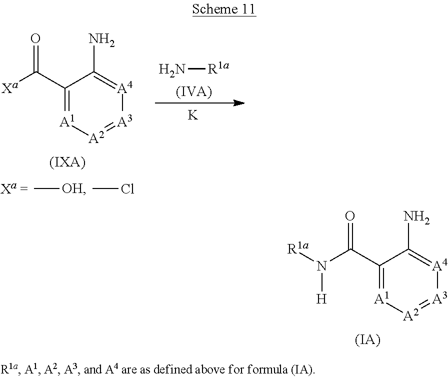

### Reaction K

In another method, according to the disclosure, a compound of formula (IXA), wherein X^{a} can be —OH or —Cl, and A¹, A², A³, and A⁴ are as defined above can be reacted with an amine compound of formula (IVA), wherein R^{1a} is as defined above, to yield compounds of formula (IA) according to the disclosure as illustrated in reaction K of the scheme above (Scheme 11) following standard conditions.

The carboxylic acid or acid chloride of the compound of formula (IXA) is converted to a substituted amide group by reaction with a compound of formula (IVA) to yield the compound of formula (IA) according to the invention as illustrated in Scheme 11.

Reaction K is carried out under standard amide coupling conditions, for example in the presence of a suitable coupling agent (e.g. 1,1′-carbonyldiimidazole, N,N′-cyclohexylcarbodiimide, 1-(3-dimethylaminopropyl)-3-ethylcarbodiimide (or hydrochloride thereof), N,N′-disuccinimidyl carbonate, benzotriazol-1-yloxytris(dimethylamino)phosphonium hexafluoro-phosphate, 2-(1H-benzotriazol-1-yl)-1,1,3,3-tetramethyluronium hexafluorophosphate (i.e. O-(1H-benzotriazol-1-yl)-N,N,N′,N′-tetramethyluronium hexafluorophosphate), benzotriazol-1-yloxytris-pyrrolidinophosphonium hexafluorophosphate, bromo-tris-pyrrolidinophosphonium hexafluorophosphate, propylphosphonic anhydride, 2-(1H-benzotriazol-1-yl)-1,1,3,3-tetramethyluronium tetra-fluorocarbonate, 1-cyclohexylcarbodiimide-3-propyloxymethyl polystyrene, 0-(7-azabenzotriazol-1-yl)-N,N,N′,N′-tetramethyluronium hexafluorophosphate, O-benzotriazol-1-yl-N,N,N′,N′-tetramethyluronium hexfluoroborate), optionally in the presence of a suitable base (e.g. sodium hydride, sodium bicarbonate, potassium carbonate, pyridine, triethylamine, dimethylaminopyridine, diisopropylamine, diisopropylethylamine, sodium hydroxide, potassium tert-butoxide and/or lithium diisopropylamide (or variants thereof) and an appropriate solvent (e.g. tetrahydrofurane, pyridine, toluene, dichloromethane, chloroform, acetonitrile, dimethylformamide, trifluoromethylbenzene, dioxane or triethylamine). Such reactions may be performed in the presence of a further additive such as 1-hydroxybenzotriazole hydrate.

The reaction mixture is stirred at low temperature or room temperature or heated until the starting materials have been consumed. The reaction may be carried out with protecting groups present and those protecting groups may be removed after the reaction. Suitable protecting groups are known to the person skilled in the art (see T. W. Greene, “Protective Groups in Organic Synthesis”, 3rd Edition, New York, 1999).

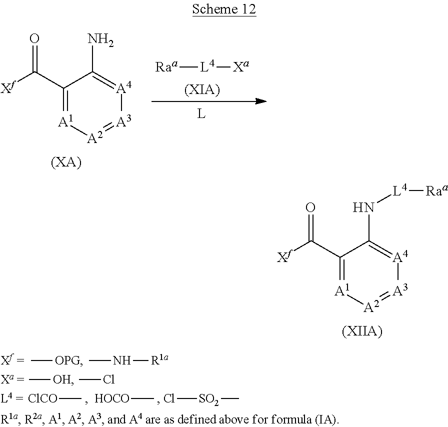

### Reaction L

In another method, according to the disclosure, a compound of formula (XA), wherein X^{f} can be —NH—R^{1a}, —OPG, where PG is a protecting group and each of A¹, A², A³, and A⁴ are as defined above, is reacted with a compound of formula (XIA), wherein L⁴ can be ClCO—, HOCO—, Cl—SO₂— and Ra^{a} is as defined above, to yield compounds of formula (XIIA), as illustrated in reaction L of the scheme above (Scheme 12).

The amine of the compound of formula (XA) is converted to a substituted amide group by reaction with a compound of formula (XIA) to yield the compound of formula (XIIA) according to the invention as illustrated in Scheme 12.

Reaction L is carried out under standard condensation conditions, for example in the presence of a suitable coupling agent (e.g. 1,1′-carbonyldiimidazole, N,N′-cyclohexylcarbodiimide, 1-(3-dimethylaminopropyl)-3-ethylcarbodiimide (or hydrochloride thereof), N,N′-disuccinimidyl carbonate, benzotriazol-1-yloxytris(dimethylamino)phosphonium hexafluoro-phosphate, 2-(1H-benzotriazol-1-yl)-1,1,3,3-tetramethyluronium hexafluorophosphate (i.e. O-(1H-benzotriazol-1-yl)-N,N,N′,N′-tetramethyluronium hexafluorophosphate), benzotriazol-1-yloxytris-pyrrolidinophosphonium hexafluorophosphate, bromo-tris-pyrrolidinophosphonium hexafluorophosphate, propylphosphonic anhydride, 2-(1H-benzotriazol-1-yl)-1,1,3,3-tetramethyluronium tetra-fluorocarbonate, 1-cyclohexylcarbodiimide-3-propyloxymethyl polystyrene, 0-(7-azabenzotriazol-1-yl)-N,N,N′,N′-tetramethyluronium hexafluorophosphate, O-benzotriazol-1-yl-N,N,N′,N′-tetramethyluronium hexfluoroborate), optionally in the presence of a suitable base (e.g. sodium hydride, sodium bicarbonate, potassium carbonate, pyridine, triethylamine, dimethylaminopyridine, diisopropylamine, diisopropylethylamine, sodium hydroxide, potassium tert-butoxide and/or lithium diisopropylamide (or variants thereof) and an appropriate solvent (e.g. tetrahydrofurane, pyridine, toluene, dichloromethane, chloroform, acetonitrile, dimethylformamide, trifluoromethylbenzene, dioxane or triethylamine). Such reactions may be performed in the presence of a further additive such as 1-hydroxybenzotriazole hydrate. The reaction mixture is stirred at low temperature or room temperature or heated until the starting materials have been consumed.

Alternatively, the reaction can be performed by applying microwave radiation in a suitable microwave oven, for example at a temperature of 100° C. for 4 h or at 85° C. for 3 h.

The reaction may be carried out with protecting groups present and those protecting groups may be removed after the reaction. Suitable protecting groups are known to the person skilled in the art (see T. W. Greene, “Protective Groups in Organic Synthesis”, 3rd Edition, New York, 1999).

### Use of the Compounds of the Disclosure

The utility of Compounds of the Disclosure, including pharmaceutically acceptable salts or solvates, in the present methods can be demonstrated in appropriate in vitro or in vivo assays. Compounds of the Disclosure have the ability to increase galactocerebrosidase. Therefore, Compounds of the Disclosure can be used/administered to treat and/or prevent conditions associated with alteration of the activity of galactocerebrosidase in a patient, such as for example lysosomal storage diseases and α-synucleinopathies. In one embodiment, the lysosomal storage disease is Krabbe's disease. In another embodiment, the α-synucleinopathy is Parkinson's disease. In another embodiment, a condition associated with alteration of the activity of galactocerebrosidase is a disease or disorder selected from the group consisting of Krabbe's disease, demyelinating disorders, galactosylsphingosine related disorders, globoid cell leukodystrophy, multiple sclerosis (MS), Parkinson's disease, peripheral neuropathy, progressive multiple sclerosis, pulmonary artery enlargement in COPD, open angle glaucoma, Lewy body dementia, and multiple system atrophy (MSA). See, e.g., Graziano A. C. E. et al., Journal of Neuroscience Research 94:1220-1230 (2016); Hill C. H. et al., Chem. Sci. 6:3075-3086 (2015); and Hossain M. A. et al., Journal of Human Genetics 60:539-545 (2015).

In another aspect, the present disclosure is directed to a method of treating or preventing a condition associated with the alteration of the activity of galactocerebrosidase in a patient in need thereof, comprising administering to the patient in need thereof an effective amount of a Compound of the Disclosure. In some embodiments, the Compound of the Disclosure is a compound of formula (IA) or formula (IIA), or a pharmaceutically acceptable salt or solvate thereof, as described herein. In some embodiments, the Compound of the Disclosure is a compound of any one of formulae (IB), (IIB), or (IIIB), or a pharmaceutically acceptable salt or solvate thereof, as described herein.

In another aspect, the present disclosure is directed to a method of treating or preventing a lysosomal storage disease, such as Krabbe's disease, in a patient in need thereof, comprising administering an effective amount of a Compound of the Disclosure. In some embodiments, the Compound of the Disclosure is a compound of formula (IA) or formula (IIA), or a pharmaceutically acceptable salt or solvate thereof, as described herein. In some embodiments, the Compound of the Disclosure is a compound of any one of formulae (IB), (IIB), or (IIIB), or a pharmaceutically acceptable salt or solvate thereof, as described herein.

In another aspect, the present disclosure is directed to a method of treating or preventing an α-synucleinopathy, such as Parkinson's disease, in a patient in need thereof, comprising administering an effective amount of a Compound of the Disclosure. In some embodiments, the Compound of the Disclosure is a compound of formula (IA) or formula (IIA), or a pharmaceutically acceptable salt or solvate thereof, as described herein. In some embodiments, the Compound of the Disclosure is a compound of any one of formulae (IB), (IIB), or (IIIB), or a pharmaceutically acceptable salt or solvate thereof, as described herein.

In another aspect, the present disclosure is directed to method of treating or preventing a disease or disorder in a patient selected from the group consisting of Krabbe's disease, demyelinating disorders, galactosylsphingosine related disorders, globoid cell leukodystrophy, multiple sclerosis (MS), Parkinson's disease, peripheral neuropathy, progressive multiple sclerosis, pulmonary artery enlargement in COPD, open angle glaucoma, Lewy body dementia, and multiple system atrophy (MSA), comprising administering an effective amount of a Compound of the Disclosure to a patient in need thereof. In some embodiments, the Compound of the Disclosure is a compound of formula (IA) or formula (IIA), or a pharmaceutically acceptable salt or solvate thereof, as described herein. In some embodiments, the Compound of the Disclosure is a compound of any one of formulae (IB), (IIB), or (IIIB), or a pharmaceutically acceptable salt or solvate thereof, as described herein.

In another aspect, any method described herein can further comprise administering to the patient at least one other therapeutic agent. In another aspect, the therapeutic agent is an effective amount of an enzyme for enzyme replacement therapy. In another aspect, the enzyme is galactocerebrosidase or an analog thereof. In another aspect, the therapeutic agent is an effective amount of a small molecule chaperone. In another aspect, the small molecule chaperone binds competitively to an enzyme. In another aspect, the small molecule chaperone is selected from the group consisting of iminoalditols, iminosugars, aminosugars, thiophenylglycosides, glycosidase, sulfatase, glycosyl transferase, phosphatase, and peptidase inhibitors.

In another aspect, the therapeutic agent is an effective amount of substrate reduction agent for substrate reduction therapy.

In another aspect, the present disclosure is directed to a Compound of the Disclosure, as described herein, for use in the prevention or treatment of a condition associated with the alteration of the activity of galactocerebrosidase in a patient in need thereof. In some embodiments, the Compound of the Disclosure is a compound of formula (IA) or formula (IIA), or a pharmaceutically acceptable salt or solvate thereof, as described herein. In some embodiments, the Compound of the Disclosure is a compound of any one of formulae (IB), (IIB), or (IIIB), or a pharmaceutically acceptable salt or solvate thereof, as described herein.

In another aspect, the present disclosure is directed to a Compound of the Disclosure, as described herein, for use in the prevention or treatment of a lysosomal storage disease, such as Krabbe's disease. In some embodiments, the Compound of the Disclosure is a compound of formula (IA) or formula (IIA), or a pharmaceutically acceptable salt or solvate thereof, as described herein. In some embodiments, the Compound of the Disclosure is a compound of any one of formulae (IB), (IIB), or (IIIB), or a pharmaceutically acceptable salt or solvate thereof, as described herein.

In another aspect, the present disclosure is directed to a Compound of the Disclosure, as described herein, for use in the prevention or treatment of an α-synucleinopathy, such as Parkinson's disease. In some embodiments, the Compound of the Disclosure is a compound of formula (IA) or formula (IIA), or a pharmaceutically acceptable salt or solvate thereof, as described herein. In some embodiments, the Compound of the Disclosure is a compound of any one of formulae (IB), (IIB), or (IIIB), or a pharmaceutically acceptable salt or solvate thereof, as described herein.

In another aspect, the present disclosure is directed to a Compound of the Disclosure, as described herein, for use in the prevention or treatment of a disease or disorder selected from the group consisting of Krabbe's disease, demyelinating disorders, galactosylsphingosine related disorders, globoid cell leukodystrophy, multiple sclerosis (MS), Parkinson's disease, peripheral neuropathy, progressive multiple sclerosis, pulmonary artery enlargement in COPD, open angle glaucoma, Lewy body dementia, and multiple system atrophy (MSA). In some embodiments, the Compound of the Disclosure is a compound of formula (IA) or formula (IIA), or a pharmaceutically acceptable salt or solvate thereof, as described herein. In some embodiments, the Compound of the Disclosure is a compound of any one of formulae (IB), (IIB), or (IIIB), or a pharmaceutically acceptable salt or solvate thereof, as described herein.

In another aspect, the present disclosure is also directed to the use of a Compound of the Disclosure, as described herein, for the treatment or prevention of a condition associated with the alteration of the activity of galactocerebrosidase in a patient in need thereof, such as those described herein. In some embodiments, the Compound of the Disclosure is a compound of formula (IA) or formula (IIA), or a pharmaceutically acceptable salt or solvate thereof, as described herein. In some embodiments, the Compound of the Disclosure is a compound of any one of formulae (IB), (IIB), or (IIIB), or a pharmaceutically acceptable salt or solvate thereof, as described herein.

In another aspect, the present disclosure is directed to a Compound of the Disclosure, as described herein, for use as a medicament. In some embodiments, the Compound of the Disclosure is a compound of formula (IA) or formula (IIA), or a pharmaceutically acceptable salt or solvate thereof, as described herein. In some embodiments, the Compound of the Disclosure is a compound of any one of formulae (IB), (IIB), or (IIIB), or a pharmaceutically acceptable salt or solvate thereof, as described herein.

In another aspect, the present disclosure is directed to use of a Compound of the Disclosure, as described herein, in the preparation of a medicament for the prevention or treatment of a condition associated with the alteration of the activity of galactocerebrosidase in a patient in need thereof, such as lysosomal storage diseases and α-synucleinopathies described herein. In some embodiments, the Compound of the Disclosure is a compound of formula (IA) or formula IIA), or a pharmaceutically acceptable salt or solvate thereof, as described herein. In some embodiments, the Compound of the Disclosure is a compound of any one of formulae (IB), (IIB), or (IIIB), or a pharmaceutically acceptable salt or solvate thereof, as described herein.

In another aspect, the present disclosure is directed to a pharmaceutical composition comprising a Compound of the Disclosure, as described herein, and at least one pharmaceutically acceptable excipient, for use in the treatment or prevention of a condition associated with the alteration of the activity of galactocerebrosidase in a patient in need thereof, such as lysosomal storage diseases and α-synucleinopathies described herein. In some embodiments, the Compound of the Disclosure is a compound of formula (IA) or formula (IIA), or a pharmaceutically acceptable salt or solvate thereof, as described herein. In some embodiments, the Compound of the Disclosure is a compound of any one of formulae (IB), (IIB), or (IIIB), or a pharmaceutically acceptable salt or solvate thereof, as described herein.

### Pharmaceutical Compositions

The present disclosure is also directed to pharmaceutical compositions, comprising an effective amount of a Compound of the Disclosure and at least one pharmaceutically acceptable excipient. In some embodiments, the composition comprises an effective amount of a compound of formula (IA) or formula (IIA), or a pharmaceutically acceptable salt or solvate thereof, as described herein, and at least one pharmaceutically acceptable excipient. In some embodiments, the composition comprises an effective amount of a compound of any one of formulae (IB), (IIB), or (IIIB), or a pharmaceutically acceptable salt or solvate thereof, as described herein, and at least one pharmaceutically acceptable excipient.

Due to their activity, Compounds of the Disclosure can be used in human medicine. As described above, Compounds of the Disclosure are useful, e.g., for treating or preventing lysosomal storage diseases, such as Krabbe's disease, and α-synucleinopathies, such as Parkinson's disease. Compounds of the Disclosure can be administered to any patient suffering any of said conditions. The term “patient” as used herein refers to any human that can experience the beneficial effects of a Compound of the Disclosure.

When administered to a patient, a Compound of the Disclosure can be administered as a component of a composition that comprises a pharmaceutically acceptable excipient or carrier.

Compounds of the Disclosure can be administered in combination with at least one other therapeutic agent. Administration of Compounds of the Disclosure with at least one other therapeutic agent can be sequential or concurrent. In another aspect, the Compound of the Invention and the at least one other therapeutic agent are administered in separate dosage forms. In another aspect, the Compound of the Invention and the at least one other therapeutic agent are administered concurrently in the same dosage form.

The term “excipient” refers to a vehicle, diluent, or adjuvant that is administered with the active ingredient. Such pharmaceutical excipients can be sterile liquids, such as water and oils, including those of petroleum, animal, vegetable, or synthetic origin, such as peanut oil, soybean oil, mineral oil, sesame oil, and similar. Water or saline aqueous solutions and aqueous dextrose and glycerol solutions, for example, for injectable solutions, can be used as vehicles. Suitable pharmaceutical vehicles are described in “Remington's Pharmaceutical Sciences” by E. W. Martin, 21^{st} Edition, 2005; or “Handbook of Pharmaceutical Excipients,” Rowe C. R.; Paul J. S.; Marian E. Q., sixth Edition, incorporated herein by reference.

Examples of pharmaceutical compositions include any solid composition (tablets, pills, capsules, granules, etc.) or liquid compositions (solutions, suspensions, or emulsions) for oral, topical, or parenteral administration.

In another embodiment, the pharmaceutical compositions are in an oral delivery form. Pharmaceutical forms suitable for oral administration can be tablets and capsules, and can contain conventional excipients known in the art, such as binders, for example syrup, gum Arabic, gelatin, sorbitol, tragacanth, or polyvinylpyrrolidone; fillers, for example lactose, sugar, cornstarch, calcium phosphate, sorbitol, or glycine; lubricants for the preparation of tablets, for example magnesium stearate; disintegrants, for example starch, polyvinylpyrrolidone, sodium starch glycolate, or microcrystalline cellulose; or pharmaceutically acceptable wetting agents, such as sodium lauryl sulphate.

Solid oral compositions can be prepared by conventional methods of blending, filling, or preparation of tablets. Repeated blending operations can be used to distribute the active ingredient in all the compositions that use large amounts of fillers. Such operations are conventional in the art. The tablets can be prepared, for example, by dry or wet granulation and optionally can be coated by well known methods in normal pharmaceutical practice, in particular using enteric coating.

Pharmaceutical compositions can also be adapted for parenteral administration, such as sterile solutions, suspensions, or lyophilized products in the appropriate unit dosage form. Suitable excipients, such as fillers, buffering agents, or surfactants can be used.

The mentioned formulations can be prepared using standard methods, such as those described or referred to in the Spanish and U.S. Pharmacopoeias and similar reference texts.

In general, the effective amount of a Compound of the Disclosure to be administered depends on the relative efficacy of the compound chosen, the severity of the condition or disorder being treated, and the patient's weight. The active compound can be administered one or more times a day, for example 1, 2, 3, or 4 times daily, with typical total daily doses in the range from about 0.01 mg/kg of body weight/day to about 1000 mg/kg of body weight/day. In another embodiment, the effective dosage amount of a Compound of the Disclosure is about 500 mg/kg of body weight/day or less. In another embodiment, the effective dosage amount of a Compound of the Disclosure is about 100 mg/kg of body weight/day or less. In another embodiment, the effective dosage amount ranges from about 0.01 mg/kg of body weight/day to about 100 mg/kg of body weight/day of a Compound of the Disclosure; in another embodiment, from about 0.02 mg/kg of body weight/day to about 50 mg/kg of body weight/day of a Compound of the Disclosure; and in another embodiment, from about 0.025 mg/kg of body weight/day to about 20 mg/kg of body weight/day of a Compound of the Disclosure.

A composition of the disclosure can be prepared by a method comprising admixing a Compound of the Disclosure with a pharmaceutically acceptable excipient or carrier. Admixing can be accomplished using methods known for admixing a compound and a pharmaceutically acceptable excipient or carrier. In another embodiment, the Compound of the Disclosure is present in the composition in an effective amount.

The following examples are illustrative, but not limiting, of the compounds, compositions and methods of the present disclosure. Suitable modifications and adaptations of the variety of conditions and parameters normally encountered in clinical therapy and which are obvious to those skilled in the art in view of this disclosure are within the spirit and scope of the disclosure.

### EXAMPLES

### Examples 1-28 Having Formula (IA)

The following Examples 1-28 were purchased and tested in the assay as described below. Examples 1-10, 13-23, 25, 27, and 28 were obtained from Enamine Ltd. (Ukraine). Examples 11, 12, and 26 were obtained from Vitas-M Laboratory (USA). Example 24 was obtained from Princeton BioMolecular Research Inc. (USA). The test results are provided in Table 1 below.

### Example 1

### 2-(((3-methyl-5-oxo-5H-thiazolo[3,2-a]pyrimidin-7-yl)methyl)amino)-N-(4-methylbenzyl)benzamide

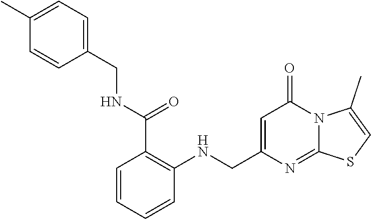 [SMILES: Cc1ccc(CNC(=O)c2ccccc2NCc2cc(=O)n3c(C)csc3n2)cc1]

### Example 2

### 2-(((7-bromo-4-oxo-4H-pyrido[1,2-a]pyrimidin-2-yl)methyl)amino)-N-(furan-2-ylmethyl)benzamide

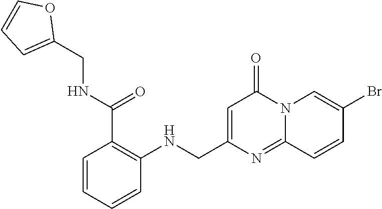 [SMILES: O=C(NCc1ccco1)c1ccccc1NCc1cc(=O)n2cc(Br)ccc2n1]

### Example 3

### 2-(((7-chloro-4-oxo-4H-pyrido[1,2-a]pyrimidin-2-yl)methyl)amino)-N-(furan-2-ylmethyl)benzamide

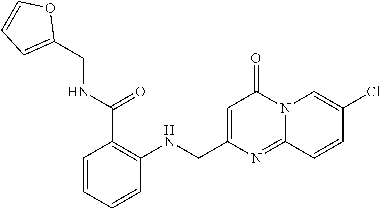 [SMILES: O=C(NCc1ccco1)c1ccccc1NCc1cc(=O)n2cc(Cl)ccc2n1]

### Example 4

### 2-((2-((2-chloro-4-methylphenyl)amino)-2-oxoethyl)amino)-N-(furan-2-ylmethyl)benzamide

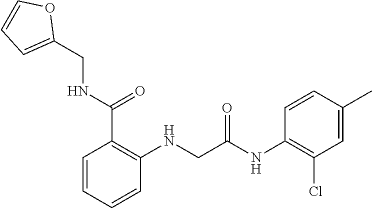 [SMILES: Cc1ccc(NC(=O)CNc2ccccc2C(=O)NCc2ccco2)c(Cl)c1]

### Example 5

### 2-((2-((4-ethylphenyl)amino)-2-oxoethyl)amino)-N-(furan-2-ylmethyl)benzamide

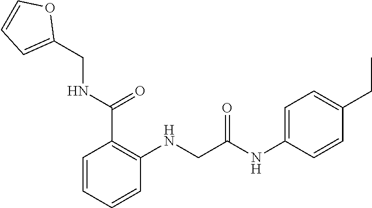 [SMILES: CCc1ccc(NC(=O)CNc2ccccc2C(=O)NCc2ccco2)cc1]

### Example 6

### 2-((2-((5-fluoro-2-methylphenyl)amino)-2-oxoethyl)amino)-N-(furan-2-ylmethyl)benzamide

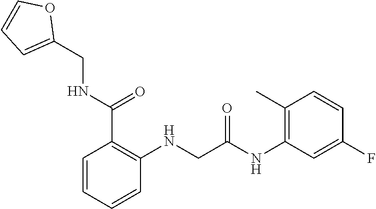 [SMILES: Cc1ccc(F)cc1NC(=O)CNc1ccccc1C(=O)NCc1ccco1]

### Example 7

### 2-((2-([1,1′-biphenyl]-4-ylamino)-2-oxoethyl)amino)-N-(furan-2-ylmethyl)benzamide

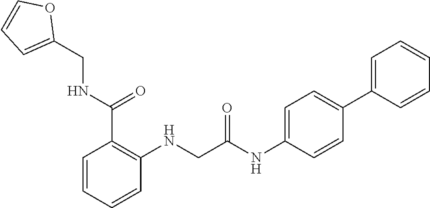 [SMILES: O=C(CNc1ccccc1C(=O)NCc1ccco1)Nc1ccc(-c2ccccc2)cc1]

### Example 8

### 2-((2,3-dihydro-1H-indene)-5-sulfonamido)-N-(furan-2-ylmethyl)benzamide

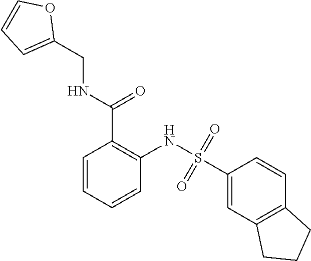 [SMILES: O=C(NCc1ccco1)c1ccccc1NS(=O)(=O)c1ccc2c(c1)CCC2]

### Example 9

### 2-((2-chloro-4-fluorophenyl)sulfonamido)-N-(furan-2-ylmethyl)benzamide

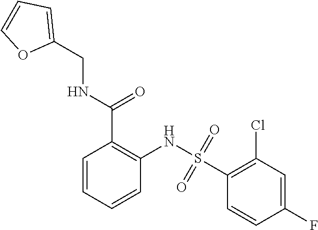 [SMILES: O=C(NCc1ccco1)c1ccccc1NS(=O)(=O)c1ccc(F)cc1Cl]

### Example 10

### 2-((3,4-dimethylphenyl)sulfonamido)-N-(furan-2-ylmethyl)benzamide

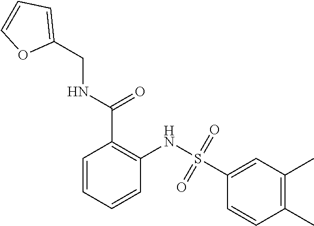 [SMILES: Cc1ccc(S(=O)(=O)Nc2ccccc2C(=O)NCc2ccco2)cc1C]

### Example 11

### 2-((4-bromophenyl)sulfonamido)-N-(furan-2-ylmethyl)benzamide

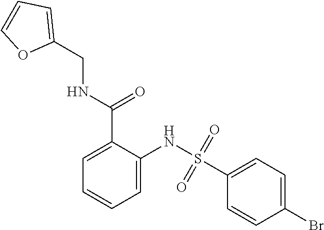 [SMILES: O=C(NCc1ccco1)c1ccccc1NS(=O)(=O)c1ccc(Br)cc1]

### Example 12

### 2-((4-chlorophenyl)sulfonamido)-N-(furan-2-ylmethyl)benzamide

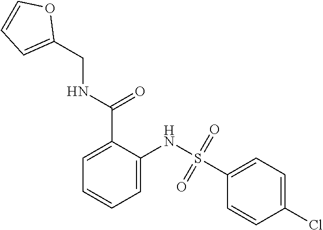 [SMILES: O=C(NCc1ccco1)c1ccccc1NS(=O)(=O)c1ccc(Cl)cc1]

### Example 13

### N-(furan-2-ylmethyl)-2-(((3-methyl-5-oxo-5H-thiazolo[3,2-a]pyrimidin-7-yl)methyl)amino)benzamide

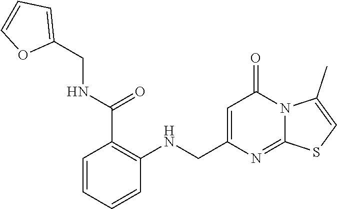 [SMILES: Cc1csc2nc(CNc3ccccc3C(=O)NCc3ccco3)cc(=O)n12]

### Example 14

### N-(furan-2-ylmethyl)-2-(((4-oxo-4H-pyrido[1,2-a]pyrimidin-2-yl)methyl)amino)benzamide

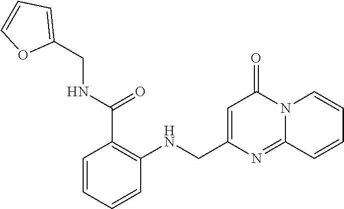 [SMILES: O=C(NCc1ccco1)c1ccccc1NCc1cc(=O)n2ccccc2n1]

### Example 15

### N-(furan-2-ylmethyl)-2-(((7-methyl-4-oxo-4H-pyrido[1,2-a]pyrimidin-2-yl)methyl)amino)benzamide

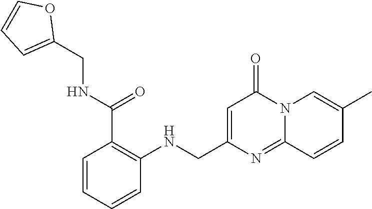 [SMILES: Cc1ccc2nc(CNc3ccccc3C(=O)NCc3ccco3)cc(=O)n2c1]

### Example 16

### N-(furan-2-ylmethyl)-2-(((9-methyl-4-oxo-4H-pyrido[1,2-a]pyrimidin-2-yl)methyl)amino)benzamide

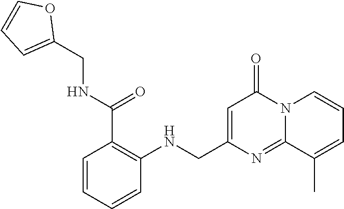 [SMILES: Cc1cccn2c(=O)cc(CNc3ccccc3C(=O)NCc3ccco3)nc12]

### Example 17

### N-(furan-2-ylmethyl)-2-((1-(naphthalen-1-ylamino)-1-oxopropan-2-yl)amino)benzamide

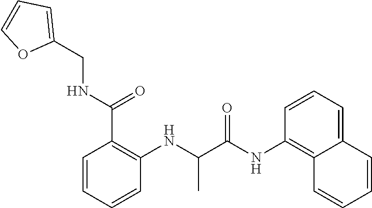 [SMILES: CC(Nc1ccccc1C(=O)NCc1ccco1)C(=O)Nc1cccc2ccccc12]

### Example 18

### N-(furan-2-ylmethyl)-2-((2-(methyl(phenyl)amino)-2-oxoethyl)amino)benzamide

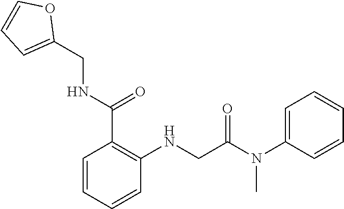 [SMILES: CN(C(=O)CNc1ccccc1C(=O)NCc1ccco1)c1ccccc1]

### Example 19

### N-(furan-2-ylmethyl)-2-((2-(naphthalen-1-ylamino)-2-oxoethyl)amino)benzamide

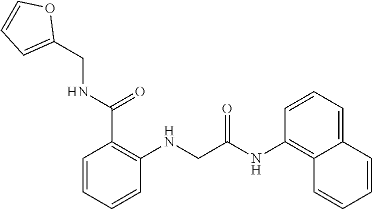 [SMILES: O=C(CNc1ccccc1C(=O)NCc1ccco1)Nc1cccc2ccccc12]

### Example 20

### N-(furan-2-ylmethyl)-2-((2-(naphthalen-2-ylamino)-2-oxoethyl)amino)benzamide

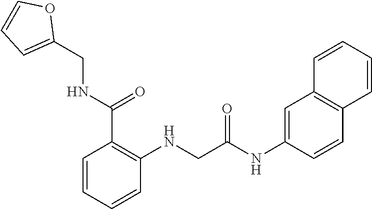 [SMILES: O=C(CNc1ccccc1C(=O)NCc1ccco1)Nc1ccc2ccccc2c1]

### Example 21

### N-(furan-2-ylmethyl)-2-((2-oxo-2-((4-(piperidin-1-yl)phenyl)amino)ethyl)amino)benzamide

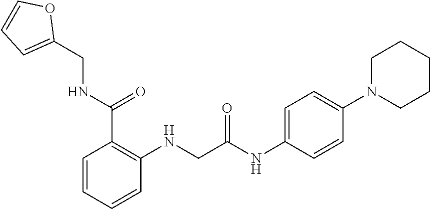 [SMILES: O=C(CNc1ccccc1C(=O)NCc1ccco1)Nc1ccc(N2CCCCC2)cc1]

### Example 22

### N-(furan-2-ylmethyl)-2-((2-oxo-2-((4-(pyrrolidin-1-yl)phenyl)amino)ethyl)amino)benzamide

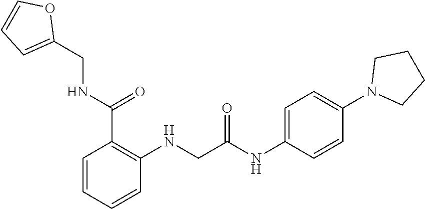 [SMILES: O=C(CNc1ccccc1C(=O)NCc1ccco1)Nc1ccc(N2CCCC2)cc1]

### Example 23

### N-(furan-2-ylmethyl)-2-((2-oxo-2-(p-tolylamino)ethyl)amino)benzamide

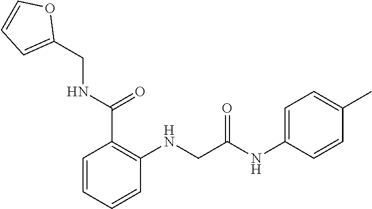 [SMILES: Cc1ccc(NC(=O)CNc2ccccc2C(=O)NCc2ccco2)cc1]

### Example 24

### N-(furan-2-ylmethyl)-2-((4-methylphenyl)sulfonamido)benzamide

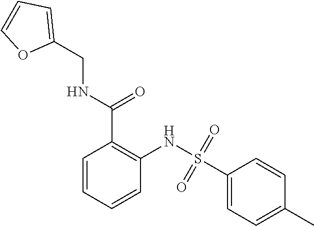 [SMILES: Cc1ccc(S(=O)(=O)Nc2ccccc2C(=O)NCc2ccco2)cc1]

### Example 25

### N-(furan-2-ylmethyl)-2-((5,6,7,8-tetrahydronaphthalene)-2-sulfonamido)benzamide

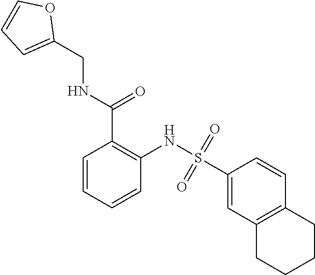 [SMILES: O=C(NCc1ccco1)c1ccccc1NS(=O)(=O)c1ccc2c(c1)CCCC2]

### Example 26

### N-(furan-2-ylmethyl)-2-(naphthalene-2-sulfonamido)benzamide

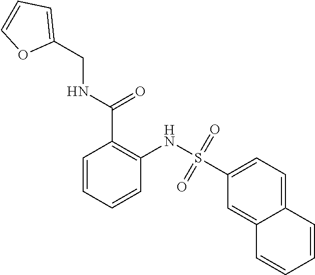 [SMILES: O=C(NCc1ccco1)c1ccccc1NS(=O)(=O)c1ccc2ccccc2c1]

### Example 27

### N-(furan-2-ylmethyl)-2-(phenylsulfonamido)benzamide

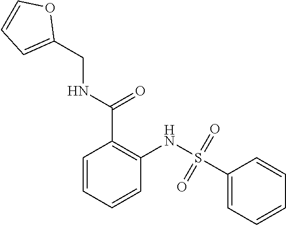 [SMILES: O=C(NCc1ccco1)c1ccccc1NS(=O)(=O)c1ccccc1]

### Example 28

### N-benzyl-2-(((3-methyl-5-oxo-5H-thiazolo[3,2-a]pyrimidin-7-yl)methyl)amino)benzamide

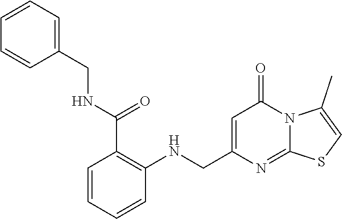 [SMILES: Cc1csc2nc(CNc3ccccc3C(=O)NCc3ccccc3)cc(=O)n12]

### Examples 29-41 Having Formula (IB)

The following Examples 29-41 were purchased and tested in the assay as described below. Examples 29-31 and 37 were obtained from Life Chemicals Inc. (Ukraine; Germany). Example 32 was obtained from Molport Inc. (Otava) (Latvia). Examples 33 and 35 were obtained from Princeton BioMolecular Research Inc. (USA). Example 34 was obtained from ChemDiv Inc. (USA). Examples 36 and 39-41 were ibtained from Enamine Ltd. (Ukraine). Example 38 was obtained from Mcule (Enamine) (Hungary). The test results are provided in Table 2 below.

### Example 29

### (E)-3-(furan-2-yl)-N-(2-(phenylsulfonyl)-1,2,3,4-tetrahydroisoquinolin-7-yl)acrylamide

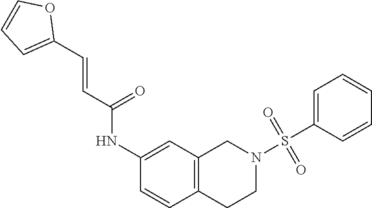 [SMILES: O=C(/C=C/c1ccco1)Nc1ccc2c(c1)CN(S(=O)(=O)c1ccccc1)CC2]

### Example 30

### (E)-3-(furan-2-yl)-N-(2-(propylsulfonyl)-1,2,3,4-tetrahydroisoquinolin-7-yl)acrylamide

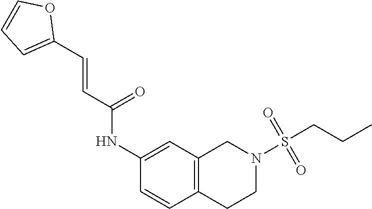 [SMILES: CCCS(=O)(=O)N1CCc2ccc(NC(=O)/C=C/c3ccco3)cc2C1]

### Example 31

### (E)-3-(furan-2-yl)-N-(3-(6-(pyrrolidin-1-yl)pyridazin-3-yl)phenyl)acrylamide

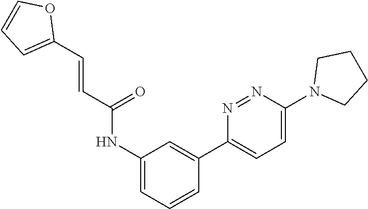 [SMILES: O=C(/C=C/c1ccco1)Nc1cccc(-c2ccc(N3CCCC3)nn2)c1]

### Example 32

### (E)-3-(furan-2-yl)-N-(3-(6-oxo-1,6-dihydropyridazin-3-yl)phenyl)acrylamide

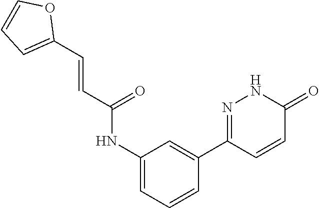 [SMILES: O=C(/C=C/c1ccco1)Nc1cccc(-c2ccc(=O)[nH]n2)c1]

### Example 33

### (E)-N-(3-(1H-benzo[d]imidazol-2-yl)phenyl)-3-(furan-2-yl)acrylamide

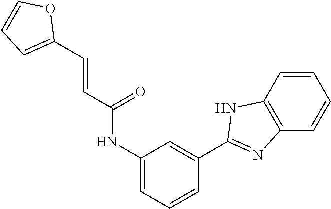 [SMILES: O=C(/C=C/c1ccco1)Nc1cccc(-c2nc3ccccc3[nH]2)c1]

### Example 34

### (E)-N-(3-(6-(ethylthio)pyridazin-3-yl)phenyl)-3-(furan-2-yl)acrylamide

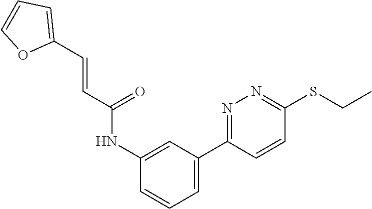 [SMILES: CCSc1ccc(-c2cccc(NC(=O)/C=C/c3ccco3)c2)nn1]

### Example 35

### (E)-N-(3-(benzo[d]thiazol-2-yl)phenyl)-3-(furan-2-yl)acrylamide

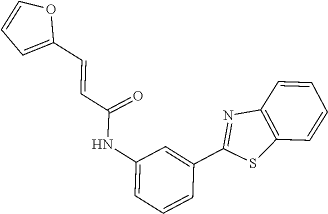 [SMILES: O=C(/C=C/c1ccco1)Nc1cccc(-c2nc3ccccc3s2)c1]

### Example 36

### 2-acetamido-N-(4′-(dimethylamino)-[1,1′-biphenyl]-3-yl)acetamide

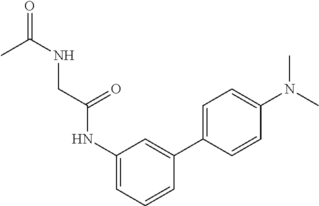 [SMILES: CC(=O)NCC(=O)Nc1cccc(-c2ccc(N(C)C)cc2)c1]

### Example 37

### N-(3-([1,2,4]triazolo[4,3-b]pyridazin-6-yl)phenyl)-2-((4-fluorophenyl)thio)acetamide

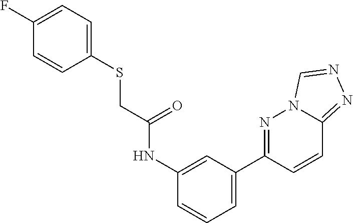 [SMILES: O=C(CSc1ccc(F)cc1)Nc1cccc(-c2ccc3nncn3n2)c1]

### Example 38

### N-(3-(N-(3-chlorophenyl)sulfamoyl)phenyl)butyramide

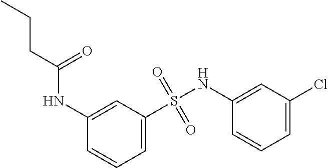 [SMILES: CCCC(=O)Nc1cccc(S(=O)(=O)Nc2cccc(Cl)c2)c1]

### Example 39

### N-(5-(N-(3-chlorophenyl)sulfamoyl)-2-hydroxyphenyl)cyclopropanecarboxamide

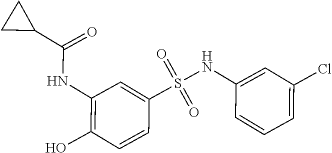 [SMILES: O=C(Nc1cc(S(=O)(=O)Nc2cccc(Cl)c2)ccc1O)C1CC1]

### Example 40

### N-(5-(N-(3-chlorophenyl)sulfamoyl)-2-hydroxyphenyl)pentanamide

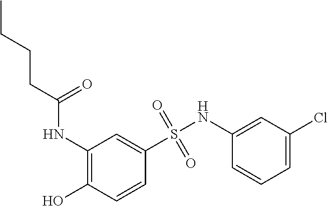 [SMILES: CCCCC(=O)Nc1cc(S(=O)(=O)Nc2cccc(Cl)c2)ccc1O]

### Example 41

### N-(5-(N-(3-chlorophenyl)sulfamoyl)-2-hydroxyphenyl)propionamide

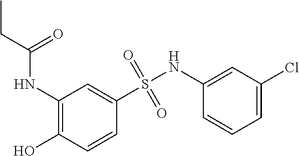 [SMILES: CCC(=O)Nc1cc(S(=O)(=O)Nc2cccc(Cl)c2)ccc1O]

### General Experimental Conditions for Examples 42-77

Hereinafter, the term “h” means hours, “eq” means equivalents, “min” means minutes, “Pd(PPh₃)₄” means palladium-tetrakis(triphenylphosphine), “Pd₂dba₃” Tris(dibenzylideneacetone)dipalladium(0), “XPhos” means 2-dicyclohexylphosphino-2′,4′,6′-triisopropylbiphenyl, “NMP” means N-Methyl-2-pyrrolidone, “HATU” means 1-[Bis(dimethylamino)methylene]-1H-1,2,3-triazolo[4,5-b]pyridinium 3-oxid hexafluorophosphate, “HPLC” means high-performance liquid chromatography, “TLC” means thin layer chromatography, “LC-MS” or “HPLC-MS” means Liquid chromatography-mass spectrometry, “CDCl₃” means deuterated chloroform, “DMSO-d₆” means deuterated dimethyl sulfoxide, “DCM” means dichloromethane, and “DMEDA” means 1,2-dimethylethylenediamine.

¹H NMR spectra were recorded on a Bruker (400 MHz and 500 MHz).

HPLC spectra were recorded on Waters 2695.

LC-MS analysis of the compounds was conducted as per one of the following methods.

Method-A: X-BRIDGE C18 (4.6 mm×75 mm 3.5 m); wavelength: 215 nm; flow: 2.0 mL/min; run time: 5.0 min; Mobile phase A: 10 mM ammonium acetate in water and B: 100% acetonitrile; Time and mobile phase-gradient (time in min/% B): 0.0/10, 0.2/10, 2.5/75, 3.0/100, 4.8/100, 5.0/10; MASS: Agilent 1200 SERIES, Mass: 6130SQD (ESI/APCI).

Method-B: Aquity UPLC BEH C18 (50 mm×2.1 mm, 1.7 m); wavelength: 215 nm; flow: 0.8 mL/min; run time: 3.0 min; Mobile phase A: 0.1% of formic acid in water and B: 1.0% formic acid in acetonitrile; Time and mobile phase-gradient (time in min/% B): 0.0/2, 0.2/2, 1.5/98, 2.6/98, 2.61/2, 3.2/2; MASS: Agilent 1290 infinity, Mass: 6150 SQD (ESI/APCI).

Method-C: Aquity UPLC BEH C18 (50 mm×2.1 mm, 1.7 m); wavelength: 215 nm; flow: 0.6 mL/min; run time: 4.0 min; Mobile phase A: 0.1% of formic acid in water and B 1.0% formic acid in acetonitrile; Time and mobile phase-gradient (time in min/% B: 0/95; 0.3/95; 2.0/5; 3.5/5; 3.6/95; MASS: Agilent 1290 infinity, Mass: 6150 SQD (ESI/APCI).

Method-D: Aquity UPLC BEH C18 (50 mm×2.1 mm, 1.7 m); wavelength: 215 nm; flow: 0.8 mL/min; run time: 3.2 min; Mobile phase A: 0.1% of formic acid in water and B: acetonitrile; Time and mobile phase-gradient (time in min/% A): 0/98, 0.5/98, 3.4/2, 4.2/2, 4.5/98, 5/98; MASS: Waters Acquity UPLC with SQD(ESI/APCI).

Method-E: SunFire C18 (3 mm×30 mm, 2.5 m); Flow rate: 1.8 mL/min. Mobile phase A: water (10 mmol Ammonium bicarbonate) and B: acetonitrile. Gradient: 5% B for 0.2 min, increase to 95% B within 1.4 min, 95% B for 1.3 min, back to 5% B within 0.01 min. Oven Temperature: 50° C. Agilent 1200 Series, Agilent 6110 Quadrupole LC/MS.

Method-F: SunFire C18 (4.6 mm×50 mm, 3.5 m); Flow rate: 2.0 mL/min. Mobile phase A: water (0.01% trifluoroacetic acid) and B: acetonitrile (0.01% trifluoroacetic acid). Gradient: 5%-95% B in 1.5 min. Oven Temperature: 50° C. Agilent 1200 Series. Agilent 6110 Quadrupole LC/MS.

Method-G: Agilent 1200 Series; Flow rate: 1.8 mL/min. Mobile phase A: water (10 mmol Ammonium bicarbonate) and B: acetonitrile. Gradient: 5%-90% B in 1.4 min. Oven Temperature: 50° C. Agilent 6110 Quadrupole LC/MS.

Method-H: X-BRIDGE C18 (4.6 mm×50 mm 3.5 m); Flow rate: 1.8 mL/min. Mobile phase A: water (10 mmol Ammonium bicarbonate) and B: acetonitrile. Gradient: 5%-90% B in 1.4 min. Oven Temperature: 50° C.

### Synthesis of Example 42

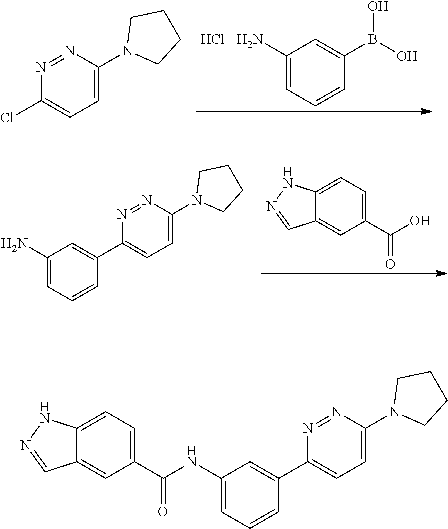

### Intermediate 1

### 3-(6-(pyrrolidin-1-yl)pyridazin-3-yl)aniline

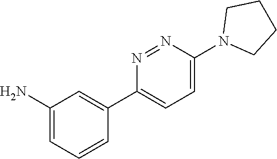 [SMILES: Nc1cccc(-c2ccc(N3CCCC3)nn2)c1]

Sodium carbonate (8.69 g, 0.082 mmol) was added to a stirred solution of 3-chloro-6-(pyrrolidin-1-yl)pyridazine (5.0 g, 0.027 2 mmol) and (3-aminophenyl)boronic acid. HCl (5.19 g, 0.030 mmol) in toluene-ethanol-water (210 mL, 1:1:01 v/v/v). The reaction mixture was purged with argon for 10 min and added Pd(PPh₃)₄ (3.1 g, 0.0027 mmol). The mixture was purged again with argon for 10 min. The reaction mixture was heated to 100° C. for 16 h. After consumption of starting materials (monitored by TLC), reaction mixture was cooled to RT and filtered through celite bed. The solvent was concentrated under reduced pressure to get crude wanted product. The crude was purified by flash chromatography (silica gel 230-400 mesh; 4-6% MeOH in DCM) to get 2.2 g of compound 3-(6-(pyrrolidin-1-yl)pyridazin-3-yl)aniline as pale yellow solid.

Yield: (2.2 g, 33%).

ES-MS [M+H]⁺: 241.2; Rt=1.20 min (Method-B).

¹H NMR (400 MHz, DMSO-d₆): δ 7.71 (d, J=9.2 Hz, 1H), 7.23 (s, 1H), 7.11-7.06 (m, 2H), 6.90 (d, J=9.6 Hz, 1H), 6.59-6.56 (m, 1H), 5.14 (s, 2H), 3.51-3.47 (m, 4H), 2.00-1.97 (m, 4H).

### Example 42

### N-(3-(6-(pyrrolidin-1-yl)pyridazin-3-yl)phenyl)-1H-indazole-5-carboxamide

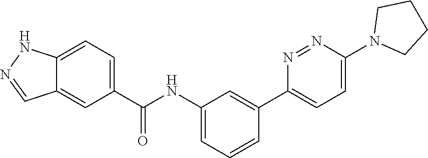 [SMILES: O=C(Nc1cccc(-c2ccc(N3CCCC3)nn2)c1)c1ccc2[nH]ncc2c1]

50% propylphosphonic anhydride (T3P) solution in EtOAc (1.1 ml, 1.6 mmol) was added to a suspension of intermediate 1 (0.20 g, 0.83 mmol), 1H-indazole-5-carboxylic acid (0.135 g, 0.83 mmol) and diisopropylethylamine (0.53 g, 4.1 mmol) in CH₂Cl₂ (10 mL) at 0° C. The reaction mixture was warmed to RT and stirred for 16 h. The reaction mixture was quenched with saturated NaHCO₃ solution, the organic product was extracted with 10% MeOH in CH₂Cl₂ (3×25 mL). The combined organic extracts were washed with water, brine, dried over anhydrous Na₂SO₄ and solvent was evaporated under reduced pressure to get crude product. The crude product was purified by column chromatography (silica gel 230-400 mesh, 2-4% MeOH in CH₂Cl₂ as eluent) to get the wanted product as an off-white solid.

Yield: (16 mg, 12%).

ES-MS [M+H]⁺: 35.2; Rt=1.46 min (Method-B).

¹H NMR (400 MHz, DMSO-d₆): δ 10.36 (s, 1H), 8.53-8.48 (m, 2H), 8.27 (s, 1H), 8.00-7.98 (m, 1H), 7.86-7.83 (m, 2H), 7.76-7.64 (m, 2H), 7.45 (t, J=8 Hz, 1H), 6.98-6.92 (m, 1H), 3.53-3.51 (m, 4H), 2.01-1.99 (m, 4H).

### Synthesis of Example 43

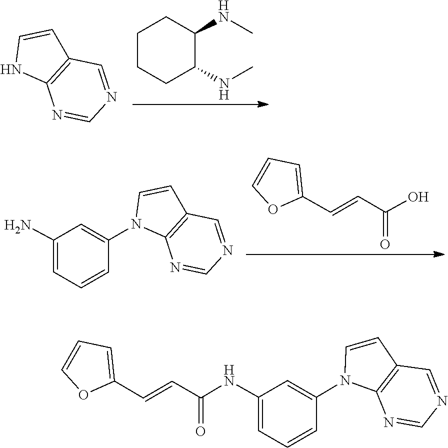

### Intermediate 2

### 3-(7H-pyrrolo[2,3-d]pyrimidin-7-yl)aniline

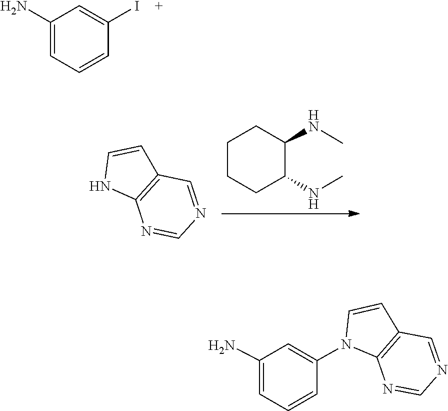

K₃PO₄ (1.95 g, 9.23 mmol, 2.2 eq) was added to a stirred solution of compound 7H-pyrrolo[2,3-d]pyrimidine (0.5 g, 4.19 mmol, 1 eq), 3-iodoaniline (1.37 g, 6.29 mmol, 1.5 eq), CuI (0.056 g, 0.293 mmol, 0.07 eq) and trans-N,N′-dimethylcyclohexane-1,2-diamine (0.09 g, 0.629 mmol, 0.15 eq) in 1,4-dioxane (20 mL). The mixture was purged again with argon for 10 min. The reaction mixture was stirred at 100° C. for 16 h. The reaction mixture was quenched with water and product was extracted using EtOAc. The combined organic layer was washed with brine, dried over anhydrous Na₂SO₄ and solvent was distilled under reduced pressure to afford intermediate 2 as crude. The crude product was purified by column chromatography (silica gel 230-400 mesh, 10-15% MeOH in DCM as eluent) to afford 0.2 g of 3-(7H-pyrrolo[2,3-d]pyrimidin-7-yl)aniline.

Yield: (0.2 g, 23%).

ES-MS [M+H]⁺: 211.03; Rt=0.87 min (Method-C).

### Example 43

### (E)-N-(3-(7H-pyrrolo[2,3-d]pyrimidin-7-yl)phenyl)-3-(furan-2-yl)acrylamide

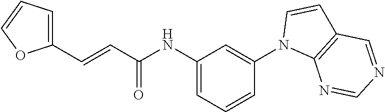 [SMILES: O=C(/C=C/c1ccco1)Nc1cccc(-n2ccc3cncnc32)c1]

The title compound was synthesized following the procedure described for Example 42 using (E)-3-(furan-2-yl)acrylic acid, and isolated as an off-white solid.

Yield: (0.02 g, 6%).

ES-MS [M+H]⁺: 331.18; Rt=1.69 min (Method-C).

¹H NMR (400 MHz, DMSO-d₆): δ 10.50 (s, 1H), 9.14 (s, 1H), 8.88 (s, 1H), 8.28 (m, 1H), 8.00 (m, 1H), 7.84 (s, 1H), 7.73-7.70 (m, 1H), 7.55-7.40 (m, 3H), 6.90-6.87 (m, 2H), 6.69-6.63 (m, 2H).

### Synthesis of Example 44

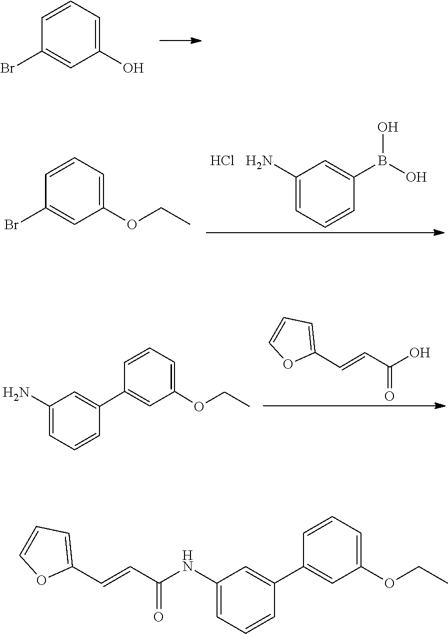

### Intermediate 3

### 3′-ethoxybiphenyl-3-amine

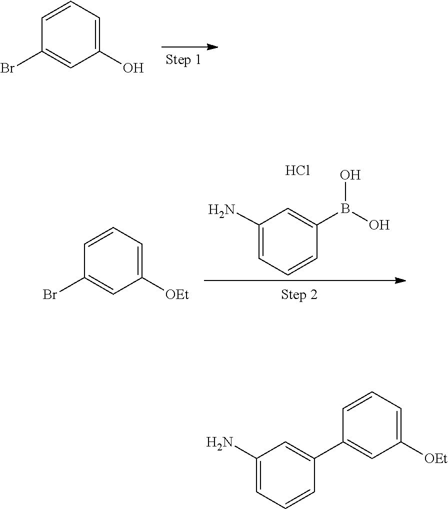 [SMILES: Oc1cccc(Br)c1 ; Cl ; CCOc1cccc(Br)c1 ; Nc1cccc(B(O)O)c1 ; CCOc1cccc(-c2cccc(N)c2)c1]

### Step 1

Sodium hydride (60% dispersion in mineral oil) (0.94 g, 39.2 mmol, 2 eq) was added to a stirred solution of 3-bromophenol (2.0 g, 11.6 mmol, 1 eq) in DMF (20 mL) at 0° C. and stirred for 30 min. To the reaction mixture was added ethyl iodide (2.7 mL, 17.3 mmol, 3 eq). The reaction mixture was stirred at room temperature (RT) for 16 h. The reaction mixture was quenched with ice water and the organic product was extracted with ethyl acetate (3×50 mL). The combined organic extracts were dried over anhydrous Na₂SO₄. Solvent was distilled under reduced pressure to give the crude compound. The crude product was purified by column chromatography (silica gel 60-120; 15% Ethyl acetate in Hexanes as eluent) to afford 1.8 g of 1-bromo-3-ethoxybenzene.

Yield: (1.8 g, 77%).

ES-MS [M+H]⁺: 202.0; Rt=3.13 min (Method-A).

¹H NMR (400 MHz, CDCl₃): δ 7.14-7.07 (m, 1H), 7.06-7.04 (m, 2H), 6.83-6.80 (m, 1H), 4.03-3.98 (q, J=7 Hz, 2H), 1.40 (t, J=7 Hz, 3H).

### Step 2

Sodium carbonate (1.43 g, 13.5 mmol, 3 eq) was added to a stirred solution of 1-bromo-3-ethoxybenzene (0.900 g, 4.5 mmol, 1 eq) and 3-aminophenylboronic acid hydrochloride (0.77 g, 4.5 mmol, 1 eq) in toluene-ethanol-water (16 mL:16 mL:1.6 mL). The reaction mixture was purged with argon for 10 min and catalytic tetrakis(triphenylphosphine)palladium(0) (1.03 g, 0.9 mmol, 0.2 eq) was added. The reaction mixture was purged again with argon for 10 min. The reaction mixture was stirred at 100° C. for 16 h. The reaction mixture was cooled to room temperature and concentrated under reduced pressure. The residue was diluted with DCM (300 mL), filtered through celite and solvent was concentrated under reduced pressure to get crude wanted compound. The crude compound was purified by column chromatography (silica gel 100-200; 25% Ethyl acetate in Hexanes as eluent) to afford 700 mg of 3′-ethoxybiphenyl-3-amine.

Yield: (0.700 g).

ES-MS [M+H]⁺: 214.2; Rt=1.78 min (Method-B).

### Example 44

### (E)-N-(3′-ethoxybiphenyl-3-yl)-3-(furan-2-yl)acrylamide

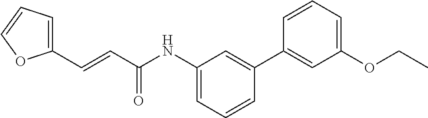 [SMILES: CCOc1cccc(-c2cccc(NC(=O)/C=C/c3ccco3)c2)c1]

The title compound was synthesized following the procedure described for Example 42 using (E)-3-(furan-2-yl)acrylic acid, and isolated as an off-white solid.

Yield: (0.049 g, 15%).

ES-MS [M+H]⁺: 334.2; Rt=2.09 min (Method-B).

¹H NMR (400 MHz, DMSO-d₆): δ 10.26 (s, 1H), 7.97 (s, 1H), 7.81 (s, 1H), 7.65 (d, J=7.6 Hz, 1H), 7.40-7.32 (m, 4H), 7.16-710 (m, 2H), 6.93-6.90 (m, 1H), 6.85-6.84 (m, 1H), 6.65-6.60 (m, 2H), 4.11-4.05 (q, J=8.5 Hz, 2H), 1.34 (t, J=8.5 Hz, 3H).

### Synthesis of Example 45

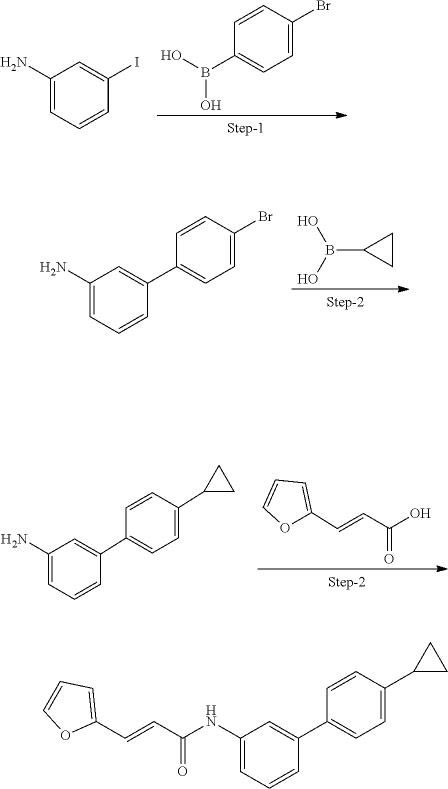

### Intermediate 4

### 4′-bromobiphenyl-3-amine

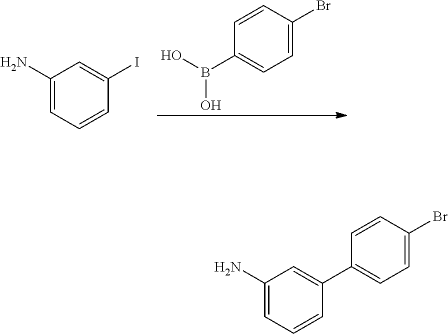

Sodium carbonate (2.9 g, 27.4 mmol, 3 eq) was added to a stirred solution of 3-iodoaniline (2.0 g, 9.1 mmol, 1 eq) and (4-bromophenyl)boronic acid (1.8 g, 9.1 mmol, 11 eq) in toluene-ethanol-water (40 mL-40 mL-4 mL). The reaction mixture was purged with argon for 10 min and added catalytic Pd(PPh₃)₄ (1.0 g, 0.91 mmol, 0.1 eq). The mixture was purged again with argon for 10 min. The reaction mixture was stirred at 100° C. for 16 h. The reaction mixture was cooled to room temperature and concentrated under reduced pressure. The residue was diluted with EtOAc (100 mL), filtered through celite and solvent was concentrated under reduced pressure to get crude wanted compound. The crude product was purified by column chromatography (silica gel 230-400 mesh, 20% EtOAc in Hexanes as eluent) to afford 600 mg of 4′-bromobiphenyl-3-amine.

Yield: (0.600 g, 26%).

ES-MS [M+H]⁺: 248.1; Rt=1.96 min (Method-B).

### Intermediate 5

### 4′-cyclopropylbiphenyl-3-amine

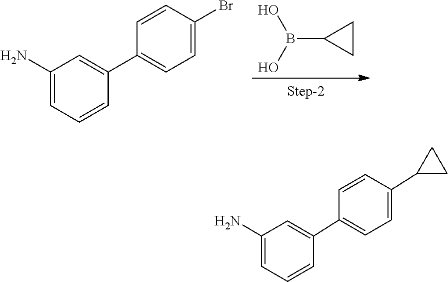 [SMILES: Nc1cccc(-c2ccc(Br)cc2)c1 ; OB(O)C1CC1 ; Nc1cccc(-c2ccc(C3CC3)cc2)c1]

K₃PO₄ (1.2 g, 5.7 mmol, 3 eq) was added to an argon purged solution of 4′-bromobiphenyl-3-amine (0.47 g, 1.9 mmol, 1 eq), cyclopropylboronic acid (0.33 g. 3.8 mmol, 2 eq), Pd(OAc)₂ (0.042 g, 0.19 mmol, 0.1 eq) and tricyclohexylphosphine (20% solution in toluene) (0.120 g, 0.19 mmol, 0.1 eq) in mixture of toluene:water (14 mL:1 mL). The mixture was purged again with argon for 10 min. The reaction mixture was stirred at 100° C. for 16 h. The reaction mixture was quenched with water and the organic product was extract with EtOAc. The combined organic layer was washed with brine, dried over anhydrous Na₂SO₄ and solvent was distilled under reduced pressure to afford the wanted compound as crude. The crude product was purified by column chromatography (silica gel 230-400 mesh, 10-15% EtOAc in Hexanes as eluent) to afford 0.25 g of 4′-cyclopropylbiphenyl-3-amine.

Yield: (0.250 g, 63%).

ES-MS [M+H]⁺: 210.2; Rt=1.90 min (Method-B).

### Example 45

### (E)-N-(4′-cyclopropylabiphenyl-3-yl)-3-(furan-2-yl)acrylamide

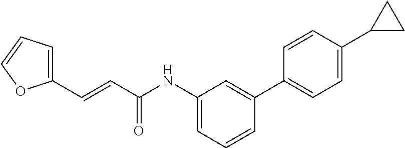 [SMILES: O=C(/C=C/c1ccco1)Nc1cccc(-c2ccc(C3CC3)cc2)c1]

The title compound was synthesized following the procedure described for Example 42 using (E)-3-(furan-2-yl)acrylic acid, and isolated as a pale yellow solid.

Yield: (0.143 g, 36%).

ES-MS [M+H]⁺: 330.2; Rt=2.15 min (Method-B).

¹H NMR (400 MHz, DMSO-d₆): δ 10.27 (s, 1H), 7.96 (s, 1H), 7.83 (s, 1H), 7.64 (d, J=8.8 Hz, 1H), 7.50 (d, J=8.4 Hz, 2H), 7.42-7.37 (m, 2H), 7.32-7.30 (m, 1H), 7.18 (d, J=7.6 Hz, 2H), 6.86 (m, 1H), 6.67-6.62 (m, 2H), 1.98-1.94 (m, 1H), 1.00-0.95 (m, 2H), 0.73-0.69 (m, 2H).

### Synthesis of Example 46

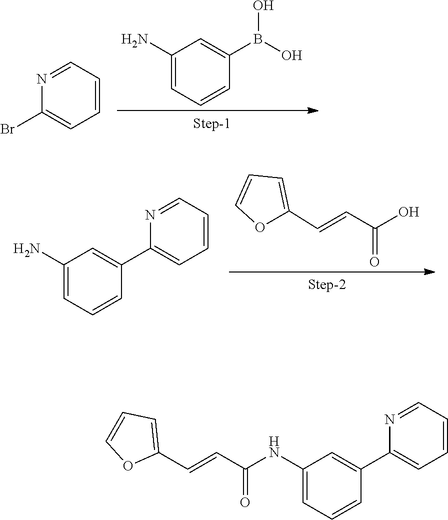

### Intermediate 6

### 3-(pyridin-2-yl)aniline

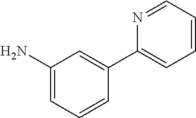 [SMILES: Nc1cccc(-c2ccccn2)c1]

Potassium carbonate (2.63 g, 19.1 mmol, 3 eq) was added to a stirred solution of 2-bromopyridine (0.600 g, 6.37 mmol, 1 eq) and 3-aminophenylboronic acid hydrochloride (0.165 g, 0.95 mmol, 0.15 eq) in DME:water (6 mL:0.15 mL). The reaction mixture was purged with argon for 10 min and catalytic amount of tetrakis(triphenylphosphine)palladium(0) (0.737 g 0.63 mmol, 0.1 eq) was added. The mixture was purged again with argon for 10 min. The reaction mixture was stirred at 90° C. for 16 h. The reaction mixture was cooled to room temperature and concentrated under reduced pressure. The residue was diluted with DCM (100 mL), filtered through celite and solvent was concentrated under reduced pressure to get crude 3-(pyridin-2-yl)aniline. The crude compound was purified by column chromatography (silica gel 100-200; 35% Ethyl acetate in Hexanes as eluent) to afford 300 mg of 3-(pyridin-2-yl)aniline.

Yield: (0.300 g, 46%).

ES-MS [M+H]⁺: 171.1; Rt=0.59 min (Method-B).

¹H NMR (400 MHz, CDCl₃): δ 8.68-8.66 (m, 1H), 7.73-7.68 (m, 2H), 7.39-7.31 (m, 1H), 7.27-7.20 (m, 2H), 6.76-6.73 (m, 1H), 3.77 (br s, 2H).

### Example 46

### (E)-3-(furan-2-yl)-N-(3-(pyridin-2-yl)phenyl)acrylamide

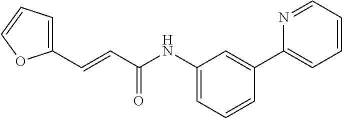 [SMILES: O=C(/C=C/c1ccco1)Nc1cccc(-c2ccccn2)c1]

The title compound was synthesized following the procedure described for Example 42 using (E)-3-(furan-2-yl)acrylic acid, and isolated as a white solid.

Yield: (0.043 g, 26%).

ES-MS [M+H]⁺: 291.2; Rt=1.69 min (Method-B).

¹H NMR (400 MHz, DMSO-d₆): δ 10.36 (s, 1H), 8.68 (d, J=5.2 Hz, 1H), 8.43 (s, 1H), 7.91-7.90 (m, 2H), 7.84-7.81 (m, 2H), 7.75 (d, J=8 Hz, 1H), 7.47-7.36 (m, 3H), 6.88-6.87 (m, 1H), 6.68-6.63 (m, 2H).

### Synthesis of Example 47

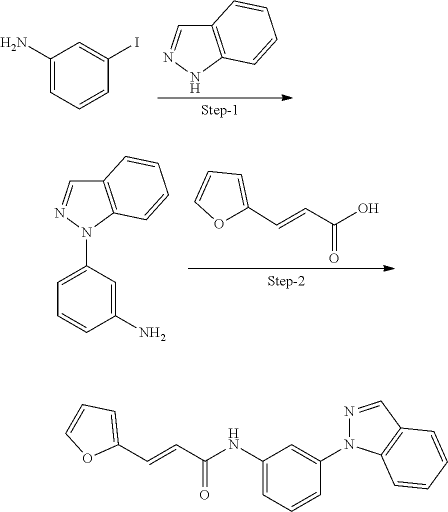

### Intermediate 7

### 3-(1H-indazol-1-yl)aniline

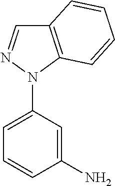 [SMILES: Nc1cccc(-n2ncc3ccccc32)c1]

Step-1: K₃PO₄ (3.74 g, 17.6 mmol, 3 eq) was added to a stirred solution of 3-iodoaniline (1.29 g, 5.9 mmol, 1 eq), indazole (0.700 g, 5.9 mmol, 1 eq), copper(I)iodide (CuI) (0.560 g, 2.9 mmol, 0.5 eq) and DMEDA (0.31 g, 3.5 mmol, 0.6 eq) in 1,4-dioxane:water (30 mL:3 mL). The mixture was purged again with argon for 10 min. The mixture was purged again with argon for 10 min. The reaction mixture was stirred at 120° C. for 24 h. The reaction mixture was quenched with water and product was extracted using EtOAc. The combined organic layer was washed with brine, dried over anhydrous Na₂SO₄ and solvent was distilled under reduced pressure to afford 3-(1H-indazol-1-yl)anilines crude. The crude product was purified by column chromatography (silica gel 230-400 mesh, 20% EtOAc in Hexanes as eluent) to afford 0.5 g of 3-(1H-indazol-1-yl)aniline.

Yield: (0.500 g, 40%).

¹H NMR (500 MHz, DMSO-d₆): δ 8.31 (s, 1H), 8.30-7.80 (m, 2H), 7.47-7.45 (m, 1H), 7.26-7.18 (m, 2H), 6.98 (s, 1H), 6.97-6.85 (m, 1H), 6.59-6.57 (m, 1H), 5.42 (br s, 2H).

### Example 47

### (E)-N-(3-(1H-indazol-1-yl)phenyl)-3-(furan-2-yl)acrylamide

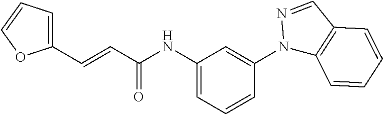 [SMILES: O=C(/C=C/c1ccco1)Nc1cccc(-n2ncc3ccccc32)c1]

The title compound was synthesized following the procedure described for Example 42 using (E)-3-(furan-2-yl)acrylic acid, and isolated as an off-white solid.

Yield: (0.066 g, 21%).

ES-MS [M+H]⁺: 33.02; Rt=2.01 min (Method-B).

¹H NMR (400 MHz, DMSO-d₆): δ 8.40 (s, 1H), 8.30 (s, 1H), 7.95-7.89 (m, 2H), 7.81 (s, 1H), 7.60-7.48 (m, 4H), 7.44 (s, 1H), 7.32 (t, J=7.2 Hz, 1H), 6.90 (d, J=3.2 Hz, 1H), 6.68-6.64 (m, 2H).

### Synthesis of Example 48

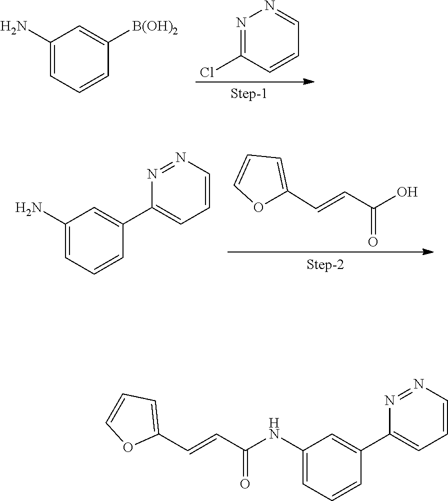

### Intermediate 8

### 3-(pyridazin-3-yl)aniline

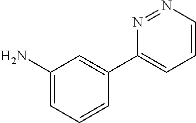 [SMILES: Nc1cccc(-c2cccnn2)c1]

Step-1: Cesium carbonate (5.5 g, 17.1 mmol, 3 eq) was added to a stirred solution of 3-chloropyridazine (0.650 g, 5.7 mmol, 1 eq) and (3-aminophenyl)boronic acid (0.859 g, 6.27 mmol, 1.1 eq) in dioxane:water (20 mL, 2 mL). The reaction mixture was purged with argon for 10 min and added Pd(PPh₃)₄ (0.658 g, 0.57 mmol, 0.1 eq). The mixture was purged again with argon for 10 min. The reaction mixture was stirred at 90° C. for 16 h. The reaction mixture was cooled to room temperature and concentrated under reduced pressure. The residue was diluted with ethyl acetate (100 mL), filtered through celite and solvent was concentrated under reduced pressure to get crude. The crude product was purified by column chromatography (silica gel 100-200 mesh; 50% EtOAc in Hexanes as eluent) to afford 60 mg of 3-(pyridazin-3-yl)aniline.

Yield: (0.220 g).

ES-MS [M+H]⁺: 171.97; Rt=0.48 min (Method-C).

### Example 48

### (E)-3-(furan-2-yl)-N-(3-(pyridazin-3-yl)phenyl)acrylamide

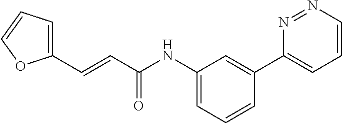 [SMILES: O=C(/C=C/c1ccco1)Nc1cccc(-c2cccnn2)c1]

The title compound was synthesized following the procedure described for Example 42 using (E)-3-(furan-2-yl)acrylic acid, and isolated as a white solid.

Yield: (0.016 g, 4%).

ES-MS [M−H]⁺: 290.09 Rt=1.74 min (Method-C).

¹H NMR (400 MHz, DMSO-d₆): δ 10.41 (s, 1H), 9.23 (s, 1H), 8.53 (s, 1H), 8.17 (d, J=8.4 Hz 1H), 7.89-7.78 (m, 4H), 7.52 (t, J=8.4 Hz, 1H), 7.44-740 (m, 1H), 6.68 (s, 1H), 6.64-6.3 (m, 2H).

### Example 49

### (E)-N-(3-(1H-pyrazol-1-yl)phenyl)-3-(furan-2-yl)acrylamide

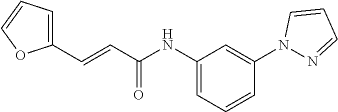 [SMILES: O=C(/C=C/c1ccco1)Nc1cccc(-n2cccn2)c1]

The title compound was synthesized following the procedure described for Example 42 using (E)-3-(furan-2-yl)acrylic acid, and isolated as a brown solid.

Yield: (0.054 g, 17%).

ES-MS [M−H]: 278.17; Rt=1.92 min (Method-C).

¹H NMR (400 MHz, DMSO-d₆): δ 10.39 (s, 1H), 8.43-8.42 (m, 1H), 8.28-8.27 (m, 1H), 7.84-7.83 (m, 1H), 7.75 (s, 1H), 7.60-7.57 (m, 1H), 7.51-7.49 (m, 1H), 7.45-7.40 (m, 2H), 6.88 (d, J=3.6 Hz, 1H), 6.66-6.62 (m, 2H), 6.55 (s, 1H).

### Synthesis of Example 50

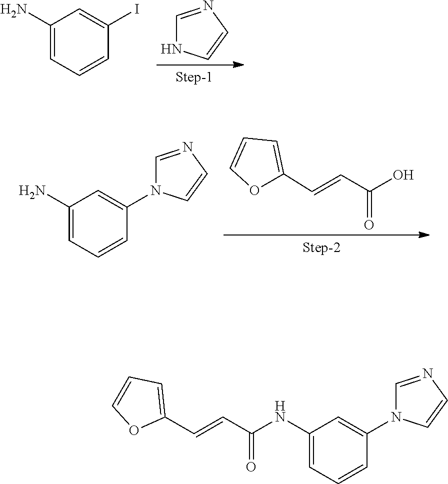

### Intermediate 9

### 3-(1H-imidazol-1-yl) aniline

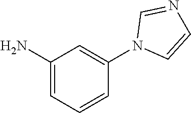 [SMILES: Nc1cccc(-n2ccnc2)c1]

Step-1: To a solution of 3-iodoaniline (0.500 g, 2.28 mmol, 1 eq) and imidazole (0.233 g, 3.42 mmol, 1.5 eq) in DMF (10 mL) were added potassium phosphate tribasic (1.45 g, 6.85 mmol, 3 eq) and copper(I)iodide (0.043 g, 0.22 mmol, 0.1 eq) and reaction mixture stirred at 120° C. for 24 h. The reaction mixture was cooled, DMF was evaporated under reduced pressure. Then it was extracted with 10% methanol in dichloromethane. The organic layer was dried over anhydrous sodium sulphate, filtered and concentrated under reduced pressure. The crude product was purified by flash column chromatography (230-400 silica) using 15% methanol in dichloromethane as eluent to get 3-(1H-imidazol-1-yl) aniline as brown gum.

Yield: (0.150 g, 41%).

ES-MS [M−H]⁺: 160.08; Rt=4.61 min (Method-B).

¹H NMR (400 MHz, DMSO-d₆): δ 7.82 (s, 1H), 7.24-7.22 (m, 1H), 7.20 (s, 1H), 7.17 (s, 1H), 6.77-6.74 (m, 1H), 6.68-6.64 (m, 2H), 3.86 (br, 2H).

### Example 50

### (E)-N-(3-(1H-imidazol-1-yl)phenyl)-3-(furan-2-yl)acrylamide

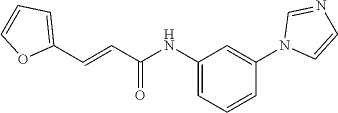 [SMILES: O=C(/C=C/c1ccco1)Nc1cccc(-n2ccnc2)c1]

The title compound was synthesized following the procedure described for Example 42 using (E)-3-(furan-2-yl)acrylic acid, and isolated as a light yellow solid.

Yield: (0.026 g, 10%).

ES-MS [M+H]⁺: 280.06; Rt=1.44 min (Method-A).

¹H NMR (400 MHz, DMSO-d₆): δ 8.00 (s, 1H), 7.89 (s, 1H), 7.56-7.52 (m, 3H), 7.49-7.40 (m, 2H), 7.32 (s, 1H), 7.21 (s, 1H), 7.16-7.13 (m, 1H), 6.64 (d, J=3.6 Hz, 1H), 6.50-6.44 (m, 2H).

### Synthesis of Example 51

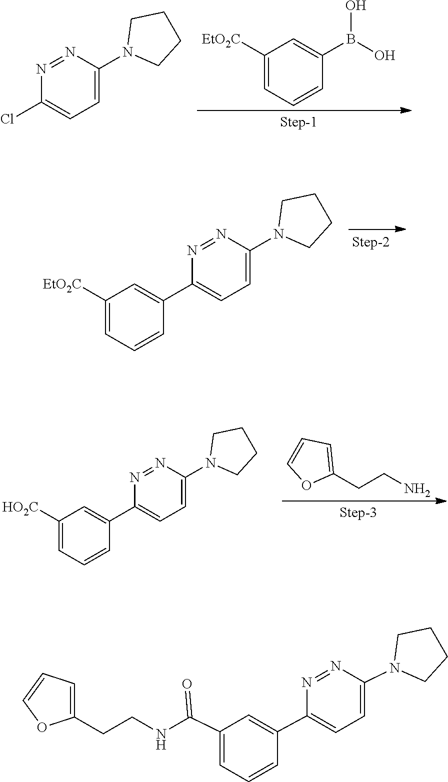

### Intermediate 10

### Ethyl 3-(6-(pyrrolidin-1-yl)pyridazin-3-yl)benzoate

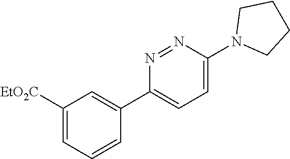 [SMILES: CCOC(=O)c1cccc(-c2ccc(N3CCCC3)nn2)c1]

Step-1: Sodium carbonate (3.48 g, 32.8 mmol, 3 eq) was added to a stirred solution of 3-chloro-6-(pyrrolidin-1-yl)pyridazine (2.0 g, 10.9 mmol, 1 eq) and (3-(ethoxycarbonyl)-phenyl)boronic acid (3.19 g, 16.4 mmol, 1.5 eq) in dioxane:water (40 ml:4 ml). The mixture was purged with argon for 10 min and added XPhos (2.08 g, 4.4 mmol, 0.4 eq) and Pd₂dba₃ (0.99 g, 1.1 mmol, 0.1 eq). The mixture was purged again with argon for 10 min. The reaction mixture was stirred at 100° C. for 16 h. The reaction mixture was cooled to room temperature and concentrated under reduced pressure. The residue was diluted with DCM (100 mL), filtered through celite and solvent was concentrated under reduced pressure to get crude compound. The crude was purified by flash chromatography (silica gel 230-400 mesh; 30-40% EtOAc in pet ether) to afford pale yellow solid 2.2 g of ethyl 3-(6-(pyrrolidin-1-yl)pyridazin-3-yl)benzoate.

Yield: (2.2 g, 67%).

ES-MS [M+H]⁺: 298.01; Rt=1.57 min (Method-C).

### Intermediate 11

### 3-(6-(pyrrolidin-1-yl)pyridazin-3-yl)benzoic acid

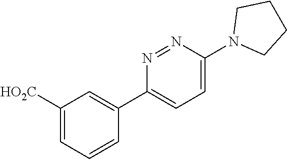 [SMILES: O=C(O)c1cccc(-c2ccc(N3CCCC3)nn2)c1]

Step-2: LiOH.H₂O (0.62 g, 14.8 mmol, 2 eq) was added to a stirred solution of ethyl 3-(6-(pyrrolidin-1-yl)pyridazin-3-yl)benzoate (2.20 g, 0.74 mmol, 1 eq) in MeOH:water (44 mL:4.4 mL) at 0° C. The reaction mixture was stirred at room temperature for 16 h. The reaction mixture was concentrated under reduced pressure. The residue was diluted with water (20 mL) and acidify by 2N HCl at 0° C. The product was precipitated out which was filtered and dried under vacuum to get crude 3-(6-(pyrrolidin-1-yl)pyridazin-3-yl)benzoic acid. The crude was used in the next step without purification.

Yield: (1.0 g, 50%).

ES-MS [M+H]⁺: 270.25; Rt=0.29 min (Method-A).

### Example 51

### N-(2-(furan-2-yl)ethyl)-3-(6-(pyrrolidin-1-yl) pyridazin-3-yl)benzamide

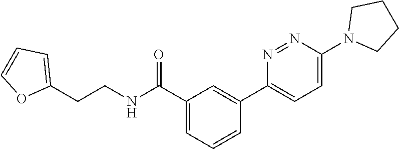 [SMILES: O=C(NCCc1ccco1)c1cccc(-c2ccc(N3CCCC3)nn2)c1]

The title compound was synthesized following the procedure described for Example 42 and isolated as a pale yellow solid.

Yield: (0.075 g, 37%).

ES-MS [M+H]⁺: 363.2; Rt=1.54 min (Method-B).

¹H NMR (400 MHz, DMSO-d₆): δ 8.74-8.71 (t, J=5.6 Hz 1H), 8.45 (s, 1H), 8.16-8.14 (d, J=7.6 Hz, 1H), 7.99-7.94 (d, J=9.2 Hz, 1H), 7.84-7.82 (d, J=7.6 Hz, 1H), 7.57-7.53 (m, 2H), 6.99-6.97 (d, J=9.6 Hz, 1H), 6.36-6.35 (m, 1H), 6.19-6.18 (m, 1H), 3.57-3.51 (m, 6H), 2.92-2.89 (t, J=6.8 Hz, 2H), 2.02-1.98 (m, 4H).

### Example 52

### N-(furan-2-ylmethyl)-3-(6-(pyrrolidin-1-yl)pyridazin-3-yl)benzamide

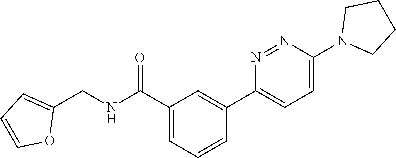 [SMILES: O=C(NCc1ccco1)c1cccc(-c2ccc(N3CCCC3)nn2)c1]

The title compound was synthesized following the procedure described for Example 42 and isolated as a pale yellow solid.

Yield: (0.060 g, 31%).

ES-MS [M+H]⁺: 349.2; Rt=1.50 min (Method-B).

¹H NMR (400 MHz, DMSO-d₆): δ 9.11-9.09 (m, 1H), 8.49 (s, 1H), 8.18-8.16 (d, J=7.6 Hz, 1H), 7.97-7.95 (d, J=9.6 Hz, 1H), 7.89-7.87 (d, J=7.6 Hz, 1H), 7.58-7.54 (m, 2H), 6.98-6.96 (d, J=9.6 Hz 1H), 6.41-6.40 (m, 1H), 6.30-6.29 (m, 1H), 4.51-4.49 (m, 2H), 3.53-3.50 (m, 4H), 2.01-1.98 (m, 4H).

### Example 53

### N-(2-(1H-pyrazol-1-yl)ethyl)-3-(6-(pyrrolidin-1-yl) pyridazin-3-yl)benzamide

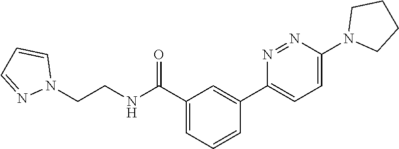 [SMILES: O=C(NCCn1cccn1)c1cccc(-c2ccc(N3CCCC3)nn2)c1]

The title compound was synthesized following the procedure described for Example 42 and isolated as a yellow solid.

Yield: (0.071 g, 35%).

ES-MS [M+H]⁺: 363.2; Rt=1.41 min (Method-B).

¹H NMR (400 MHz, DMSO-d₆): δ 8.72-8.69 (t, J=5.2 Hz, 1H), 8.43 (s, 1H), 8.16-8.14 (d, J=8.4 Hz, 1H), 7.95-7.92 (d, J=9.6 Hz, 1H), 7.82-7.80 (d, J=8 Hz, 1H), 7.71 (s, 1H), 7.57-7.53 (t, J=8 Hz 1H), 7.46-7.45 (s, 1H), 6.98 (d, J=9.2 Hz, 1H), 6.23-6.21 (m, 1H), 4.32 (t, J=6.4 Hz, 2H), 3.69-3.64 (q, J=6.4 Hz, 2H). 3.54-3.50 (m, 4H), 2.01-1.98 (m, 4H).

### Example 54

### 3-(6-(pyrrolidin-1-yl)pyridazin-3-yl)-N-(2-(thiazol-2-yl)ethyl)benzamide

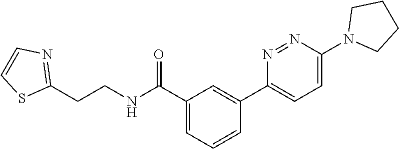 [SMILES: O=C(NCCc1nccs1)c1cccc(-c2ccc(N3CCCC3)nn2)c1]

The title compound was synthesized following the procedure described for Example 42 and isolated as an off-white solid.

Yield: (0.0148 g, 6.56%).

ES-MS [M+H]⁺: 380.20; Rt=1.41 min (Method-C).

¹H NMR (400 MHz, DMSO-d₆): δ 8.40 (s, 1H), 8.42 (s, 1H), 8.20-8.19 (d, J=7.6 Hz, 1H), 7.80-7.78 (d, J=7.6 Hz, 1H), 7.74-7.73 (m, 1H), 7.70-7.68 (d, J=9.6 Hz, 1H), 7.54-7.50 (t, J=8 Hz, 1H), 7.26-7.23 (m, 2H), 6.73-6.71 (d, J=9.6 Hz, 1H), 3.96-3.91 (q, J=6 Hz, 2H), 3.61 (m, 4H), 3.36-3.33 (t, J=6 Hz, 2H), 2.10-2.06 (m, 4H).

### Example 55

### N-(oxazol-2-ylmethyl)-3-(6-(pyrrolidin-1-yl)pyridazin-3-yl)benzamide

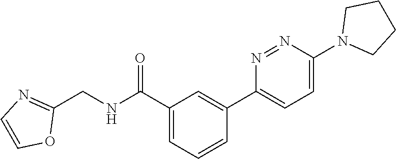 [SMILES: O=C(NCc1ncco1)c1cccc(-c2ccc(N3CCCC3)nn2)c1]

The title compound was synthesized following the procedure described for Example 42 and isolated as a white solid.

Yield: (0.025 g, 12%).

ES-MS [M+H]⁺: 350.25; Rt=1.36 min (Method-C).

¹H NMR (400 MHz, DMSO-d₆): δ 9.29 (t, J=5.6 Hz, 1H), 8.52 (s, 1H), 8.19 (d, J=8 Hz, 1H), 8.06 (s, 1H), 7.97 (d, J=9.2 Hz, 1H), 7.89 (d, J=7.6 Hz, 1H), 7.60-7.56 (m, 1H), 7.17 (s, 4H), 6.98 (d, J=9.6 Hz, 1H), 4.62 (d, J=6 Hz 2H), 3.52-3.51 (m, 4H), 2.01-1.98 (m, 4H).

### Example 56

### N-(isoxazol-5-ylmethyl)-3-(6-(pyrrolidin-1-yl)pyridazin-3-yl)benzamide

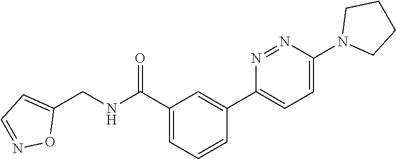 [SMILES: O=C(NCc1ccno1)c1cccc(-c2ccc(N3CCCC3)nn2)c1]

The title compound was synthesized following the procedure described for Example 42 and isolated as an off-white solid.

Yield: (0.048 g, 23%).

ES-MS [M+H]⁺: 350.21; Rt=1.38 min (Method-C).

¹H NMR (400 MHz, DMSO-d₆): δ 9.32-9.29 (t, J=6 Hz, 1H), 8.52-8.49 (m, 2H), 8.20-8.18 (m, 1H), 7.98-7.96 (d, J=9.6 Hz, 1H), 7.90-7.88 (m, 1H), 7.60-7.56 (t, J=7.6 Hz, 1H), 6.99-6.97 (d, J=9.6 Hz, 1H), 6.39 (s, 1H), 4.67-4.65 (d, J=6 Hz, 2H), 3.52 (m, 4H), 2.00 (m, 4H).

### Example 57

### 3-(6-(pyrrolidin-1-yl)pyridazin-3-yl)-N-(thiazol-2-ylmethyl)benzamide

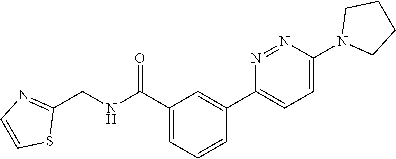 [SMILES: O=C(NCc1nccs1)c1cccc(-c2ccc(N3CCCC3)nn2)c1]

The title compound was synthesized following the procedure described for Example 42 and isolated as off-white solid.

Yield: (0.022 g, 10%).

ES-MS [M+H]⁺: 366.21; Rt=1.41 min (Method-C).

¹H NMR (400 MHz, DMSO-d₆): δ 9.55-9.52 (t, J=5.6 Hz, 1H), 8.55 (s, 1H), 8.21-8.19 (d, J=8 Hz, 1H), 7.98-7.96 (d, J=9.2 Hz, 1H), 7.92-7.90 (d, J=8 Hz, 1H), 7.75-7.74 (m, 1H), 7.64-7.58 (m, 2H), 7.00-6.97 (d, J=9.6 Hz, 1H), 4.79-4.78 (d, J=6 Hz, 2H), 3.54-3.51 (m, 4H), 2.01-1.98 (m, 4H).

### Example 58

(E)-N-(3-([1,2,4]triazolo[4,3-b]pyridazin-6-yl)phenyl)-3-(furan-2-yl)acrylamide

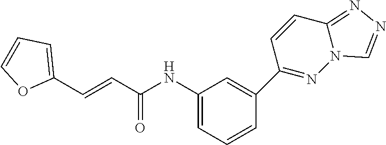 [SMILES: O=C(/C=C/c1ccco1)Nc1cccc(-c2ccc3nncn3n2)c1]

The title compound was synthesized following the procedure described for Example 42 using (E)-3-(furan-2-yl)acrylic acid, and isolated as a light brown solid.

Yield: (0.018 g, 4%).

ES-MS [M+H]⁺: 332.14; Rt=1.72 min (Method-A).

¹H NMR (400 MHz, DMSO-d₆): δ 10.48 (s, 1H), 9.73 (s, 1H), 8.49-8.47 (m, 2H), 7.91-9.85 (m, 3H), 7.78 (d, J=8 Hz, 1H), 7.55 (t, J=8 Hz, 1H), 7.45-7.41 (m, 1H), 6.88 (d, J=3.6 Hz, 1H), 6.68-6.63 (m, 2H).

### Example 59

### 2-(1H-pyrazol-1-yl)-N-(3-(6-(pyrrolidin-1-yl)pyridazin-3-yl)phenyl)acetamide

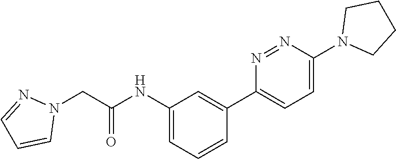 [SMILES: O=C(Cn1cccn1)Nc1cccc(-c2ccc(N3CCCC3)nn2)c1]

The title compound was synthesized following the procedure described for Example 42 and isolated as an off-white solid.

Yield: (57 mg, 26%).

ES-MS [M+H]⁺: 349.24; Rt=1.38 min (Method-C).

¹H NMR (400 MHz, DMSO-d₆): δ 10.40 (s, 1H), 8.26-8.25 (m, 1H), 7.81-7.77 (m, 2H), 7.68-7.61 (m, 2H), 7.47 (s, 1H), 7.43-7.39 (t, J=16 Hz, 1H), 6.95-6.93 (d, J=8 Hz, 1H), 6.29-6.28 (m, 1H), 5.04 (s, 2H), 3.52-3.49 (m, 4H), 2.01-1.97 (m, 4H).

### Example 60

### N-(3-(6-(pyrrolidin-1-yl)pyridazin-3-yl)phenyl)-3-(thiazol-2-yl)propanamide

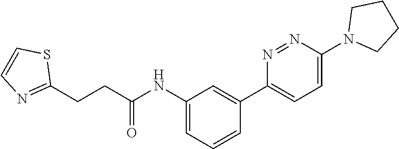 [SMILES: O=C(CCc1nccs1)Nc1cccc(-c2ccc(N3CCCC3)nn2)c1]

HATU (0.35 g, 0.93 mmol) was added portion wise to a solution of DIPEA (0.32 mL, 1.87 mmol) and 3-(thiazol-2-yl)propanoic acid (0.1 g, 0.625 mmol) in DCM (10 mL) at 0° C. The reaction mixture was stirred for 10 min at same temperature. Then, Intermediate 1 (3-(6-(pyrrolidin-1-yl)pyridazin-3-yl)aniline) (0.15 g, 0.625 mmol) was added to the reaction mixture at 0° C. The reaction mixture was stirred at room temperature for 16 h. The reaction mixture was quenched with minimum amount of aqueous natrium bicarbonate solution, the organic product was extracted with DCM (2×25 mL). The combined organic extracts were dried over anhydrous natrium sulphate. Solvent was distilled under reduced pressure to give the crude compound. The crude product was purified by column chromatography (silica gel 230-400 mesh, 2-4% methanol in DCM as eluent) to get the desired compound 3-(1H-pyrazol-1-yl)-N-(3-(6-(pyrrolidin-1-yl)pyridazin-3-yl)phenyl)propanamide as an off-white solid.

Yield: (70 mg, 29%).

ES-MS [M+H]⁺: 380.24; Rt=1.45 min (Method-C).

¹H NMR (400 MHz, DMSO-d₆): δ 10.13 (s, 1H), 8.26 (s, 1H), 7.80-7.77 (d, J=12 Hz, 1H), 7.69 (s, 1H), 7.63-7.56 (m, 3H), 7.40-7.36 (t, J=16 Hz, 1H), 6.95-6.93 (d, J=8 Hz, 1H), 3.52-3.49 (m, 4H), 3.34-3.30 (m, 2H), 2.87-2.84 (t, J=8 Hz, 2H), 2.01-1.97 (m, 4H).

### Example 61

### N-(3-(6-(pyrrolidin-1-yl)pyridazin-3-yl)phenyl)pentanamide

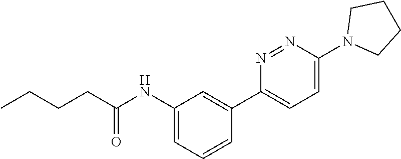 [SMILES: CCCCC(=O)Nc1cccc(-c2ccc(N3CCCC3)nn2)c1]

The title compound was synthesized following the procedure described for Example 42 and isolated as an off-white solid.

Yield: (68 mg).

ES-MS [M+H]⁺: 325.17; Rt=1.58 min (Method-B).

¹H NMR (400 MHz, DMSO-d₆): δ 9.95 (s, 1H), 8.26 (s, 1H), 7.80-7.77 (d, J=9.6 Hz, 1H), 7.63-7.60 (m, 2H), 7.39-7.35 (t, J=8 Hz, 1H), 6.95-6.935 (d, J=9.2 Hz, 1H), 3.52-3.49 (t, J=6.8 Hz, 4H), 2.34-2.31 (t, J=7.6 Hz, 2H), 2.01-1.97 (m, 4H), 1.61-1.57 (m, 2H), 1.36-1.31 (m, 2H), 0.92-0.89 (t, J=7.6 Hz, 3H).

### Intermediate 12

### 2-methoxy-5-(pyridin-2-yl)aniline

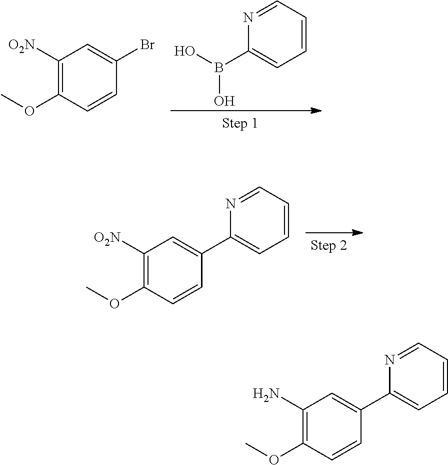

### Step 1

Potassium carbonate (2.95 g, 21.7 mmol) was added to a stirred solution of compound 4-bromo-1-methoxy-2-nitrobenzene (1.0 g, 4.34 mmol) and compound pyridin-2-ylboronic acid (0.636 g, 4.77 mmol) in toluene-ethanol (21 mL, 1:1 v/v). The reaction mixture was purged with argon for 10 min and added Pd(PPh₃)₄ (0.150 g, 0.130 mmol). The mixture was purged again with argon for 10 min. The reaction mixture was heated to 100° C. for 16 h. After consumption of starting materials (monitored by TLC), reaction mixture was cooled to room temperature and filtered through celite bed. The solvent was concentrated under reduced pressure to get crude compound. The crude product was purified by flash chromatography (silica gel 230-400 mesh; 0-10% MeOH in DCM) to get 0.1 g of compound 2-(4-methoxy-3-nitrophenyl)pyridine.

Yield: (0.1 g, 10.8%).

ES-MS [M+H]⁺: 230.94; Rt=1.85 min (Method-B).

### Step 2

To a solution of compound 2-(4-methoxy-3-nitrophenyl)pyridine (0.1 g, 0.431 mmol) in methanol (10 mL) was purged with argon for 10 min. To the reaction mixture was added 10% palladium on carbon (100 mg). The reaction mixture was hydrogenated under balloon pressure for 2 h till the completion of reaction (monitored by TLC). The reaction mixture was filtered through celite bed. The solvent was concentrated under reduced pressure to get crude compound 7.5-3. The crude product was taken as such for next stage.

Yield: (0.1 g, crude).

ES-MS [M+H]⁺: 201.08; Rt=0.77 min (Method-A).

### Example 62

### N-(2-hydroxy-5-(pyridin-2-yl)phenyl)pentanamide

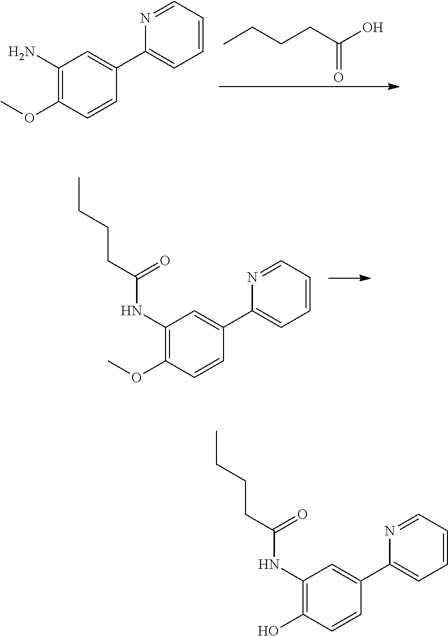

N-(2-methoxy-5-(pyridin-2-yl)phenyl)pentanamide was synthesized following the procedure described for Example 42 and isolated as a light brown solid.

Yield: (0.06 g, 42%).

ES-MS [M+H]⁺: 285.02; Rt=1.66 min (Method-B).

Sequentially, to a stirred solution of N-(2-methoxy-5-(pyridin-2-yl)phenyl)pentanamide (0.06 g, 0.211 mmol) in DCM (10 mL) was added BBr₃ 1M solution in tetrahydrofurane (1 mL) at 0° C. The reaction was warmed to room temperature and stirred for 2 h (till the completion of reaction, monitored by TLC). The reaction mixture was quenched with ice cold water and solid was filtered off, washed with saturated solution of natrium bicarbonate and dried under vacuum to give product N-(2-hydroxy-5-(pyridin-2-yl)phenyl)pentanamide as an off-white solid.

Yield: (0.02 g, 35%).

ES-MS [M+H]⁺: 271.16; Rt=1.53 min (Method-B).

¹H NMR (400 MHz, DMSO-d₆): δ 10.14 (s, 1H), 9.35 (s, 1H), 8.59-8.58 (m, 1H), 8.48-8.47 (d, J=2 Hz, 1H), 7.83-7.76 (m, 2H), 7.69-7.66 (m, 1H), 7.27-7.23 (m, 1H), 6.95-6.93 (d, J=8.4 Hz, 1H), 2.44-2.40 (m, 2H), 1.62-1.55 (m, 2H), 1.39-1.33 (m, 2H), 0.92-0.89 (m, 3H).

### Intermediate 13

### 2-methoxy-5-{6-(pyrrolidin-1-yl) pyridazin-3-yl}aniline

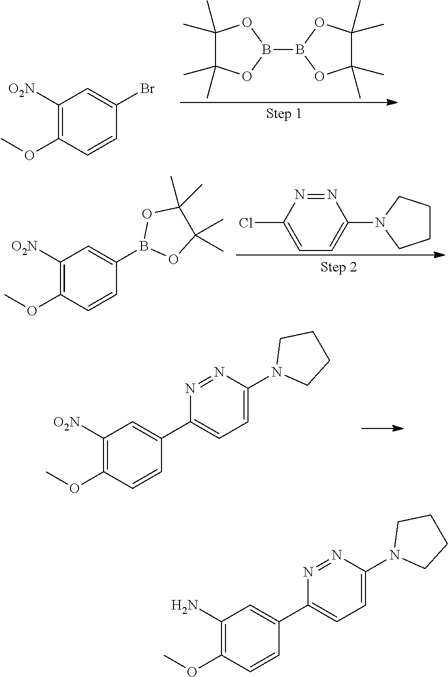

### Step 1

Potassium acetate (3.8 g, 0.0448 mmol) was added to a stirred solution of compound 4-bromo-1-methoxy-2-nitrobenzene (2.0 g, 0.0078 mmol) and bis(pinacolato)diborane (3.9 g, 0.0157 mmol) in 1,4-dioxane (60 mL). The reaction mixture was purged with argon for 10 min and added Pd(dppf)Cl₂ (0.172 g, 0.00023 mmol). The mixture was purged again with argon for 10 min. The reaction mixture was heated to 100° C. for 16 h. After consumption of starting materials (monitored by TLC), reaction mixture was cooled to room temperature and filtered through celite bed. The solvent was concentrated under reduced pressure to get crude compound 3-(4-methoxy-3-nitrophenyl-4,4,5,5-tetramethyl-1,3,2-dioxaborolane. The crude product was taken as such for next stage.

Yield: (2.5 g, crude).

ES-MS [M+H]⁻: 280; Rt=3.40 min (Method-B).

### Step 2

Potassium carbonate (2.47 g, 0.179 mmol) was added to a stirred solution of compound 3-(4-methoxy-3-nitrophenyl-4,4,5,5-tetramethyl-1,3,2-dioxaborolane (2.5 g, 0.089 mmol) and compound 3-chloro-6-(pyrrolidin-1-yl)pyridazine (1.6 g, 0.089 mmol) in 1,4 dioxane-ethanol-water (21 mL, 1:1:0.1 v/v/v). The reaction mixture was purged with argon for 10 min and added Pd(PPh₃)₄ (0.310 g, 0.0027 mmol). The mixture was purged again with argon for 10 min. The reaction mixture was heated to 100° C. for 16 h. After consumption of starting materials (monitored by TLC), reaction mixture was cooled to room temperature and filtered through celite bed. The solvent was concentrated under reduced pressure to get crude compound which was purified by flash chromatography (silica gel 230-400 mesh; 4-6% methanol in DCM) to get compound 3-(4-methoxy-3-nitrophenyl)-6-(pyrrolidin-1-yl)pyridazine as a pale yellow solid.

Yield: (1.0 g, 41%).

ES-MS [M+H]⁺: 301; Rt=1.46 min (Method-B).

¹H NMR (400 MHz, DMSO-d₆): δ 8.49 (s, 1H), 8.33-8.30 (m, 1H), 7.99-7.96 (d, J=9.2 Hz, 1H), 7.62-7.54 (m, 3H), 7.448-7.46 (d, J=9.2 Hz, 1H), 6.97-6.94 (m, 1H), 3.98 (s, 3H), 3.52-3.49 (m, 4H), 2.01-1.97 (m, 4H).

### Step 3

To a solution of compound 3-(4-methoxy-3-nitrophenyl)-6-(pyrrolidin-1-yl)pyridazine (0.5 g, 0.018 mmol) in methanol (10 mL) was purged with argon for 10 min and added 10% palladium on carbon (200 mg). The reaction mixture was stirred under hydrogenated atmosphere for 16 h. The reaction mixture was filtered through celite bed. The solvent was concentrated under reduced pressure to get crude compound 2-methoxy-5-{6-(pyrrolidin-1-yl) pyridazin-3-yl}aniline. The crude product was taken as such for next step.

Yield: (400 mg, 88.8%).

ES-MS [M+H]⁺: 271.17; Rt=1.23 min (Method-B).

### Example 63

### N-(2-hydroxy-5(6-(pyrrolidin-1-yl) pyridazin-3-yl)phenyl)pentanamide

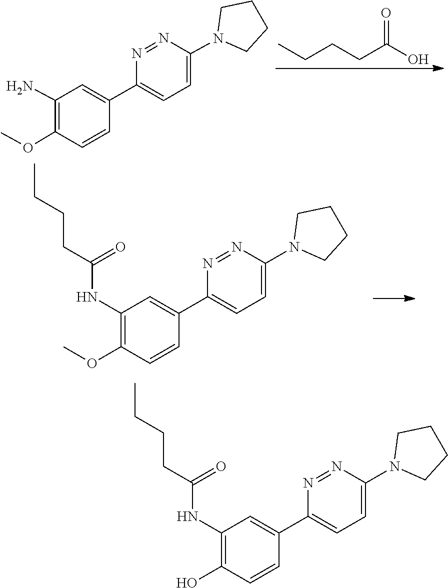

HATU (1.125 g, 2.962 mmol) was added to a suspension of compound 2-methoxy-5-{6-(pyrrolidin-1-yl) pyridazin-3-yl}aniline (0.40 g, 1.48 mmol), pentanoic acid (0.181 g, 1.77 mmol) and diisopropylethylamine (0.72 mL, 4.44 mmol) in DMF (5 mL) at 0° C. The reaction mixture was warmed to room temperature and stirred for 16 h. The reaction mixture was quenched with saturated natrium bicarbonate solution, the organic product was extracted with DCM (3×25 mL). The combined organic extracts were washed with water, brine, dried over anhydrous sodium sulfate and solvent was evaporated under reduced pressure to get crude product. The crude product was purified by column chromatography (silica gel 230-400 mesh, 2-4% methanol in DCM as eluent) to get the product N-(2-methoxy-5(6-(pyrrolidin-1-yl) pyridazin-3-yl)phenyl)pentanamide as an off-white solid.

Yield: (0.4 g, 76%).

ES-MS [M+H]⁻: 355; Rt=1.67 min (Method-B).

Sequentially, to a stirred solution of compound N-(2-methoxy-5(6-(pyrrolidin-1-yl) pyridazin-3-yl)phenyl)pentanamide (0.4 g, 0.11 mmol) in DCM (10 mL) was added BBr₃ 1M solution in tetrahydrofurane (2.2 mL, 0.22 mmol) at 0° C. The reaction was warmed to room temperature and stirred for 2 h till the completion of reaction (monitored by TLC). After completion of the reaction, ice cold water was added and solid was filtered off, washed with saturated natrium bicarbonate solution and dried under vacuum to get N-(2-hydroxy-5(6-(pyrrolidin-1-yl) pyridazin-3-yl)phenyl)pentanamide as an off-white solid.

Yield: (0.150 mg, 40%).

ES-MS [M+H]⁺: 341.25; Rt=1.50 min (Method-B).

¹H NMR (400 MHz, DMSO-d₆): δ 9.13 (br s, 1H), 8.52-8.51 (d, J=2.4 Hz, 1H), 7.55-7.53 (d, J=9.2 Hz, 1H), 7.31-7.28 (m, 1H), 6.82-6.80 (d, J=9.6 Hz, 1H), 6.36-6.34 (m, 1H), 3.47-3.43 (m, 4H), 2.33-2.30 (m, 2H), 1.98-1.95 (m, 4H), 1.59-1.55 (m, 2H), 1.33-1.31 (m, 2H), 0.92-0.88 (t, J=7.2 Hz, 3H).

### Intermediate 14

### N-(5-amino-2-hydroxyphenyl)pentanamide

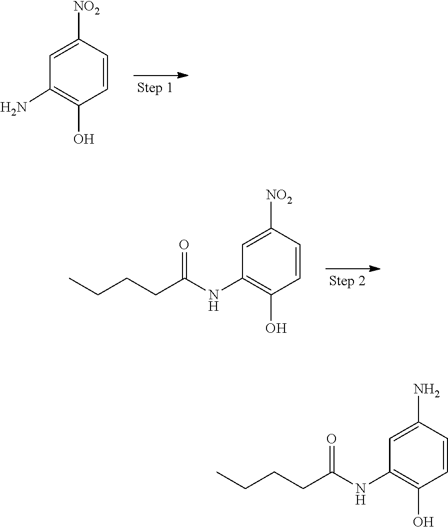 [SMILES: Nc1cc([N+](=O)[O-])ccc1O ; CCCCC(=O)Nc1cc([N+](=O)[O-])ccc1O ; CCCCC(=O)Nc1cc(N)ccc1O]

### Step 2

HATU (7.40 g, 19.47 mmol) was added to a suspension compound 2-amino-4-nitrophenol (1.5 g, 9.74 mmol), pentanoic acid (1.19 g, 11.66 mmol) and diisopropylethylamine (8.91 mL, 48.70 mmol) in DCM (20 mL) at 0° C. The reaction mixture was warmed to room temperature and stirred for 16 h. The reaction mixture was quenched with saturated natrium bicarbonate solution, the organic product was extracted with DCM (3×25 mL). The combined organic extracts were washed with water, brine, dried over anhydrous sodium sulfate and solvent was evaporated under reduced pressure to get crude product. The crude product was purified by column chromatography (silica gel 230-400 mesh, 2-4% methanol in DCM as eluent) to get the product N-(2-hydroxy-5-nitrophenyl)pentanamide as an off-white solid.

Yield: (1.0 g, 43%).

ES-MS [M−H]*: 237.08; Rt=2.012 min (Method-C).

### Step 2

10% Pd on carbon (200 mg) was added to a solution of compound N-(2-hydroxy-5-nitrophenyl)pentanamide (0.380 g, 1.59 mmol) in methanol under argon atmosphere. The hydrogen gas was purged to a reaction for 2 h until the completion of reaction. The reaction was filtered through celite bed. The solvent was concentrated under reduced pressure to get crude compound N-(5-amino-2-hydroxyphenyl)pentanamide. The crude product was taken as such for next step.

Yield: (0.3 g, crude).

ES-MS [M+H]⁺:209.08; Rt=1.34 min (Method-B).

### Example 64

### 3-chloro-N-(4-hydroxy-3-pentanamidophenyl)benzamide

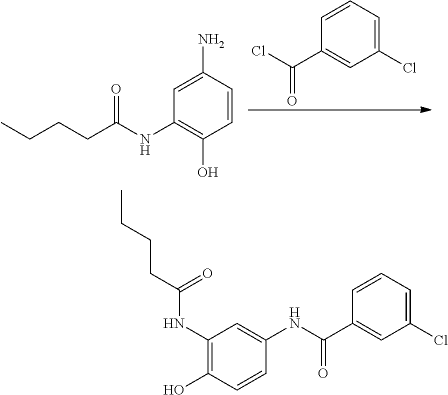 [SMILES: CCCCC(=O)Nc1cc(N)ccc1O ; O=C(Cl)c1cccc(Cl)c1 ; CCCCC(=O)Nc1cc(NC(=O)c2cccc(Cl)c2)ccc1O]

3-Chlorobenzene-1-sulfonyl chloride, (0.24 mL, 1.6 mmol) was added to a stirred solution of N-(5-amino-2-hydroxyphenyl)pentanamide (0.300 g, 1.79 mmol) in pyridine (10 mL) at 0° C. then the reaction mixture was stirred at room temperature for 16 h. The reaction mixture was quenched with minimum amount of aqueous natrium bicarbonate solution, the organic product was extracted with DCM (3×25 mL). The combined organic extracts were dried over anhydrous sodium sulfate. Solvent was distilled under reduced pressure to give the crude compound. The crude product was purified by column chromatography (silica gel 230-400 mesh, 2-4% methanol in DCM as eluent) to get the product 3-chloro-N-(4-hydroxy-3-pentanamidophenyl)benzamide.

Yield: (76 mg).

ES-MS [M−H]⁺:345.14; Rt=2.13 min (Method-C).

¹H NMR (400 MHz, DMSO-d₆): δ 10.16 (s, 1H), 9-10 (br s, 1H), 9.28 (s, 1H), 8.10 (s, 1H), 7.99-7.98 (m, 1H), 7.91-7.89 (m, 1H), 6.65-7.64 (m, 1H), 7.56-7.52 (t, J=8 Hz, 1H), 7.38-7.35 (m, 1H), 6.83-6.81 (d, J=8.4 Hz, 1H), 2.35-2.45 (t, 2H), 1.59-1.55 (m, 2H), 1.36-1.30 (m, 2H), 0.92-0.88 (t, J=7.6 Hz, 3H).

### Example 65

### N-(5-(3-chlorophenylsulfonamido)-2-hydroxyphenyl)pentanamide

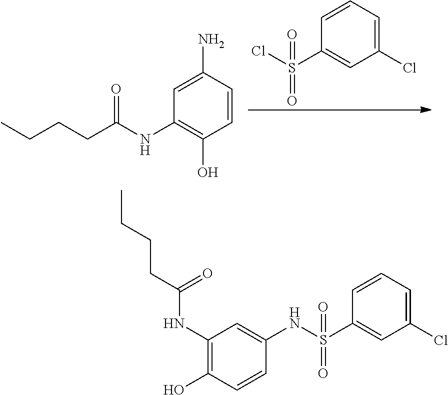 [SMILES: CCCCC(=O)Nc1cc(N)ccc1O ; O=S(=O)(Cl)c1cccc(Cl)c1 ; CCCCC(=O)Nc1cc(NS(=O)(=O)c2cccc(Cl)c2)ccc1O]

Compound N-(5-amino-2-hydroxyphenyl)pentanamide (0.3 g, 1.44 mmol) was added in pyridine (5 mL). The reaction mass was stirred cooled to 0° C. and then 3-chlorobenzene-1-sulfonyl chloride (0.344 g, 1.58 mmol) was added. The reaction mixture was stirred for 3 h. The reaction mixture was quenched with saturated natrium bicarbonate solution, the organic product was extracted with DCM (3×25 mL). The combined organic extracts were washed with water, brine, dried over anhydrous sodium sulfate and solvent was evaporated under reduced pressure to get crude product. The crude product was purified by column chromatography (silica gel 230-400 mesh, 2-4% methanol in DCM as eluent) to get the product N-(5-(3-chlorophenylsulfonamido)-2-hydroxyphenyl)pentanamide.

Yield: (60 mg).

ES-MS [M+H]⁺:383.14; Rt=2.02 min (Method-C).

¹H NMR (400 MHz, DMSO-d₆): δ 9.89 (s, 1H), 9.72 (s, 1H), 9.13 (s, 1H), 7.69-7.67 (m, 2H), 7.62-7.53 (m, 3H), 6.71-6.69 (d, J=8 Hz, 1H), 6.62-6.60 (dd, J=11 Hz, 1H), 2.37-2.33 (t, J=7.2 Hz, 2H), 1.55-1.51 (m, 2H), 1.32-1.26 (m, 2H), 0.90-0.86 (t, J=7.2 Hz, 3H).

### Intermediate 15

### N-(5-amino-2-chlorophenyl)pentanamide

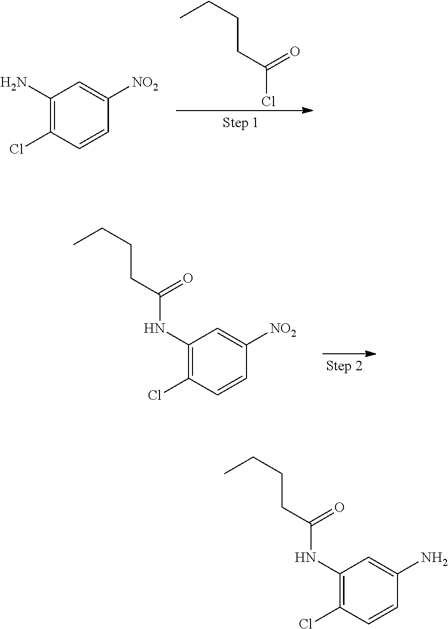

### Step 1

A mixture of 3-chloro-6-nitropyridin-2-amine (500 mg, 2.88 mmol), pentanoyl chloride (381 mg, 3.16 mmol) and pyridine (340 mg, 4.31 mmol) in DCM (20 mL) was stirred at 25° C. for 12 h. The mixture was extracted with ethyl acetate (3×50 mL). The combined organic extracts were washed with Brine (30 mL), dried over anhydrous natrium sulphate and evaporated under reduced pressure and purified by Biotage (Petroleum ether:Ethyl acetate=8:1) to give N-(3-chloro-6-nitropyridin-2-yl)pentanamide as a yellow solid.

Yield: (690 mg, 88.2%).

ES-MS [M+H]⁺: 257.0 (Method-F).

¹H NMR (400 MHz, DMSO-d₆): δ 9.80 (1H, s), 8.72 (1H, d, J=2.8 Hz), 8.02-7.99 (1H, dd, J=2.8 Hz, 8.8 Hz), 7.80 (1H, d, J=9.2 Hz), 2.48 (2H, t, J=7.6 Hz), 1.64-1.57 (2H, m), 1.40-1.31 (2H, m), 0.91 (3H, t, J=7.2 Hz).

### Step 2

To the solution of N-(2-chloro-5-nitrophenyl)pentanamide (450 mg, 1.75 mmol) in ethanol (25 mL) was added Raney-nickel (513 mg, 8.75 mmol) and the resulting mixture was stirred for 12 h under hydrogen. The mixture was filtered through the celite, the filtrate was concentrated in vacuo and purified by Biotage (petroleum ether:ethyl acetate=3:2) to give N-(5-amino-2-chlorophenyl)pentanamide as a yellow oil.

Yield: (120 mg, 29.2%).

ES-MS [M+H]⁺: 227.1 (Method-G).

¹H NMR (400 MHz, DMSO-d₆): δ 9.08 (1H, s), 7.04 (1H, d, J=8.8 Hz), 6.93 (1H, s), 6.37-6.35 (1H, dd, J=2.4 Hz, 8.4 Hz), 5.25 (2H, s), 2.32 (2H, t, J=7.2 Hz), 1.60-1.53 (2H, m), 1.38-1.28 (2H, m), 0.90 (3H, t, J=7.2 Hz).

### Example 66

### 3-chloro-N-(4-chloro-3-pentanamidophenyl)benzamide

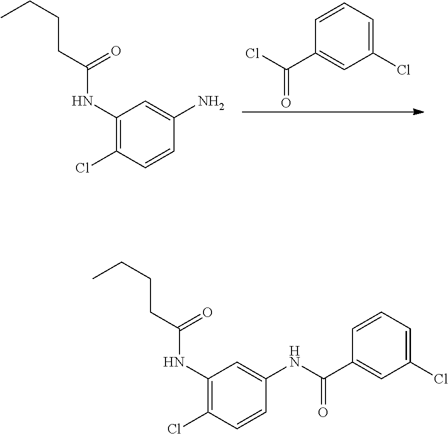 [SMILES: CCCCC(=O)Nc1cc(N)ccc1Cl ; O=C(Cl)c1cccc(Cl)c1 ; CCCCC(=O)Nc1cc(NC(=O)c2cccc(Cl)c2)ccc1Cl]

A mixture of N-(5-amino-2-chlorophenyl)pentanamide (80 mg, 0.352 mmol), 3-chlorobenzoyl chloride (67.7 mg, 0.387 mmol) and pyridine (83 mg, 1.05 mmol) in tetrahydrofurane (10 mL) was stirred at 25° C. for 3 h. The mixture was extracted with ethyl acetate (3×25 mL). The combined organic extracts were washed with Brine (25 mL), dried over anhydrous natrium sulphate and solvent was evaporated under reduced pressure to get crude product. The crude product was purified by preparative-HPLC (ammonium bicarbonate/water/acetonitrile) to give 3-chloro-N-(4-chloro-3-pentanamidophenyl)-benzamide as a white solid.

Yield: (60.5 mg, 47.2%).

ES-MS [M+H]⁺: 365.1 (Method-H).

¹H NMR (400 MHz, DMSO-d₆): δ 10.47 (1H, s), 9.46 (1H, s), 8.13 (1H, d, J=2.4 Hz), 8.02 (1H, t, J=2.0 Hz), 7.93-7.91 (1H, m), 7.70-7.66 (1H, m), 7.58 (1H, d, J=8.0 Hz), 7.46 (1H, d, J=8.8 Hz), 2.39 (2H, t, J=7.2 Hz), 1.63-1.56 (2H, m), 1.40-1.31 (2H, m), 0.92 (3H, t, J=7.2 Hz).

### Intermediate 16

### N-(5-amino-2-(trifluoromethyl)phenyl)pentanamide

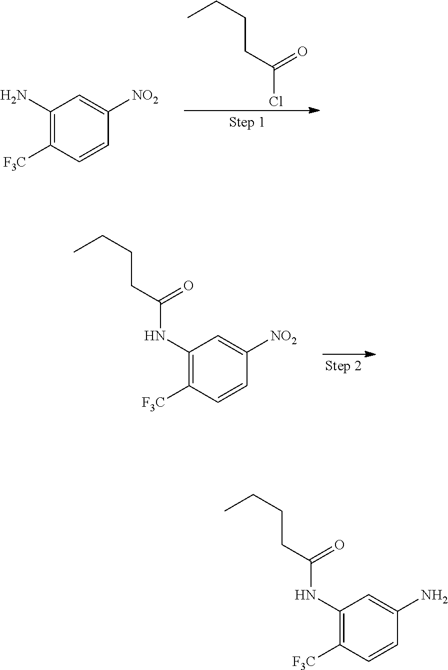

### Step 1

A mixture of 5-nitro-2-(trifluoromethyl)aniline (170 mg, 0.82 mmol) and pyridine (148 mg, 1.23 mmol) in DCM (4 mL) was added a solution of pentanoyl chloride (129 mg, 1.64 mmol) in DCM (2 mL) at 0° C. After the mixture was stirred at room temperature overnight, the mixture was concentrated and purified by silica gel column chromatography (petroleum ether:ethyl acetate=73:27) to give N-(5-nitro-2-(trifluoromethyl)-phenyl)pentanamide as a white solid.

Yield: (0.21 g, 87.8%).

ES-MS [M+H]⁺: 291.1 (Method-E).

¹H NMR (400 MHz, DMSO-d₆): δ 9.84 (1H, s), 8.37 (1H, s), 8.21 (1H, d, J=8.8 Hz), 8.04 (1H, d, J=8.8 Hz), 2.41 (2H, t, J=7.2 Hz), 1.60-1.57 (2H, m). 1.36-1.31 (2H, m), 0.90 (3H, t, J=7.2 Hz).

### Step 2

A mixture of N-[5-nitro-2-(trifluoromethyl)phenyl]pentanamide (210 mg, 0.72 mmol) and Raney Niquel (50 mg) in methanol (20 mL) was stirred under H₂ at room temperature for 6 hrs. The mixture was filtered to remove Raney Niquel. The filtrate was concentrated and purified by silica gel column chromatography (petroleum ether:ethyl acetate=1:1) to give N-(5-amino-2-(trifluoromethyl)phenyl)pentanamide as a yellow solid.

Yield: (0.15 g, 79.7%).

ES-MS [M+H]⁺: 261.0 (Method-E).

¹H NMR (400 MHz, DMSO-d₆): δ 9.13 (1H, s), 7.27 (1H, d, J=8.4 Hz), 6.56 (1H, s), 6.48 (1H, d, J=8.4 Hz), 5.83 (2H, s), 2.26 (2H, t, J=6.8 Hz), 1.55-1.52 (2H, m), 1.34-1.28 (2H, m), 0.89 (3H, t, J=7.2 Hz).

### Example 67

### 3-chloro-N-(3-pentanamido-4-(trifluoromethyl)phenyl)benzamide

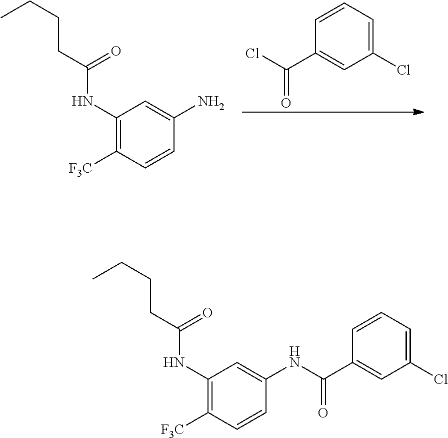 [SMILES: CCCCC(=O)Nc1cc(N)ccc1C(F)(F)F ; O=C(Cl)c1cccc(Cl)c1 ; CCCCC(=O)Nc1cc(NC(=O)c2cccc(Cl)c2)ccc1C(F)(F)F]

A mixture of N-[5-amino-2-(trifluoromethyl)phenyl]pentanamide (150 mg, 0.57 mmol) and pyridine (136 mg, 1.72 mmol) in DCM (8 mL) was added 3-chlorobenzoyl chloride (151 mg, 864 μmol) in DCM (3 mL) at 0° C. After the mixture was stirred at room temperature for 3 hrs, the mixture was concentrated and purified by PREP-HPLC (high pH) to give 3-chloro-N-(3-pentanamido-4-(trifluoromethyl)phenyl)benzamide as a white solid.

Yield: (0.0944 g, yield 41.2%).

ES-MS [M+H]⁺: 399.1 (Method-E).

¹H NMR (400 MHz, DMSO-d₆): δ 10.67 (1H, s), 9.54 (1H, s), 8.04 (1H, s), 7.94-7.90 (3H, m), 7.71 (2H, t, J=9.2 Hz), 7.59 (1H, t, J=8.0 Hz), 2.34 (2H, t, J=6.8 Hz), 1.60-1.56 (2H, m), 1.37-1.32 (2H, m), 0.81 (3H, t, J=7.6 Hz).

### Intermediate 17

### 2. N-(5-amino-2-fluorophenyl)pentanamide

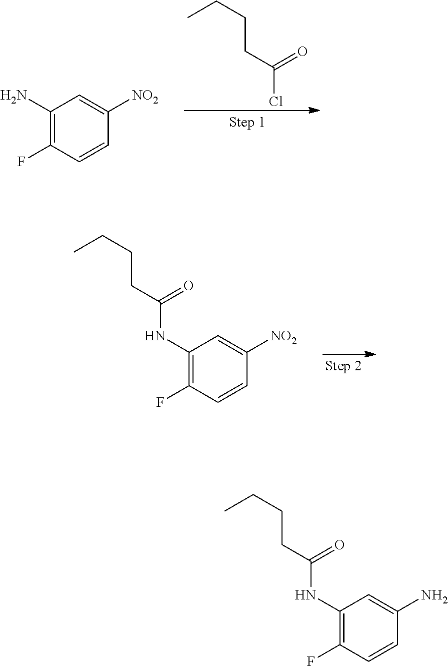

### Step 1

A mixture of 2-fluoro-5-nitroaniline (468 mg, 3.0 mmol) and pyridine (474 mg, 6.0 mmol) in DCM (15 mL) was added a solution of pentanoyl chloride (540 mg, 4.5 mmol) in DCM (2 mL) at 0° C. After the mixture was stirred at room temperature overnight, the mixture was dissolved in DCM (100 mL). The organic layer was washed with water and dried over anhydrous sodium sulfate and concentrated. The residue was purified by silica gel column chromatography (petroleum ether:ethyl acetate=90:10) to give N-(2-fluoro-5-nitrophenyl)pentanamide as a white solid.

Yield: (500 mg, 69.4%).

ES-MS [M+H]⁺: 241.1 (Method-E).

¹H NMR (400 MHz, DMSO-d₆): δ 10.12 (1H, s), 9.00-8.98 (1H, m), 8.03-8.01 (1H, m). 7.55 (1H, t, J=10 Hz), 2.50-2.43 (2H, m). 1.60-1.56 (2H, m), 1.36-1.30 (2H, m). 0.92-0.86 (3H, m).

### Step 2

A mixture of N-(2-fluoro-5-nitrophenyl)pentanamide (500 mg, 2.08 mmol) and Raney Niquel (50 mg) in methanol (40 mL) was stirred under hydrogen at room temperature for 5 h. The mixture was filtered to remove Raney Niquel. The filtrate was concentrated and purified by silica gel column (petroleum ether:ethyl acetate=35:65) to afford N-(5-amino-2-fluorophenyl)pentanamide as a white solid.

Yield: (350 mg, 80%).

ES-MS [M+H]⁺: 211.1 (Method-E).

¹H NMR (400 MHz, DMSO-d₆): δ 9.32 (1H, s), 7.10-7.08 (1H, m), 6.86-6.81 (1H, m). 6.27-6.24 (1H, m). 4.92 (2H, s), 2.32 (2H, t, J=6.8 Hz), 1.56-1.50 (2H, m). 1.33-1.28 (2H, m), 0.88 (3H, t, J=6.8 Hz).

### Example 68

### 3-chloro-N-(4-fluoro-3-pentanamidophenyl)benzamide

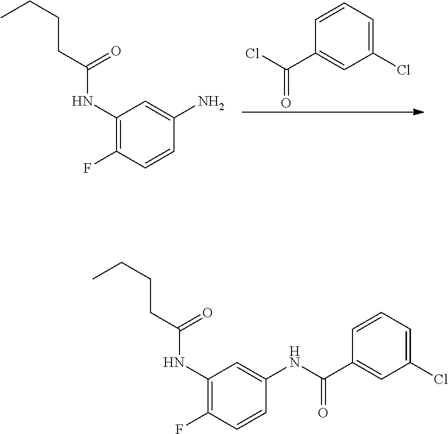 [SMILES: CCCCC(=O)Nc1cc(N)ccc1F ; O=C(Cl)c1cccc(Cl)c1 ; CCCCC(=O)Nc1cc(NC(=O)c2cccc(Cl)c2)ccc1F]

A mixture of N-(5-amino-2-fluorophenyl)pentanamide (106 mg, 0.504 mmol) and pyridine (79.1 mg, 1.00 mmol) in DCM (4 mL) was added a solution of 3-chlorobenzoyl chloride (132 mg, 756 μmol) in DCM (1 mL) at 0° C. After the mixture was stirred at room temperature overnight, the mixture was concentrated and purified by preparative-HPLC (high pH) to give 3-chloro-N-(4-fluoro-3-pentanamidophenyl)benzamide as a white solid.

Yield: (64.1 mg, 36.6%).

ES-MS [M+H]⁺: 349.1 (Method-E).

¹H NMR (400 MHz, DMSO-d₆): δ 10.4 (1H, s), 9.68 (1H, s), 8.28-8.26 (1H, m), 8.01 (1H, s), 7.92-7.90 (1H, m). 7.68-7.65 (1H, m), 7.61-7.55 (2H, m), 7.25-7.20 (1H, m), 2.38 (2H, t, J=7.6 Hz), 1.60-1.54 (2H, m). 1.36-1.31 (2H, m), 0.90 (3H, t, J=7.6 Hz).

### Intermediate 18

### 3-amino-N-(3-chlorophenyl)-4-methoxybenzamide

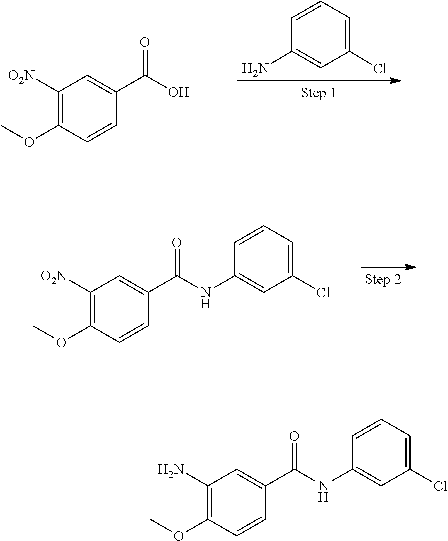 [SMILES: COc1ccc(C(=O)O)cc1[N+](=O)[O-] ; Nc1cccc(Cl)c1 ; COc1ccc(C(=O)Nc2cccc(Cl)c2)cc1[N+](=O)[O-] ; COc1ccc(C(=O)Nc2cccc(Cl)c2)cc1N]

### Step 1

HATU (5.7 g, 15.2 mmol) was added portion wise to a solution of DIPEA (5.23 mL, 30.4 mmol) and compound 4-methoxy-3-nitrobenzoic acid (2.0 g, 10.1 mmol) in DCM (30 mL) at 0° C. The reaction mixture was stirred for 10 min at same temperature. Then, 3-chloroaniline (1.4 g, 11.1 mmol) was added to the reaction mixture at 0° C. and the reaction mixture was stirred at room temperature for 16 h. The reaction mixture was quenched with minimum amount of aqueous natrium bicarbonate solution, the organic product was extracted with DCM (2×25 mL). The combined organic extracts were dried over anhydrous sodium sulfate. Solvent was distilled under reduced pressure to give the crude compound. The crude product was purified by column chromatography (100-200 silica) using 50-60% ethyl acetate in petroleum ether as eluent to get N-(3-chlorophenyl)-4-methoxy-3-nitrobenzamide as an off-white solid.

Yield: (1.75 g, 56%).

ES-MS [M+H]⁺: 307.08; Rt=2.23 min (Method-C).

### Step 2

Acetic acid (1.75 mL, 1.0 v.) was added slowly to a suspension of Iron (Fe) powder (3.1 g, 57.1 mmol) and compound N-(3-chlorophenyl)-4-methoxy-3-nitrobenzamide (1.75 g, 5.7 mmol, 1.0 eq) in ethanol (10 mL) and tetrahydrofurane (10 mL) at room temperature. The reaction mixture was heated at 80° C. for 16 h. The reaction mixture was filtered through celite and filtrate was evaporated under reduced pressure to get pure compound 3-amino-N-(3-chlorophenyl)-4-methoxybenzamide as a thick liquid.

Yield: (1.2 g, 76%).

ES-MS [M+H]⁺: 277.03; Rt=1.86 min (Method-C).

### Example 69

### N-(3-chlorophenyl)-4-hydroxy-3-pentanamidobenzamide

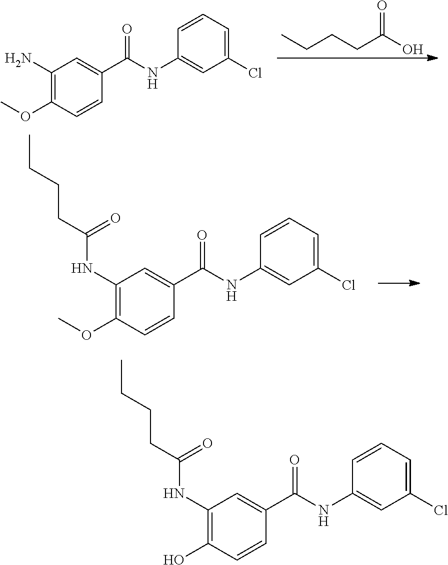

HATU (0.165 g, 0.4 mmol) was added portion wise to a solution of DIPEA (0.12 mL, 0.7 mmol) and compound 3-amino-N-(3-chlorophenyl)-4-methoxybenzamide (0.1 g, 0.36 mmol) in DMF (2 mL) at 0° C. The reaction mixture was stirred for 10 minutes at same temperature. Then pentanoic acid (0.045 g, 0.4 mmol) was added to the reaction mixture at 0° C. then, the reaction mixture was stirred at room temperature for 16 h. The reaction mixture was quenched with minimum amount of aqueous natrium bicarbonate solution, the organic product was extracted with ethyl acetate (2×10 mL). The combined organic extracts were dried over anhydrous sodium sulfate. Solvent was distilled under reduced pressure to give the crude compound N-(3-chlorophenyl)-4-methoxy-3-pentanamidobenzamide. The crude product was used for next as such without any purification.

Yield: (0.08 g, Crude).

ES-MS [M+H]⁺: 359.24; Rt=2.19 min (Method-C).

Sequentially, BBr₃ (1 M in DCM; 1.1 mL, 1.1 mmol) was added slowly to a solution of compound N-(3-chlorophenyl)-4-methoxy-3-pentanamidobenzamide (0.08 g, 0.22 mmol) in DCM (2 mL) at 0° C. The reaction mixture was stirred for 16 h at room temperature. The reaction mixture was quenched with minimum amount of aqueous natrium bicarbonate solution, the organic product was extracted with DCM (2×5 mL). The combined organic extracts were dried over anhydrous sodium sulfate. Solvent was distilled under reduced pressure to give the crude compound. The crude product was purified by column chromatography (100-200 silica) using 2-3% methanol in DCM as eluent to get N-(3-chlorophenyl)-4-hydroxy-3-pentanamidobenzamide as an off-white solid.

Yield: (0.020 g).

ES-MS [M+H]⁺: 345.22; Rt=2.08 min (Method-C).

¹H NMR (400 MHz, DMSO-d₆): δ 10.50 (s, 1H), 10.18 (s, 1H), 9.33 (s, 1H), 8.33-8.33 (d, J=2 Hz, 1H), 7.94-7.93 (t, J=4 Hz, 1H), 7.69-7.67 (m, 1H), 7.63-7.60 (d, J=4 Hz, 1H), 7.37-7.33 (t, J=8 Hz, 1H), 7.13-7.11 (m, 1H), 6.96-6.94 (d, J=8 Hz, 1H), 2.43-2.39 (t, J=8 Hz, 2H), 1.60-1.56 (m, 2H), 1.36-1.31 (m, 2H), 0.92-0.88 (t, J=8 Hz, 3H).

### Intermediate 19

### 6-(7H-pyrrolo[2,3-d]pyrimidin-7-yl)pyridin-2-amine

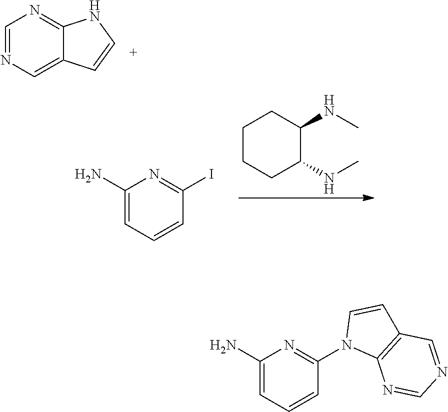

A mixture of 7H-pyrrolo[2,3-d]pyrimidine (100 mg, 0.839 mmol), 6-iodopyridin-2-amine (200 mg, 1.00 mmol), lambda1-copper(1+) iodide (15.9 mg, 0.0839 mmol), tripotassium phosphate (390 mg, 1.84 mmol) and (1R,2R)—N1,N2-dimethylcyclohexane-1,2-diamine (23.7 mg, 0.167 mmol) in 1,4-dioxane was replaced the air with Argon and stirred at 100° C. for 12 h. It was concentrated, the residue was purified by silica gel column (petroleum ether:ethyl acetate=1:1) to give 6-(7H-pyrrolo[2,3-d]pyrimidin-7-yl)pyridin-2-amine as a white solid.

Yield: (0.070 g, 33.2%).

ES-MS [M+H]⁺: 212.1 (Method-E).

¹H NMR (400 MHz, MeOD-d₄): δ 9.02 (1H, s), 8.74 (1H, s), 8.38 (1H, d, J=8.0 Hz), 7.87 (1H, d, J=7.6 Hz), 7.63 (1H, t, J=8.0 Hz), 6.81 (1H, d, J=4.0H), 6.52 (1H, t, J=7.6 Hz).

### Example 70

### (E)-N-(6-(7H-pyrrolo[2,3-d]pyrimidin-7-yl)pyridin-2-yl)-3-(furan-2-yl)acrylamide

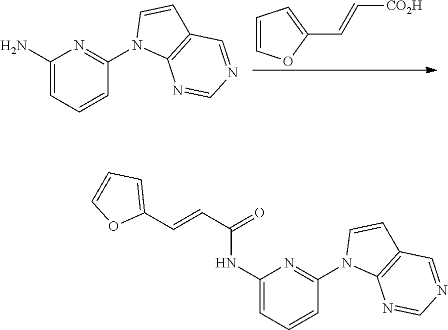

A solution of (2E)-3-(furan-2-yl)prop-2-enoic acid (214 mg, 1.55 mmol), HATU (589 mg, 1.55 mmol), and pyridine (0.2 mL) in NMP (10 mL) was stirred at room temperature for 10 min. It was added 6-{7H-pyrrolo[2,3-d]pyrimidin-7-yl}pyridin-2-amine (55 mg, 0.260 mmol) and stirred in microwave at 120° C. for 2 h. It was purified with reverse phase column (0.01% ammonia and ammonium bicarbonate in water and acetonitrile) to give (E)-N-(6-(7H-pyrrolo[2,3-d]pyrimidin-7-yl)pyridin-2-yl)-3-(furan-2-yl)acrylamide as a white solid.

Yield: (19.9 mg, 25.2%).

ES-MS [M+H]⁺: 332.1 (Method-E).

¹H NMR (400 MHz, DMSO-d₆): δ 10.9 (1H, s), 9.18 (1H, s), 8.99 (1H, s), 8.43 (1H, d, J=7.6 Hz), 8.38 (1H, d, J=4.0 Hz), 8.19 (1H, d, J=8.4 Hz), 8.08 (1H, t, J=8.0 Hz), 7.87 (1H, s), 7.49 (1H, d, J=15.6 Hz), 6.96 (1H, d, J=4.0H), 6.91 (1H, d, J=3.2 Hz), 6.85 (1H, d, J=15.6 Hz), 6.66-6.45 (1H, m).

### Intermediate 20

### 4-(7H-pyrrolo[2,3-d]pyrimidin-7-yl)pyridin-2-amine

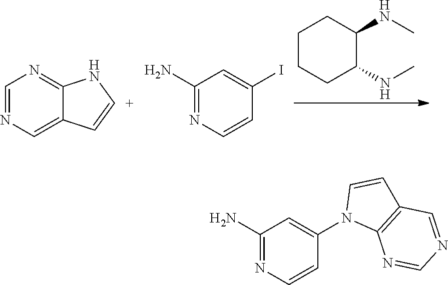

A mixture of 7H-pyrrolo[2,3-d]pyrimidine (300 mg, 2.5 mmol), 4-iodopyridin-2-amine (832 mg, 3.78 mmol), lambda1-copper(1+) iodide (48.0 mg, 0.25 mmol), tripotassium phosphate (1.16 g, 5.5 mmol) and (1R,2R)—N1,N2-dimethylcyclohexane-1,2-diamine (71.0 mg, 0.5 mmol) in 1,4-dioxane was replaced the air with argon and was stirred at 100° C. for 12 h. It was concentrated and purified by silica gel column (DCM:ethyl acetate=2:1,) to give 4-(7H-pyrrolo[2,3-d]pyrimidin-7-yl)pyridin-2-amine (494 mg, 93.6%) as a white solid.

Yield: (0.494 g, 93.6%).

ES-MS [M+H]⁺: 212.1 (Method-E).

¹H NMR (400 MHz, DMSO-d₆): δ 9.15 (1H, s), 8.94 (1H, s), 8.09-8.06 (2H, m), 7.26 (1H, s), 7.10-7.08 (1H, m), 6.90 (1H, t, J=4.0 Hz), 6.25 (2H, s).

### Example 71

### (E)-N-(4-(7H-pyrrolo[2,3-d]pyrimidin-7-yl)pyridin-2-yl)-3-(furan-2-yl)acrylamide

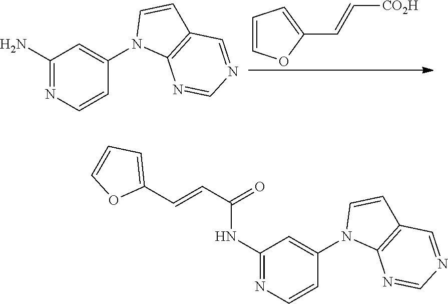

A solution of (2E)-3-(furan-2-yl)prop-2-enoic acid (392 mg, 2.80 mmol), HATU (1.06 g, 2.8 mmol), diisopropylethylamine (361 mg, 200 mg) and pyridine (0.2 mL) in NMP (10 mL) was stirred at room temperature for 10 min. It was added 6-{7H-pyrrolo[2,3-d]pyrimidin-7-yl}pyridin-2-amine (100 mg, 0.47 mmol). The mixture was stirred in microwave at 120° C. for 2 h. It was purified with reverse phase column (C18, 0.01% ammonia and ammonium bicarbonate in water and acetonitrile) to give (E)-N-(4-(7H-pyrrolo[2,3-d]pyrimidin-7-yl) pyridin-2-yl)-3-(furan-2-yl)acrylamide as a white solid.

Yield: (68.6 mg, 44.1%).

ES-MS [M+H]⁺: 332.1 (Method-E).

¹H NMR (400 MHz, DMSO-d₆): δ 10.9 (1H, s), 9.18 (1H, s), 9.05 (1H, d, J=2.0 Hz), 8.97 (1H, s), 8.49 (1H, d, J=5.2 Hz), 8.18 (1H, d, J=4.0 Hz), 7.86 (1H, d, J=1.6 Hz), 7.80 (1H, dd, J=2.0, 2.0 Hz), 7.49 (1H, d, J=15.2 Hz), 6.98 (1H, d, J=3.6H), 6.90 (1H, d, J=3.6 Hz), 6.85 (1H, d, J=15.6 Hz), 6.65 (1H, dd, J=1.6, 2.0 Hz).

### Intermediate 21

### 3-amino-N-(furan-2-ylmethyl)isonicotinamide

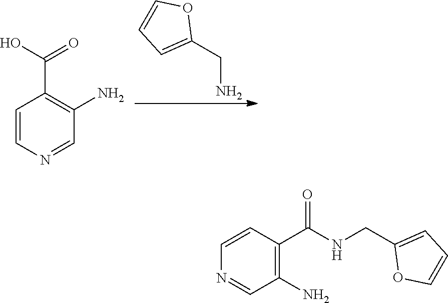 [SMILES: Nc1cnccc1C(=O)O ; NCc1ccco1 ; Nc1cnccc1C(=O)NCc1ccco1]

A mixture of 3-aminopyridine-4-carboxylic acid (690 mg, 4.99 mmol), 1-(furan-2-yl)methanamine (580 mg, 5.98 mmol), HATU (570 mg, 1.5 mmol) and ethylbis(propan-2-yl)amine (1.92 g, 14.9 mmol) in dimethylformamide (5 mL) was stirred at room temperature overnight. The mixture was purified preparative-HPLC (high pH) to give 3-amino-N-(furan-2-ylmethyl)isonicotinamide as a yellow oil.

Yield: (300 mg, 27.7%).

ES-MS [M+H]⁺: 218.1 (Method-E).

¹H NMR (400 MHz, DMSO-d₆): δ 9.01-8.98 (1H, m), 8.13 (1H, s), 7.73 (1H, d, J=5.2 Hz), 7.57 (1H, s), 7.38 (1H, d, J=5.2 Hz), 6.49 (1H, s), 6.40-6.39 (1H, m), 6.28-6.27 (1H, m), 4.42 (2H, d, J=5.6 Hz).

### Example 72

### N-(furan-2-ylmethyl)-3-(naphthalene-2-sulfonamido)isonicotinamide

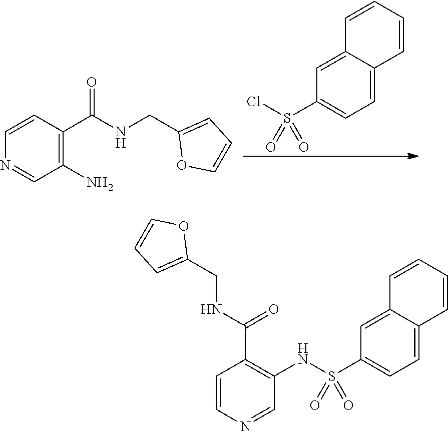

A mixture of 3-amino-N-[(furan-2-yl)methyl]pyridine-4-carboxamide (300 mg, 1.38 mmol) and naphthalene-2-sulfonyl chloride (405 mg, 1.79 mmol) in pyridine (1 mL) was irradiated in the microwave on a Biotage Smith Synthesis at 100° C. for 4 h. The mixture was concentrated and the residue was purified by preparative-HPLC (high pH) to afford N-(furan-2-ylmethyl)-3-(naphthalene-2-sulfonamido)isonicotinamide as a white solid.

Yield: (91.7 mg, 16.3%).

ES-MS [M+H]⁺: 408.1 (Method-E).

¹H NMR (400 MHz, DMSO-d₆): δ 11.03 (1H, brs), 9.55 (1H, brs), 8.70 (1H, s), 8.48 (1H, s), 8.33 (1H, brs) 8.15-8.13 (1H, m), 8.03-8.01 (2H, m), 7.72-7.64 (3H, m), 7.60-7.57 (2H, m), 6.42-6.40 (1H, m), 6.28-6.27 (1H, m), 4.36-4.35 (2H, m).

### Intermediate 22

### 4-(naphthalene-2-sulfonamido)nicotinic acid

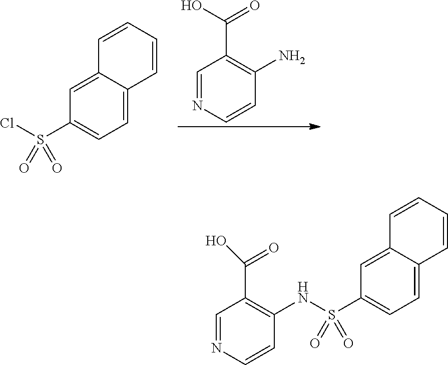

A solution of naphthalene-2-sulfonyl chloride (393 mg, 1.74 mmol), 4-aminonicotinic acid (200 mg, 1.0 μmol) and diisopropylethylamine (935 mg, 7.25 mmol) in ethanol/water=3:1 (12 mL) was stirred in microwave at 85° C. for 3 h. It was concentrated and purified with reverse phase column (C18, 0.01% ammonia and Ammonium bicarbonate in water and acetonitrile) to give 4-(naphthalene-2-sulfonamido)nicotinic acid as a solid.

Yield: (139 mg, 42.3%).

ES-MS [M+H]⁺: 329.1 (Method-E).

¹H NMR (400 MHz, DMSO-d₆): δ 8.75 (1H, s), 8.53 (1H, s), 8.17-8.12 (2H, m), 8.04 (1H, d, J=8.8 Hz), 8.00-7.98 (1H, m), 7.81 (1H, dd, J=1.6, 2.0 Hz), 7.68-7.61 (2H, m), 7.33 (1H, d, J=6.0 Hz).

### Example 73

### N-(furan-2-ylmethyl)-4-(naphthalene-2-sulfonamido)nicotinamide

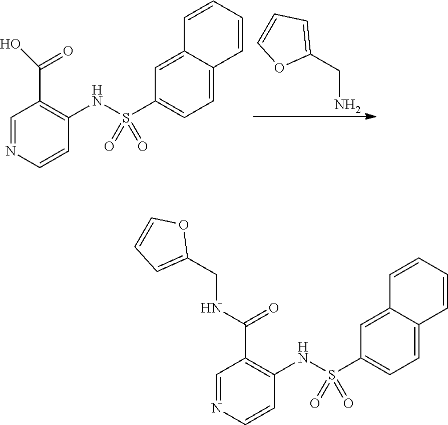

A solution of 4-(naphthalene-2-sulfonamido)nicotinic acid (139 mg, 0.42 mmol), HATU (192 mg, 0.51 mmol) and diisopropylethylamine (163 mg, 1.26 mmol) in dimethylformamide (10 ml) was stirred at room temperature for 10 mins. To the solution was added 1-(furan-2-yl)methanamine (62 mg, 0.64 mmol) and stirred at room temperature overnight. It was purified with reverse phase column (C18, 0.01% ammonia and ammonium bicarbonate in water and acetonitrile) to give N-(furan-2-ylmethyl)-4-(naphthalene-2-sulfonamido)nicotinamide as a white solid.

Yield: (55.4 mg, 16.5%).

ES-MS [M+H]⁺: 408.2 (Method-F).

¹H NMR (400 MHz, DMSO-d₆): δ 10.9 (1H, brs), 8.76 (1H, s), 8.43 (1H, m), 8.16-8.09 (2H, m), 7.98 (3H, d, J=8.0 Hz), 7.75-7.73 (1H, m), 7.66-7.60 (2H, m), 7.50 (1H, s), 7.38 (1H, s), 6.27 (2H, d, J=23.2H), 4.51 (2H, d, J=6.0 Hz).

### Intermediate 23

### 3-(naphthalene-2-sulfonamido)picolinic acid

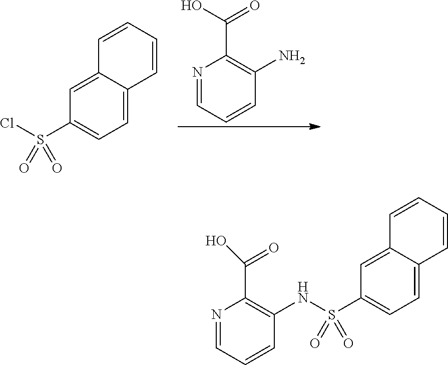

A solution of naphthalene-2-sulfonyl chloride (339 mg, 1.50 mmol), 3-aminonicotinic acid (138 mg, 1.0 mmol) and diisopropylethylamine (774 mg, 6.0 mmol) in ethanol/water=3:1 (12 mL) was in microwave at 85° C. stirred for 3 h. It was concentrated and purified with reverse phase column (C18, 0.01% ammonia and ammonium bicarbonate in water and acetonitrile) to give 3-(naphthalene-2-sulfonamido)picolinic acid as an oil.

Yield: (310 mg, crude).

ES-MS [M+H]⁺: 329.1 (Method-F).

### Example 74

### N-(furan-2-ylmethyl)-3-(naphthalene-2-sulfonamido)picolinamide

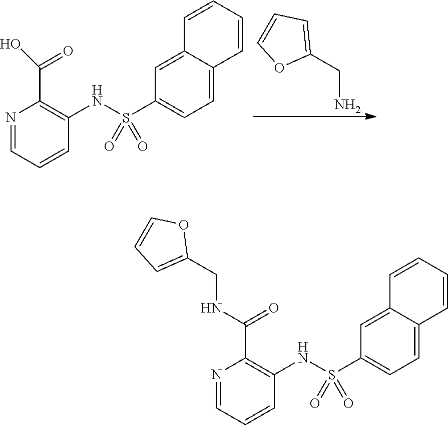

A solution of 3-(naphthalene-2-sulfonamido)picolinic acid (74.6 mg, 0.22 mmol, 22% purity), HATU (83.6 mg, 0.44 mmol) and diisopropylethylamine (85.1 mg, 0.66 mmol) in dimethylformamide (5 mL) was stirred at room temperature for 10 min. To the reaction mixture was added 1-(furan-2-yl)methanamine (42.7 mg, 0.44 mmol) and stirred at room temperature overnight. It was purified with reverse phase column (C18, 0.01% ammonia and ammonium bicarbonate in water and acetonitrile) to give N-(furan-2-ylmethyl)-3-(naphthalene-2-sulfonamido)picolinamide as a white solid.

Yield: (14.4 mg, 16.0%).

ES-MS [M+H]⁺: 408.1 (Method-E).

¹H NMR (400 MHz, DMSO-d₆): δ 12.3 (1H, s), 9.59 (1H, s), 8.59 (1H, s), 8.24 (1H, d, J=9.2 Hz), 8.15 (1H, d, J=7.2 Hz), 8.07-8.00 (3H, m), 7.77 (1H, dd, J=1.6, 2.0 Hz), 7.73-7.65 (2H, m), 7.57 (2H, t, J=12.0 Hz), 6.39 (1H, dd, J=1.6, 2.0 Hz), 6.23 (1H, s), 4.44 (2H, d, J=6.0 Hz).

### Intermediate 24

### 2-((2-(naphthalen-2-ylamino)-2-oxoethyl)amino)benzoic acid

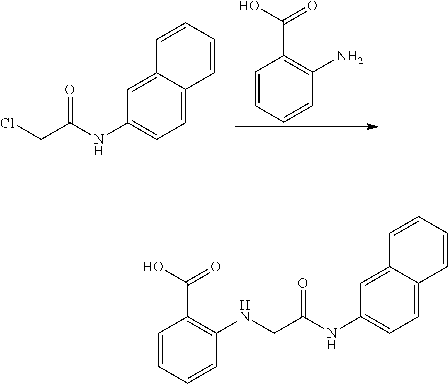

To a solution of 2-chloro-N-(naphthalen-2-yl)acetamide (657 mg, 3.00 mmol) and 2-aminobenzoic acid (452 mg, 3.3 mmol) in ethanol (10 mL) was added N,N-Diisopropylethylamine (1.55 g, 12.0 mmol). The reaction mixture was stirred for 3 h at 100° C. in microwave. The reaction mixture was concentrated in vacuo. The residue was purified with reverse phase column (C₁₈, acetonitrile/water (FA)) to give 2-((2-(naphthalen-2-ylamino)-2-oxoethyl)amino)benzoic acid as a brown solid.

Yield: (115 mg, 11.9%).

ES-MS [M+H]⁺: 321.1 (Method-E).

¹H NMR (400 MHz, DMSO-d₆): δ 12.6 (1H, s), 10.5 (1H, s), 8.33 (2H, s), 7.89-7.81 (4H, m), 7.65-7.62 (1H, m), 7.50-7.46 (1H, m), 7.43-7.38 (2H, m), 6.65-6.60 (2H, m), 4.15 (2H, s).

### Example 75

### N-benzyl-2-((2-(naphthalen-2-ylamino)-2-oxoethyl)amino)benzamide

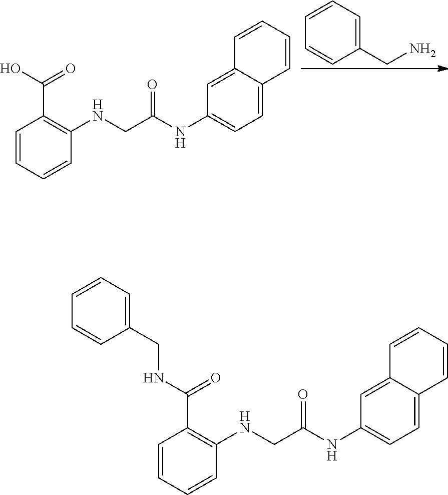

A mixture of 2-((2-(naphthalen-2-ylamino)-2-oxoethyl)amino)benzoic acid (30 mg, 94 μmol), HATU (41.8 mg, 110 μmol) and DIPEA (36 mg, 280 μmol) in DMF (5 mL) was stirred at room temperature for 10 min. To the solution was added phenylmethanamine (15.0 mg, 140 μmol). The mixture was stirred at room temperature overnight and purified with reverse phase column (C18, acetonitrile/water (ammonium bicarbonate)) to give N-benzyl-2-((2-(naphthalen-2-ylamino)-2-oxoethyl)amino)benzamide as a white solid.

Yield: (15.7 mg, 40.8%).

ES-MS [M+H]⁺: 410.1 (Method-E).

¹H NMR (400 MHz, MeOD-d₄): δ 8.22 (1H, d, J=2.0 Hz), 7.83-7.77 (3H, m), 7.60-7.57 (2H, m), 7.47-7.34 (7H, m), 7.28 (1H, d, J=7.2 Hz), 6.76-6.69 (2H, m), 4.61 (2H, s), 4.09 (2H, s).

### Example 76

### 2-((2-(naphthalen-2-ylamino)-2-oxoethyl)amino)-N-(pyridin-3-ylmethyl)benzamide

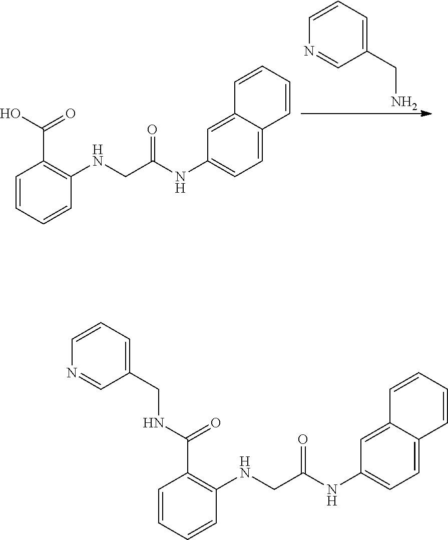

A mixture of 2-((2-(naphthalen-2-ylamino)-2-oxoethyl)amino)benzoic acid (35 mg, 110 μmol), HATU (46.0 mg, 120 μmol) and DIPEA (42.6 mg, 330 μmol) in DMF (5 ml) was stirred at room temperature for 10 mins. To the solution was added pyridin-3-ylmethanamine (17.3 mg, 160 μmol). The mixture was stirred at room temperature overnight and purified with reverse phase column (C₁₈, acetonitrile/water (ammonium bicarbonate)) to give 2-((2-(naphthalen-2-ylamino)-2-oxoethyl)amino)-N-(pyridin-3-ylmethyl)benzamide as a white solid.

Yield: (25.1 mg, 55.3%).

ES-MS [M+H]⁺: 411.2 (Method-E).

¹H NMR (400 MHz, DMSO-d₆): δ 10.4 (1H, s), 9.02 (1H, s), 8.68 (1H, s), 8.57 (1H, d, J=4.8 Hz), 8.32 (2H, s), 7.98 (1H, d, J=8.0 Hz), 7.89-7.80 (3H, m), 7.67 (1H, d, J=7.6 Hz), 7.61-7.56 (2H, m), 7.48 (1H, t, J=7.2 Hz), 7.42 (1H, t, J=8.0 Hz), 7.35-7.32 (1H, m), 6.66-6.61 (2H, m), 4.53 (2H, d, J=5.6 Hz), 4.06 (2H, s).

### Intermediate 25

### Naphthalene-2-sulfonamide

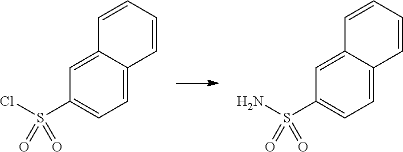 [SMILES: O=S(=O)(Cl)c1ccc2ccccc2c1 ; NS(=O)(=O)c1ccc2ccccc2c1]

A mixture of naphthalene-2-sulfonyl chloride (226 mg, 1.0 mmol) and ammonium hydroxide (424 mg, 4 mmol, 33% in water) in DCM (10 mL) was stirred at room temperature overnight. After the mixture was concentrated under vacuum, the residue was added to water. The solid precipitated was filtered and dried to give naphthalene-2-sulfonamide as a white solid.

Yield: (173 mg, 83.6%).

ES-MS [M+H]⁺: 208.1 (Method-E).

¹H NMR (400 MHz, DMSO-d₆): δ 8.44 (1H, s), 8.14 (2H, t, J=7.6 Hz), 8.04 (1H, t, J=7.2 Hz), 7.89 (1H, dd, J=1.6, 1.6 Hz), 7.71-7.64 (2H, m), 7.46 (2H, s).

### Intermediate 26

### 2-fluoro-N-(furan-2-ylmethyl)nicotinamide

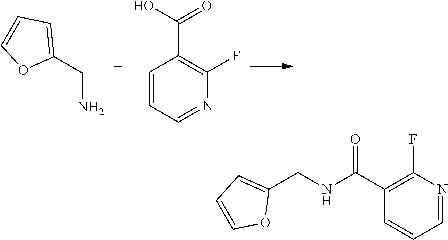 [SMILES: NCc1ccco1 ; O=C(O)c1cccnc1F ; O=C(NCc1ccco1)c1cccnc1F]

A mixture of 2-fluoronicotinic acid (100 mg, 710 μmol), HATU (269 mg, 780 μmol) and DIPEA (2374 mg, 2.13 mmol) in DMF (5 mL) was stirred at room temperature for 10 mins. To the solution was added furan-2-ylmethanamine (69.0 mg, 710 μmol). The mixture was stirred at room temperature overnight and purified with reverse phase column (C₁₈, acetonitrile/water (ammonium bicarbonate)) to give 2-fluoro-N-(furan-2-ylmethyl)nicotinamide as a white solid.

Yield: (97.0 mg, 62.1%).

ES-MS [M+H]⁺: 221.1 (Method-E).

¹H NMR (400 MHz, DMSO-d₆): δ 9.00 (1H, s), 8.35 (1H, d, J=5.2 Hz), 8.19-8.14 (1H, m), 7.61 (1H, d, J=0.8 Hz), 7.48-7.44 (1H, m), 7.43-6.42 (1H, m), 6.31 (1H, d, J=2.4 Hz), 4.76 (2H, d, J=6.0 Hz).

### Example 77

### N-(furan-2-ylmethyl)-2-(naphthalene-2-sulfonamido)nicotinamide

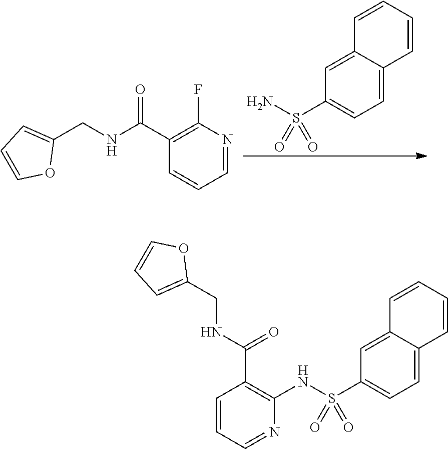 [SMILES: O=C(NCc1ccco1)c1cccnc1F ; NS(=O)(=O)c1ccc2ccccc2c1 ; O=C(NCc1ccco1)c1cccnc1NS(=O)(=O)c1ccc2ccccc2c1]

A mixture of 2-fluoro-N-(furan-2-ylmethyl)nicotinamide (77 mg, 350 μmol), naphthalene-2-sulfonamide (73 mg, 350 μmol) and cessium carbonate (341 mg, 1.05 mmol) in dioxane (7 mL) was stirred at 110° C. overnight. The mixture was added to water and extracted with ethyl acetate (4×50 mL). The organic layer was dried over anhydrous sodium sulfate and concentrated in vacuo. The residue was purified by silicagel chromatography (DCM:methanol=20:1) and reverse phase column (C18, acetonitrile/water (FA)) to give 2-fluoro-N-(furan-2-ylmethyl)nicotinamide as a white solid.

Yield: (24.6 mg, 17.3%).

ES-MS [M+H]⁺: 408.1 (Method-E).

¹H NMR (400 MHz, MeOD-d₄): δ 8.56 (1H, s), 8.40-8.29 (1H, m), 8.10-7.93 (5H, m), 7.67-7.59 (2H, m), 7.36 (1H, s), 6.95 (1H, t, J=6.0 Hz), 6.26 (2H, d, J=19.2 Hz), 4.54 (2H, s).

### Examples 78-84 Having Formula (IA)

The following Examples 78-84 were purchased and tested in the assay as described below. These compounds were obtained from Specs (the Netherlands). The test results are provided in Table 5 below.

### Example 78

### N-(furan-2-ylmethyl)-2-(2-methylbenzamido)benzamide

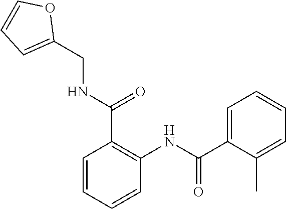 [SMILES: Cc1ccccc1C(=O)Nc1ccccc1C(=O)NCc1ccco1]

### Example 79

### N-(2-(diethylamino)ethyl)-2-(3-(3-fluorophenyl)ureido)benzamide

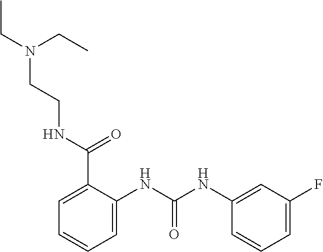 [SMILES: CCN(CC)CCNC(=O)c1ccccc1NC(=O)Nc1cccc(F)c1]

### Example 80

### 2-benzamido-N-((tetrahydrofuran-2-yl)methyl)benzamide

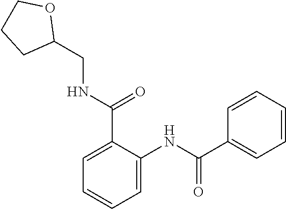 [SMILES: O=C(Nc1ccccc1C(=O)NCC1CCCO1)c1ccccc1]

### Example 81

### N-allyl-2-(4-methylbenzamido)benzamide

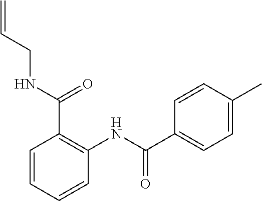 [SMILES: C=CCNC(=O)c1ccccc1NC(=O)c1ccc(C)cc1]

### Example 82

### N-(2-(pyridin-4-ylcarbamoyl)phenyl)furan-2-carboxamide

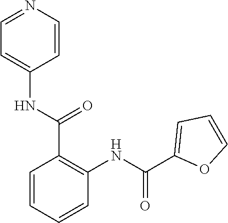 [SMILES: O=C(Nc1ccccc1C(=O)Nc1ccncc1)c1ccco1]

### Example 83

### 2-(3-methylbenzamido)-N-(2-morpholinoethyl)benzamide

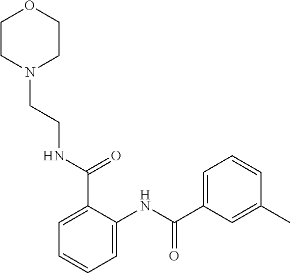 [SMILES: Cc1cccc(C(=O)Nc2ccccc2C(=O)NCCN2CCOCC2)c1]

### Example 84

### N-(2-(benzylcarbamoyl)phenyl)furan-2-carboxamide

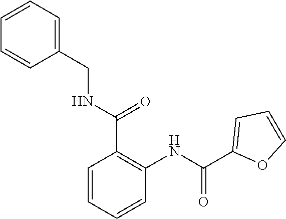 [SMILES: O=C(Nc1ccccc1C(=O)NCc1ccccc1)c1ccco1]

### Biological Assays

Compounds of the Disclosure are capable of binding allosterically to galactocerebrosidase enzyme thereby stabilizing the enzyme against denaturation and are expected to enhance its catalytic activity.

### Differential Scanning Fluorimetry (DSF).

The capacity of the Compounds of the Disclosure to stabilize galactocerebrosidase was assessed by differential scanning fluorimetry technique. The thermal denaturation of purified human native enzyme was monitored in the presence of the extrinsic fluorescent probe SYPRO Orange (Sigma-Aldrich, St. Louis, Mo.). Compounds were dissolved in 100% DMSO and diluted into the protein buffer to achieve final concentrations of 1% DMSO.

Galactocerebrosidase pure protein (two sources: gift from Chiesi and R&D Systems commercial supplier) 12.5 microl of 1.5 μM in 50 mM Hepes 100 mM NaCl pH 7.06 (final concentration 0.75 μM) with Sypro Orange 20× and 12.5 μl of the different compound solutions were dispensed into 96-well PCR-plates (LightCycler480 Multiwell Plate 96, Roche Diagnostics).

Plates were loaded into a LightCycler 480 System II (Roche Applied Science, Indianapolis) for thermal denaturation. The increase in SYPRO Orange fluorescence intensity associated with protein unfolding (λexcitation=465 nm, λemission=580 nm) was monitored as a measure of thermal denaturation. Unfolding curves were recorded from 20 to 95° C., at a scan rate of 2° C./min. The experimental unfolding curves were smoothed, normalized, and analyzed using in-house software. The melting temperature (Tm) was calculated as the temperature at which half the protein is in the unfolded state. ΔTm is calculated as the value of Tm of the protein in the presence of compound substrating the value of Tm in the absence of compound.

Compounds of the Disclosure were tested in one of the recombinant protein available or in both of them, and their activity is referred to one and/or both of the proteins.

The capacity to stabilize galactocerebrosidase against denaturation at 30 μM is denoted as follows:
ΔTm GALC>1 is shown as A
ΔTm GALC between 0.5 and 1 is shown as B
ΔTm GALC between 0.1 and 0.5 is shown as C
ND means not determined

TABLE 1
Assay results for commercially available
Examples 1-28 having formula (IA)

| Example # | Range |
|---|---|
| 1 | C |
| 2 | C |
| 3 | C |
| 4 | C |
| 5 | B |
| 6 | C |
| 7 | C |
| 8 | B |
| 9 | A |
| 10 | B |
| 11 | B |
| 12 | A |
| 13 | C |
| 14 | C |
| 15 | C |
| 16 | C |
| 17 | B |
| 18 | C |
| 19 | B |
| 20 | A |
| 21 | A |
| 22 | B |
| 23 | B |
| 24 | B |
| 25 | A |
| 26 | A |
| 27 | C |
| 28 | C |

TABLE 2
Assay results for commercially available
Examples 29-41 having formula (IB)

| Example # | Range |
|---|---|
| 29 | B |
| 30 | B |
| 31 | A |
| 32 | C |
| 33 | C |
| 34 | A |
| 35 | A |
| 36 | C |
| 37 | C |
| 38 | B |
| 39 | B |
| 40 | B |
| 41 | C |

TABLE 3
Assay results for synthetized Examples 42-71 having formula (IB)

| Example # | Range |
|---|---|
| 42 | B |
| 43 | A |
| 44 | A |
| 45 | A |
| 46 | B |
| 47 | A |
| 48 | A |
| 49 | A |
| 50 | C |
| 51 | B |
| 52 | ND |
| 53 | ND |
| 54 | B |
| 55 | C |
| 56 | C |
| 57 | C |
| 58 | C |
| 59 | C |
| 60 | B |
| 61 | B |
| 62 | A |
| 63 | B |
| 64 | B |
| 65 | A |
| 66 | ND |
| 67 | ND |
| 68 | ND |
| 69 | A |
| 70 | B |
| 71 | C |

TABLE 4
Assay results for synthetized Examples 72-77 having formula (IA)

| Example # | Range |
|---|---|
| 72 | B |
| 73 | A |
| 74 | B |
| 75 | ND |
| 76 | ND |
| 77 | ND |

TABLE 5
Assay results for commercially available
Examples 78-84 having formula (IA)

| Example # | Range |
|---|---|
| 78 | C |
| 79 | C |
| 80 | C |
| 81 | C |
| 82 | C |
| 83 | C |
| 84 | C |

All publications cited in this specification are incorporated herein by reference. While the disclosure has been described with reference to particular embodiments, it will be appreciated that modifications can be made without departing from the spirit of the disclosure. Such modifications are intended to fall within the scope of the appended claims.

The disclosure also relates to the following particular embodiments designated as [1] for the first embodiment, [2] for the second embodiment, and so on:

[1] A method of treating or preventing a condition associated with the alteration of the activity of galactocerebrosidase in a patient, comprising administering to the patient in need thereof an effective amount of a compound of formula (IA):

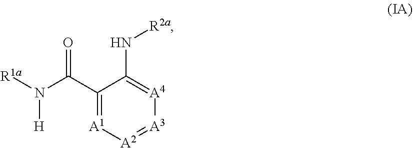

or a pharmaceutically acceptable salt or solvate thereof, wherein

A¹, A², A³, and A⁴ are each independently selected from the group consisting of N, CH and C(R^{3a});

each R^{3a} is independently selected from the group consisting of halogen, —C₁₋₄ alkyl, —C₁₋₄ alkoxy, and —CN;

R^{1a} is selected from the group consisting of —C₁₋₄ alkyl, —C₃₋₁₀ cycloalkyl, —C₁₋₄ alkyl-C₃₋₁₀ cycloalkyl, —C₆₋₁₀ aryl, —C₁₋₄ alkyl-C₆₋₁₀ aryl, -(5- to 10-membered)-C₁₋₉ heteroaryl, —C₁₋₄ alkyl-(5- to 10-membered)-C₁₋₉ heteroaryl, -(5- to 10-membered)-C₂₋₉ heterocyclyl, and —C₁₋₄ alkyl-(5- to 10-membered)-C₂₋₉ heterocyclyl, wherein said alkyl, cycloalkyl, alkylcycloalkyl, aryl, alkylaryl, heteroaryl, alkylheteroaryl, heterocyclyl and alkylheterocyclyl groups are optionally substituted with 1, 2 or 3 substituents each independently selected from the group consisting of halogen, hydroxy, CN, —ORb^{a}, —SRb^{a}, —N(Rb^{a})₂, —C₁₋₄ alkyl optionally substituted with 1, 2, or 3 halogen atoms, optionally substituted —C₆₋₁₀ aryl, optionally substituted -(5- to 10-membered)-C₁₋₉ heteroaryl, and -(5- to 10-membered)-C₂₋₉ heterocyclyl; and wherein said cycloalkyl, alkylcycloalkyl, aryl, alkylaryl, heteroaryl, alkylheteroaryl, heterocyclyl and alkylheterocyclyl is optionally fused to a further (second) ring; and

R^{2a} is selected from the group consisting of —C₁₋₄ alkyl, —C(═O)Ra^{a}, —S(═O)₂Ra^{a}, —C₁₋₄ alkyl-C(═O)Ra^{a}, —C₁₋₄ alkyl-C(═O)NHRa^{a}, —C₁₋₄ alkyl-C(═O)N(Ra^{a})₂, —C₁₋₄ alkyl-S(═O)₂Ra^{a}, —C₁₋₄ alkyl-S(═O)₂—N(Ra^{a})₂, —C₁₋₄ alkyl-C₃₋₁₀ cycloalkyl, —C₁₋₄ alkyl-C₆₋₁₀ aryl, -(5- to 10-membered)-C₁₋₉ heteroaryl, —C₁₋₄ alkyl-(5- to 10-membered)-C₁₋₉ heteroaryl, (5- to 10-membered)-C₂₋₉ heterocyclyl, and —C₁₋₄ alkyl-(5- to 10-membered)-C₂₋₉ heterocyclyl, wherein said alkyl, cycloalkyl, alkylcycloalkyl, aryl, alkylaryl, heteroaryl, alkylheteroaryl, heterocyclyl and alkylheterocyclyl groups are optionally substituted with 1, 2 or 3 substituents each independently selected from the group consisting of halogen, hydroxy, —CN, —C(═O)Ra^{a}, —ORb^{a}, —SRb^{a}, —N(Rb^{a})₂, (═O), —C₁₋₄ alkyl optionally substituted with 1, 2, or 3 halogen atoms, optionally substituted C₆₋₁₀ aryl, optionally substituted -(5- to 10-membered)-C₁₋₉ heteroaryl, and -(5- to 10-membered)-C₂₋₉ heterocyclyl; and wherein said cycloalkyl, alkylcycloalkyl, aryl, alkylaryl, heteroaryl, alkylheteroaryl, heterocyclyl and alkylheterocyclyl is optionally fused to a further (second) ring;

Ra^{a} is selected from the group consisting of —C₁₋₄ alkyl, —C₃₋₁₀ cycloalkyl, —C₁₋₄ alkyl-C₃₋₁₀ cycloalkyl, —C₆₋₁₀ aryl, —C₁₋₄ alkyl-C₆₋₁₀ aryl, -(5- to 10-membered)-C₁₋₉ heteroaryl, —C₁₋₄ alkyl-(5- to 10-membered)-C₁₋₉ heteroaryl, -(5- to 10-membered)-C₂₋₉ heterocyclyl, and —C₁₋₄ alkyl-(5- to 10-membered)-C₂₋₉ heterocyclyl, wherein said alkyl, cycloalkyl, alkylcycloalkyl, aryl, alkylaryl, heteroaryl, alkylheteroaryl, heterocyclyl and alkylheterocyclyl groups are optionally substituted with 1, 2 or 3 substituents each independently selected from the group consisting of halogen, hydroxy, —CN, —ORb^{a}, —SRb^{a}, —N(Rb^{a})₂, —C₁₋₄ alkyl optionally substituted with 1, 2, or 3 halogen atoms, optionally substituted —C₆₋₁₀ aryl, optionally substituted -(5- to 10-membered)-C₁₋₉ heteroaryl, and -(5- to 10-membered)-C₂₋₉ heterocyclyl; and wherein said cycloalkyl, alkylcycloalkyl, aryl, alkylaryl, heteroaryl, alkylheteroaryl, heterocyclyl and alkylheterocyclyl is optionally fused to a further (second) ring; and

each Rb^{a} is independently hydrogen, —C₁₋₄ alkyl, —C₃₋₁₀ cycloalkyl, or -(5- to 10-membered)-C₂₋₉ heterocyclyl, wherein said alkyl, cycloalkyl or heterocyclyl group is optionally substituted by 1, 2 or 3 fluorine atoms.

[2] A method of treating or preventing a lysosomal storage disease or an α-synucleinopathy, comprising administering to a patient in need thereof an effective amount of a compound of formula (IA):

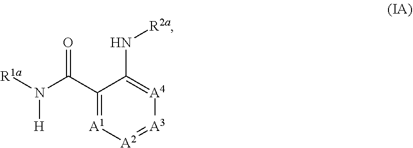

or a pharmaceutically acceptable salt or solvate thereof, wherein

A¹, A², A³, and A⁴ are each independently selected from the group consisting of N, CH and C(R^{3a});

each R^{3a} is independently selected from the group consisting of halogen, —C₁₋₄ alkyl, —C₁₋₄ alkoxy, and —CN;

R^{1a} is selected from the group consisting of —C₁₋₄ alkyl, —C₃₋₁₀ cycloalkyl, —C₁₋₄ alkyl-C₃₋₁₀ cycloalkyl, —C₆₋₁₀ aryl, —C₁₋₄ alkyl-C₆₋₁₀ aryl, -(5- to 10-membered)-C₁₋₉ heteroaryl, —C₁₋₄ alkyl-(5- to 10-membered)-C₁₋₉ heteroaryl, -(5- to 10-membered)-C₂₋₉ heterocyclyl, and —C₁₋₄ alkyl-(5- to 10-membered)-C₂₋₉ heterocyclyl, wherein said alkyl, cycloalkyl, alkylcycloalkyl, aryl, alkylaryl, heteroaryl, alkylheteroaryl, heterocyclyl and alkylheterocyclyl groups are optionally substituted with 1, 2 or 3 substituents each independently selected from the group consisting of halogen, hydroxy, CN, —ORb^{a}, —SRb^{a}, —N(Rb^{a})₂, —C₁₋₄ alkyl optionally substituted with 1, 2, or 3 halogen atoms, optionally substituted —C₆₋₁₀ aryl, optionally substituted -(5- to 10-membered)-C₁₋₉ heteroaryl, and -(5- to 10-membered)-C₂₋₉ heterocyclyl; and wherein said cycloalkyl, alkylcycloalkyl, aryl, alkylaryl, heteroaryl, alkylheteroaryl, heterocyclyl and alkylheterocyclyl is optionally fused to a further (second) ring; and

R^{2a} is selected from the group consisting of —C₁₋₄ alkyl, —C(═O)Ra^{a}, —S(═O)₂Ra^{a}, —C₁₋₄ alkyl-C(═O)Ra^{a}, —C₁₋₄ alkyl-C(═O)NHRa^{a}, —C₁₋₄ alkyl-C(═O)N(Ra^{a})₂, —C₁₋₄ alkyl-S(═O)₂Ra^{a}, —C₁₋₄ alkyl-S(═O)₂—N(Ra^{a})₂, —C₁₋₄ alkyl-C₃₋₁₀ cycloalkyl, —C₁₋₄ alkyl-C₆₋₁₀ aryl, -(5- to 10-membered)-C₁₋₉ heteroaryl, —C₁₋₄ alkyl-(5- to 10-membered)-C₁₋₉ heteroaryl, (5- to 10-membered)-C₂₋₉ heterocyclyl, and —C₁₋₄ alkyl-(5- to 10-membered)-C₂₋₉ heterocyclyl, wherein said alkyl, cycloalkyl, alkylcycloalkyl, aryl, alkylaryl, heteroaryl, alkylheteroaryl, heterocyclyl and alkylheterocyclyl groups are optionally substituted with 1, 2 or 3 substituents each independently selected from the group consisting of halogen, hydroxy, —CN, —C(═O)Ra^{a}, —ORb^{a}, —SRb^{a}, —N(Rb^{a})₂, (═O), —C₁₋₄ alkyl optionally substituted with 1, 2, or 3 halogen atoms, optionally substituted C₆₋₁₀ aryl, optionally substituted -(5- to 10-membered)-C₁₋₉ heteroaryl, and -(5- to 10-membered)-C₂₋₉ heterocyclyl; and wherein said cycloalkyl, alkylcycloalkyl, aryl, alkylaryl, heteroaryl, alkylheteroaryl, heterocyclyl and alkylheterocyclyl is optionally fused to a further (second) ring;

Ra^{a} is selected from the group consisting of —C₁₋₄ alkyl, —C₃₋₁₀ cycloalkyl, —C₁₋₄ alkyl-C₃₋₁₀ cycloalkyl, —C₆₋₁₀ aryl, —C₁₋₄ alkyl-C₆₋₁₀ aryl, -(5- to 10-membered)-C₁₋₉ heteroaryl, —C₁₋₄ alkyl-(5- to 10-membered)-C₁₋₉ heteroaryl, -(5- to 10-membered)-C₂₋₉ heterocyclyl, and —C₁₋₄ alkyl-(5- to 10-membered)-C₂₋₉ heterocyclyl, wherein said alkyl, cycloalkyl, alkylcycloalkyl, aryl, alkylaryl, heteroaryl, alkylheteroaryl, heterocyclyl and alkylheterocyclyl groups are optionally substituted with 1, 2 or 3 substituents each independently selected from the group consisting of halogen, hydroxy, —CN, —ORb^{a}, —SRb^{a}, —N(Rb^{a})₂, —C₁₋₄ alkyl optionally substituted with 1, 2, or 3 halogen atoms, optionally substituted —C₆₋₁₀ aryl, optionally substituted -(5- to 10-membered)-C₁₋₉ heteroaryl, and -(5- to 10-membered)-C₂₋₉ heterocyclyl; and wherein said cycloalkyl, alkylcycloalkyl, aryl, alkylaryl, heteroaryl, alkylheteroaryl, heterocyclyl and alkylheterocyclyl is optionally fused to a further (second) ring; and

each Rb^{a} is independently hydrogen, —C₁₋₄ alkyl, —C₃₋₁₀ cycloalkyl, or -(5- to 10-membered)-C₂₋₉ heterocyclyl, wherein said alkyl, cycloalkyl or heterocyclyl group is optionally substituted by 1, 2 or 3 fluorine atoms.

[3] The method of [2], wherein a lysosomal storage disease is treated or prevented.
[4] The method of [2] or [3], wherein the lysosomal storage disease is Krabbe's disease.
[5] The method of [2], wherein an α-synucleinopathy is treated or prevented.
[6] A method of treating or preventing a disease or disorder, comprising administering to a patient in need thereof an effective amount of a compound of formula (IA):

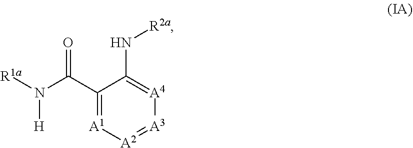

or a pharmaceutically acceptable salt or solvate thereof, wherein

A¹, A², A³, and A⁴ are each independently selected from the group consisting of N, CH and C(R^{3a});

each R^{3a} is independently selected from the group consisting of halogen, —C₁₋₄ alkyl, —C₁₋₄ alkoxy, and —CN;

R^{1a} is selected from the group consisting of —C₁₋₄ alkyl, —C₃₋₁₀ cycloalkyl, —C₁₋₄ alkyl-C₃₋₁₀ cycloalkyl, —C₆₋₁₀ aryl, —C₁₋₄ alkyl-C₆₋₁₀ aryl, -(5- to 10-membered)-C₁₋₉ heteroaryl, —C₁₋₄ alkyl-(5- to 10-membered)-C₁₋₉ heteroaryl, -(5- to 10-membered)-C₂₋₉ heterocyclyl, and —C₁₋₄ alkyl-(5- to 10-membered)-C₂₋₉ heterocyclyl, wherein said alkyl, cycloalkyl, alkylcycloalkyl, aryl, alkylaryl, heteroaryl, alkylheteroaryl, heterocyclyl and alkylheterocyclyl groups are optionally substituted with 1, 2 or 3 substituents each independently selected from the group consisting of halogen, hydroxy, CN, —ORb^{a}, —SRb^{a}, —N(Rb^{a})₂, —C₁₋₄ alkyl optionally substituted with 1, 2, or 3 halogen atoms, optionally substituted —C₆₋₁₀ aryl, optionally substituted -(5- to 10-membered)-C₁₋₉ heteroaryl, and -(5- to 10-membered)-C₂₋₉ heterocyclyl; and wherein said cycloalkyl, alkylcycloalkyl, aryl, alkylaryl, heteroaryl, alkylheteroaryl, heterocyclyl and alkylheterocyclyl is optionally fused to a further (second) ring; and

R^{2a} is selected from the group consisting of —C₁₋₄ alkyl, —C(═O)Ra^{a}, —S(═O)₂Ra^{a}, —C₁₋₄ alkyl-C(═O)Ra^{a}, —C₁₋₄ alkyl-C(═O)NHRa^{a}, —C₁₋₄ alkyl-C(═O)N(Ra^{a})₂, —C₁₋₄ alkyl-S(═O)₂Ra^{a}, —C₁₋₄ alkyl-S(═O)₂—N(Ra^{a})₂, —C₁₋₄ alkyl-C₃₋₁₀ cycloalkyl, —C₁₋₄ alkyl-C₆₋₁₀ aryl, -(5- to 10-membered)-C₁₋₉ heteroaryl, —C₁₋₄ alkyl-(5- to 10-membered)-C₁₋₉ heteroaryl, (5- to 10-membered)-C₂₋₉ heterocyclyl, and —C₁₋₄ alkyl-(5- to 10-membered)-C₂₋₉ heterocyclyl, wherein said alkyl, cycloalkyl, alkylcycloalkyl, aryl, alkylaryl, heteroaryl, alkylheteroaryl, heterocyclyl and alkylheterocyclyl groups are optionally substituted with 1, 2 or 3 substituents each independently selected from the group consisting of halogen, hydroxy, —CN, —C(═O)Ra^{a}, —ORb^{a}, —SRb^{a}, —N(Rb^{a})₂, (═O), —C₁₋₄ alkyl optionally substituted with 1, 2, or 3 halogen atoms, optionally substituted C₆₋₁₀ aryl, optionally substituted -(5- to 10-membered)-C₁₋₉ heteroaryl, and -(5- to 10-membered)-C₂₋₉ heterocyclyl; and wherein said cycloalkyl, alkylcycloalkyl, aryl, alkylaryl, heteroaryl, alkylheteroaryl, heterocyclyl and alkylheterocyclyl is optionally fused to a further (second) ring;

Ra^{a} is selected from the group consisting of —C₁₋₄ alkyl, —C₃₋₁₀ cycloalkyl, —C₁₋₄ alkyl-C₃₋₁₀ cycloalkyl, —C₆₋₁₀ aryl, —C₁₋₄ alkyl-C₆₋₁₀ aryl, -(5- to 10-membered)-C₁₋₉ heteroaryl, —C₁₋₄ alkyl-(5- to 10-membered)-C₁₋₉ heteroaryl, -(5- to 10-membered)-C₂₋₉ heterocyclyl, and —C₁₋₄ alkyl-(5- to 10-membered)-C₂₋₉ heterocyclyl, wherein said alkyl, cycloalkyl, alkylcycloalkyl, aryl, alkylaryl, heteroaryl, alkylheteroaryl, heterocyclyl and alkylheterocyclyl groups are optionally substituted with 1, 2 or 3 substituents each independently selected from the group consisting of halogen, hydroxy, —CN, —ORb^{a}, —SRb^{a}, —N(Rb^{a})₂, —C₁₋₄ alkyl optionally substituted with 1, 2, or 3 halogen atoms, optionally substituted —C₆₋₁₀ aryl, optionally substituted -(5- to 10-membered)-C₁₋₉ heteroaryl, and -(5- to 10-membered)-C₂₋₉ heterocyclyl; and wherein said cycloalkyl, alkylcycloalkyl, aryl, alkylaryl, heteroaryl, alkylheteroaryl, heterocyclyl and alkylheterocyclyl is optionally fused to a further (second) ring; and

each Rb^{a} is independently hydrogen, —C₁₋₄ alkyl, —C₃₋₁₀ cycloalkyl, or -(5- to 10-membered)-C₂₋₉ heterocyclyl, wherein said alkyl, cycloalkyl or heterocyclyl group is optionally substituted by 1, 2 or 3 fluorine atoms,

wherein said disease or disorder is selected from the group consisting of Krabbe's disease, demyelinating disorders, galactosylsphingosine related disorders, globoid cell leukodystrophy, multiple sclerosis (MS), Parkinson's disease, peripheral neuropathy, progressive multiple sclerosis, pulmonary artery enlargement in COPD, open angle glaucoma, Lewy body dementia, and multiple system atrophy (MSA).

[7] The method of any one of [1] to [6], wherein A¹, A², A³, and A⁴ are CH.
[8] The method of any one of [1] to [6], wherein one of A¹, A², A³, and A⁴ is C(R^{3a}) and the ones not C(R^{3a}) are CH.
[9] The method of any one of [1] to [6], wherein two of A¹, A², A³, and A⁴ is C(R^{3a}) and the ones not C(R^{3a}) are CH.
[10] The method of any one of [1] to [6], wherein A¹ is N and A², A³, and A⁴ are each independently selected from the group consisting of CH and C(R^{3a}).
[11] The method of any one of [1] to [6], wherein A² is N and A¹, A³, and A⁴ are each independently selected from the group consisting of CH and C(R^{3a}).
[12] The method of any one of [1] to [6], wherein A³ is N and A¹, A², and A⁴ are each independently selected from the group consisting of CH and C(R^{3a}).
[13] The method of any one of [1] to [6], wherein A⁴ is N and A¹, A², and A³ are each independently selected from the group consisting of CH and C(R^{3a}).

The method of any one of [1] to [6], wherein two of A¹, A², A³, and A⁴ are N, and those that are not N are each independently selected from the group consisting of CH and C(R^{3a}).

[15] The method of any one of [1] to [6], wherein three of A¹, A², A³, and A⁴ are N, and the one not N is selected from the group consisting of CH and C(R^{3a}).
[16] The method of any one of [1] to [6], wherein the compound of formula (IA) is a compound of formula (IIA):

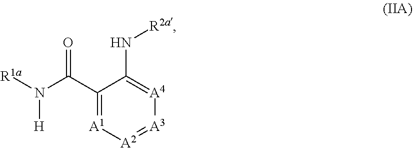

or a pharmaceutically acceptable salt or solvate thereof, wherein

A¹, A², A³, and A⁴ are each independently selected from the group consisting of N, CH and C(R^{3a}), provided that no more than one of A¹, A², A³, or A⁴ is N;

each R^{3a} is independently selected from the group consisting of halogen, —OH, C₁₋₄ alkyl, C₁₋₄ alkoxy, and CN;

R^{1a} is selected from the group consisting of —C₁₋₄ alkyl, —C₃₋₁₀ cycloalkyl, —C₁₋₄ alkyl-C₃₋₁₀ cycloalkyl, —C₆₋₁₀ aryl, —C₁₋₄ alkyl-C₆₋₁₀ aryl, (5- to 10-membered)-C₁₋₉ heteroaryl, —C₁₋₄ alkyl-(5- to 10-membered)-C₁₋₉ heteroaryl, (5- to 10-membered)-C₂₋₉ heterocyclyl, and —C₁₋₄ alkyl-(5- to 10-membered)-C₂₋₉ heterocyclyl, wherein said alkyl, cycloalkyl, alkylcycloalkyl, aryl, alkylaryl, heteroaryl, alkylheteroaryl, heterocyclyl and alkylheterocyclyl groups are optionally substituted with 1, 2 or 3 substituents each independently selected from the group consisting of halogen, hydroxo, —CN, —ORb^{a}, —SRb^{a}, —N(Rb^{a})₂, —C₁₋₄ alkyl optionally substituted with 1, 2, or 3 halogen atoms, optionally substituted C₆₋₁₀ aryl, optionally substituted (5- to 10-membered)-C₁₋₉ heteroaryl, and (5- to 10-membered)-C₂₋₉ heterocyclyl; and wherein said cycloalkyl, alkylcycloalkyl, aryl, alkylaryl, heteroaryl, alkylheteroaryl, heterocyclyl and alkylheterocyclyl is optionally fused to a further (second) ring;

R^{2a′} is selected from the group consisting of —C(═O)Ra^{a′}, —S(═O)₂Ra^{a′}, —C₁₋₄ alkyl-C(═O)NHRa^{a′}, —C₁₋₄ alkyl-C(═O)N(Ra^{a′})₂, —C₁₋₄ alkyl-S(═O)₂—N(Ra^{a′})₂, wherein said alkyl group is optionally substituted with 1, 2 or 3 substituents each independently selected from the group consisting of halogen, hydroxy, —CN, —C₁₋₄ alkyl optionally substituted with 1, 2, or 3 halogen atoms;

Ra^{a′} is selected from the group consisting of —C₆₋₁₀ aryl, —C₁₋₄ alkyl-C₆₋₁₀ aryl, (5- to 10-membered)-C₁₋₉ heteroaryl, —C₁₋₄ alkyl-(5- to 10-membered)-C₁₋₉ heteroaryl, -(5- to 10-membered)-C₂₋₉ heterocyclyl, and —C₁₋₄ alkyl-(5- to 10-membered)-C₂₋₉ heterocyclyl, wherein said aryl, alkylaryl, heteroaryl, alkylheteroaryl, heterocyclyl and alkylheterocyclyl groups are optionally substituted with 1, 2 or 3 substituents each independently selected from the group consisting of halogen, hydroxy, —CN, —ORb^{a}, —SRb^{a}, —N(Rb^{a})₂, —C₁₋₄ alkyl optionally substituted with 1, 2, or 3 halogen atoms, optionally substituted C₆₋₁₀ aryl, optionally substituted (5- to 10-membered)-C₁₋₉ heteroaryl, and (5- to 10-membered)-C₂₋₉ heterocyclyl; and wherein said cycloalkyl, alkylcycloalkyl, aryl, alkylaryl, heteroaryl, alkylheteroaryl, heterocyclyl and alkylheterocyclyl is optionally fused to a further (second) ring; and

each Rb^{a} is independently hydrogen, —C₁₋₄ alkyl, —C₃₋₁₀ cycloalkyl, or -(5- to 10-membered)-C₂₋₉ heterocyclyl, wherein said alkyl, cycloalkyl or heterocyclyl group is optionally substituted by 1, 2 or 3 fluorine atoms.

[17] The method of [16], wherein 1) when A¹ is N and R^{2a′} is —C₁₋₄ alkyl-C(═O)NHRa^{a′}, then Ra^{a′} is other than -(5- to 10-membered)-C₂₋₉ heterocyclyl; or 2) when A⁴ is N, then R^{2a′} is other than —C(═O)Ra^{a′}.
[18] The method of any one of [1] to [17], wherein R^{1a} is —C₆₋₁₀ aryl or —C₁₋₄ alkyl-C₆₋₁₀ aryl, wherein said aryl or alkylaryl is optionally substituted with 1, 2 or 3 substituents each independently selected from the group consisting of halogen, hydroxy, —CN, —ORb^{a}, —SRb^{a}, —N(Rb^{a})₂, —C₁₋₄ alkyl optionally substituted with 1, 2, or 3 halogen atoms, optionally substituted —C₆₋₁₀ aryl, optionally substituted -(5- to 10-membered)-C₁₋₉ heteroaryl, and -(5- to 10-membered)-C₂₋₉ heterocyclyl, wherein Rb^{a} is as defined in [1].
[19] The method of any one of [1] to [17], wherein R^{1a} is unsubstituted —C₁₋₄ alkyl-C₆₋₁₀ aryl or —C₁₋₄ alkyl-C₆₋₁₀ aryl optionally substituted with 1, 2 or 3 substituents each independently selected from the group consisting of halogen, hydroxy, —CN, —ORb^{a}, —SRb^{a}, —N(Rb^{a})₂, —C₁₋₄alkyl optionally substituted with 1, 2, or 3 halogen atoms, optionally substituted —C₆₋₁₀ aryl, optionally substituted -(5- to 10-membered)-C₁₋₉ heteroaryl and -(5- to 10-membered)-C₂₋₉ heterocyclyl, wherein Rb^{a} is as defined in [1].
[20] The method of any one of [1] to [17] or [19], wherein R^{1a} is unsubstituted benzyl or unsubstituted phenethyl.
[21] The method of any one of [1] to [17] or [19], wherein R^{1a} is —C₁₋₄ alkyl-C₆₋₁₀ aryl substituted with 1 or 2 substituents each independently selected from the group consisting of halogen, hydroxy, —CN, —O(C₁₋₄)alkyl, —S(C₁₋₄)alkyl, —N(C₁₋₄ alkyl)₂, —NH(C₁₋₄ alkyl), and —C₁₋₄ alkyl optionally substituted with 1, 2, or 3 halogen atoms.
[22] The method of any one of [1] to [17], [19], or [20], wherein R^{1a} is benzyl substituted with 1 or 2 substituents each independently selected from the group consisting of halogen, hydroxy, —CN, —O(C₁₋₄)alkyl, —S(C₁₋₄)alkyl, —N(C₁₋₄ alkyl)₂, —NH(C₁₋₄ alkyl), and —C₁₋₄ alkyl optionally substituted with 1, 2, or 3 halogen atoms.
[23] The method of any one of [1] to [17], wherein R^{1a} is —C₃₋₁₀ cycloalkyl or —C₁₋₄ alkyl-C₃₋₁₀ cycloalkyl, wherein said cycloalkyl or alkylcycloalkyl is optionally substituted with 1, 2 or 3 substituents each independently selected from the group consisting of halogen, hydroxy, —CN, —ORb^{a}, —SRb^{a}, —N(Rb^{a})₂, —C₁₋₄ alkyl optionally substituted with 1, 2, or 3 halogen atoms, optionally substituted —C₆₋₁₀ aryl, optionally substituted -(5- to 10-membered)-C₁₋₉ heteroaryl, and -(5- to 10-membered)-C₂₋₉ heterocyclyl, wherein Rb^{a} is as defined above; and wherein said cycloalkyl is optionally fused to a further (second) ring, and wherein Rb^{a} is as defined in [1].
[24] The method of any one of [1] to [17], wherein R^{1a} is -(5- to 10-membered)-C₁₋₉ heteroaryl or —C₁₋₄ alkyl-(5- to 10-membered)-C₁₋₉ heteroaryl, wherein said heteroaryl or alkylheteroaryl is optionally substituted with 1, 2 or 3 substituents each independently selected from the group consisting of halogen, hydroxy, —CN, —ORb^{a}, —SRb^{a}, —N(Rb^{a})₂, —C₁₋₄ alkyl optionally substituted with 1, 2, or 3 halogen atoms, optionally substituted —C₆₋₁₀ aryl, optionally substituted -(5- to 10-membered)-C₁₋₉ heteroaryl, and -(5- to 10-membered)-C₂₋₉ heterocyclyl, wherein Rb^{a} is as defined in [1].
[25] The method of any one of [1] to [17] or [24], wherein R^{1a} is unsubstituted -(5- to 10-membered)-C₁₋₉ heteroaryl or -(5- to 10-membered)-C₁₋₉ heteroaryl substituted with 1 or 2 substituents each independently selected from the group consisting of halogen, hydroxy, —CN, —O(C₁₋₄)alkyl, —S(C₁₋₄)alkyl, —N(C₁₋₄alkyl)₂, —NH(C₁₋₄ alkyl), and —C₁₋₄ alkyl optionally substituted with 1, 2, or 3 halogen atoms.
[26] The method of any one of [1] to [17] or [24], wherein R^{1a} is unsubstituted —C₁₋₄ alkyl-(5- to 10-membered)-C₁₋₉ heteroaryl or —C₁₋₄ alkyl-(5- to 10-membered)-C₁₋₉ heteroaryl optionally substituted with 1, 2 or 3 substituents each independently selected from the group consisting of halogen, hydroxy, —CN, —ORb^{a}, —SRb^{a}, —N(Rb^{a})₂, —C₁₋₄alkyl optionally substituted with 1, 2, or 3 halogen atoms, optionally substituted —C₆₋₁₀ aryl, optionally substituted -(5- to 10-membered)-C₁₋₉ heteroaryl and -(5- to 10-membered)-C₂₋₉ heterocyclyl, wherein Rb^{a} is as defined in [1].
[27] The method of any one of [1] to [17], [24], or [26], wherein R^{1a} is unsubstituted furan-2-ylmethyl.
[28] The method of any one of [1] to [17], [24], or [26], wherein R^{1a} is —C₁₋₄ alkyl-(5- to 10-membered)-C₁₋₉ heteroaryl substituted with 1 or 2 substituents each independently selected from the group consisting of halogen, hydroxy, —CN, —O(C₁₋₄)alkyl, —S(C₁₋₄)alkyl, —N(C₁₋₄ alkyl)₂, —NH(C₁₋₄ alkyl), and —C₁₋₄ alkyl optionally substituted with 1, 2, or 3 halogen atoms.
[29] The method of any one of [1] to [20], [23], [24], [27], or [27], wherein Rb^{a} is hydrogen or —C₁₋₄ alkyl.
[30] The method of any one of [1] to [29], wherein R^{2a} is —C₁₋₄ alkyl-(5- to 10-membered)-C₁₋₉ heteroaryl, wherein said alkylheteroaryl group is optionally substituted with 1, 2 or 3 substituents each independently selected from the group consisting of halogen, hydroxy, —CN, —C(═O)Ra^{a}, —ORb^{a}, —SRb^{a}, —N(Rb^{a})₂, (═O), —C₁₋₄ alkyl optionally substituted with 1, 2, or 3 halogen atoms, optionally substituted C₆₋₁₀ aryl, optionally substituted -(5- to 10-membered)-C₁₋₉ heteroaryl, and -(5- to 10-membered)-C₂₋₉ heterocyclyl; and wherein said cycloalkyl, alkylcycloalkyl, aryl, alkylaryl, heteroaryl, alkylheteroaryl, heterocyclyl and alkylheterocyclyl is optionally fused to a further (second) ring, wherein Ra^{a} and Rb^{a} are as claimed in [1].
[31] The method of any one of [1] to [29], wherein R^{2a} is —C₁₋₄ alkyl-C(═O)NHRa^{a} or —C₁₋₄ alkyl-C(═O)N(Ra^{a})₂, wherein Ra^{a} is as defined in [1].
[32] The method of any one of [1] to [29], wherein R^{2a} is —S(═O)₂Ra^{a}, wherein Ra^{a} is as defined in [1].
[33] The method of any one of [1] to [29], wherein Ra^{a} is selected from the group consisting of —C₆₋₁₀ aryl, -(5- to 10-membered)-C₁₋₉ heteroaryl, —C₃₋₁₀ cycloalkyl, and -(5- to 10-membered)-C₂₋₉ heterocyclyl, wherein said aryl, heteroaryl, cycloalkyl, and heterocyclyl groups are optionally substituted with 1, 2 or 3 substituents each independently selected from the group consisting of halogen, hydroxy, —CN, —ORb^{a}, —SRb^{a}, —N(Rb^{a})₂, —C₁₋₄alkyl optionally substituted with 1, 2, or 3 halogen atoms, optionally substituted —C₆₋₁₀ aryl, optionally substituted -(5- to 10-membered)-C₁₋₉ heteroaryl and -(5- to 10-membered)-C₂₋₉ heterocyclyl, and wherein said aryl, heteroaryl, cycloalkyl, and heterocyclyl is optionally fused to a further (second) ring.
[34] The method of any one of [1] to [6], wherein the compound of formula (IA) is selected from the group consisting of

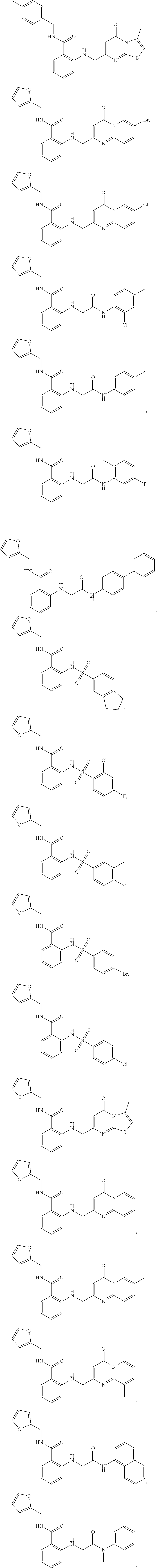

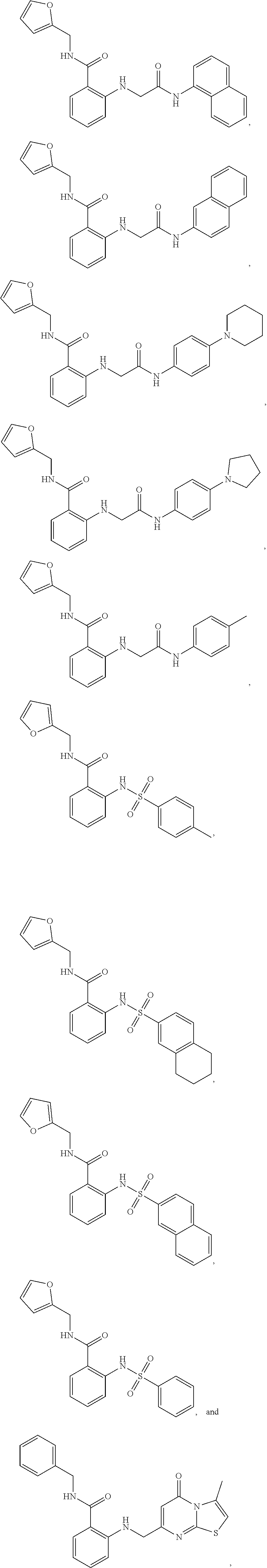

or a pharmaceutically acceptable salt or solvate thereof.

A method of treating or preventing a condition associated with the alteration of the activity of galactocerebrosidase in a patient, comprising administering to the patient in need thereof an effective amount of a compound of formula (IB):

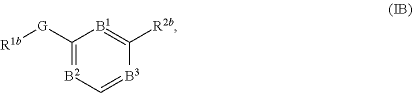

or a pharmaceutically acceptable salt or solvate thereof, wherein

G is —C(═O)—NH— or —NH—C(═O)—;

B¹, B², and B³ are each independently selected from the group consisting of N, CH and C(R^{3b});

each R^{3b} is independently selected from the group consisting of halogen, C₁₋₄ alkyl, —OH, C₁₋₄ alkoxy, and CN;

R^{1b} is selected from the group consisting of —C₁₋₄ alkyl, —C₃₋₁₀ cycloalkyl, —C₁₋₄ alkyl-C₃₋₁₀ cycloalkyl, —C₆₋₁₀ aryl, —C₁₋₄ alkyl-C₆₋₁₀ aryl, —C₂₋₄ alkylene-C₆₋₁₀ aryl, (5- to 10-membered)-C₁₋₉ heteroaryl, —C₁₋₄ alkyl-(5- to 10-membered)-C₁₋₉ heteroaryl, —C₂₋₄ alkylene-(5- to 10-membered)-C₁₋₉ heteroaryl, (5- to 10-membered)-C₂₋₉ heterocyclyl, —C₁₋₄ alkyl-(5- to 10-membered)-C₂₋₉ heterocyclyl, and —C₂₋₄ alkenyl-(5- to 10-membered)-C₂₋₉ heterocyclyl, wherein said alkyl, cycloalkyl, alkylcycloalkyl, aryl, alkylaryl, heteroaryl, alkylheteroaryl, alkenylheteroaryl, heterocyclyl and alkylheterocyclyl groups are optionally substituted with 1, 2 or 3 substituents each independently selected from the group consisting of halogen, hydroxy, —CN, —ORb^{b}, —SRb^{b}, —N(Rb^{b})₂, —C₁₋₄ alkyl optionally substituted with 1, 2, or 3 halogen atoms, optionally substituted C₆₋₁₀ aryl, optionally substituted (5- to 10-membered)-C₁₋₉ heteroaryl, and (5- to 10-membered)-C₂₋₉ heterocyclyl; and wherein said cycloalkyl, alkylcycloalkyl, aryl, alkylaryl, heteroaryl, alkylheteroaryl, alkenylheteroaryl, heterocyclyl and alkylheterocyclyl is optionally fused to a further (second) ring;

R^{2b} is —C₆₋₁₀ aryl, -(5- to 10-membered)-C₁₋₉ heteroaryl, —C(═O)Ra^{b}, —S(═O)₂Ra^{b}, —C(═O)—NH—Ra^{b}, —S(═O)₂—NH—Ra^{b}, —C₁₋₄ alkyl-C(═O)Ra^{b}, —C₁₋₄ alkyl-S(═O)₂Ra^{b}, or —N(Rb^{b})₂, wherein said aryl and heteroaryl groups are optionally substituted with 1, 2 or 3 substituents each independently selected from the group consisting of halogen, hydroxy, CN, —ORb^{b}, —SRb^{b}, —N(Rb^{b})₂, (═O), —C₁₋₄alkyl optionally substituted with 1, 2, or 3 substituents each independently selected from the group consisting of halogen, CN, —ORb^{b}, and —N(Rb^{b})₂, optionally substituted —C₆₋₁₀ aryl, optionally substituted -(5- to 10-membered)-C₁₋₉ heteroaryl, -(5- to 10-membered)-C₂₋₉ heterocyclyl, and —C₃₋₁₀ cycloalkyl; and wherein said aryl, heteroaryl, and heterocyclyl are optionally fused to a further (second) ring; or

R^{2b} and R^{3b} attached to an adjacent carbon atom together form a 5- or 6-membered heterocyclic ring containing one N-atom substituted with —S(═O)₂Ra^{b};

Ra^{b} is selected from the group consisting of —C₁₋₄ alkyl, —C₃₋₁₀ cycloalkyl, —C₁₋₄ alkyl-C₃₋₁₀ cycloalkyl, —C₆₋₁₀ aryl, —C₁₋₄ alkyl-C₆₋₁₀ aryl, (5- to 10-membered)-C₁₋₉ heteroaryl, —C₁₋₄ alkyl-(5- to 10-membered)-C₁₋₉ heteroaryl, (5- to 10-membered)-C₂₋₉ heterocyclyl, and —C₁₋₄ alkyl-(5- to 10-membered)-C₂₋₉ heterocyclyl, wherein said alkyl, cycloalkyl, alkylcycloalkyl, aryl, alkylaryl, heteroaryl, alkylheteroaryl, heterocyclyl and alkylheterocyclyl groups are optionally substituted with 1, 2 or 3 substituents each independently selected from the group consisting of halogen, hydroxy, —CN, —ORb^{b}, —SRb^{b}, —N(Rb^{b})₂, —C₁₋₄ alkyl optionally substituted with 1, 2, or 3 halogen atoms, optionally substituted C₆₋₁₀ aryl, optionally substituted (5- to 10-membered)-C₁₋₉ heteroaryl, and (5- to 10-membered)-C₂₋₉ heterocyclyl; and wherein said cycloalkyl, alkylcycloalkyl, aryl, alkylaryl, heteroaryl, alkylheteroaryl, heterocyclyl and alkylheterocyclyl is optionally fused to a further (second) ring; and

each Rb^{b} is independently hydrogen, —C(═O)Ra^{b}, —S(═O)₂Ra^{b}, —C₁₋₄ alkyl, —C₃₋₁₀ cycloalkyl, -(5- to 10-membered)-C₂₋₉ heterocyclyl, or optionally substituted —C₆₋₁₀ aryl, wherein said alkyl, cycloalkyl or heterocyclyl group is optionally substituted by 1, 2 or 3 fluorine atoms.

[36] A method of treating or preventing a lysosomal storage disease or an α-synucleinopathy, comprising administering to a patient in need thereof an effective amount of a compound of formula (IB):

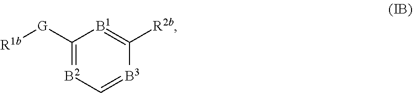

or a pharmaceutically acceptable salt or solvate thereof, wherein

G is —C(═O)—NH— or —NH—C(═O)—;

B¹, B², and B³ are each independently selected from the group consisting of N, CH and C(R^{3b});

each R^{3b} is independently selected from the group consisting of halogen, C₁₋₄ alkyl, —OH, C₁₋₄ alkoxy, and CN;

R^{1b} is selected from the group consisting of —C₁₋₄ alkyl, —C₃₋₁₀ cycloalkyl, —C₁₋₄ alkyl-C₃₋₁₀ cycloalkyl, —C₆₋₁₀ aryl, —C₁₋₄ alkyl-C₆₋₁₀ aryl, —C₂₋₄ alkylene-C₆₋₁₀ aryl, (5- to 10-membered)-C₁₋₉ heteroaryl, —C₁₋₄ alkyl-(5- to 10-membered)-C₁₋₉ heteroaryl, —C₂₋₄ alkylene-(5- to 10-membered)-C₁₋₉ heteroaryl, (5- to 10-membered)-C₂₋₉ heterocyclyl, —C₁₋₄ alkyl-(5- to 10-membered)-C₂₋₉ heterocyclyl, and —C₂₋₄ alkenyl-(5- to 10-membered)-C₂₋₉ heterocyclyl, wherein said alkyl, cycloalkyl, alkylcycloalkyl, aryl, alkylaryl, heteroaryl, alkylheteroaryl, alkenylheteroaryl, heterocyclyl and alkylheterocyclyl groups are optionally substituted with 1, 2 or 3 substituents each independently selected from the group consisting of halogen, hydroxy, —CN, —ORb^{b}, —SRb^{b}, —N(Rb^{b})₂, —C₁₋₄ alkyl optionally substituted with 1, 2, or 3 halogen atoms, optionally substituted C₆₋₁₀ aryl, optionally substituted (5- to 10-membered)-C₁₋₉ heteroaryl, and (5- to 10-membered)-C₂₋₉ heterocyclyl; and wherein said cycloalkyl, alkylcycloalkyl, aryl, alkylaryl, heteroaryl, alkylheteroaryl, alkenylheteroaryl, heterocyclyl and alkylheterocyclyl is optionally fused to a further (second) ring;

R^{2b} is —C₆₋₁₀ aryl, -(5- to 10-membered)-C₁₋₉ heteroaryl, —C(═O)Ra^{b}, —S(═O)₂Ra^{b}, —C(═O)—NH—Ra^{b}, —S(═O)₂—NH—Ra^{b}, —C₁₋₄ alkyl-C(═O)Ra^{b}, —C₁₋₄ alkyl-S(═O)₂Ra^{b}, or —N(Rb^{b})₂, wherein said aryl and heteroaryl groups are optionally substituted with 1, 2 or 3 substituents each independently selected from the group consisting of halogen, hydroxy, CN, —ORb^{b}, —SRb^{b}, —N(Rb^{b})₂, (═O), —C₁₋₄alkyl optionally substituted with 1, 2, or 3 substituents each independently selected from the group consisting of halogen, CN, —ORb^{b}, and —N(Rb^{b})₂, optionally substituted —C₆₋₁₀ aryl, optionally substituted -(5- to 10-membered)-C₁₋₉ heteroaryl, -(5- to 10-membered)-C₂₋₉ heterocyclyl, and —C₃₋₁₀ cycloalkyl; and wherein said aryl, heteroaryl, and heterocyclyl are optionally fused to a further (second) ring; or

R^{2b} and R^{3b} attached to an adjacent carbon atom together form a 5- or 6-membered heterocyclic ring containing one N-atom substituted with —S(═O)₂Ra^{b};

Ra^{b} is selected from the group consisting of —C₁₋₄ alkyl, —C₃₋₁₀ cycloalkyl, —C₁₋₄ alkyl-C₃₋₁₀ cycloalkyl, —C₆₋₁₀ aryl, —C₁₋₄ alkyl-C₆₋₁₀ aryl, (5- to 10-membered)-C₁₋₉ heteroaryl, —C₁₋₄ alkyl-(5- to 10-membered)-C₁₋₉ heteroaryl, (5- to 10-membered)-C₂₋₉ heterocyclyl, and —C₁₋₄ alkyl-(5- to 10-membered)-C₂₋₉ heterocyclyl, wherein said alkyl, cycloalkyl, alkylcycloalkyl, aryl, alkylaryl, heteroaryl, alkylheteroaryl, heterocyclyl and alkylheterocyclyl groups are optionally substituted with 1, 2 or 3 substituents each independently selected from the group consisting of halogen, hydroxy, —CN, —ORb^{b}, —SRb^{b}, —N(Rb^{b})₂, —C₁₋₄ alkyl optionally substituted with 1, 2, or 3 halogen atoms, optionally substituted C₆₋₁₀ aryl, optionally substituted (5- to 10-membered)-C₁₋₉ heteroaryl, and (5- to 10-membered)-C₂₋₉ heterocyclyl; and wherein said cycloalkyl, alkylcycloalkyl, aryl, alkylaryl, heteroaryl, alkylheteroaryl, heterocyclyl and alkylheterocyclyl is optionally fused to a further (second) ring; and

each Rb^{b} is independently hydrogen, —C(═O)Ra^{b}, —S(═O)₂Ra^{b}, —C₁₋₄ alkyl, —C₃₋₁₀ cycloalkyl, -(5- to 10-membered)-C₂₋₉ heterocyclyl, or optionally substituted —C₆₋₁₀ aryl, wherein said alkyl, cycloalkyl or heterocyclyl group is optionally substituted by 1, 2 or 3 fluorine atoms.

[37] The method of [36], wherein a lysosomal storage disease is treated or prevented.
[38] The method of [36] or [37], wherein the lysosomal storage disease is Krabbe's disease.
[39] The method of [36], wherein an α-synucleinopathy is treated or prevented.
[40] A method of treating or preventing a disease or disorder, comprising administering to a patient in need thereof an effective amount of a compound of formula (IB).

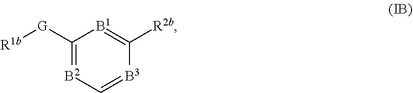

or a pharmaceutically acceptable salt or solvate thereof, wherein

G is —C(═O)—NH— or —NH—C(═O)—;

B¹, B², and B³ are each independently selected from the group consisting of N, CH and C(R^{3b});

each R^{3b} is independently selected from the group consisting of halogen, C₁₋₄ alkyl, —OH, C₁₋₄ alkoxy, and CN;

R^{1b} is selected from the group consisting of —C₁₋₄ alkyl, —C₃₋₁₀ cycloalkyl, —C₁₋₄ alkyl-C₃₋₁₀ cycloalkyl, —C₆₋₁₀ aryl, —C₁₋₄ alkyl-C₆₋₁₀ aryl, —C₂₋₄ alkylene-C₆₋₁₀ aryl, (5- to 10-membered)-C₁₋₉ heteroaryl, —C₁₋₄ alkyl-(5- to 10-membered)-C₁₋₉ heteroaryl, —C₂₋₄ alkylene-(5- to 10-membered)-C₁₋₉ heteroaryl, (5- to 10-membered)-C₂₋₉ heterocyclyl, —C₁₋₄ alkyl-(5- to 10-membered)-C₂₋₉ heterocyclyl, and —C₂₋₄ alkenyl-(5- to 10-membered)-C₂₋₉ heterocyclyl, wherein said alkyl, cycloalkyl, alkylcycloalkyl, aryl, alkylaryl, heteroaryl, alkylheteroaryl, alkenylheteroaryl, heterocyclyl and alkylheterocyclyl groups are optionally substituted with 1, 2 or 3 substituents each independently selected from the group consisting of halogen, hydroxy, —CN, —ORb^{b}, —SRb^{b}, —N(Rb^{b})₂, —C₁₋₄ alkyl optionally substituted with 1, 2, or 3 halogen atoms, optionally substituted C₆₋₁₀ aryl, optionally substituted (5- to 10-membered)-C₁₋₉ heteroaryl, and (5- to 10-membered)-C₂₋₉ heterocyclyl; and wherein said cycloalkyl, alkylcycloalkyl, aryl, alkylaryl, heteroaryl, alkylheteroaryl, alkenylheteroaryl, heterocyclyl and alkylheterocyclyl is optionally fused to a further (second) ring;

R^{2b} is —C₆₋₁₀ aryl, -(5- to 10-membered)-C₁₋₉ heteroaryl, —C(═O)Ra^{b}, —S(═O)₂Ra^{b}, —C(═O)—NH—Ra^{b}, —S(═O)₂—NH—Ra^{b}, —C₁₋₄ alkyl-C(═O)Ra^{b}, —C₁₋₄ alkyl-S(═O)₂Ra^{b}, or —N(Rb^{b})₂, wherein said aryl and heteroaryl groups are optionally substituted with 1, 2 or 3 substituents each independently selected from the group consisting of halogen, hydroxy, CN, —ORb^{b}, —SRb^{b}, —N(Rb^{b})₂, (═O), —C₁₋₄alkyl optionally substituted with 1, 2, or 3 substituents each independently selected from the group consisting of halogen, CN, —ORb^{b}, and —N(Rb^{b})₂, optionally substituted —C₆₋₁₀ aryl, optionally substituted -(5- to 10-membered)-C₁₋₉ heteroaryl, -(5- to 10-membered)-C₂₋₉ heterocyclyl, and —C₃₋₁₀ cycloalkyl; and wherein said aryl, heteroaryl, and heterocyclyl are optionally fused to a further (second) ring; or

R^{2b} and R^{3b} attached to an adjacent carbon atom together form a 5- or 6-membered heterocyclic ring containing one N-atom substituted with —S(═O)₂Ra^{b};

Ra^{b} is selected from the group consisting of —C₁₋₄ alkyl, —C₃₋₁₀ cycloalkyl, —C₁₋₄ alkyl-C₃₋₁₀ cycloalkyl, —C₆₋₁₀ aryl, —C₁₋₄ alkyl-C₆₋₁₀ aryl, (5- to 10-membered)-C₁₋₉ heteroaryl, —C₁₋₄ alkyl-(5- to 10-membered)-C₁₋₉ heteroaryl, (5- to 10-membered)-C₂₋₉ heterocyclyl, and —C₁₋₄ alkyl-(5- to 10-membered)-C₂₋₉ heterocyclyl, wherein said alkyl, cycloalkyl, alkylcycloalkyl, aryl, alkylaryl, heteroaryl, alkylheteroaryl, heterocyclyl and alkylheterocyclyl groups are optionally substituted with 1, 2 or 3 substituents each independently selected from the group consisting of halogen, hydroxy, —CN, —ORb^{b}, —SRb^{b}, —N(Rb^{b})₂, —C₁₋₄ alkyl optionally substituted with 1, 2, or 3 halogen atoms, optionally substituted C₆₋₁₀ aryl, optionally substituted (5- to 10-membered)-C₁₋₉ heteroaryl, and (5- to 10-membered)-C₂₋₉ heterocyclyl; and wherein said cycloalkyl, alkylcycloalkyl, aryl, alkylaryl, heteroaryl, alkylheteroaryl, heterocyclyl and alkylheterocyclyl is optionally fused to a further (second) ring; and

each Rb^{b} is independently hydrogen, —C(═O)Ra^{b}, —S(═O)₂Ra^{b}, —C₁₋₄ alkyl, —C₃₋₁₀ cycloalkyl, -(5- to 10-membered)-C₂₋₉ heterocyclyl, or optionally substituted —C₆₋₁₀ aryl, wherein said alkyl, cycloalkyl or heterocyclyl group is optionally substituted by 1, 2 or 3 fluorine atoms.

The method of any one of [35] to [40], comprising administering to a patient in need thereof an effective amount of a compound of formula (IB) where G is —C(═O)—NH—, which is a compound of formula (IIB):

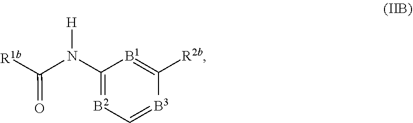

or a pharmaceutically acceptable salt or solvate thereof, wherein B¹, B², B³, R^{1b}, and R^{2b} are as defined in [35].
[42] The method of any one of [35] to [40], comprising administering to a patient in need thereof an effective amount of a compound of formula (IB) where G is —NH—C(═O)—, which is a compound of formula (IIIB):

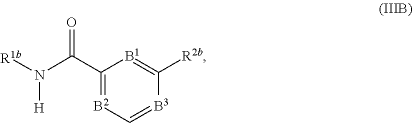

or a pharmaceutically acceptable salt or solvate thereof, wherein B¹, B², B³, R^{1b}, and R^{2b} are as defined in [35].
[43] The method of any one of [35] to [42], wherein B¹, B², and B³ are CH.
[44] The method of any one of [35] to [42], wherein one of B¹, B², and B³ is C(R^{3b}) and the ones not C(R^{3b}) are CH.
[45] The method of any one of [35] to [42], wherein two of B¹, B², and B³ is C(R^{3b}) and the one not C(R^{3b}) is CH.
[46] The method of any one of [35] to [42], wherein one of B¹, B² and B³ is N.
[47] The method of any one of [35]-[42], wherein two of B¹, B² and B³ are N.
[48] The method of any one of [35] to [42], wherein B¹, B² and B³ are N.
[49] The method of any one of [35] to [42], wherein B¹ is N and B² and B³ are each independently selected from the group consisting of CH and C(R^{3b}).
[50] The method of any one of [35] to [42], wherein B² is N and B¹ and B³ are each independently selected from the group consisting of CH and C(R^{3b}).
[51] The method of any one of [35] to [42], wherein B³ is N and B¹ and B² are each independently selected from the group consisting of CH and C(R^{3b}).
[52] The method of any one of [35] to [42], wherein B¹ and B² are both N and B³ is CH or C(R^{3b}).
[53] The method of any one of [35] to [42], wherein B¹ and B³ are both N and B² is CH or C(R^{3b}).
[54] The method of any one of [35] to [42], wherein B² and B³ are both N and B¹ is CH or C(R^{3b}).
[55] The method of any one of [35] to [54], wherein R^{1b} is —C₆₋₁₀ aryl or —C₁₋₄ alkyl-C₆₋₁₀ aryl, wherein said aryl or alkylaryl is optionally substituted with 1, 2 or 3 substituents each independently selected from the group consisting of halogen, hydroxy, —CN, —ORb^{b}, —SRb^{b}, —N(Rb^{b})₂, —C₁₋₄ alkyl optionally substituted with 1, 2, or 3 halogen atoms, optionally substituted —C₆₋₁₀ aryl, optionally substituted -(5- to 10-membered)-C₁₋₉ heteroaryl, and -(5- to 10-membered)-C₂₋₉ heterocyclyl, wherein Rb^{b} is as defined in [35].
[56] The method of any one of [35] to [54], wherein R^{1b} is unsubstituted —C₁₋₄ alkyl-C₆₋₁₀ aryl or —C₁₋₄ alkyl-C₆₋₁₀ aryl optionally substituted with 1, 2 or 3 substituents each independently selected from the group consisting of halogen, hydroxy, —CN, —ORb^{b}, —SRb^{b}, —N(Rb^{b})₂, —C₁₋₄alkyl optionally substituted with 1, 2, or 3 halogen atoms, optionally substituted —C₆₋₁₀ aryl, optionally substituted -(5- to 10-membered)-C₁₋₉ heteroaryl and -(5- to 10-membered)-C₂₋₉ heterocyclyl, wherein Rb^{b} is as defined in [35].
[57] The method of any one of [35] to [54] or [56], wherein R^{1b} is —C₁₋₄ alkyl-C₆₋₁₀ aryl substituted with 1 or 2 substituents each independently selected from the group consisting of halogen, hydroxy, —CN, —O(C₁₋₄)alkyl, —S(C₁₋₄)alkyl, —N(C₁₋₄ alkyl)₂, —NH(C₁₋₄ alkyl), and —C₁₋₄ alkyl optionally substituted with 1, 2, or 3 halogen atoms.
[58] The method of any one of [35] to [54], wherein R^{1b} is —C₃₋₁₀ cycloalkyl or —C₁₋₄ alkyl-C₃₋₁₀ cycloalkyl, wherein said cycloalkyl or alkylcycloalkyl is optionally substituted with 1, 2 or 3 substituents each independently selected from the group consisting of halogen, hydroxy, —CN, —ORb^{b}, —SRb^{b}, —N(Rb^{b})₂, —C₁₋₄ alkyl optionally substituted with 1, 2, or 3 halogen atoms, optionally substituted —C₆₋₁₀ aryl, optionally substituted -(5- to 10-membered)-C₁₋₉ heteroaryl, and -(5- to 10-membered)-C₂₋₉ heterocyclyl, wherein Rb^{b} is as defined in [35]; and wherein said cycloalkyl is optionally fused to a further (second) ring.
[59] The method of any one of [35] to [54], wherein R^{1b} is -(5- to 10-membered)-C₁₋₉ heteroaryl, —C₁₋₄ alkyl-(5- to 10-membered)-C₁₋₉ heteroaryl, or —C₂₋₄ alkenyl-(5- to 10-membered)-C₁₋₉ heteroaryl, wherein said heteroaryl, alkylheteroaryl, or alkenylheteroaryl is optionally substituted with 1, 2 or 3 substituents each independently selected from the group consisting of halogen, hydroxy, —CN, —ORb^{b}, —SRb^{b}, —N(Rb^{b})₂, —C₁₋₄ alkyl optionally substituted with 1, 2, or 3 halogen atoms, optionally substituted —C₆₋₁₀ aryl, optionally substituted -(5- to 10-membered)-C₁₋₉ heteroaryl, and -(5- to 10-membered)-C₂₋₉ heterocyclyl, wherein Rb^{b} is as defined in [35].
[60] The method of any one of [35] to [54] or [59], wherein R^{1b} is unsubstituted -(5- to 10-membered)-C₁₋₉ heteroaryl or -(5- to 10-membered)-C₁₋₉ heteroaryl substituted with 1 or 2 substituents each independently selected from the group consisting of halogen, hydroxy, —CN, —O(C₁₋₄)alkyl, —S(C₁₋₄)alkyl, —N(C₁₋₄alkyl)₂, —NH(C₁₋₄ alkyl), and —C₁₋₄ alkyl optionally substituted with 1, 2, or 3 halogen atoms.
[61] The method of any one of [35] to [54] or [59], wherein R^{1b} is unsubstituted —C₁₋₄ alkyl-(5- to 10-membered)-C₁₋₉ heteroaryl or —C₁₋₄ alkyl-(5- to 10-membered)-C₁₋₉ heteroaryl optionally substituted with 1, 2 or 3 substituents each independently selected from the group consisting of halogen, hydroxy, —CN, —ORb^{a}, —SRb^{a}, —N(Rb^{a})₂, —C₁₋₄alkyl optionally substituted with 1, 2, or 3 halogen atoms, optionally substituted —C₆₋₁₀ aryl, optionally substituted -(5- to 10-membered)-C₁₋₉ heteroaryl and -(5- to 10-membered)-C₂₋₉ heterocyclyl, wherein Rb^{b} is as defined in claim 35.
[62] The method of any one of [35] to [54] or [59], wherein R^{1b} is unsubstituted —C₂₋₄ alkenyl-(5- to 10-membered)-C₁₋₉ heteroaryl or —C₂₋₄ alkenyl-(5- to 10-membered)-C₁₋₉ heteroaryl optionally substituted with 1, 2 or 3 substituents each independently selected from the group consisting of halogen, hydroxy, —CN, —ORb^{a}, —SRb^{a}, —N(Rb^{a})₂, —C₁₋₄alkyl optionally substituted with 1, 2, or 3 halogen atoms, optionally substituted —C₆₋₁₀ aryl, optionally substituted -(5- to 10-membered)-C₁₋₉ heteroaryl and -(5- to 10-membered)-C₂₋₉ heterocyclyl, wherein Rb^{b} is as defined in [35].
[63] The method of any one of [35] to [54], [59], or [62], wherein R^{1b} is unsubstituted furan-2-yl-ethenyl.
[64] The method of any one of [35] to [54], wherein R^{1b} is —C₁₋₄ alkyl optionally substituted with 1, 2 or 3 substituents each independently selected from the group consisting of halogen, hydroxy, —CN, —ORb^{b}, —SRb^{b}, —N(Rb^{b})₂, —C₁₋₄ alkyl optionally substituted with 1, 2, or 3 halogen atoms, optionally substituted C₆₋₁₀ aryl, optionally substituted (5- to 10-membered)-C₁₋₉ heteroaryl, and (5- to 10-membered)-C₂₋₉ heterocyclyl; and wherein said cycloalkyl, alkylcycloalkyl, aryl, alkylaryl, heteroaryl, alkylheteroaryl, alkenylheteroaryl, heterocyclyl and alkylheterocyclyl is optionally fused to a further (second) ring, wherein Rb^{b} is as defined in [35].
[65] The method of any one of [35] to [54] or [64], wherein R^{1b} is unsubstituted —C₁₋₄ alkyl.
[66] The method of any one of [35] to [54] or [64], wherein R^{1b} is —C₁₋₄ alkyl substituted with —ORb^{b}, —SRb^{b}, or —N(Rb^{b})₂, wherein Rb^{b} is as defined in [35].
[67] The method of any one of [35] to [54], [64], or [66], wherein each Rb^{b} is independently hydrogen, —C(═O)Ra^{b}, —S(═O)₂Ra^{b}, —C₁₋₄ alkyl, —C₃₋₁₀ cycloalkyl, -(5- to 10-membered)-C₂₋₉ heterocyclyl, or optionally substituted —C₆₋₁₀ aryl, wherein said alkyl, cycloalkyl or heterocyclyl group is optionally substituted by 1, 2 or 3 fluorine atoms.
[68] The method of any one of [35] to [67], wherein R^{2b} is —C₆₋₁₀ aryl, -(5- to 10-membered)-C₁₋₉ heteroaryl, —C(═O)Ra^{b}, —S(═O)₂Ra^{b}, —C(═O)—NH—Ra^{b}, —S(═O)₂—NH—Ra^{b}, —C₁₋₄ alkyl-C(═O)Ra^{b}, —C₁₋₄ alkyl-S(═O)₂Ra^{b}, or —N(Rb^{b})₂, wherein said aryl and heteroaryl groups are optionally substituted with 1, 2 or 3 substituents each independently selected from the group consisting of halogen, hydroxy, CN, —ORb^{b}, —SRb^{b}, —N(Rb^{b})₂, (═O), —C₁₋₄alkyl optionally substituted with 1, 2, or 3 substituents each independently selected from the group consisting of halogen, CN, —ORb^{b}, and —N(Rb^{b})₂, optionally substituted —C₆₋₁₀ aryl, optionally substituted -(5- to 10-membered)-C₁₋₉ heteroaryl, -(5- to 10-membered)-C₂₋₉ heterocyclyl, and —C₃₋₁₀ cycloalkyl; and wherein said aryl, heteroaryl, and heterocyclyl is optionally fused to a further (second) ring.
[69] The method of any one of [35] to [68], wherein R^{2b} is —C₆₋₁₀ aryl or -(5- to 10-membered)-C₁₋₉ heteroaryl, wherein said aryl and heteroaryl groups are optionally substituted with 1, 2 or 3 substituents each independently selected from the group consisting of halogen, hydroxy, CN, —ORb^{b}, —SRb^{b}, —N(Rb^{b})₂, (═O), —C₁₋₄alkyl optionally substituted with 1, 2, or 3 substituents each independently selected from the group consisting of halogen, CN, —ORb^{b}, and —N(Rb^{b})₂, optionally substituted —C₆₋₁₀ aryl, optionally substituted -(5- to 10-membered)-C₁₋₉ heteroaryl, -(5- to 10-membered)-C₂₋₉ heterocyclyl, and —C₃₋₁₀ cycloalkyl; and wherein said aryl, heteroaryl, and heterocyclyl is optionally fused to a further (second) ring; wherein Rb^{b} is as defined in [35].
[70] The method of any one of [35] to [67], wherein R^{2b} is —S(═O)₂Ra^{b}, —C(═O)—NH—Ra^{b}, —S(═O)₂—NH—Ra^{b}, —C₁₋₄ alkyl-C(═O)Ra^{b}, —C₁₋₄ alkyl-S(═O)₂Ra^{b}, or —N(Rb^{b})₂, wherein wherein Ra^{b} and Rb^{b} are as defined in [35].
[71] The method of any one of [35] to [67] or [70], wherein R^{2b} is —C(═O)—NH—Ra^{b} or —S(═O)₂—NH—Ra^{b}, wherein Ra^{b} is —C₆₋₁₀ aryl optionally substituted with 1, 2 or 3 substituents each independently selected from the group consisting of halogen, hydroxy, —CN, —ORb^{b}, —SRb^{b}, —N(Rb^{b})₂, and —C₁₋₄ alkyl optionally substituted with 1, 2, or 3 halogen atoms.
[72] The method of any one of [35] to [67], wherein R^{2b} and R^{3b} attached to an adjacent carbon atom together form a 5- or 6-membered N-containing heterocyclic ring substituted at the N-atom with —S(═O)₂Ra^{b}; wherein Ra^{b} is as defined in [35].
[73] The method of any one of [35] to [72], wherein Rb^{b} is hydrogen or —C₁₋₄ alkyl.
[74] The method of any one of [35] to [72], wherein Rb^{b} is hydrogen, —C(═O)Ra^{b}, —S(═O)₂Ra^{b}, —C₁₋₄ alkyl, —C₃₋₆ cycloalkyl, -(5- to 6-membered)-C₂₋₉ heterocyclyl, or —C₆₋₁₀ aryl optionally substituted with 1, 2 or 3 substituents each independently selected from the group consisting of halogen, hydroxy, CN, —O(C₁₋₄ alkyl), —S(C₁₋₄ alkyl), —NH(C₁₋₄ alkyl), —N(C₁₋₄ alkyl)₂, and —C₁₋₄alkyl optionally substituted by 1, 2 or 3 fluorine atoms.
[75] The method of any one of [35] to [41], wherein the compound is selected from the group consisting of

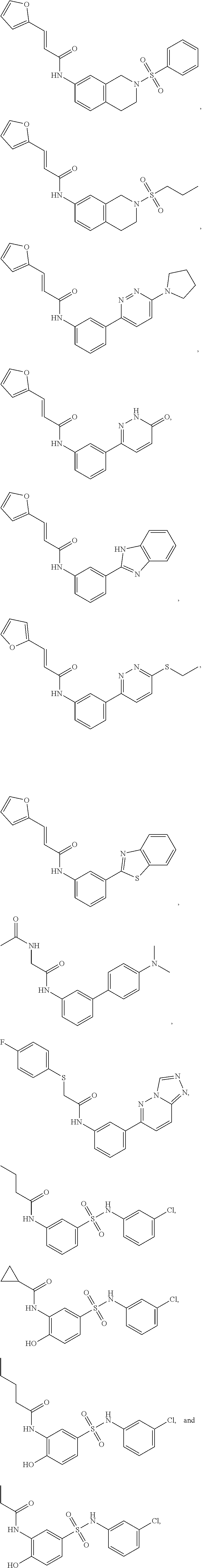

or a pharmaceutically acceptable salt or solvate thereof.
[76] The method of any one of [35] to [41], wherein the compound is selected from the group consisting of

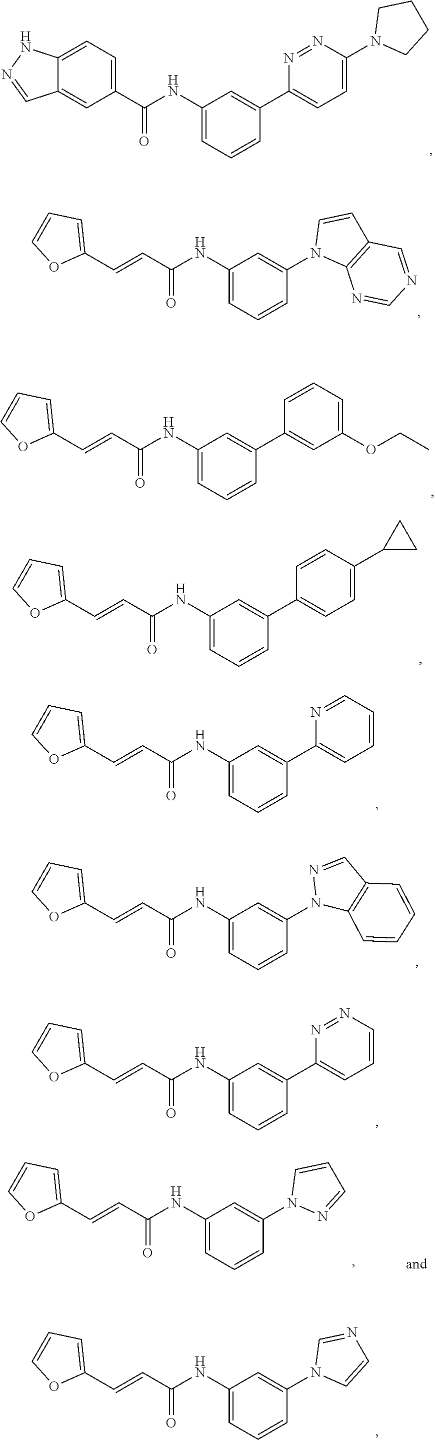

or a pharmaceutically acceptable salt or solvate thereof.
[77] The method of any one of [35] to [40], or [42], wherein the compound is selected from the group consisting of

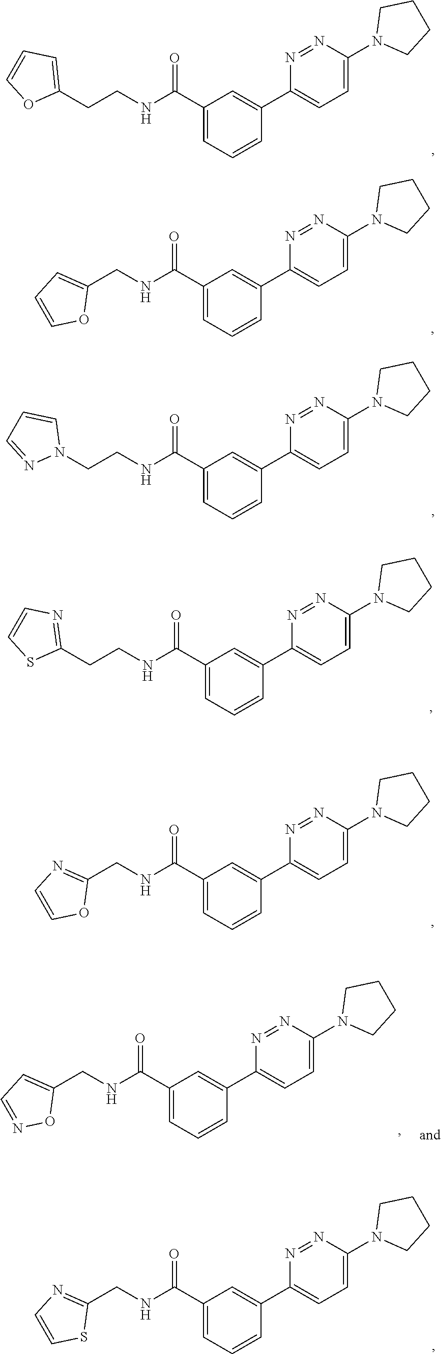

or a pharmaceutically acceptable salt or solvate thereof.
[78] The method of any one of [1] to [77], further comprising administering to the patient at least one other therapeutic agent.
[79] The method of [78], wherein the therapeutic agent is an effective amount of an enzyme for enzyme replacement therapy.
[80] The method of [79], wherein the enzyme is galactocerebrosidase or an analog thereof.
[81] The method of [78], wherein the therapeutic agent is an effective amount of a small molecule chaperone.
[82] The method of [81], wherein the small molecule chaperone binds competitively to an enzyme.
[83] The method of [81] or [82], wherein the small molecule chaperone is selected from the group consisting of iminoalditols, iminosugars, aminosugars, thiophenylglycosides, glycosidase, sulfatase, glycosyl transferase, phosphatase, and peptidase inhibitors.
[84] A compound of formula (IIA):

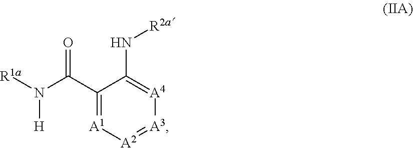

or a pharmaceutically acceptable salt or solvate thereof, wherein

A¹, A², A³, and A⁴ are each independently selected from the group consisting of N, CH and C(R^{3a}), provided that no more than one of A¹, A², A³, or A⁴ is N;

each R^{3a} is independently selected from the group consisting of halogen, —OH, C₁₋₄ alkyl, C₁₋₄ alkoxy, and CN;

R^{1a} is selected from the group consisting of —C₁₋₄ alkyl, —C₃₋₁₀ cycloalkyl, —C₁₋₄ alkyl-C₃₋₁₀ cycloalkyl, —C₆₋₁₀ aryl, —C₁₋₄ alkyl-C₆₋₁₀ aryl, (5- to 10-membered)-C₁₋₉ heteroaryl, —C₁₋₄ alkyl-(5- to 10-membered)-C₁₋₉ heteroaryl, (5- to 10-membered)-C₂₋₉ heterocyclyl, and —C₁₋₄ alkyl-(5- to 10-membered)-C₂₋₉ heterocyclyl, wherein said alkyl, cycloalkyl, alkylcycloalkyl, aryl, alkylaryl, heteroaryl, alkylheteroaryl, heterocyclyl and alkylheterocyclyl groups are optionally substituted with 1, 2 or 3 substituents each independently selected from the group consisting of halogen, hydroxo, —CN, —ORb^{a}, —SRb^{a}, —N(Rb^{a})₂, —C₁₋₄ alkyl optionally substituted with 1, 2, or 3 halogen atoms, optionally substituted C₆₋₁₀ aryl, optionally substituted (5- to 10-membered)-C₁₋₉ heteroaryl, and (5- to 10-membered)-C₂₋₉ heterocyclyl; and wherein said cycloalkyl, alkylcycloalkyl, aryl, alkylaryl, heteroaryl, alkylheteroaryl, heterocyclyl and alkylheterocyclyl is optionally fused to a further (second) ring;

R^{2a} is selected from the group consisting of —C(═O)Ra^{a′}, —S(═O)₂Ra^{a′}, —C₁₋₄ alkyl-C(═O)NHRa^{a′}, —C₁₋₄ alkyl-C(═O)N(Ra^{a′})₂, —C₁₋₄ alkyl-S(═O)₂—N(Ra^{a′})₂, wherein said alkyl group is optionally substituted with 1, 2 or 3 substituents each independently selected from the group consisting of halogen, hydroxy, —CN, —C₁₋₄ alkyl optionally substituted with 1, 2, or 3 halogen atoms;

Ra^{a′} is selected from the group consisting of —C₆₋₁₀ aryl, —C₁₋₄ alkyl-C₆₋₁₀ aryl, (5- to 10-membered)-C₁₋₉ heteroaryl, —C₁₋₄ alkyl-(5- to 10-membered)-C₁₋₉ heteroaryl, -(5- to 10-membered)-C₂₋₉ heterocyclyl, and —C₁₋₄ alkyl-(5- to 10-membered)-C₂₋₉ heterocyclyl, wherein said aryl, alkylaryl, heteroaryl, alkylheteroaryl, heterocyclyl and alkylheterocyclyl groups are optionally substituted with 1, 2 or 3 substituents each independently selected from the group consisting of halogen, hydroxy, —CN, —ORb^{a}, —SRb^{a}, —N(Rb^{a})₂, —C₁₋₄ alkyl optionally substituted with 1, 2, or 3 halogen atoms, optionally substituted C₆₋₁₀ aryl, optionally substituted (5- to 10-membered)-C₁₋₉ heteroaryl, and (5- to 10-membered)-C₂₋₉ heterocyclyl; and wherein said cycloalkyl, alkylcycloalkyl, aryl, alkylaryl, heteroaryl, alkylheteroaryl, heterocyclyl and alkylheterocyclyl is optionally fused to a further (second) ring; and

each Rb^{a} is independently hydrogen, —C₁₋₄ alkyl, —C₃₋₁₀ cycloalkyl, or -(5- to 10-membered)-C₂₋₉ heterocyclyl, wherein said alkyl, cycloalkyl or heterocyclyl group is optionally substituted by 1, 2 or 3 fluorine atoms.

[85] The compound of [84], wherein 1) when A¹ is N and R^{2a} is —C₁₋₄ alkyl-C(═O)NHRa^{a′}, then Ra^{a′} is other than -(5- to 10-membered)-C₂₋₉ heterocyclyl; or 2) when A⁴ is N, then R^{2a} is other than —C(═O)Ra^{a′}.
[86] A compound selected from the group consisting of compound is selected from the group consisting of

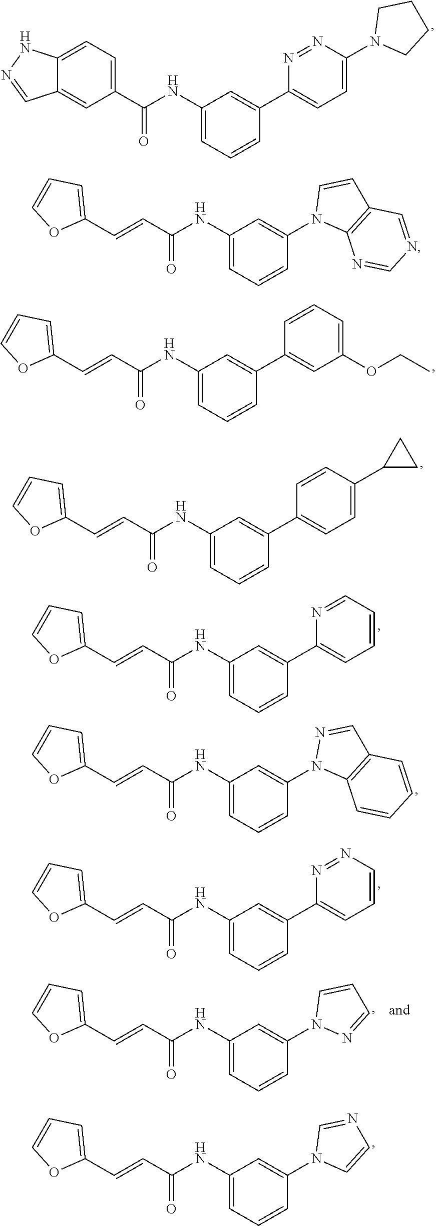

or a pharmaceutically acceptable salt or solvate thereof.
[87] A compound selected from the group consisting of

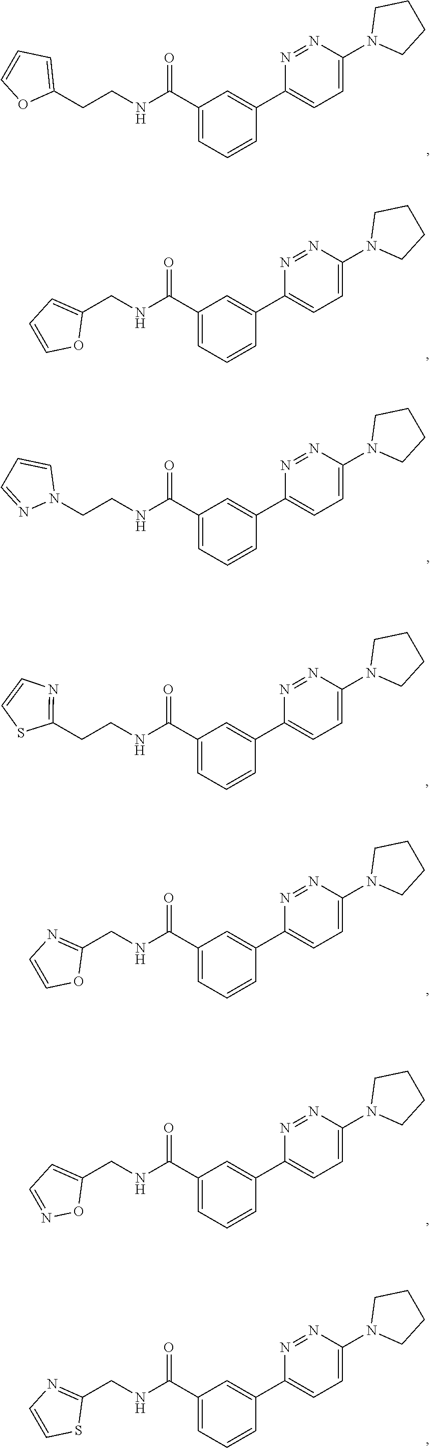

or a pharmaceutically acceptable salt or solvate thereof.
[88] A pharmaceutical composition, comprising an effective amount of a compound of formula (IA), or a pharmaceutically acceptable salt or solvate thereof, and at least one pharmaceutically acceptable excipient, wherein the compound of formula (IA) has the structure:

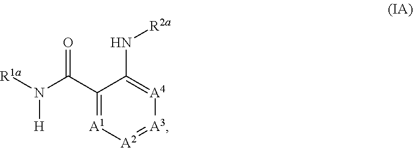

or a pharmaceutically acceptable salt or solvate thereof, wherein

A¹, A², A³, and A⁴ are each independently selected from the group consisting of N, CH and C(R^{3a});

each R^{3a} is independently selected from the group consisting of halogen, —C₁₋₄ alkyl, —C₁₋₄ alkoxy, and —CN;

R^{1a} is selected from the group consisting of —C₁₋₄ alkyl, —C₃₋₁₀ cycloalkyl, —C₁₋₄ alkyl-C₃₋₁₀ cycloalkyl, —C₆₋₁₀ aryl, —C₁₋₄ alkyl-C₆₋₁₀ aryl, -(5- to 10-membered)-C₁₋₉ heteroaryl, —C₁₋₄ alkyl-(5- to 10-membered)-C₁₋₉ heteroaryl, -(5- to 10-membered)-C₂₋₉ heterocyclyl, and —C₁₋₄ alkyl-(5- to 10-membered)-C₂₋₉ heterocyclyl, wherein said alkyl, cycloalkyl, alkylcycloalkyl, aryl, alkylaryl, heteroaryl, alkylheteroaryl, heterocyclyl and alkylheterocyclyl groups are optionally substituted with 1, 2 or 3 substituents each independently selected from the group consisting of halogen, hydroxy, CN, —ORb^{a}, —SRb^{a}, —N(Rb^{a})₂, —C₁₋₄ alkyl optionally substituted with 1, 2, or 3 halogen atoms, optionally substituted —C₆₋₁₀ aryl, optionally substituted -(5- to 10-membered)-C₁₋₉ heteroaryl, and -(5- to 10-membered)-C₂₋₉ heterocyclyl; and wherein said cycloalkyl, alkylcycloalkyl, aryl, alkylaryl, heteroaryl, alkylheteroaryl, heterocyclyl and alkylheterocyclyl is optionally fused to a further (second) ring; and

R^{2a} is selected from the group consisting of —C₁₋₄ alkyl, —C(═O)Ra^{a}, —S(═O)₂Ra^{a}, —C₁₋₄ alkyl-C(═O)Ra^{a}, —C₁₋₄ alkyl-C(═O)NHRa^{a}, —C₁₋₄ alkyl-C(═O)N(Ra^{a})₂, —C₁₋₄ alkyl-S(═O)₂Ra^{a}, —C₁₋₄ alkyl-S(═O)₂—N(Ra^{a})₂, —C₁₋₄ alkyl-C₃₋₁₀ cycloalkyl, —C₁₋₄ alkyl-C₆₋₁₀ aryl, -(5- to 10-membered)-C₁₋₉ heteroaryl, —C₁₋₄ alkyl-(5- to 10-membered)-C₁₋₉ heteroaryl, (5- to 10-membered)-C₂₋₉ heterocyclyl, and —C₁₋₄ alkyl-(5- to 10-membered)-C₂₋₉ heterocyclyl, wherein said alkyl, cycloalkyl, alkylcycloalkyl, aryl, alkylaryl, heteroaryl, alkylheteroaryl, heterocyclyl and alkylheterocyclyl groups are optionally substituted with 1, 2 or 3 substituents each independently selected from the group consisting of halogen, hydroxy, —CN, —C(═O)Ra^{a}, —ORb^{a}, —SRb^{a}, —N(Rb^{a})₂, (═O), —C₁₋₄ alkyl optionally substituted with 1, 2, or 3 halogen atoms, optionally substituted C₆₋₁₀ aryl, optionally substituted -(5- to 10-membered)-C₁₋₉ heteroaryl, and -(5- to 10-membered)-C₂₋₉ heterocyclyl; and wherein said cycloalkyl, alkylcycloalkyl, aryl, alkylaryl, heteroaryl, alkylheteroaryl, heterocyclyl and alkylheterocyclyl is optionally fused to a further (second) ring;

Ra^{a} is selected from the group consisting of —C₁₋₄ alkyl, —C₃₋₁₀ cycloalkyl, —C₁₋₄ alkyl-C₃₋₁₀ cycloalkyl, —C₆₋₁₀ aryl, —C₁₋₄ alkyl-C₆₋₁₀ aryl, -(5- to 10-membered)-C₁₋₉ heteroaryl, —C₁₋₄ alkyl-(5- to 10-membered)-C₁₋₉ heteroaryl, -(5- to 10-membered)-C₂₋₉ heterocyclyl, and —C₁₋₄ alkyl-(5- to 10-membered)-C₂₋₉ heterocyclyl, wherein said alkyl, cycloalkyl, alkylcycloalkyl, aryl, alkylaryl, heteroaryl, alkylheteroaryl, heterocyclyl and alkylheterocyclyl groups are optionally substituted with 1, 2 or 3 substituents each independently selected from the group consisting of halogen, hydroxy, —CN, —ORb^{a}, —SRb^{a}, —N(Rb^{a})₂, —C₁₋₄ alkyl optionally substituted with 1, 2, or 3 halogen atoms, optionally substituted —C₆₋₁₀ aryl, optionally substituted -(5- to 10-membered)-C₁₋₉ heteroaryl, and -(5- to 10-membered)-C₂₋₉ heterocyclyl; and wherein said cycloalkyl, alkylcycloalkyl, aryl, alkylaryl, heteroaryl, alkylheteroaryl, heterocyclyl and alkylheterocyclyl is optionally fused to a further (second) ring; and

each Rb^{a} is independently hydrogen, —C₁₋₄ alkyl, —C₃₋₁₀ cycloalkyl, or -(5- to 10-membered)-C₂₋₉ heterocyclyl, wherein said alkyl, cycloalkyl or heterocyclyl group is optionally substituted by 1, 2 or 3 fluorine atoms.

[89] The pharmaceutical composition of [88], wherein the compound of formula (IA) is a compound of formula (IIA) having the structure:

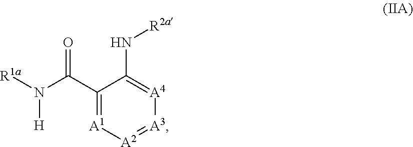

or a pharmaceutically acceptable salt or solvate thereof, wherein

A¹, A², A³, and A⁴ are each independently selected from the group consisting of N, CH and C(R^{3a}), provided that no more than one of A¹, A², A³, or A⁴ is N;

each R^{3a} is independently selected from the group consisting of halogen, —OH, C₁₋₄ alkyl, C₁₋₄ alkoxy, and CN;

R^{1a} is selected from the group consisting of —C₁₋₄ alkyl, —C₃₋₁₀ cycloalkyl, —C₁₋₄ alkyl-C₃₋₁₀ cycloalkyl, —C₆₋₁₀ aryl, —C₁₋₄ alkyl-C₆₋₁₀ aryl, (5- to 10-membered)-C₁₋₉ heteroaryl, —C₁₋₄ alkyl-(5- to 10-membered)-C₁₋₉ heteroaryl, (5- to 10-membered)-C₂₋₉ heterocyclyl, and —C₁₋₄ alkyl-(5- to 10-membered)-C₂₋₉ heterocyclyl, wherein said alkyl, cycloalkyl, alkylcycloalkyl, aryl, alkylaryl, heteroaryl, alkylheteroaryl, heterocyclyl and alkylheterocyclyl groups are optionally substituted with 1, 2 or 3 substituents each independently selected from the group consisting of halogen, hydroxo, —CN, —ORb^{a}, —SRb^{a}, —N(Rb^{a})₂, —C₁₋₄ alkyl optionally substituted with 1, 2, or 3 halogen atoms, optionally substituted C₆₋₁₀ aryl, optionally substituted (5- to 10-membered)-C₁₋₉ heteroaryl, and (5- to 10-membered)-C₂₋₉ heterocyclyl; and wherein said cycloalkyl, alkylcycloalkyl, aryl, alkylaryl, heteroaryl, alkylheteroaryl, heterocyclyl and alkylheterocyclyl is optionally fused to a further (second) ring;

R^{2a′} is selected from the group consisting of —C(═O)Ra^{a′}, —S(═O)₂Ra^{a′}, —C₁₋₄ alkyl-C(═O)NHRa^{a′}, —C₁₋₄ alkyl-C(═O)N(Ra^{a′})₂, —C₁₋₄ alkyl-S(═O)₂—N(Ra^{a′})₂, wherein said alkyl group is optionally substituted with 1, 2 or 3 substituents each independently selected from the group consisting of halogen, hydroxy, —CN, —C₁₋₄ alkyl optionally substituted with 1, 2, or 3 halogen atoms;

Ra^{a′} is selected from the group consisting of —C₆₋₁₀ aryl, —C₁₋₄ alkyl-C₆₋₁₀ aryl, (5- to 10-membered)-C₁₋₉ heteroaryl, —C₁₋₄ alkyl-(5- to 10-membered)-C₁₋₉ heteroaryl, -(5- to 10-membered)-C₂₋₉ heterocyclyl, and —C₁₋₄ alkyl-(5- to 10-membered)-C₂₋₉ heterocyclyl, wherein said aryl, alkylaryl, heteroaryl, alkylheteroaryl, heterocyclyl and alkylheterocyclyl groups are optionally substituted with 1, 2 or 3 substituents each independently selected from the group consisting of halogen, hydroxy, —CN, —ORb^{a}, —SRb^{a}, —N(Rb^{a})₂, —C₁₋₄ alkyl optionally substituted with 1, 2, or 3 halogen atoms, optionally substituted C₆₋₁₀ aryl, optionally substituted (5- to 10-membered)-C₁₋₉ heteroaryl, and (5- to 10-membered)-C₂₋₉ heterocyclyl; and wherein said cycloalkyl, alkylcycloalkyl, aryl, alkylaryl, heteroaryl, alkylheteroaryl, heterocyclyl and alkylheterocyclyl is optionally fused to a further (second) ring; and

each Rb^{a} is independently hydrogen, —C₁₋₄ alkyl, —C₃₋₁₀ cycloalkyl, or -(5- to 10-membered)-C₂₋₉ heterocyclyl, wherein said alkyl, cycloalkyl or heterocyclyl group is optionally substituted by 1, 2 or 3 fluorine atoms.

[90] The pharmaceutical composition of claim 88, wherein the compound of formula (IA) is selected from the group consisting of

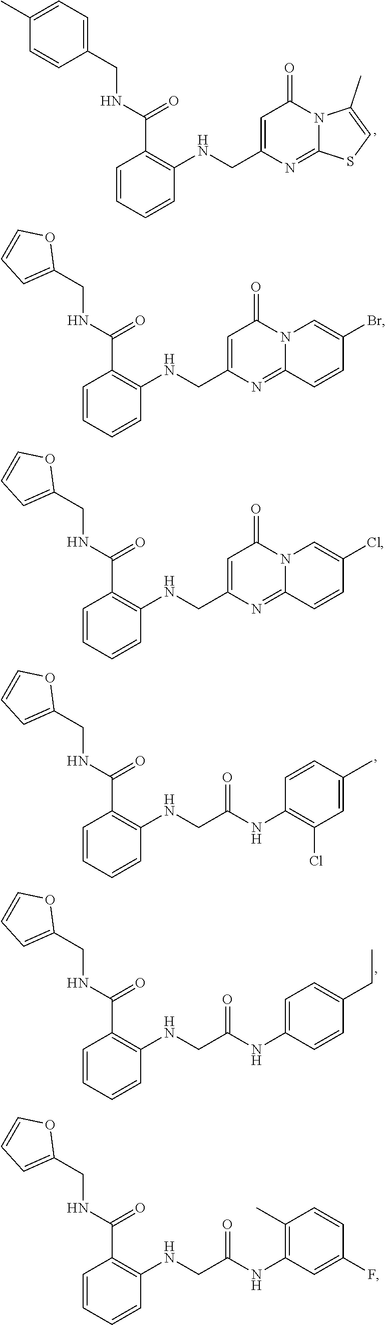 [SMILES: Cc1ccc(CNC(=O)c2ccccc2NCc2cc(=O)n3c(C)csc3n2)cc1 ; O=C(NCc1ccco1)c1ccccc1NCc1cc(=O)n2cc(Br)ccc2n1 ; O=C(NCc1ccco1)c1ccccc1NCc1cc(=O)n2cc(Cl)ccc2n1 ; Cc1ccc(NC(=O)CNc2ccccc2C(=O)NCc2ccco2)c(Cl)c1 ; CCc1ccc(NC(=O)CNc2ccccc2C(=O)NCc2ccco2)cc1 ; Cc1ccc(F)cc1NC(=O)CNc1ccccc1C(=O)NCc1ccco1]

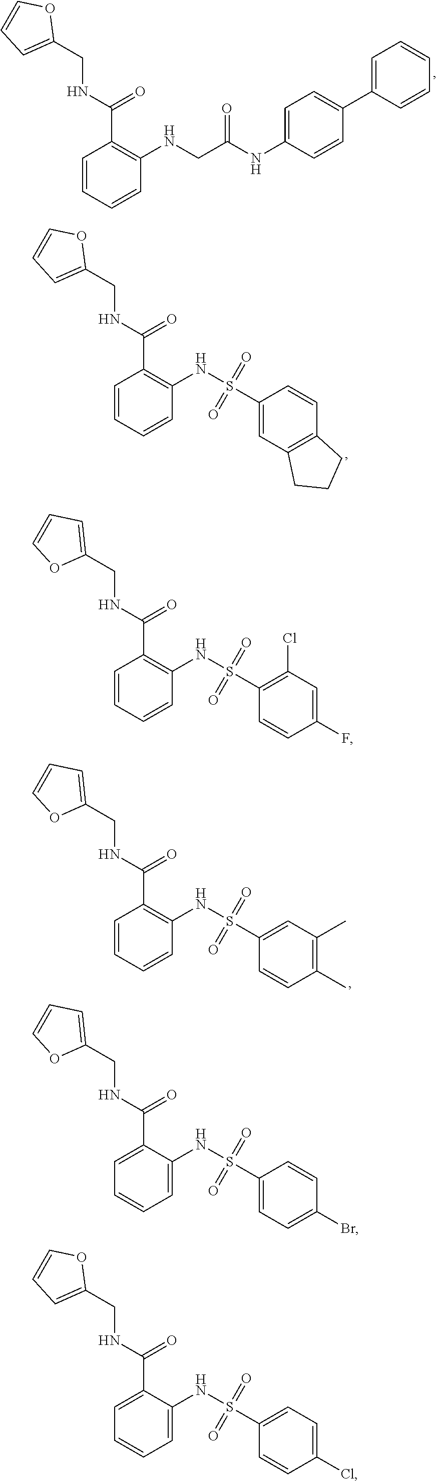

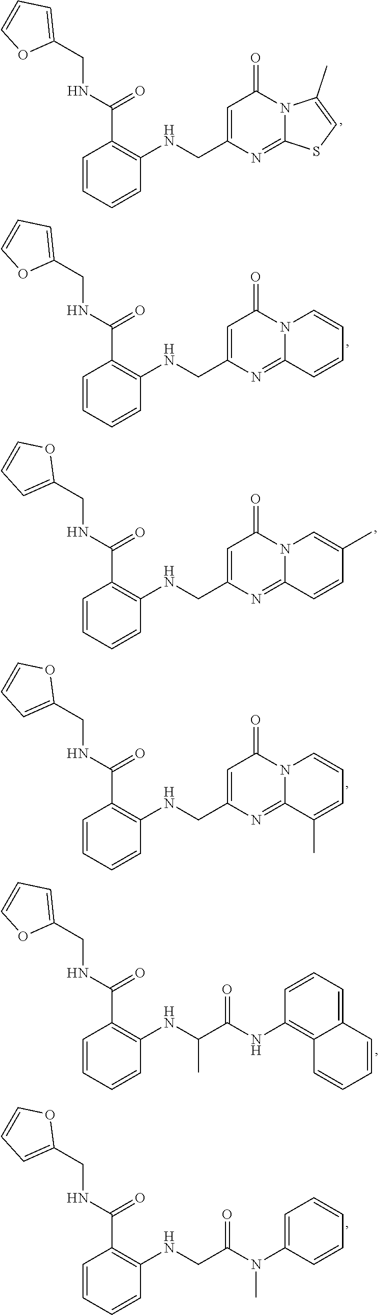 [SMILES: Cc1csc2nc(CNc3ccccc3C(=O)NCc3ccco3)cc(=O)n12 ; O=C(NCc1ccco1)c1ccccc1NCc1cc(=O)n2ccccc2n1 ; Cc1ccc2nc(CNc3ccccc3C(=O)NCc3ccco3)cc(=O)n2c1 ; Cc1cccn2c(=O)cc(CNc3ccccc3C(=O)NCc3ccco3)nc12 ; CC(Nc1ccccc1C(=O)NCc1ccco1)C(=O)Nc1cccc2ccccc12 ; CN(C(=O)CNc1ccccc1C(=O)NCc1ccco1)c1ccccc1]

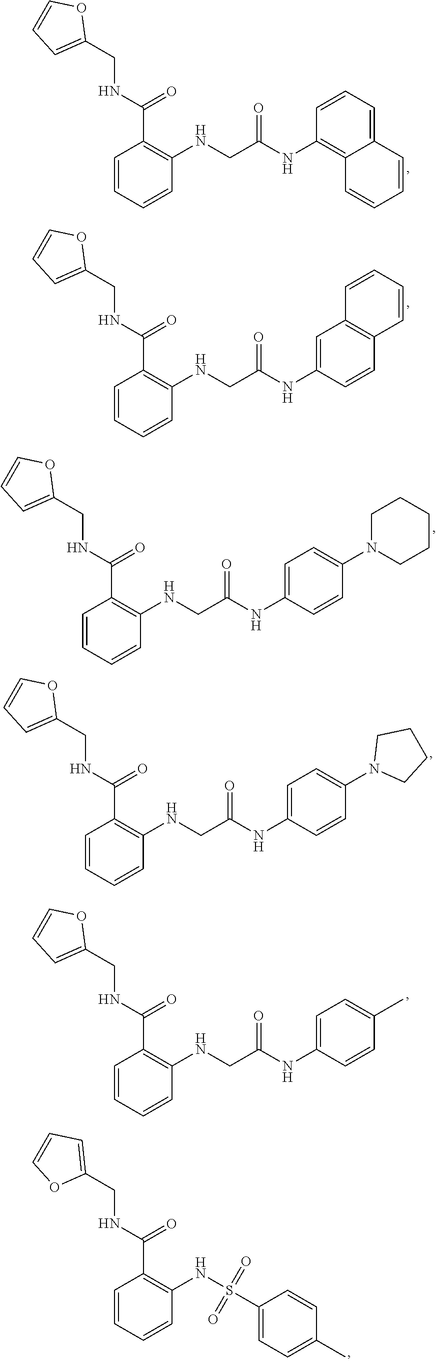

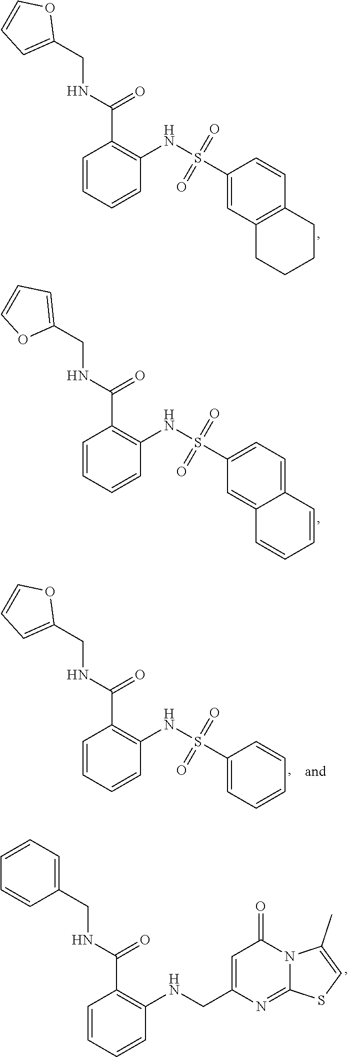 [SMILES: O=C(NCc1ccco1)c1ccccc1NS(=O)(=O)c1ccc2c(c1)CCCC2 ; O=C(NCc1ccco1)c1ccccc1NS(=O)(=O)c1ccc2ccccc2c1 ; O=C(NCc1ccco1)c1ccccc1NS(=O)(=O)c1ccccc1 ; Cc1csc2nc(CNc3ccccc3C(=O)NCc3ccccc3)cc(=O)n12]

or a pharmaceutically acceptable salt or solvate thereof.
[91] A pharmaceutical composition, comprising an effective amount of a compound of formula (IB), or a pharmaceutically acceptable salt or solvate thereof, and at least one pharmaceutically acceptable excipient, wherein the compound of formula (IB) has the structure:

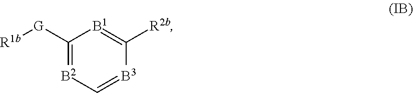

or a pharmaceutically acceptable salt or solvate thereof, wherein

G is —C(═O)—NH— or —NH—C(═O)—;

B¹, B², and B³ are each independently selected from the group consisting of N, CH and C(R^{3b});

each R^{3b} is independently selected from the group consisting of halogen, C₁₋₄ alkyl, —OH, C₁₋₄ alkoxy, and CN;

R^{1b} is selected from the group consisting of —C₁₋₄ alkyl, —C₃₋₁₀ cycloalkyl, —C₁₋₄ alkyl-C₃₋₁₀ cycloalkyl, —C₆₋₁₀ aryl, —C₁₋₄ alkyl-C₆₋₁₀ aryl, —C₂₋₄ alkylene-C₆₋₁₀ aryl, (5- to 10-membered)-C₁₋₉ heteroaryl, —C₁₋₄ alkyl-(5- to 10-membered)-C₁₋₉ heteroaryl, —C₂₋₄ alkylene-(5- to 10-membered)-C₁₋₉ heteroaryl, (5- to 10-membered)-C₂₋₉ heterocyclyl, —C₁₋₄ alkyl-(5- to 10-membered)-C₂₋₉ heterocyclyl, and —C₂₋₄ alkenyl-(5- to 10-membered)-C₂₋₉ heterocyclyl, wherein said alkyl, cycloalkyl, alkylcycloalkyl, aryl, alkylaryl, heteroaryl, alkylheteroaryl, alkenylheteroaryl, heterocyclyl and alkylheterocyclyl groups are optionally substituted with 1, 2 or 3 substituents each independently selected from the group consisting of halogen, hydroxy, —CN, —ORb^{b}, —SRb^{b}, —N(Rb^{b})₂, —C₁₋₄ alkyl optionally substituted with 1, 2, or 3 halogen atoms, optionally substituted C₆₋₁₀ aryl, optionally substituted (5- to 10-membered)-C₁₋₉ heteroaryl, and (5- to 10-membered)-C₂₋₉ heterocyclyl; and wherein said cycloalkyl, alkylcycloalkyl, aryl, alkylaryl, heteroaryl, alkylheteroaryl, alkenylheteroaryl, heterocyclyl and alkylheterocyclyl is optionally fused to a further (second) ring;

R^{2b} is —C₆₋₁₀ aryl, -(5- to 10-membered)-C₁₋₉ heteroaryl, —C(═O)Ra^{b}, —S(═O)₂Ra^{b}, —C(═O)—NH—Ra^{b}, —S(═O)₂—NH—Ra^{b}, —C₁₋₄ alkyl-C(═O)Ra^{b}, —C₁₋₄ alkyl-S(═O)₂Ra^{b}, or —N(Rb^{b})₂, wherein said aryl and heteroaryl groups are optionally substituted with 1, 2 or 3 substituents each independently selected from the group consisting of halogen, hydroxy, CN, —ORb^{b}, —SRb^{b}, —N(Rb^{b})₂, (═O), —C₁₋₄alkyl optionally substituted with 1, 2, or 3 substituents each independently selected from the group consisting of halogen, CN, —ORb^{b}, and —N(Rb^{b})₂, optionally substituted —C₆₋₁₀ aryl, optionally substituted -(5- to 10-membered)-C₁₋₉ heteroaryl, -(5- to 10-membered)-C₂₋₉ heterocyclyl, and —C₃₋₁₀ cycloalkyl; and wherein said aryl, heteroaryl, and heterocyclyl are optionally fused to a further (second) ring; or

R^{2b} and R^{3b} attached to an adjacent carbon atom together form a 5- or 6-membered heterocyclic ring containing one N-atom substituted with —S(═O)₂Ra^{b};

Ra^{b} is selected from the group consisting of —C₁₋₄ alkyl, —C₃₋₁₀ cycloalkyl, —C₁₋₄ alkyl-C₃₋₁₀ cycloalkyl, —C₆₋₁₀ aryl, —C₁₋₄ alkyl-C₆₋₁₀ aryl, (5- to 10-membered)-C₁₋₉ heteroaryl, —C₁₋₄ alkyl-(5- to 10-membered)-C₁₋₉ heteroaryl, (5- to 10-membered)-C₂₋₉ heterocyclyl, and —C₁₋₄ alkyl-(5- to 10-membered)-C₂₋₉ heterocyclyl, wherein said alkyl, cycloalkyl, alkylcycloalkyl, aryl, alkylaryl, heteroaryl, alkylheteroaryl, heterocyclyl and alkylheterocyclyl groups are optionally substituted with 1, 2 or 3 substituents each independently selected from the group consisting of halogen, hydroxy, —CN, —ORb^{b}, —SRb^{b}, —N(Rb^{b})₂, —C₁₋₄ alkyl optionally substituted with 1, 2, or 3 halogen atoms, optionally substituted C₆₋₁₀ aryl, optionally substituted (5- to 10-membered)-C₁₋₉ heteroaryl, and (5- to 10-membered)-C₂₋₉ heterocyclyl; and wherein said cycloalkyl, alkylcycloalkyl, aryl, alkylaryl, heteroaryl, alkylheteroaryl, heterocyclyl and alkylheterocyclyl is optionally fused to a further (second) ring; and

each Rb^{b} is independently hydrogen, —C(═O)Ra^{b}, —S(═O)₂Ra^{b}, —C₁₋₄ alkyl, —C₃₋₁₀ cycloalkyl, -(5- to 10-membered)-C₂₋₉ heterocyclyl, or optionally substituted —C₆₋₁₀ aryl, wherein said alkyl, cycloalkyl or heterocyclyl group is optionally substituted by 1, 2 or 3 fluorine atoms.

[92] The pharmaceutical composition of [91], comprising an effective amount of a compound of formula (IB) where G is —C(═O)—NH—, which is a compound of formula (IIB):

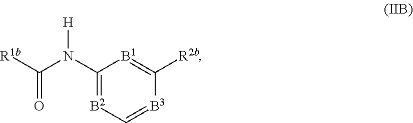

or a pharmaceutically acceptable salt or solvate thereof, wherein B¹, B², B³, R^{1b}, and R^{2b} are as defined in [91].
[93] The pharmaceutical composition of [91], comprising an effective amount of a compound of formula (IB) where G is —NH—C(═O)—, which is a compound of formula (IIIB):

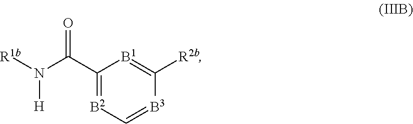

or a pharmaceutically acceptable salt or solvate thereof, wherein B¹, B², B³, R^{1b}, and R^{2b} are as defined in [91].
[94] The pharmaceutical composition of [91] or [92], wherein the compound is selected from the group consisting of

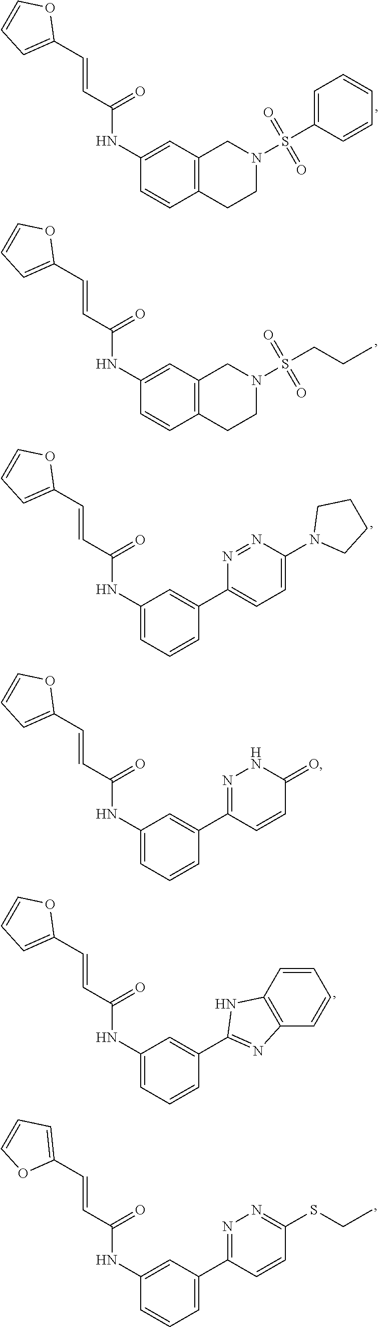 [SMILES: O=C(/C=C/c1ccco1)Nc1ccc2c(c1)CN(S(=O)(=O)c1ccccc1)CC2 ; CCCS(=O)(=O)N1CCc2ccc(NC(=O)/C=C/c3ccco3)cc2C1 ; O=C(/C=C/c1ccco1)Nc1cccc(-c2ccc(N3CCCC3)nn2)c1 ; O=C(/C=C/c1ccco1)Nc1cccc(-c2ccc(=O)[nH]n2)c1 ; O=C(/C=C/c1ccco1)Nc1cccc(-c2nc3ccccc3[nH]2)c1 ; CCSc1ccc(-c2cccc(NC(=O)/C=C/c3ccco3)c2)nn1]

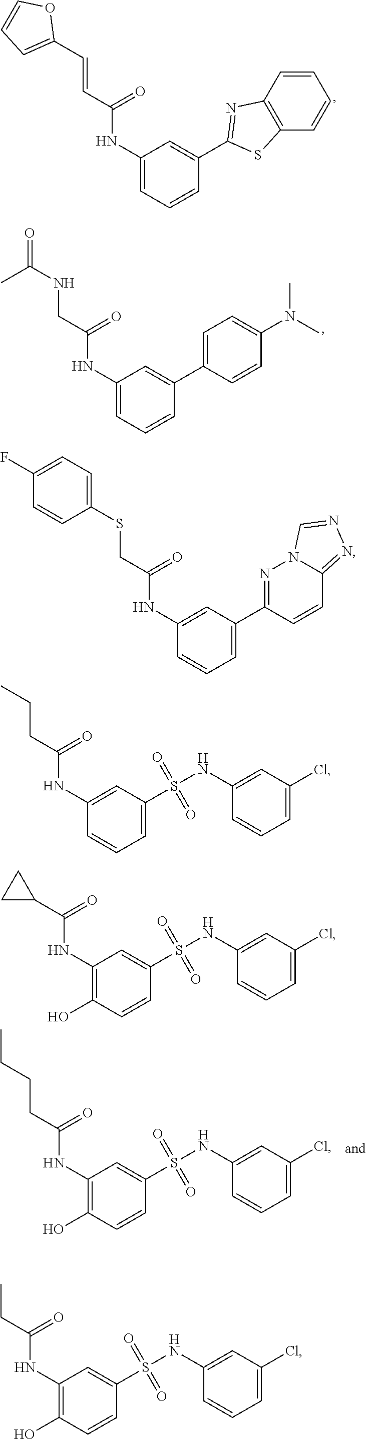

or a pharmaceutically acceptable salt or solvate thereof.
[95] The pharmaceutical composition of [91] or [92], wherein the compound is selected from the group consisting of

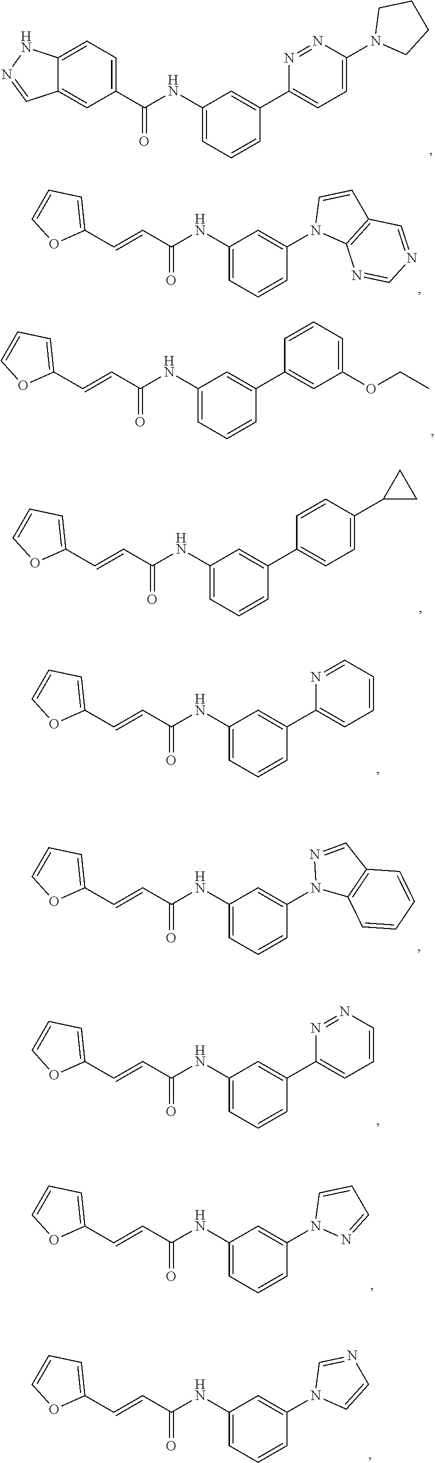

or a pharmaceutically acceptable salt or solvate thereof.
[96] The pharmaceutical composition of [91] or [93], wherein the compound is selected from the group consisting of

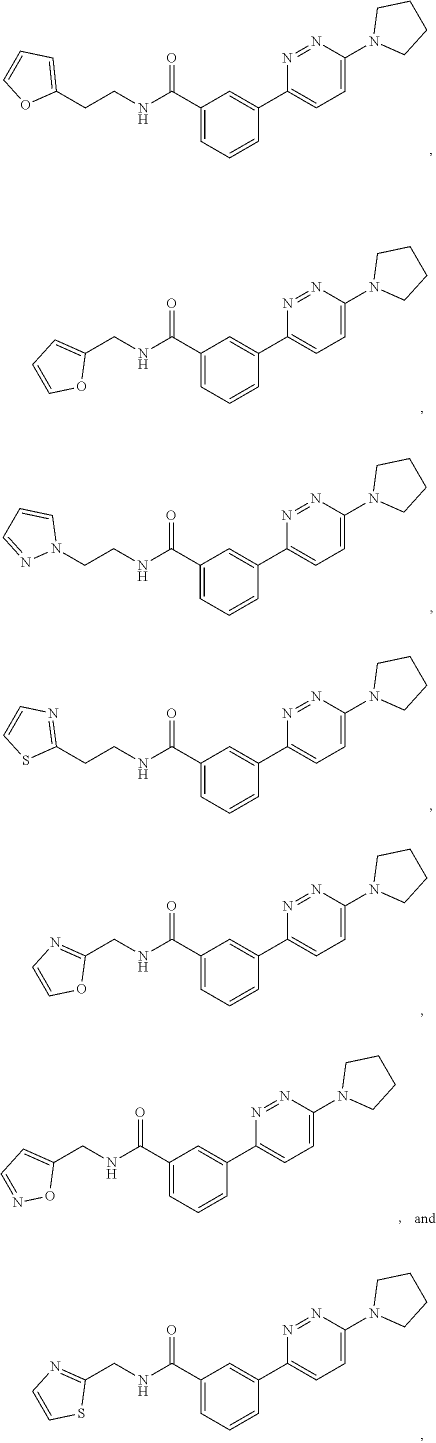

or a pharmaceutically acceptable salt or solvate thereof.
[97] A compound of formula (IA):

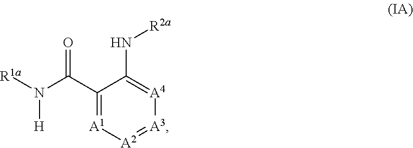

or a pharmaceutically acceptable salt or solvate thereof, for use as a medicament, wherein

A¹, A², A³, and A⁴ are each independently selected from the group consisting of N, CH and C(R^{3a});

each R^{3a} is independently selected from the group consisting of halogen, —C₁₋₄ alkyl, —C₁₋₄ alkoxy, and —CN;

R^{1a} is selected from the group consisting of —C₁₋₄ alkyl, —C₃₋₁₀ cycloalkyl, —C₁₋₄ alkyl-C₃₋₁₀ cycloalkyl, —C₆₋₁₀ aryl, —C₁₋₄ alkyl-C₆₋₁₀ aryl, -(5- to 10-membered)-C₁₋₉ heteroaryl, —C₁₋₄ alkyl-(5- to 10-membered)-C₁₋₉ heteroaryl, -(5- to 10-membered)-C₂₋₉ heterocyclyl, and —C₁₋₄ alkyl-(5- to 10-membered)-C₂₋₉ heterocyclyl, wherein said alkyl, cycloalkyl, alkylcycloalkyl, aryl, alkylaryl, heteroaryl, alkylheteroaryl, heterocyclyl and alkylheterocyclyl groups are optionally substituted with 1, 2 or 3 substituents each independently selected from the group consisting of halogen, hydroxy, CN, —ORb^{a}, —SRb^{a}, —N(Rb^{a})₂, —C₁₋₄ alkyl optionally substituted with 1, 2, or 3 halogen atoms, optionally substituted —C₆₋₁₀ aryl, optionally substituted -(5- to 10-membered)-C₁₋₉ heteroaryl, and -(5- to 10-membered)-C₂₋₉ heterocyclyl; and wherein said cycloalkyl, alkylcycloalkyl, aryl, alkylaryl, heteroaryl, alkylheteroaryl, heterocyclyl and alkylheterocyclyl is optionally fused to a further (second) ring; and

R^{2a} is selected from the group consisting of —C₁₋₄ alkyl, —C(═O)Ra^{a}, —S(═O)₂Ra^{a}, —C₁₋₄ alkyl-C(═O)Ra^{a}, —C₁₋₄ alkyl-C(═O)NHRa^{a}, —C₁₋₄ alkyl-C(═O)N(Ra^{a})₂, —C₁₋₄ alkyl-S(═O)₂Ra^{a}, —C₁₋₄ alkyl-S(═O)₂—N(Ra^{a})₂, —C₁₋₄ alkyl-C₃₋₁₀ cycloalkyl, —C₁₋₄ alkyl-C₆₋₁₀ aryl, -(5- to 10-membered)-C₁₋₉ heteroaryl, —C₁₋₄ alkyl-(5- to 10-membered)-C₁₋₉ heteroaryl, (5- to 10-membered)-C₂₋₉ heterocyclyl, and —C₁₋₄ alkyl-(5- to 10-membered)-C₂₋₉ heterocyclyl, wherein said alkyl, cycloalkyl, alkylcycloalkyl, aryl, alkylaryl, heteroaryl, alkylheteroaryl, heterocyclyl and alkylheterocyclyl groups are optionally substituted with 1, 2 or 3 substituents each independently selected from the group consisting of halogen, hydroxy, —CN, —C(═O)Ra^{a}, —ORb^{a}, —SRb^{a}, —N(Rb^{a})₂, (═O), —C₁₋₄ alkyl optionally substituted with 1, 2, or 3 halogen atoms, optionally substituted C₆₋₁₀ aryl, optionally substituted -(5- to 10-membered)-C₁₋₉ heteroaryl, and -(5- to 10-membered)-C₂₋₉ heterocyclyl; and wherein said cycloalkyl, alkylcycloalkyl, aryl, alkylaryl, heteroaryl, alkylheteroaryl, heterocyclyl and alkylheterocyclyl is optionally fused to a further (second) ring;

Ra^{a} is selected from the group consisting of —C₁₋₄ alkyl, —C₃₋₁₀ cycloalkyl, —C₁₋₄ alkyl-C₃₋₁₀ cycloalkyl, —C₆₋₁₀ aryl, —C₁₋₄ alkyl-C₆₋₁₀ aryl, -(5- to 10-membered)-C₁₋₉ heteroaryl, —C₁₋₄ alkyl-(5- to 10-membered)-C₁₋₉ heteroaryl, -(5- to 10-membered)-C₂₋₉ heterocyclyl, and —C₁₋₄ alkyl-(5- to 10-membered)-C₂₋₉ heterocyclyl, wherein said alkyl, cycloalkyl, alkylcycloalkyl, aryl, alkylaryl, heteroaryl, alkylheteroaryl, heterocyclyl and alkylheterocyclyl groups are optionally substituted with 1, 2 or 3 substituents each independently selected from the group consisting of halogen, hydroxy, —CN, —ORb^{a}, —SRb^{a}, —N(Rb^{a})₂, —C₁₋₄ alkyl optionally substituted with 1, 2, or 3 halogen atoms, optionally substituted —C₆₋₁₀ aryl, optionally substituted -(5- to 10-membered)-C₁₋₉ heteroaryl, and -(5- to 10-membered)-C₂₋₉ heterocyclyl; and wherein said cycloalkyl, alkylcycloalkyl, aryl, alkylaryl, heteroaryl, alkylheteroaryl, heterocyclyl and alkylheterocyclyl is optionally fused to a further (second) ring; and

each Rb^{a} is independently hydrogen, —C₁₋₄ alkyl, —C₃₋₁₀ cycloalkyl, or -(5- to 10-membered)-C₂₋₉ heterocyclyl, wherein said alkyl, cycloalkyl or heterocyclyl group is optionally substituted by 1, 2 or 3 fluorine atoms.

[98] The compound for use according to [97], wherein the compound is selected from the group consisting of

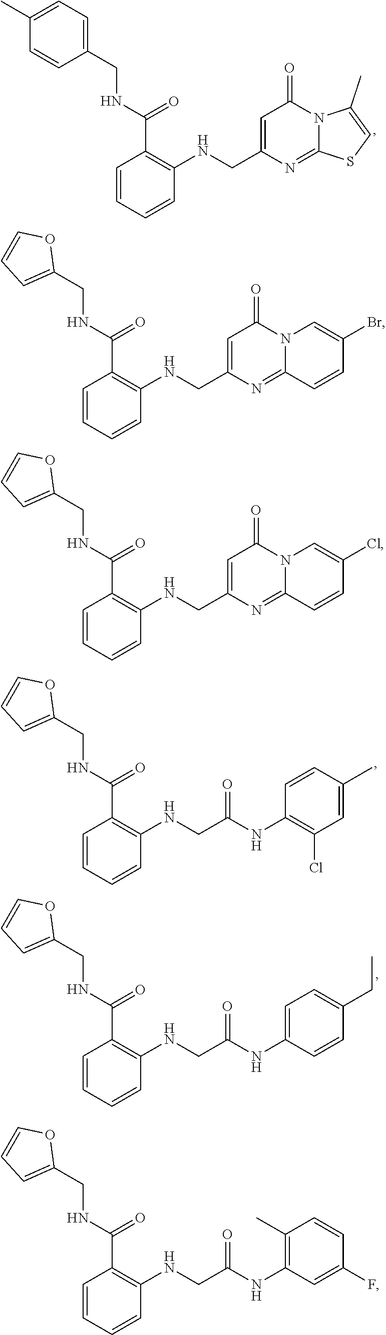 [SMILES: Cc1ccc(CNC(=O)c2ccccc2NCc2cc(=O)n3c(C)csc3n2)cc1 ; O=C(NCc1ccco1)c1ccccc1NCc1cc(=O)n2cc(Br)ccc2n1 ; O=C(NCc1ccco1)c1ccccc1NCc1cc(=O)n2cc(Cl)ccc2n1 ; Cc1ccc(NC(=O)CNc2ccccc2C(=O)NCc2ccco2)c(Cl)c1 ; CCc1ccc(NC(=O)CNc2ccccc2C(=O)NCc2ccco2)cc1 ; Cc1ccc(F)cc1NC(=O)CNc1ccccc1C(=O)NCc1ccco1]

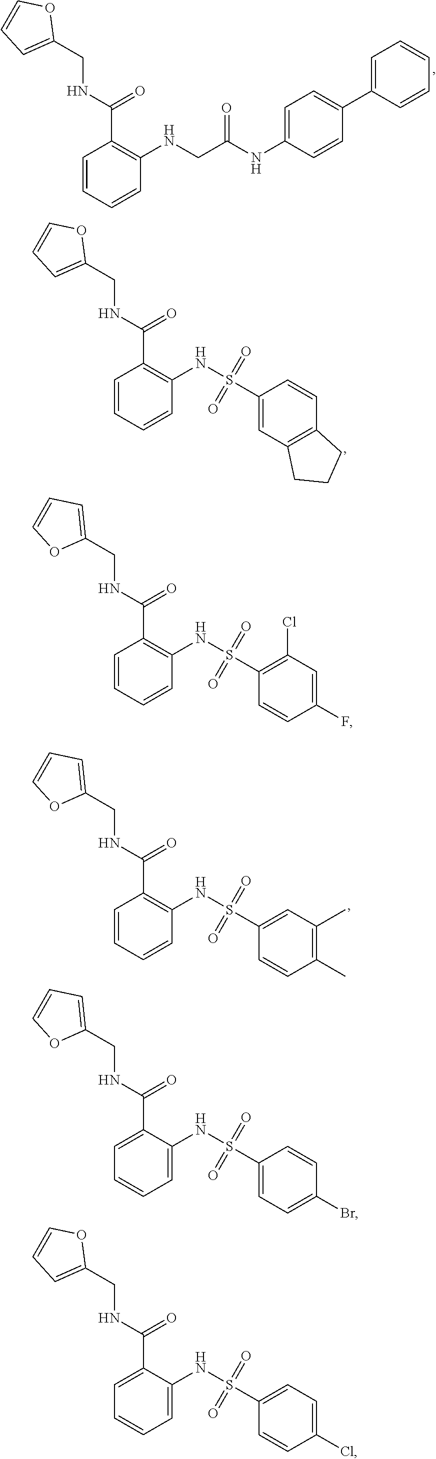

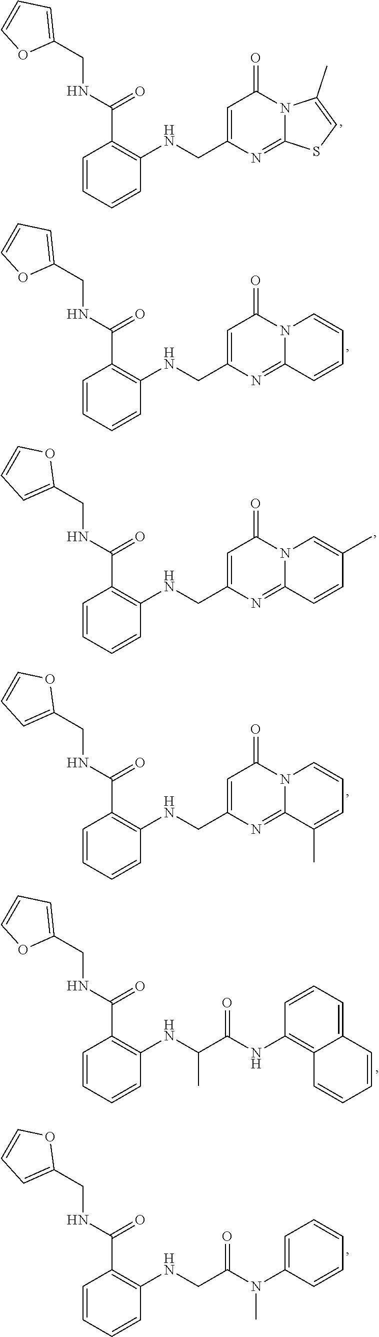 [SMILES: Cc1csc2nc(CNc3ccccc3C(=O)NCc3ccco3)cc(=O)n12 ; O=C(NCc1ccco1)c1ccccc1NCc1cc(=O)n2ccccc2n1 ; Cc1ccc2nc(CNc3ccccc3C(=O)NCc3ccco3)cc(=O)n2c1 ; Cc1cccn2c(=O)cc(CNc3ccccc3C(=O)NCc3ccco3)nc12 ; CC(Nc1ccccc1C(=O)NCc1ccco1)C(=O)Nc1cccc2ccccc12 ; CN(C(=O)CNc1ccccc1C(=O)NCc1ccco1)c1ccccc1]

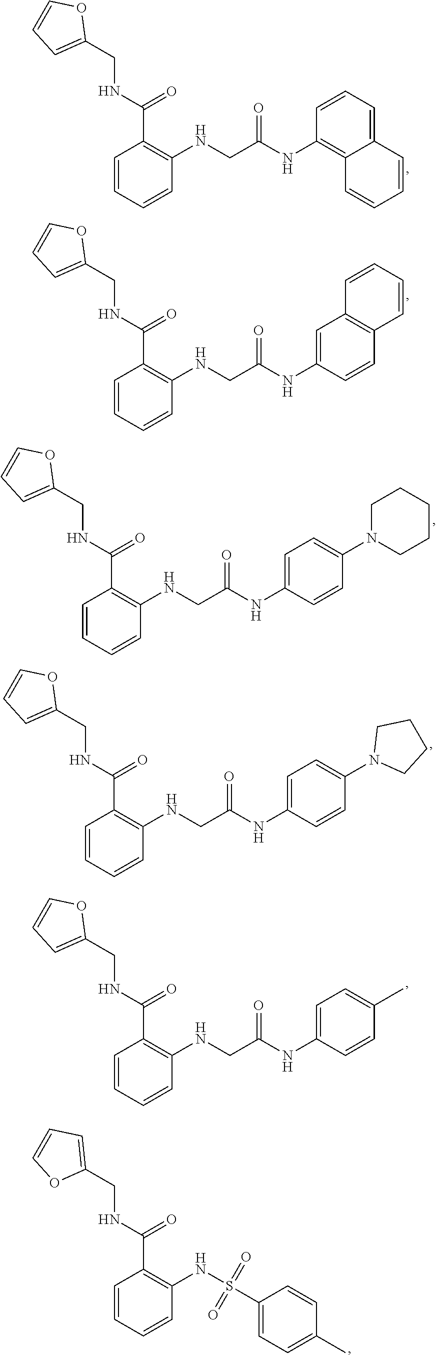

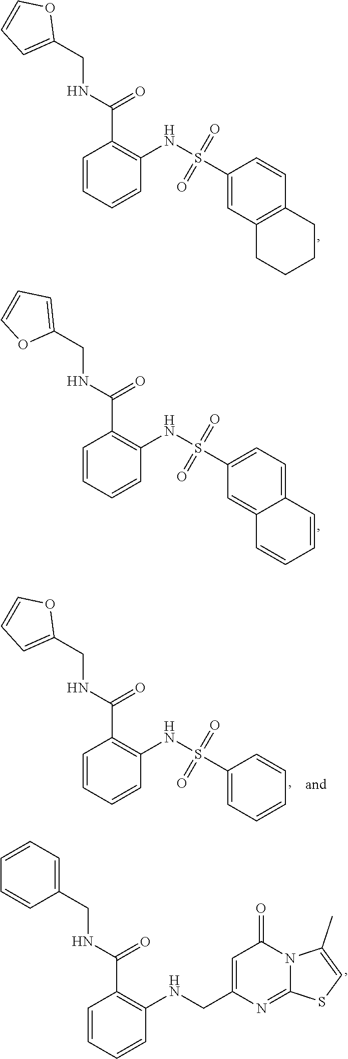 [SMILES: O=C(NCc1ccco1)c1ccccc1NS(=O)(=O)c1ccc2c(c1)CCCC2 ; O=C(NCc1ccco1)c1ccccc1NS(=O)(=O)c1ccc2ccccc2c1 ; O=C(NCc1ccco1)c1ccccc1NS(=O)(=O)c1ccccc1 ; Cc1csc2nc(CNc3ccccc3C(=O)NCc3ccccc3)cc(=O)n12]

or a pharmaceutically acceptable salt or solvate thereof.
[99] A compound of formula (IB) having the structure:

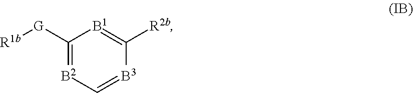

or a pharmaceutically acceptable salt or solvate thereof, for use as a medicament, wherein

G is —C(═O)—NH— or —NH—C(═O)—;

B¹, B², and B³ are each independently selected from the group consisting of N, CH and C(R^{3b});

each R^{3b} is independently selected from the group consisting of halogen, C₁₋₄ alkyl, —OH, C₁₋₄ alkoxy, and CN;

R^{1b} is selected from the group consisting of —C₁₋₄ alkyl, —C₃₋₁₀ cycloalkyl, —C₁₋₄ alkyl-C₃₋₁₀ cycloalkyl, —C₆₋₁₀ aryl, —C₁₋₄ alkyl-C₆₋₁₀ aryl, —C₂₋₄ alkylene-C₆₋₁₀ aryl, (5- to 10-membered)-C₁₋₉ heteroaryl, —C₁₋₄ alkyl-(5- to 10-membered)-C₁₋₉ heteroaryl, —C₂₋₄ alkylene-(5- to 10-membered)-C₁₋₉ heteroaryl, (5- to 10-membered)-C₂₋₉ heterocyclyl, —C₁₋₄ alkyl-(5- to 10-membered)-C₂₋₉ heterocyclyl, and —C₂₋₄ alkenyl-(5- to 10-membered)-C₂₋₉ heterocyclyl, wherein said alkyl, cycloalkyl, alkylcycloalkyl, aryl, alkylaryl, heteroaryl, alkylheteroaryl, alkenylheteroaryl, heterocyclyl and alkylheterocyclyl groups are optionally substituted with 1, 2 or 3 substituents each independently selected from the group consisting of halogen, hydroxy, —CN, —ORb^{b}, —SRb^{b}, —N(Rb^{b})₂, —C₁₋₄ alkyl optionally substituted with 1, 2, or 3 halogen atoms, optionally substituted C₆₋₁₀ aryl, optionally substituted (5- to 10-membered)-C₁₋₉ heteroaryl, and (5- to 10-membered)-C₂₋₉ heterocyclyl; and wherein said cycloalkyl, alkylcycloalkyl, aryl, alkylaryl, heteroaryl, alkylheteroaryl, alkenylheteroaryl, heterocyclyl and alkylheterocyclyl is optionally fused to a further (second) ring;

R^{2b} is —C₆₋₁₀ aryl, -(5- to 10-membered)-C₁₋₉ heteroaryl, —C(═O)Ra^{b}, —S(═O)₂Ra^{b}, —C(═O)—NH—Ra^{b}, —S(═O)₂—NH—Ra^{b}, —C₁₋₄ alkyl-C(═O)Ra^{b}, —C₁₋₄ alkyl-S(═O)₂Ra^{b}, or —N(Rb^{b})₂, wherein said aryl and heteroaryl groups are optionally substituted with 1, 2 or 3 substituents each independently selected from the group consisting of halogen, hydroxy, CN, —ORb^{b}, —SRb^{b}, —N(Rb^{b})₂, (═O), —C₁₋₄alkyl optionally substituted with 1, 2, or 3 substituents each independently selected from the group consisting of halogen, CN, —ORb^{b}, and —N(Rb^{b})₂, optionally substituted —C₆₋₁₀ aryl, optionally substituted -(5- to 10-membered)-C₁₋₉ heteroaryl, -(5- to 10-membered)-C₂₋₉ heterocyclyl, and —C₃₋₁₀ cycloalkyl; and wherein said aryl, heteroaryl, and heterocyclyl are optionally fused to a further (second) ring; or

R^{2b} and R^{3b} attached to an adjacent carbon atom together form a 5- or 6-membered heterocyclic ring containing one N-atom substituted with —S(═O)₂Ra^{b};

Ra^{b} is selected from the group consisting of —C₁₋₄ alkyl, —C₃₋₁₀ cycloalkyl, —C₁₋₄ alkyl-C₃₋₁₀ cycloalkyl, —C₆₋₁₀ aryl, —C₁₋₄ alkyl-C₆₋₁₀ aryl, (5- to 10-membered)-C₁₋₉ heteroaryl, —C₁₋₄ alkyl-(5- to 10-membered)-C₁₋₉ heteroaryl, (5- to 10-membered)-C₂₋₉ heterocyclyl, and —C₁₋₄ alkyl-(5- to 10-membered)-C₂₋₉ heterocyclyl, wherein said alkyl, cycloalkyl, alkylcycloalkyl, aryl, alkylaryl, heteroaryl, alkylheteroaryl, heterocyclyl and alkylheterocyclyl groups are optionally substituted with 1, 2 or 3 substituents each independently selected from the group consisting of halogen, hydroxy, —CN, —ORb^{b}, —SRb^{b}, —N(Rb^{b})₂, —C₁₋₄ alkyl optionally substituted with 1, 2, or 3 halogen atoms, optionally substituted C₆₋₁₀ aryl, optionally substituted (5- to 10-membered)-C₁₋₉ heteroaryl, and (5- to 10-membered)-C₂₋₉ heterocyclyl; and wherein said cycloalkyl, alkylcycloalkyl, aryl, alkylaryl, heteroaryl, alkylheteroaryl, heterocyclyl and alkylheterocyclyl is optionally fused to a further (second) ring; and

each Rb^{b} is independently hydrogen, —C(═O)Ra^{b}, —S(═O)₂Ra^{b}, —C₁₋₄ alkyl, —C₃₋₁₀ cycloalkyl, -(5- to 10-membered)-C₂₋₉ heterocyclyl, or optionally substituted —C₆₋₁₀ aryl, wherein said alkyl, cycloalkyl or heterocyclyl group is optionally substituted by 1, 2 or 3 fluorine atoms.

The compound for use according to [99], which is compound of formula (IIB):

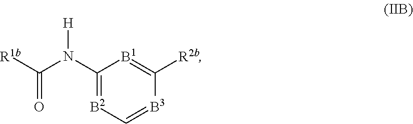

or a pharmaceutically acceptable salt or solvate thereof, wherein B¹, B², B³, R^{1b}, and R^{2b} are as defined in [99].

The compound for use according to [99], which is a compound of formula (IIIB):

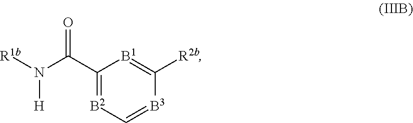

or a pharmaceutically acceptable salt or solvate thereof, wherein B¹, B², B³, R^{1b}, and R^{2b} are as defined in [99].
[102] The compound for use according to [99] or [100], wherein the compound is selected from the group consisting of

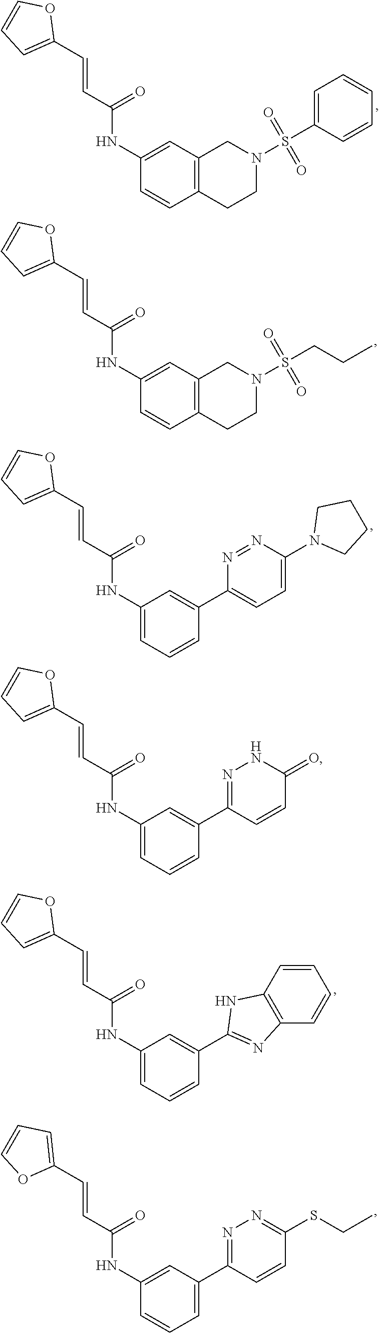 [SMILES: O=C(/C=C/c1ccco1)Nc1ccc2c(c1)CN(S(=O)(=O)c1ccccc1)CC2 ; CCCS(=O)(=O)N1CCc2ccc(NC(=O)/C=C/c3ccco3)cc2C1 ; O=C(/C=C/c1ccco1)Nc1cccc(-c2ccc(N3CCCC3)nn2)c1 ; O=C(/C=C/c1ccco1)Nc1cccc(-c2ccc(=O)[nH]n2)c1 ; O=C(/C=C/c1ccco1)Nc1cccc(-c2nc3ccccc3[nH]2)c1 ; CCSc1ccc(-c2cccc(NC(=O)/C=C/c3ccco3)c2)nn1]

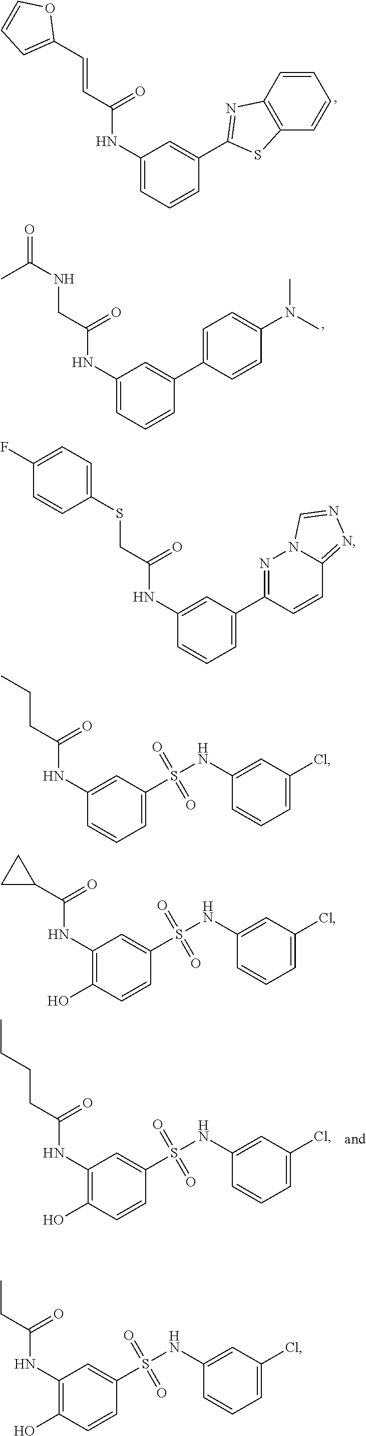

or a pharmaceutically acceptable salt or solvate thereof.
[103] The compound for use according to [99] or [100], wherein the compound is selected from the group consisting of

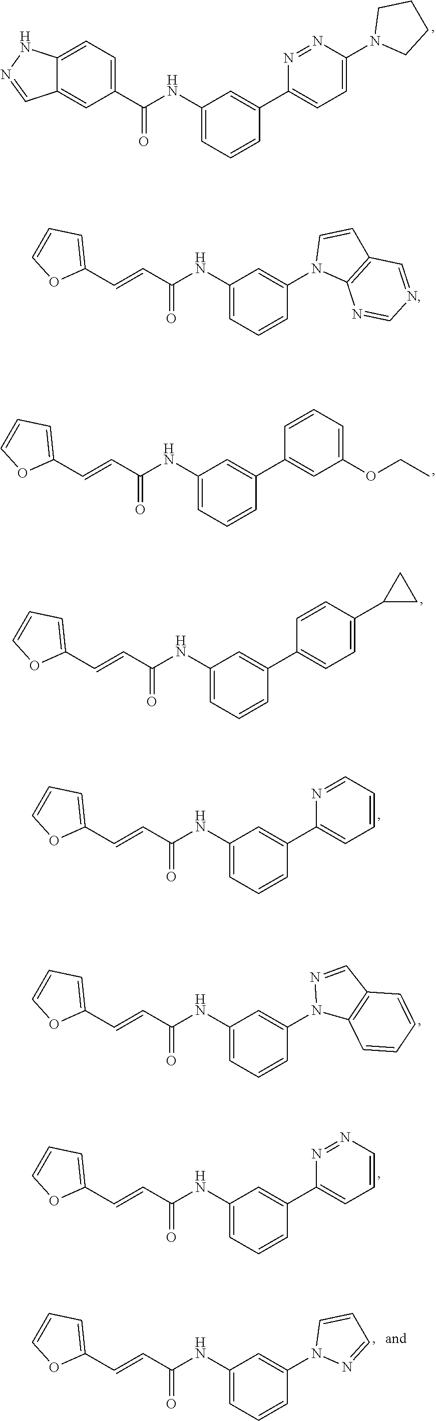

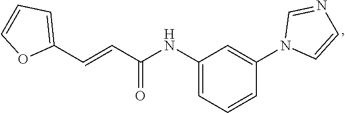 [SMILES: O=C(/C=C/c1ccco1)Nc1cccc(-n2ccnc2)c1]

or a pharmaceutically acceptable salt or solvate thereof.
[104] The compound for use according to [99] or [101], wherein the compound is selected from the group consisting of

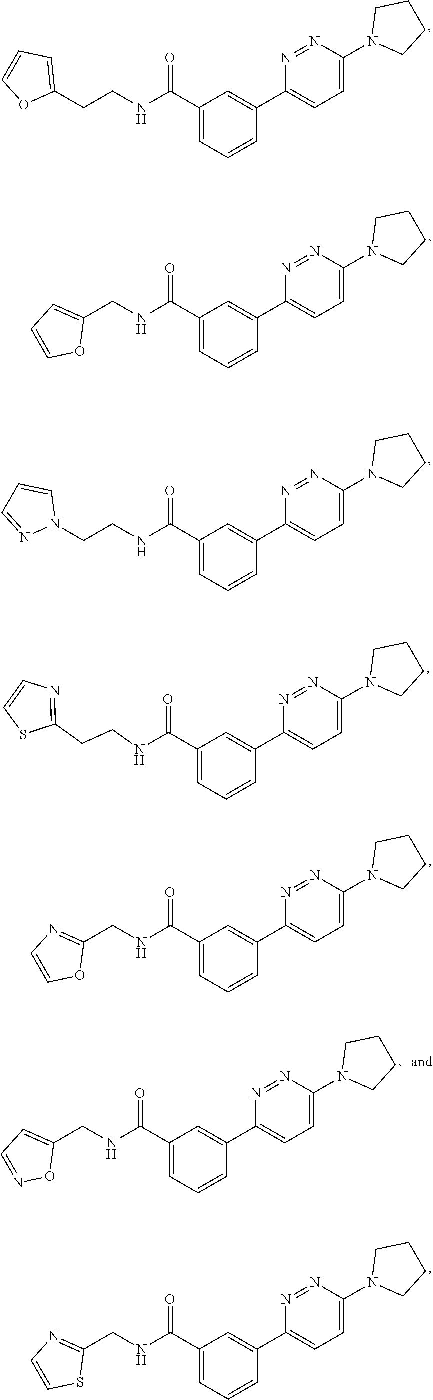

or a pharmaceutically acceptable salt or solvate thereof.
[105] The compound for use according to any one of [97] to [104], wherein the medicament is for use in the treatment or prevention of a lysosomal storage disease.
[106] The compound for use according to [105], wherein the lysosomal storage disease is Krabbe's disease.
[107] The compound for use according to any one of [97] to [104], wherein the medicament is for use in the treatment or prevention of an α-synucleinopathy.
[108] The compound for use according to any one of [97] to [104], wherein the medicament is for use in the treatment or prevention of a disease or disorder selected from the group consisting of Krabbe's disease, demyelinating disorders, galactosylsphingosine related disorders, globoid cell leukodystrophy, multiple sclerosis (MS), Parkinson's disease, peripheral neuropathy, progressive multiple sclerosis, pulmonary artery enlargement in COPD, open angle glaucoma, Lewy body dementia, and multiple system atrophy (MSA).

What is claimed is:

1. A method of treating or preventing a condition associated with the alteration of the activity of galactocerebrosidase in a patient, comprising administering to the patient in need thereof an effective amount of a compound of formula (IA): 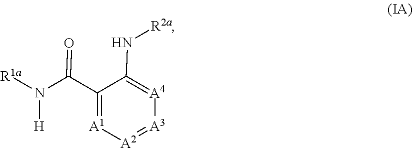
or a pharmaceutically acceptable salt or solvate thereof, wherein
A¹, A², A³, and A⁴ are each independently selected from the group consisting of N, CH and C(R^{3a});
each R^{3a} is independently selected from the group consisting of halogen, —OH, —C₁₋₄ alkyl, halo(C₁₋₄ alkyl), —C₁₋₄ alkoxy, halo(C₁₋₄ alkoxy), and —CN;
R^{1a} is selected from the group consisting of —C₁₋₄ alkyl, —C₃₋₁₀ cycloalkyl, —C₁₋₄ alkyl-C₃₋₁₀ cycloalkyl, —C₆₋₁₀ aryl, —C₁₋₄ alkyl-C₆₋₁₀ aryl, -(5- to 10-membered)-C₁₋₉ heteroaryl, —C₁₋₄ alkyl-(5- to 10-membered)-C₁₋₉ heteroaryl, -(5- to 10-membered)-C₂₋₉ heterocyclyl, and —C₁₋₄ alkyl-(5- to 10-membered)-C₂₋₉ heterocyclyl, wherein said alkyl, cycloalkyl, alkylcycloalkyl, aryl, alkylaryl, heteroaryl, alkylheteroaryl, heterocyclyl and alkylheterocyclyl groups are optionally substituted with 1, 2 or 3 substituents each independently selected from the group consisting of halogen, hydroxy, CN, —ORb^{a}, —SRb^{a}, —N(Rb^{a})₂, —C₁₋₄ alkyl optionally substituted with 1, 2, or 3 halogen atoms, optionally substituted —C₆₋₁₀ aryl, optionally substituted -(5- to 10-membered)-C₁₋₉ heteroaryl, and -(5- to 10-membered)-C₂₋₉ heterocyclyl; and wherein said cycloalkyl, alkylcycloalkyl, aryl, alkylaryl, heteroaryl, alkylheteroaryl, heterocyclyl and alkylheterocyclyl is optionally fused to a further (second) ring; and
R^{2a} is selected from the group consisting of —C₁₋₄ alkyl, —C(═O)Ra^{a}, —C(═O)NHRa^{a}, —S(═O)₂Ra^{a}, —C₁₋₄ alkyl-C(═O)Ra^{a}, —C₁₋₄ alkyl-C(═O)NHRa^{a}, —C₁₋₄ alkyl-C(═O)N(Ra^{a})₂, —C₁₋₄ alkyl-S(═O)₂Ra^{a}, —C₁₋₄ alkyl-S(═O)₂—N(Ra^{a})₂, —C₁₋₄ alkyl-C₃₋₁₀ cycloalkyl, —C₁₋₄ alkyl-C₆₋₁₀ aryl, -(5- to 10-membered)-C₁₋₉ heteroaryl, —C₁₋₄ alkyl-(5- to 10-membered)-C₁₋₉ heteroaryl, (5- to 10-membered)-C₂₋₉ heterocyclyl, and —C₁₋₄ alkyl-(5- to 10-membered)-C₂₋₉ heterocyclyl, wherein said alkyl, cycloalkyl, alkylcycloalkyl, aryl, alkylaryl, heteroaryl, alkylheteroaryl, heterocyclyl and alkylheterocyclyl groups are optionally substituted with 1, 2 or 3 substituents each independently selected from the group consisting of halogen, hydroxy, —CN, —C(═O)Ra^{a}, —ORb^{a}, —SRb^{a}, —N(Rb^{a})₂, (═O), —C₁₋₄ alkyl optionally substituted with 1, 2, or 3 halogen atoms, optionally substituted C₆₋₁₀ aryl, optionally substituted -(5- to 10-membered)-C₁₋₉ heteroaryl, and -(5- to 10-membered)-C₂₋₉ heterocyclyl; and wherein said cycloalkyl, alkylcycloalkyl, aryl, alkylaryl, heteroaryl, alkylheteroaryl, heterocyclyl and alkylheterocyclyl is optionally fused to a further (second) ring;
Ra^{a} is selected from the group consisting of —C₁₋₄ alkyl, —C₃₋₁₀ cycloalkyl, —C₁₋₄ alkyl-C₃₋₁₀ cycloalkyl, —C₆₋₁₀ aryl, —C₁₋₄ alkyl-C₆₋₁₀ aryl, -(5- to 10-membered)-C₁₋₉ heteroaryl, —C₁₋₄ alkyl-(5- to 10-membered)-C₁₋₉ heteroaryl, -(5- to 10-membered)-C₂₋₉ heterocyclyl, and —C₁₋₄ alkyl-(5- to 10-membered)-C₂₋₉ heterocyclyl, wherein said alkyl, cycloalkyl, alkylcycloalkyl, aryl, alkylaryl, heteroaryl, alkylheteroaryl, heterocyclyl and alkylheterocyclyl groups are optionally substituted with 1, 2 or 3 substituents each independently selected from the group consisting of halogen, hydroxy, —CN, —ORb^{a}, —SRb^{a}, —N(Rb^{a})₂, —C₁₋₄ alkyl optionally substituted with 1, 2, or 3 halogen atoms, optionally substituted —C₆₋₁₀ aryl, optionally substituted -(5- to 10-membered)-C₁₋₉ heteroaryl, and -(5- to 10-membered)-C₂₋₉ heterocyclyl; and wherein said cycloalkyl, alkylcycloalkyl, aryl, alkylaryl, heteroaryl, alkylheteroaryl, heterocyclyl and alkylheterocyclyl is optionally fused to a further (second) ring; and
each Rb^{a} is independently hydrogen, —C₁₋₄ alkyl, —C₃₋₁₀ cycloalkyl, or -(5- to 10-membered)-C₂₋₉ heterocyclyl, wherein said alkyl, cycloalkyl or heterocyclyl group is optionally substituted by 1, 2 or 3 fluorine atoms.

2. A method of treating or preventing a lysosomal storage disease or an α-synucleinopathy, comprising administering to a patient in need thereof an effective amount of a compound of formula (IA): 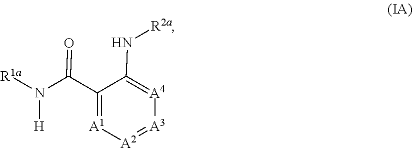
or a pharmaceutically acceptable salt or solvate thereof, wherein
A¹, A², A³, and A⁴ are each independently selected from the group consisting of N, CH and C(R^{3a});
each R^{3a} is independently selected from the group consisting of halogen, —OH, —C₁₋₄ alkyl, halo(C₁₋₄ alkyl), —C₁₋₄ alkoxy, halo(C₁₋₄ alkoxy), and —CN;
R^{1a} is selected from the group consisting of —C₁₋₄ alkyl, —C₃₋₁₀ cycloalkyl, —C₁₋₄ alkyl-C₃₋₁₀ cycloalkyl, —C₆₋₁₀ aryl, —C₁₋₄ alkyl-C₆₋₁₀ aryl, -(5- to 10-membered)-C₁₋₉ heteroaryl, —C₁₋₄ alkyl-(5- to 10-membered)-C₁₋₉ heteroaryl, -(5- to 10-membered)-C₂₋₉ heterocyclyl, and —C₁₋₄ alkyl-(5- to 10-membered)-C₂₋₉ heterocyclyl, wherein said alkyl, cycloalkyl, alkylcycloalkyl, aryl, alkylaryl, heteroaryl, alkylheteroaryl, heterocyclyl and alkylheterocyclyl groups are optionally substituted with 1, 2 or 3 substituents each independently selected from the group consisting of halogen, hydroxy, CN, —ORb^{a}, —SRb^{a}, —N(Rb^{a})₂, —C₁₋₄ alkyl optionally substituted with 1, 2, or 3 halogen atoms, optionally substituted —C₆₋₁₀ aryl, optionally substituted -(5- to 10-membered)-C₁₋₉ heteroaryl, and -(5- to 10-membered)-C₂₋₉ heterocyclyl; and wherein said cycloalkyl, alkylcycloalkyl, aryl, alkylaryl, heteroaryl, alkylheteroaryl, heterocyclyl and alkylheterocyclyl is optionally fused to a further (second) ring; and
R^{2a} is selected from the group consisting of —C₁₋₄ alkyl, —C(═O)Ra^{a}, —C(═O)NHRa^{a}, —S(═O)₂Ra^{a}, —C₁₋₄ alkyl-C(═O)Ra^{a}, —C₁₋₄ alkyl-C(═O)NHRa^{a}, —C₁₋₄ alkyl-C(═O)N(Ra^{a})₂, —C₁₋₄ alkyl-S(═O)₂Ra^{a}, —C₁₋₄ alkyl-S(═O)₂—N(Ra^{a})₂, —C₁₋₄ alkyl-C₃₋₁₀ cycloalkyl, —C₁₋₄ alkyl-C₆₋₁₀ aryl, -(5- to 10-membered)-C₁₋₉ heteroaryl, —C₁₋₄ alkyl-(5- to 10-membered)-C₁₋₉ heteroaryl, (5- to 10-membered)-C₂₋₉ heterocyclyl, and —C₁₋₄ alkyl-(5- to 10-membered)-C₂₋₉ heterocyclyl, wherein said alkyl, cycloalkyl, alkylcycloalkyl, aryl, alkylaryl, heteroaryl, alkylheteroaryl, heterocyclyl and alkylheterocyclyl groups are optionally substituted with 1, 2 or 3 substituents each independently selected from the group consisting of halogen, hydroxy, —CN, —C(═O)Ra^{a}, —ORb^{a}, —SRb^{a}, —N(Rb^{a})₂, (═O), —C₁₋₄ alkyl optionally substituted with 1, 2, or 3 halogen atoms, optionally substituted C₆₋₁₀ aryl, optionally substituted -(5- to 10-membered)-C₁₋₉ heteroaryl, and -(5- to 10-membered)-C₂₋₉ heterocyclyl; and wherein said cycloalkyl, alkylcycloalkyl, aryl, alkylaryl, heteroaryl, alkylheteroaryl, heterocyclyl and alkylheterocyclyl is optionally fused to a further (second) ring;
Ra^{a} is selected from the group consisting of —C₁₋₄ alkyl, —C₃₋₁₀ cycloalkyl, —C₁₋₄ alkyl-C₃₋₁₀ cycloalkyl, —C₆₋₁₀ aryl, —C₁₋₄ alkyl-C₆₋₁₀ aryl, -(5- to 10-membered)-C₁₋₉ heteroaryl, —C₁₋₄ alkyl-(5- to 10-membered)-C₁₋₉ heteroaryl, -(5- to 10-membered)-C₂₋₉ heterocyclyl, and —C₁₋₄ alkyl-(5- to 10-membered)-C₂₋₉ heterocyclyl, wherein said alkyl, cycloalkyl, alkylcycloalkyl, aryl, alkylaryl, heteroaryl, alkylheteroaryl, heterocyclyl and alkylheterocyclyl groups are optionally substituted with 1, 2 or 3 substituents each independently selected from the group consisting of halogen, hydroxy, —CN, —ORb^{a}, —SRb^{a}, —N(Rb^{a})₂, —C₁₋₄ alkyl optionally substituted with 1, 2, or 3 halogen atoms, optionally substituted —C₆₋₁₀ aryl, optionally substituted -(5- to 10-membered)-C₁₋₉ heteroaryl, and -(5- to 10-membered)-C₂₋₉ heterocyclyl; and wherein said cycloalkyl, alkylcycloalkyl, aryl, alkylaryl, heteroaryl, alkylheteroaryl, heterocyclyl and alkylheterocyclyl is optionally fused to a further (second) ring; and
each Rb^{a} is independently hydrogen, —C₁₋₄ alkyl, —C₃₋₁₀ cycloalkyl, or -(5- to 10-membered)-C₂₋₉ heterocyclyl, wherein said alkyl, cycloalkyl or heterocyclyl group is optionally substituted by 1, 2 or 3 fluorine atoms.

3. The method of claim 2, wherein a lysosomal storage disease is treated or prevented.

4. The method of claim 2 or 3, wherein the lysosomal storage disease is Krabbe's disease.

5. The method of claim 2, wherein an α-synucleinopathy is treated or prevented.

6. A method of treating or preventing a disease or disorder, comprising administering to a patient in need thereof an effective amount of a compound of formula (IA): 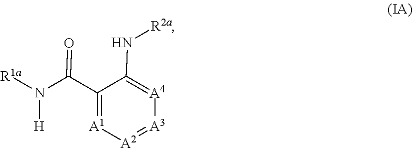
or a pharmaceutically acceptable salt or solvate thereof, wherein
A¹, A², A³, and A⁴ are each independently selected from the group consisting of N, CH and C(R^{3a});
each R^{3a} is independently selected from the group consisting of halogen, —OH, —C₁₋₄ alkyl, halo(C₁₋₄ alkyl), —C₁₋₄ alkoxy, halo(C₁₋₄ alkoxy), and —CN;
R^{1a} is selected from the group consisting of —C₁₋₄ alkyl, —C₃₋₁₀ cycloalkyl, —C₁₋₄ alkyl-C₃₋₁₀ cycloalkyl, —C₆₋₁₀ aryl, —C₁₋₄ alkyl-C₆₋₁₀ aryl, -(5- to 10-membered)-C₁₋₉ heteroaryl, —C₁₋₄ alkyl-(5- to 10-membered)-C₁₋₉ heteroaryl, -(5- to 10-membered)-C₂₋₉ heterocyclyl, and —C₁₋₄ alkyl-(5- to 10-membered)-C₂₋₉ heterocyclyl, wherein said alkyl, cycloalkyl, alkylcycloalkyl, aryl, alkylaryl, heteroaryl, alkylheteroaryl, heterocyclyl and alkylheterocyclyl groups are optionally substituted with 1, 2 or 3 substituents each independently selected from the group consisting of halogen, hydroxy, CN, —ORb^{a}, —SRb^{a}, —N(Rb^{a})₂, —C₁₋₄ alkyl optionally substituted with 1, 2, or 3 halogen atoms, optionally substituted —C₆₋₁₀ aryl, optionally substituted -(5- to 10-membered)-C₁₋₉ heteroaryl, and -(5- to 10-membered)-C₂₋₉ heterocyclyl; and wherein said cycloalkyl, alkylcycloalkyl, aryl, alkylaryl, heteroaryl, alkylheteroaryl, heterocyclyl and alkylheterocyclyl is optionally fused to a further (second) ring; and
R^{2a} is selected from the group consisting of —C₁₋₄ alkyl, —C(═O)Ra^{a}, —C(═O)NHRa^{a}, —S(═O)₂Ra^{a}, —C₁₋₄ alkyl-C(═O)Ra^{a}, —C₁₋₄ alkyl-C(═O)NHRa^{a}, —C₁₋₄ alkyl-C(═O)N(Ra^{a})₂, —C₁₋₄ alkyl-S(═O)₂Ra^{a}, —C₁₋₄ alkyl-S(═O)₂—N(Ra^{a})₂, —C₁₋₄ alkyl-C₃₋₁₀ cycloalkyl, —C₁₋₄ alkyl-C₆₋₁₀ aryl, -(5- to 10-membered)-C₁₋₉ heteroaryl, —C₁₋₄ alkyl-(5- to 10-membered)-C₁₋₉ heteroaryl, (5- to 10-membered)-C₂₋₉ heterocyclyl, and —C₁₋₄ alkyl-(5- to 10-membered)-C₂₋₉ heterocyclyl, wherein said alkyl, cycloalkyl, alkylcycloalkyl, aryl, alkylaryl, heteroaryl, alkylheteroaryl, heterocyclyl and alkylheterocyclyl groups are optionally substituted with 1, 2 or 3 substituents each independently selected from the group consisting of halogen, hydroxy, —CN, —C(═O)Ra^{a}, —ORb^{a}, —SRb^{a}, —N(Rb^{a})₂, (═O), —C₁₋₄ alkyl optionally substituted with 1, 2, or 3 halogen atoms, optionally substituted C₆₋₁₀ aryl, optionally substituted -(5- to 10-membered)-C₁₋₉ heteroaryl, and -(5- to 10-membered)-C₂₋₉ heterocyclyl; and wherein said cycloalkyl, alkylcycloalkyl, aryl, alkylaryl, heteroaryl, alkylheteroaryl, heterocyclyl and alkylheterocyclyl is optionally fused to a further (second) ring;
Ra^{a} is selected from the group consisting of —C₁₋₄ alkyl, —C₃₋₁₀ cycloalkyl, —C₁₋₄ alkyl-C₃₋₁₀ cycloalkyl, —C₆₋₁₀ aryl, —C₁₋₄ alkyl-C₆₋₁₀ aryl, -(5- to 10-membered)-C₁₋₉ heteroaryl, —C₁₋₄ alkyl-(5- to 10-membered)-C₁₋₉ heteroaryl, -(5- to 10-membered)-C₂₋₉ heterocyclyl, and —C₁₋₄ alkyl-(5- to 10-membered)-C₂₋₉ heterocyclyl, wherein said alkyl, cycloalkyl, alkylcycloalkyl, aryl, alkylaryl, heteroaryl, alkylheteroaryl, heterocyclyl and alkylheterocyclyl groups are optionally substituted with 1, 2 or 3 substituents each independently selected from the group consisting of halogen, hydroxy, —CN, —ORb^{a}, —SRb^{a}, —N(Rb^{a})₂, —C₁₋₄ alkyl optionally substituted with 1, 2, or 3 halogen atoms, optionally substituted —C₆₋₁₀ aryl, optionally substituted -(5- to 10-membered)-C₁₋₉ heteroaryl, and -(5- to 10-membered)-C₂₋₉ heterocyclyl; and wherein said cycloalkyl, alkylcycloalkyl, aryl, alkylaryl, heteroaryl, alkylheteroaryl, heterocyclyl and alkylheterocyclyl is optionally fused to a further (second) ring; and
each Rb^{a} is independently hydrogen, —C₁₋₄ alkyl, —C₃₋₁₀ cycloalkyl, or -(5- to 10-membered)-C₂₋₉ heterocyclyl, wherein said alkyl, cycloalkyl or heterocyclyl group is optionally substituted by 1, 2 or 3 fluorine atoms,
wherein said disease or disorder is selected from the group consisting of Krabbe's disease, demyelinating disorders, galactosylsphingosine related disorders, globoid cell leukodystrophy, multiple sclerosis (MS), Parkinson's disease, peripheral neuropathy, progressive multiple sclerosis, pulmonary artery enlargement in COPD, open angle glaucoma, Lewy body dementia, and multiple system atrophy (MSA).

7. The method of any one of claims 1-6, wherein A¹, A², A³, and A⁴ are CH.

8. The method of any one of claims 1-6, wherein one of A¹, A², A³, and A⁴ is C(R^{3a}) and the ones not C(R^{3a}) are CH.

9. The method of any one of claims 1-6, wherein two of A¹, A², A³, and A⁴ is C(R^{3a}) and the ones not C(R^{3a}) are CH.

10. The method of any one of claims 1-6, wherein A¹ is N and A², A³, and A⁴ are each independently selected from the group consisting of CH and C(R^{3a}).

11. The method of any one of claims 1-6, wherein A² is N and A¹, A³, and A⁴ are each independently selected from the group consisting of CH and C(R^{3a}).

12. The method of any one of claims 1-6, wherein A³ is N and A¹, A², and A⁴ are each independently selected from the group consisting of CH and C(R^{3a}).

13. The method of any one of claims 1-6, wherein A⁴ is N and A¹, A², and A³ are each independently selected from the group consisting of CH and C(R^{3a}).

14. The method of any one of claims 1-6, wherein two of A¹, A², A³, and A⁴ are N, and those that are not N are each independently selected from the group consisting of CH and C(R^{3a}).

15. The method of any one of claims 1-6, wherein three of A¹, A², A³, and A⁴ are N, and the one not N is selected from the group consisting of CH and C(R^{3a}).

16. The method of any one of claims 1-6, wherein the compound of formula (IA) is a compound of formula (IIA): 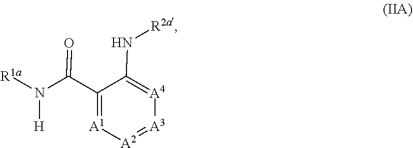
or a pharmaceutically acceptable salt or solvate thereof, wherein
A¹, A², A³, and A⁴ are each independently selected from the group consisting of N, CH and C(R^{3a}), provided that no more than one of A¹, A², A³, or A⁴ is N;
each R^{3a} is independently selected from the group consisting of halogen, —OH, —C₁₋₄ alkyl, halo(C₁₋₄ alkyl), —C₁₋₄ alkoxy, halo(C₁₋₄ alkoxy), and —CN;
R^{1a} is selected from the group consisting of —C₁₋₄ alkyl, —C₃₋₁₀ cycloalkyl, —C₁₋₄ alkyl-C₃₋₁₀ cycloalkyl, —C₆₋₁₀ aryl, —C₁₋₄ alkyl-C₆₋₁₀ aryl, (5- to 10-membered)-C₁₋₉ heteroaryl, —C₁₋₄ alkyl-(5- to 10-membered)-C₁₋₉ heteroaryl, (5- to 10-membered)-C₂₋₉ heterocyclyl, and —C₁₋₄ alkyl-(5- to 10-membered)-C₂₋₉ heterocyclyl, wherein said alkyl, cycloalkyl, alkylcycloalkyl, aryl, alkylaryl, heteroaryl, alkylheteroaryl, heterocyclyl and alkylheterocyclyl groups are optionally substituted with 1, 2 or 3 substituents each independently selected from the group consisting of halogen, hydroxo, —CN, —ORb^{a}, —SRb^{a}, —N(Rb^{a})₂, —C₁₋₄ alkyl optionally substituted with 1, 2, or 3 halogen atoms, optionally substituted C₆₋₁₀ aryl, optionally substituted (5- to 10-membered)-C₁₋₉ heteroaryl, and (5- to 10-membered)-C₂₋₉ heterocyclyl; and wherein said cycloalkyl, alkylcycloalkyl, aryl, alkylaryl, heteroaryl, alkylheteroaryl, heterocyclyl and alkylheterocyclyl is optionally fused to a further (second) ring;
R^{2a′} is selected from the group consisting of —C(═O)Ra^{a′}, —S(═O)₂Ra^{a′}, —C₁₋₄ alkyl-C(═O)NHRa^{a′}, —C₁₋₄ alkyl-C(═O)N(Ra^{a′})₂, —C₁₋₄ alkyl-S(═O)₂—N(Ra^{a′})₂, wherein said alkyl group is optionally substituted with 1, 2 or 3 substituents each independently selected from the group consisting of halogen, hydroxy, —CN, —C₁₋₄ alkyl optionally substituted with 1, 2, or 3 halogen atoms;
Ra^{a′} is selected from the group consisting of —C₆₋₁₀ aryl, —C₁₋₄ alkyl-C₆₋₁₀ aryl, (5- to 10-membered)-C₁₋₉ heteroaryl, —C₁₋₄ alkyl-(5- to 10-membered)-C₁₋₉ heteroaryl, -(5- to 10-membered)-C₂₋₉ heterocyclyl, and —C₁₋₄ alkyl-(5- to 10-membered)-C₂₋₉ heterocyclyl, wherein said aryl, alkylaryl, heteroaryl, alkylheteroaryl, heterocyclyl and alkylheterocyclyl groups are optionally substituted with 1, 2 or 3 substituents each independently selected from the group consisting of halogen, hydroxy, —CN, —ORb^{a}, —SRb^{a}, —N(Rb^{a})₂, —C₁₋₄ alkyl optionally substituted with 1, 2, or 3 halogen atoms, optionally substituted C₆₋₁₀ aryl, optionally substituted (5- to 10-membered)-C₁₋₉ heteroaryl, and (5- to 10-membered)-C₂₋₉ heterocyclyl; and wherein said cycloalkyl, alkylcycloalkyl, aryl, alkylaryl, heteroaryl, alkylheteroaryl, heterocyclyl and alkylheterocyclyl is optionally fused to a further (second) ring; and
each Rb^{a} is independently hydrogen, —C₁₋₄ alkyl, —C₃₋₁₀ cycloalkyl, or -(5- to 10-membered)-C₂₋₉ heterocyclyl, wherein said alkyl, cycloalkyl or heterocyclyl group is optionally substituted by 1, 2 or 3 fluorine atoms.

17. The method of claim 16, wherein 1) when A¹ is N and R^{2a} is —C₁₋₄ alkyl-C(═O)NHRa^{a′}, then Ra^{a′} is other than -(5- to 10-membered)-C₂₋₉ heterocyclyl; or 2) when A⁴ is N, then R^{2a} is other than —C(═O)Ra^{a′}.

18. The method of any one of claims 1-17, wherein R^{1a} is —C₆₋₁₀ aryl or —C₁₋₄ alkyl-C₆₋₁₀ aryl, wherein said aryl or alkylaryl is optionally substituted with 1, 2 or 3 substituents each independently selected from the group consisting of halogen, hydroxy, —CN, —ORb^{a}, —SRb^{a}, —N(Rb^{a})₂, —C₁₋₄ alkyl optionally substituted with 1, 2, or 3 halogen atoms, optionally substituted —C₆₋₁₀ aryl, optionally substituted -(5- to 10-membered)-C₁₋₉ heteroaryl, and -(5- to 10-membered)-C₂₋₉ heterocyclyl, wherein Rb^{a} is as defined in claim 1.

19. The method of any one of claims 1-17, wherein R^{1a} is unsubstituted —C₁₋₄ alkyl-C₆₋₁₀ aryl or —C₁₋₄ alkyl-C₆₋₁₀ aryl optionally substituted with 1, 2 or 3 substituents each independently selected from the group consisting of halogen, hydroxy, —CN, —ORb^{a}, —SRb^{a}, —N(Rb^{a})₂, —C₁₋₄alkyl optionally substituted with 1, 2, or 3 halogen atoms, optionally substituted —C₆₋₁₀ aryl, optionally substituted -(5- to 10-membered)-C₁₋₉ heteroaryl and -(5- to 10-membered)-C₂₋₉ heterocyclyl, wherein Rb^{a} is as defined in claim 1.

20. The method of any one of claims 1-17 or 19, wherein R^{1a} is unsubstituted benzyl or unsubstituted phenethyl.

21. The method of any one of claims 1-17 or 19, wherein R^{1a} is —C₁₋₄ alkyl-C₆₋₁₀ aryl substituted with 1 or 2 substituents each independently selected from the group consisting of halogen, hydroxy, —CN, —O(C₁₋₄)alkyl, —S(C₁₋₄)alkyl, —N(C₁₋₄ alkyl)₂, —NH(C₁₋₄ alkyl), and —C₁₋₄ alkyl optionally substituted with 1, 2, or 3 halogen atoms.

22. The method of any one of claims 1-17, 19, or 20, wherein R^{1a} is benzyl substituted with 1 or 2 substituents each independently selected from the group consisting of halogen, hydroxy, —CN, —O(C₁₋₄)alkyl, —S(C₁₋₄)alkyl, —N(C₁₋₄ alkyl)₂, —NH(C₁₋₄ alkyl), and —C₁₋₄ alkyl optionally substituted with 1, 2, or 3 halogen atoms.

23. The method of any one of claims 1-17, wherein R^{1a} is —C₃₋₁₀ cycloalkyl or —C₁₋₄ alkyl-C₃₋₁₀ cycloalkyl, wherein said cycloalkyl or alkylcycloalkyl is optionally substituted with 1, 2 or 3 substituents each independently selected from the group consisting of halogen, hydroxy, —CN, —ORb^{a}, —SRb^{a}, —N(Rb^{a})₂, —C₁₋₄ alkyl optionally substituted with 1, 2, or 3 halogen atoms, optionally substituted —C₆₋₁₀ aryl, optionally substituted -(5- to 10-membered)-C₁₋₉ heteroaryl, and -(5- to 10-membered)-C₂₋₉ heterocyclyl, wherein Rb^{a} is as defined above; and wherein said cycloalkyl is optionally fused to a further (second) ring, and wherein Rb^{a} is as defined in claim 1.

24. The method of any one of claims 1-17, wherein R^{1a} is -(5- to 10-membered)-C₁₋₉ heteroaryl or —C₁₋₄ alkyl-(5- to 10-membered)-C₁₋₉ heteroaryl, wherein said heteroaryl or alkylheteroaryl is optionally substituted with 1, 2 or 3 substituents each independently selected from the group consisting of halogen, hydroxy, —CN, —ORb^{a}, —SRb^{a}, —N(Rb^{a})₂, —C₁₋₄ alkyl optionally substituted with 1, 2, or 3 halogen atoms, optionally substituted —C₆₋₁₀ aryl, optionally substituted -(5- to 10-membered)-C₁₋₉ heteroaryl, and -(5- to 10-membered)-C₂₋₉ heterocyclyl, wherein Rb^{a} is as defined in claim 1.

25. The method of any one of claims 1-17 or 24, wherein R^{1a} is unsubstituted -(5- to 10-membered)-C₁₋₉ heteroaryl or -(5- to 10-membered)-C₁₋₉ heteroaryl substituted with 1 or 2 substituents each independently selected from the group consisting of halogen, hydroxy, —CN, —O(C₁₋₄)alkyl, —S(C₁₋₄)alkyl, —N(C₁₋₄alkyl)₂, —NH(C₁₋₄ alkyl), and —C₁₋₄ alkyl optionally substituted with 1, 2, or 3 halogen atoms.

26. The method of any one of claims 1-17 or 24, wherein R^{1a} is unsubstituted —C₁₋₄ alkyl-(5- to 10-membered)-C₁₋₉ heteroaryl or —C₁₋₄ alkyl-(5- to 10-membered)-C₁₋₉ heteroaryl optionally substituted with 1, 2 or 3 substituents each independently selected from the group consisting of halogen, hydroxy, —CN, —ORb^{a}, —SRb^{a}, —N(Rb^{a})₂, —C₁₋₄alkyl optionally substituted with 1, 2, or 3 halogen atoms, optionally substituted —C₆₋₁₀ aryl, optionally substituted -(5- to 10-membered)-C₁₋₉ heteroaryl and -(5- to 10-membered)-C₂₋₉ heterocyclyl, wherein Rb^{a} is as defined in claim 1.

27. The method of any one of claims 1-17, 24, or 26, wherein R^{1a} is unsubstituted furan-2-ylmethyl.

28. The method of any one of claims 1-17, 24, or 26, wherein R^{1a} is —C₁₋₄ alkyl-(5- to 10-membered)-C₁₋₉ heteroaryl substituted with 1 or 2 substituents each independently selected from the group consisting of halogen, hydroxy, —CN, —O(C₁₋₄)alkyl, —S(C₁₋₄)alkyl, —N(C₁₋₄ alkyl)₂, —NH(C₁₋₄ alkyl), and —C₁₋₄ alkyl optionally substituted with 1, 2, or 3 halogen atoms.

29. The method of any one of claims 1-20, 23, 24, 27, or 27, wherein Rb^{a} is hydrogen or —C₁₋₄ alkyl.

30. The method of any one of claims 1-29, wherein R^{2a} is —C₁₋₄ alkyl-(5- to 10-membered)-C₁₋₉ heteroaryl, wherein said alkylheteroaryl group is optionally substituted with 1, 2 or 3 substituents each independently selected from the group consisting of halogen, hydroxy, —CN, —C(═O)Ra^{a}, —ORb^{a}, —SRb^{a}, —N(Rb^{a})₂, (═O), —C₁₋₄ alkyl optionally substituted with 1, 2, or 3 halogen atoms, optionally substituted C₆₋₁₀ aryl, optionally substituted -(5- to 10-membered)-C₁₋₉ heteroaryl, and -(5- to 10-membered)-C₂₋₉ heterocyclyl; and wherein said cycloalkyl, alkylcycloalkyl, aryl, alkylaryl, heteroaryl, alkylheteroaryl, heterocyclyl and alkylheterocyclyl is optionally fused to a further (second) ring, wherein Ra^{a} and Rb^{a} are as claimed in claim 1.

31. The method of any one of claims 1-29, wherein R^{2a} is —C₁₋₄ alkyl-C(═O)NHRa^{a} or —C₁₋₄ alkyl-C(═O)N(Ra^{a})₂, wherein Ra^{a} is as defined in claim 1.

32. The method of any one of claims 1-29, wherein R^{2a} is —S(═O)₂Ra^{a}, wherein Ra^{a} is as defined in claim 1.

33. The method of any one of claims 1-29, wherein Ra^{a} is selected from the group consisting of —C₆₋₁₀ aryl, -(5- to 10-membered)-C₁₋₉ heteroaryl, —C₃₋₁₀ cycloalkyl, and -(5- to 10-membered)-C₂₋₉ heterocyclyl, wherein said aryl, heteroaryl, cycloalkyl, and heterocyclyl groups are optionally substituted with 1, 2 or 3 substituents each independently selected from the group consisting of halogen, hydroxy, —CN, —ORb^{a}, —SRb^{a}, —N(Rb^{a})₂, —C₁₋₄alkyl optionally substituted with 1, 2, or 3 halogen atoms, optionally substituted —C₆₋₁₀ aryl, optionally substituted -(5- to 10-membered)-C₁₋₉ heteroaryl and -(5- to 10-membered)-C₂₋₉ heterocyclyl, and wherein said aryl, heteroaryl, cycloalkyl, and heterocyclyl is optionally fused to a further (second) ring.

34. The method of claim 16, wherein the compound is selected from the group consisting of 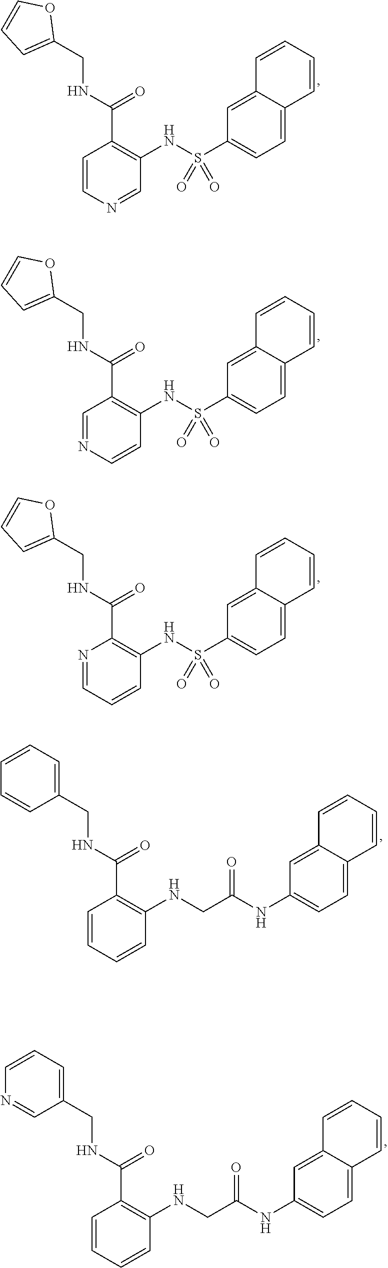 [SMILES: O=C(NCc1ccco1)c1ccncc1NS(=O)(=O)c1ccc2ccccc2c1 ; O=C(NCc1ccco1)c1cnccc1NS(=O)(=O)c1ccc2ccccc2c1 ; O=C(NCc1ccco1)c1ncccc1NS(=O)(=O)c1ccc2ccccc2c1 ; O=C(CNc1ccccc1C(=O)NCc1ccccc1)Nc1ccc2ccccc2c1 ; O=C(CNc1ccccc1C(=O)NCc1cccnc1)Nc1ccc2ccccc2c1]
or a pharmaceutically acceptable salt or solvate thereof.

35. The method of any one of claims 1-6, wherein the compound of formula (IA) is selected from the group consisting of 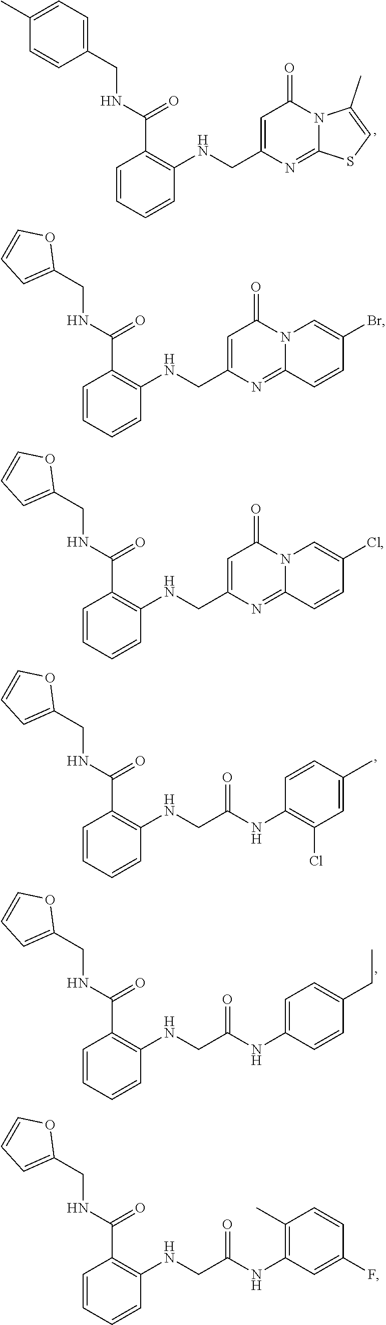 [SMILES: Cc1ccc(CNC(=O)c2ccccc2NCc2cc(=O)n3c(C)csc3n2)cc1 ; O=C(NCc1ccco1)c1ccccc1NCc1cc(=O)n2cc(Br)ccc2n1 ; O=C(NCc1ccco1)c1ccccc1NCc1cc(=O)n2cc(Cl)ccc2n1 ; Cc1ccc(NC(=O)CNc2ccccc2C(=O)NCc2ccco2)c(Cl)c1 ; CCc1ccc(NC(=O)CNc2ccccc2C(=O)NCc2ccco2)cc1 ; Cc1ccc(F)cc1NC(=O)CNc1ccccc1C(=O)NCc1ccco1] 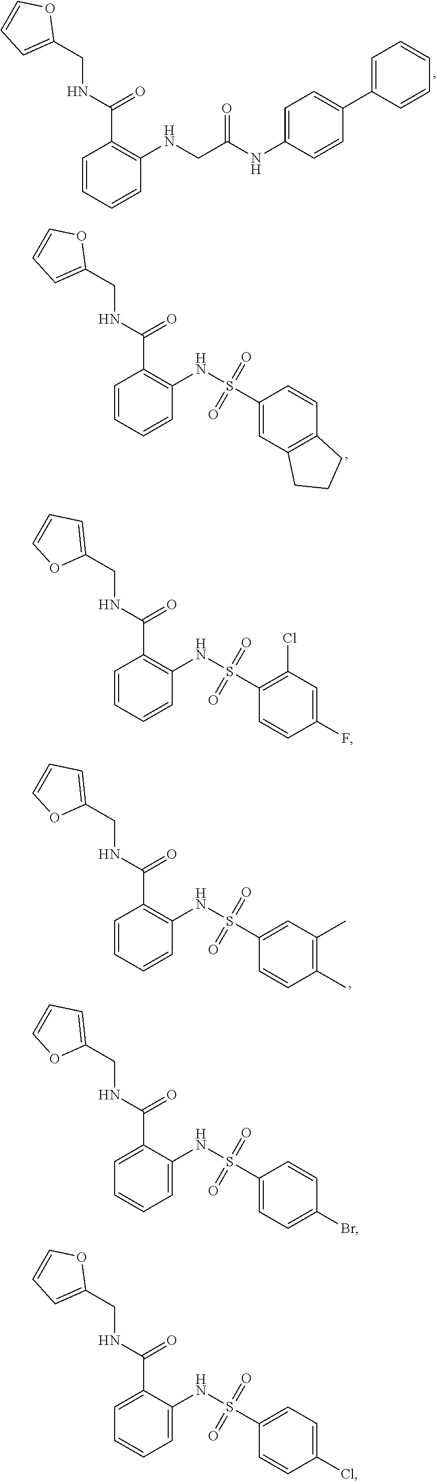 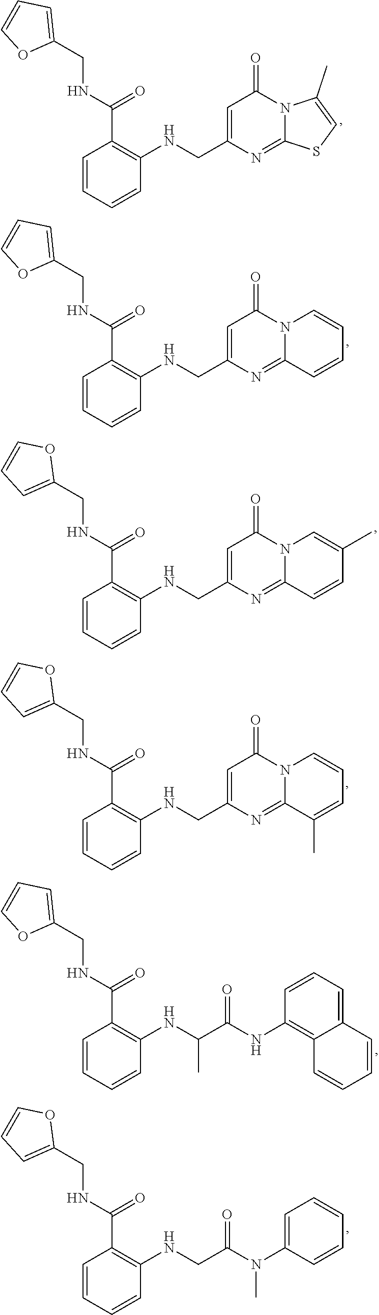 [SMILES: Cc1csc2nc(CNc3ccccc3C(=O)NCc3ccco3)cc(=O)n12 ; O=C(NCc1ccco1)c1ccccc1NCc1cc(=O)n2ccccc2n1 ; Cc1ccc2nc(CNc3ccccc3C(=O)NCc3ccco3)cc(=O)n2c1 ; Cc1cccn2c(=O)cc(CNc3ccccc3C(=O)NCc3ccco3)nc12 ; CC(Nc1ccccc1C(=O)NCc1ccco1)C(=O)Nc1cccc2ccccc12 ; CN(C(=O)CNc1ccccc1C(=O)NCc1ccco1)c1ccccc1] 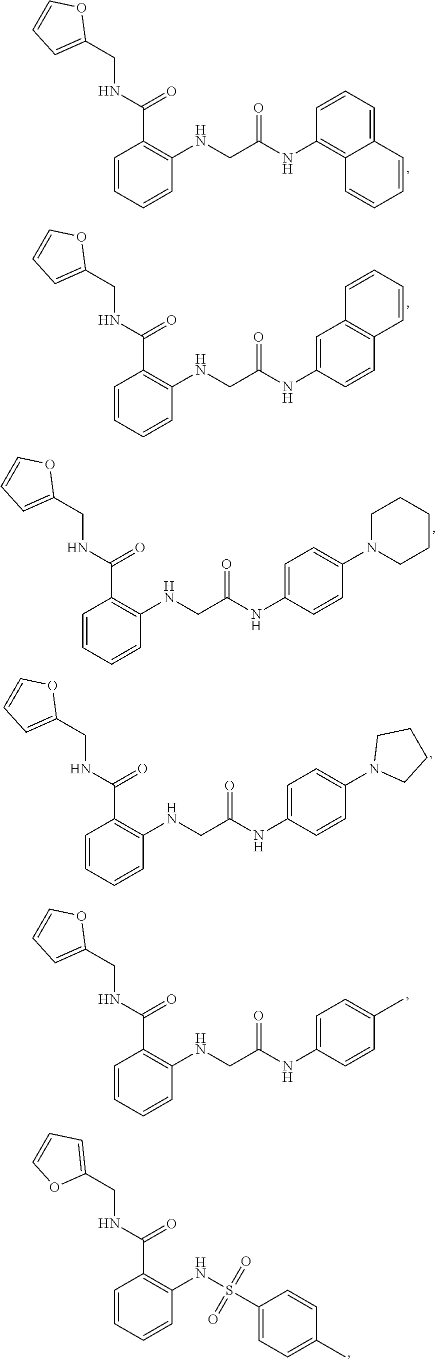 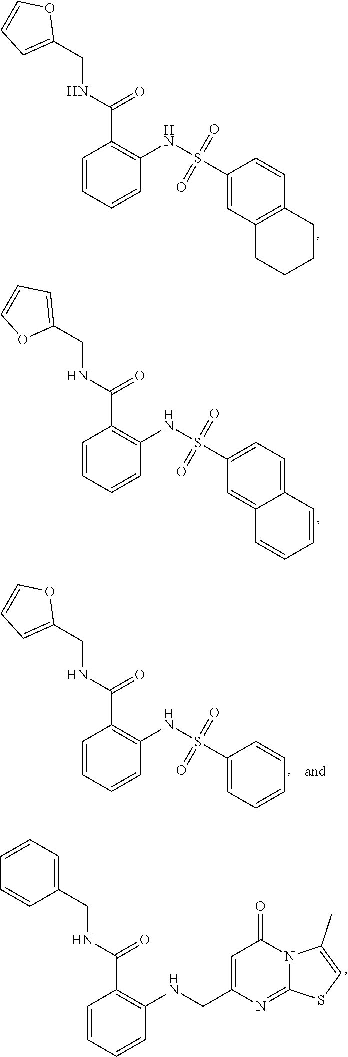 [SMILES: O=C(NCc1ccco1)c1ccccc1NS(=O)(=O)c1ccc2c(c1)CCCC2 ; O=C(NCc1ccco1)c1ccccc1NS(=O)(=O)c1ccc2ccccc2c1 ; O=C(NCc1ccco1)c1ccccc1NS(=O)(=O)c1ccccc1 ; Cc1csc2nc(CNc3ccccc3C(=O)NCc3ccccc3)cc(=O)n12]
or a pharmaceutically acceptable salt or solvate thereof.

36. The method of any one of claims 1-6, wherein the compound of formula (IA) is selected from the group consisting of 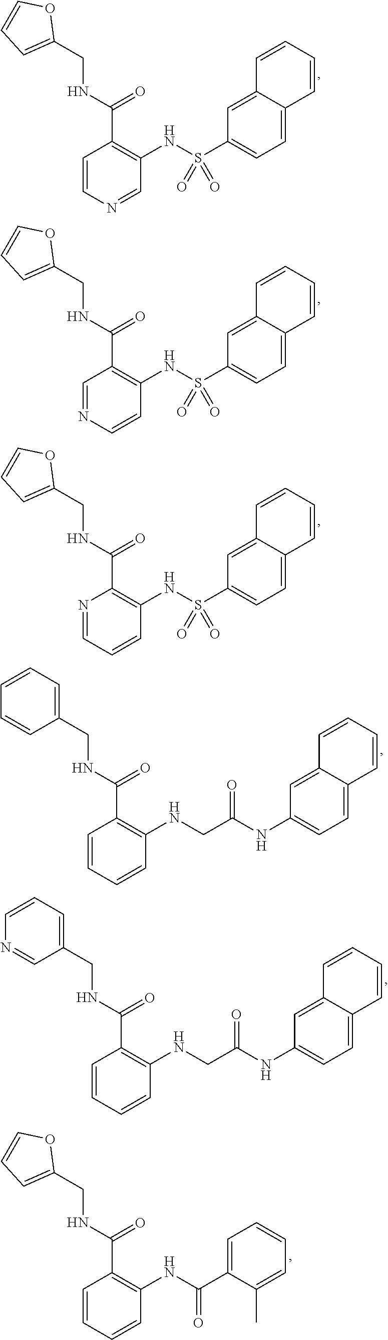 [SMILES: O=C(NCc1ccco1)c1ccncc1NS(=O)(=O)c1ccc2ccccc2c1 ; O=C(NCc1ccco1)c1cnccc1NS(=O)(=O)c1ccc2ccccc2c1 ; O=C(NCc1ccco1)c1ncccc1NS(=O)(=O)c1ccc2ccccc2c1 ; O=C(CNc1ccccc1C(=O)NCc1ccccc1)Nc1ccc2ccccc2c1 ; O=C(CNc1ccccc1C(=O)NCc1cccnc1)Nc1ccc2ccccc2c1 ; Cc1ccccc1C(=O)Nc1ccccc1C(=O)NCc1ccco1] 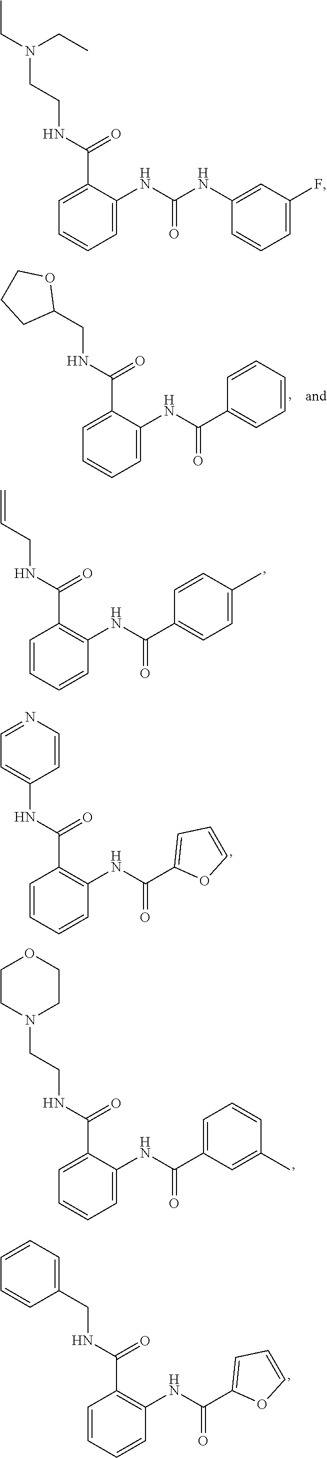 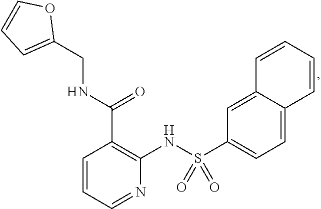 [SMILES: O=C(NCc1ccco1)c1cccnc1NS(=O)(=O)c1ccc2ccccc2c1]
or a pharmaceutically acceptable salt or solvate thereof.

37. A method of treating or preventing a condition associated with the alteration of the activity of galactocerebrosidase in a patient, comprising administering to the patient in need thereof an effective amount of a compound of formula (IB): 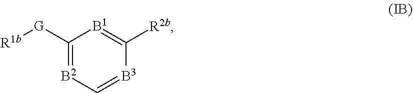
or a pharmaceutically acceptable salt or solvate thereof, wherein
G is —C(═O)—NH— or —NH—C(═O)—;
B¹, B², and B³ are each independently selected from the group consisting of N, CH and C(R^{3b});
each R^{3b} is independently selected from the group consisting of halogen, C₁₋₄ alkyl, halo(C₁₋₄ alkyl), —OH, C₁₋₄ alkoxy, halo(C₁₋₄ alkoxy), and CN;
R^{1b} is selected from the group consisting of —C₁₋₄ alkyl, —C₃₋₁₀ cycloalkyl, —C₁₋₄ alkyl-C₃₋₁₀ cycloalkyl, —C₆₋₁₀ aryl, —C₁₋₄ alkyl-C₆₋₁₀ aryl, —C₂₋₄ alkylene-C₆₋₁₀ aryl, (5- to 10-membered)-C₁₋₉ heteroaryl, —C₁₋₄ alkyl-(5- to 10-membered)-C₁₋₉ heteroaryl, —C₂₋₄ alkylene-(5- to 10-membered)-C₁₋₉ heteroaryl, (5- to 10-membered)-C₂₋₉ heterocyclyl, —C₁₋₄ alkyl-(5- to 10-membered)-C₂₋₉ heterocyclyl, and —C₂₋₄ alkenyl-(5- to 10-membered)-C₂₋₉ heterocyclyl, wherein said alkyl, cycloalkyl, alkylcycloalkyl, aryl, alkylaryl, heteroaryl, alkylheteroaryl, alkenylheteroaryl, heterocyclyl and alkylheterocyclyl groups are optionally substituted with 1, 2 or 3 substituents each independently selected from the group consisting of halogen, hydroxy, —CN, —ORb^{b}, —SRb^{b}, —N(Rb^{b})₂, —C₁₋₄ alkyl optionally substituted with 1, 2, or 3 halogen atoms, optionally substituted C₆₋₁₀ aryl, optionally substituted (5- to 10-membered)-C₁₋₉ heteroaryl, and (5- to 10-membered)-C₂₋₉ heterocyclyl; and wherein said cycloalkyl, alkylcycloalkyl, aryl, alkylaryl, heteroaryl, alkylheteroaryl, alkenylheteroaryl, heterocyclyl and alkylheterocyclyl is optionally fused to a further (second) ring;
R^{2b} is —C₆₋₁₀ aryl, -(5- to 10-membered)-C₁₋₉ heteroaryl, —C(═O)Ra^{b}, —S(═O)₂Ra^{b}, —C(═O)—NH—Ra^{b}, —S(═O)₂—NH—Ra^{b}, —C₁₋₄ alkyl-C(═O)Ra^{b}, —C₁₋₄ alkyl-S(═O)₂Ra^{b}, or —N(Rb^{b})₂, wherein said aryl and heteroaryl groups are optionally substituted with 1, 2 or 3 substituents each independently selected from the group consisting of halogen, hydroxy, CN, —ORb^{b}, —SRb^{b}, —N(Rb^{b})₂, (═O), —C₁₋₄alkyl optionally substituted with 1, 2, or 3 substituents each independently selected from the group consisting of halogen, CN, —ORb^{b}, and —N(Rb^{b})₂, optionally substituted —C₆₋₁₀ aryl, optionally substituted -(5- to 10-membered)-C₁₋₉ heteroaryl, -(5- to 10-membered)-C₂₋₉ heterocyclyl, and —C₃₋₁₀ cycloalkyl; and wherein said aryl, heteroaryl, and heterocyclyl are optionally fused to a further (second) ring; or
R^{2b} and R^{3b} attached to an adjacent carbon atom together form a 5- or 6-membered heterocyclic ring containing one N-atom substituted with —S(═O)₂Ra^{b};
Ra^{b} is selected from the group consisting of —C₁₋₄ alkyl, —C₃₋₁₀ cycloalkyl, —C₁₋₄ alkyl-C₃₋₁₀ cycloalkyl, —C₆₋₁₀ aryl, —C₁₋₄ alkyl-C₆₋₁₀ aryl, (5- to 10-membered)-C₁₋₉ heteroaryl, —C₁₋₄ alkyl-(5- to 10-membered)-C₁₋₉ heteroaryl, (5- to 10-membered)-C₂₋₉ heterocyclyl, and —C₁₋₄ alkyl-(5- to 10-membered)-C₂₋₉ heterocyclyl, wherein said alkyl, cycloalkyl, alkylcycloalkyl, aryl, alkylaryl, heteroaryl, alkylheteroaryl, heterocyclyl and alkylheterocyclyl groups are optionally substituted with 1, 2 or 3 substituents each independently selected from the group consisting of halogen, hydroxy, —CN, —ORb^{b}, —SRb^{b}, —N(Rb^{b})₂, —C₁₋₄ alkyl optionally substituted with 1, 2, or 3 halogen atoms, optionally substituted C₆₋₁₀ aryl, optionally substituted (5- to 10-membered)-C₁₋₉ heteroaryl, and (5- to 10-membered)-C₂₋₉ heterocyclyl; and wherein said cycloalkyl, alkylcycloalkyl, aryl, alkylaryl, heteroaryl, alkylheteroaryl, heterocyclyl and alkylheterocyclyl is optionally fused to a further (second) ring; and
each Rb^{b} is independently hydrogen, —C(═O)Ra^{b}, —S(═O)₂Ra^{b}, —C₁₋₄ alkyl, —C₃₋₁₀ cycloalkyl, -(5- to 10-membered)-C₂₋₉ heterocyclyl, or optionally substituted —C₆₋₁₀ aryl, wherein said alkyl, cycloalkyl or heterocyclyl group is optionally substituted by 1, 2 or 3 fluorine atoms.

38. A method of treating or preventing a lysosomal storage disease or an α-synucleinopathy, comprising administering to a patient in need thereof an effective amount of a compound of formula (IB): 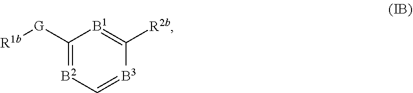
or a pharmaceutically acceptable salt or solvate thereof, wherein
G is —C(═O)—NH— or —NH—C(═O)—;
B¹, B², and B³ are each independently selected from the group consisting of N, CH and C(R^{3b});
each R^{3b} is independently selected from the group consisting of halogen, C₁₋₄ alkyl, halo(C₁₋₄ alkyl), —OH, C₁₋₄ alkoxy, halo(C₁₋₄ alkoxy), and CN;
R^{1b} is selected from the group consisting of —C₁₋₄ alkyl, —C₃₋₁₀ cycloalkyl, —C₁₋₄ alkyl-C₃₋₁₀ cycloalkyl, —C₆₋₁₀ aryl, —C₁₋₄ alkyl-C₆₋₁₀ aryl, —C₂₋₄ alkylene-C₆₋₁₀ aryl, (5- to 10-membered)-C₁₋₉ heteroaryl, —C₁₋₄ alkyl-(5- to 10-membered)-C₁₋₉ heteroaryl, —C₂₋₄ alkylene-(5- to 10-membered)-C₁₋₉ heteroaryl, (5- to 10-membered)-C₂₋₉ heterocyclyl, —C₁₋₄ alkyl-(5- to 10-membered)-C₂₋₉ heterocyclyl, and —C₂₋₄ alkenyl-(5- to 10-membered)-C₂₋₉ heterocyclyl, wherein said alkyl, cycloalkyl, alkylcycloalkyl, aryl, alkylaryl, heteroaryl, alkylheteroaryl, alkenylheteroaryl, heterocyclyl and alkylheterocyclyl groups are optionally substituted with 1, 2 or 3 substituents each independently selected from the group consisting of halogen, hydroxy, —CN, —ORb^{b}, —SRb^{b}, —N(Rb^{b})₂, —C₁₋₄ alkyl optionally substituted with 1, 2, or 3 halogen atoms, optionally substituted C₆₋₁₀ aryl, optionally substituted (5- to 10-membered)-C₁₋₉ heteroaryl, and (5- to 10-membered)-C₂₋₉ heterocyclyl; and wherein said cycloalkyl, alkylcycloalkyl, aryl, alkylaryl, heteroaryl, alkylheteroaryl, alkenylheteroaryl, heterocyclyl and alkylheterocyclyl is optionally fused to a further (second) ring;
R^{2b} is —C₆₋₁₀ aryl, -(5- to 10-membered)-C₁₋₉ heteroaryl, —C(═O)Ra^{b}, —S(═O)₂Ra^{b}, —C(═O)—NH—Ra^{b}, —S(═O)₂—NH—Ra^{b}, —C₁₋₄ alkyl-C(═O)Ra^{b}, —C₁₋₄ alkyl-S(═O)₂Ra^{b}, or —N(Rb^{b})₂, wherein said aryl and heteroaryl groups are optionally substituted with 1, 2 or 3 substituents each independently selected from the group consisting of halogen, hydroxy, CN, —ORb^{b}, —SRb^{b}, —N(Rb^{b})₂, (═O), —C₁₋₄alkyl optionally substituted with 1, 2, or 3 substituents each independently selected from the group consisting of halogen, CN, —ORb^{b}, and —N(Rb^{b})₂, optionally substituted —C₆₋₁₀ aryl, optionally substituted -(5- to 10-membered)-C₁₋₉ heteroaryl, -(5- to 10-membered)-C₂₋₉ heterocyclyl, and —C₃₋₁₀ cycloalkyl; and wherein said aryl, heteroaryl, and heterocyclyl are optionally fused to a further (second) ring; or
R^{2b} and R^{3b} attached to an adjacent carbon atom together form a 5- or 6-membered heterocyclic ring containing one N-atom substituted with —S(═O)₂Ra^{b};
Ra^{b} is selected from the group consisting of —C₁₋₄ alkyl, —C₃₋₁₀ cycloalkyl, —C₁₋₄ alkyl-C₃₋₁₀ cycloalkyl, —C₆₋₁₀ aryl, —C₁₋₄ alkyl-C₆₋₁₀ aryl, (5- to 10-membered)-C₁₋₉ heteroaryl, —C₁₋₄ alkyl-(5- to 10-membered)-C₁₋₉ heteroaryl, (5- to 10-membered)-C₂₋₉ heterocyclyl, and —C₁₋₄ alkyl-(5- to 10-membered)-C₂₋₉ heterocyclyl, wherein said alkyl, cycloalkyl, alkylcycloalkyl, aryl, alkylaryl, heteroaryl, alkylheteroaryl, heterocyclyl and alkylheterocyclyl groups are optionally substituted with 1, 2 or 3 substituents each independently selected from the group consisting of halogen, hydroxy, —CN, —ORb^{b}, —SRb^{b}, —N(Rb^{b})₂, —C₁₋₄ alkyl optionally substituted with 1, 2, or 3 halogen atoms, optionally substituted C₆₋₁₀ aryl, optionally substituted (5- to 10-membered)-C₁₋₉ heteroaryl, and (5- to 10-membered)-C₂₋₉ heterocyclyl; and wherein said cycloalkyl, alkylcycloalkyl, aryl, alkylaryl, heteroaryl, alkylheteroaryl, heterocyclyl and alkylheterocyclyl is optionally fused to a further (second) ring; and
each Rb^{b} is independently hydrogen, —C(═O)Ra^{b}, —S(═O)₂Ra^{b}, —C₁₋₄ alkyl, —C₃₋₁₀ cycloalkyl, -(5- to 10-membered)-C₂₋₉ heterocyclyl, or optionally substituted —C₆₋₁₀ aryl, wherein said alkyl, cycloalkyl or heterocyclyl group is optionally substituted by 1, 2 or 3 fluorine atoms.

39. The method of claim 38, wherein a lysosomal storage disease is treated or prevented.

40. The method of claim 38 or 39, wherein the lysosomal storage disease is Krabbe's disease.

41. The method of claim 38, wherein an α-synucleinopathy is treated or prevented.

42. A method of treating or preventing a disease or disorder, comprising administering to a patient in need thereof an effective amount of a compound of formula (IB): 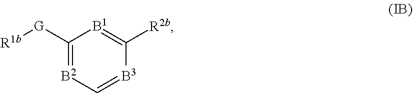
or a pharmaceutically acceptable salt or solvate thereof, wherein
G is —C(═O)—NH— or —NH—C(═O)—;
B¹, B², and B³ are each independently selected from the group consisting of N, CH and C(R^{3b});
each R^{3b} is independently selected from the group consisting of halogen, C₁₋₄ alkyl, halo(C₁₋₄ alkyl), —OH, C₁₋₄ alkoxy, halo(C₁₋₄ alkoxy), and CN;
R^{1b} is selected from the group consisting of —C₁₋₄ alkyl, —C₃₋₁₀ cycloalkyl, —C₁₋₄ alkyl-C₃₋₁₀ cycloalkyl, —C₆₋₁₀ aryl, —C₁₋₄ alkyl-C₆₋₁₀ aryl, —C₂₋₄ alkylene-C₆₋₁₀ aryl, (5- to 10-membered)-C₁₋₉ heteroaryl, —C₁₋₄ alkyl-(5- to 10-membered)-C₁₋₉ heteroaryl, —C₂₋₄ alkylene-(5- to 10-membered)-C₁₋₉ heteroaryl, (5- to 10-membered)-C₂₋₉ heterocyclyl, —C₁₋₄ alkyl-(5- to 10-membered)-C₂₋₉ heterocyclyl, and —C₂₋₄ alkenyl-(5- to 10-membered)-C₂₋₉ heterocyclyl, wherein said alkyl, cycloalkyl, alkylcycloalkyl, aryl, alkylaryl, heteroaryl, alkylheteroaryl, alkenylheteroaryl, heterocyclyl and alkylheterocyclyl groups are optionally substituted with 1, 2 or 3 substituents each independently selected from the group consisting of halogen, hydroxy, —CN, —ORb^{b}, —SRb^{b}, —N(Rb^{b})₂, —C₁₋₄ alkyl optionally substituted with 1, 2, or 3 halogen atoms, optionally substituted C₆₋₁₀ aryl, optionally substituted (5- to 10-membered)-C₁₋₉ heteroaryl, and (5- to 10-membered)-C₂₋₉ heterocyclyl; and wherein said cycloalkyl, alkylcycloalkyl, aryl, alkylaryl, heteroaryl, alkylheteroaryl, alkenylheteroaryl, heterocyclyl and alkylheterocyclyl is optionally fused to a further (second) ring;
R^{2b} is —C₆₋₁₀ aryl, -(5- to 10-membered)-C₁₋₉ heteroaryl, —C(═O)Ra^{b}, —S(═O)₂Ra^{b}, —C(═O)—NH—Ra^{b}, —S(═O)₂—NH—Ra^{b}, —C₁₋₄ alkyl-C(═O)Ra^{b}, —C₁₋₄ alkyl-S(═O)₂Ra^{b}, or —N(Rb^{b})₂, wherein said aryl and heteroaryl groups are optionally substituted with 1, 2 or 3 substituents each independently selected from the group consisting of halogen, hydroxy, CN, —ORb^{b}, —SRb^{b}, —N(Rb^{b})₂, (═O), —C₁₋₄alkyl optionally substituted with 1, 2, or 3 substituents each independently selected from the group consisting of halogen, CN, —ORb^{b}, and —N(Rb^{b})₂, optionally substituted —C₆₋₁₀ aryl, optionally substituted -(5- to 10-membered)-C₁₋₉ heteroaryl, -(5- to 10-membered)-C₂₋₉ heterocyclyl, and —C₃₋₁₀ cycloalkyl; and wherein said aryl, heteroaryl, and heterocyclyl are optionally fused to a further (second) ring; or
R^{2b} and R^{3b} attached to an adjacent carbon atom together form a 5- or 6-membered heterocyclic ring containing one N-atom substituted with —S(═O)₂Ra^{b};
Ra^{b} is selected from the group consisting of —C₁₋₄ alkyl, —C₃₋₁₀ cycloalkyl, —C₁₋₄ alkyl-C₃₋₁₀ cycloalkyl, —C₆₋₁₀ aryl, —C₁₋₄ alkyl-C₆₋₁₀ aryl, (5- to 10-membered)-C₁₋₉ heteroaryl, —C₁₋₄ alkyl-(5- to 10-membered)-C₁₋₉ heteroaryl, (5- to 10-membered)-C₂₋₉ heterocyclyl, and —C₁₋₄ alkyl-(5- to 10-membered)-C₂₋₉ heterocyclyl, wherein said alkyl, cycloalkyl, alkylcycloalkyl, aryl, alkylaryl, heteroaryl, alkylheteroaryl, heterocyclyl and alkylheterocyclyl groups are optionally substituted with 1, 2 or 3 substituents each independently selected from the group consisting of halogen, hydroxy, —CN, —ORb^{b}, —SRb^{b}, —N(Rb^{b})₂, —C₁₋₄ alkyl optionally substituted with 1, 2, or 3 halogen atoms, optionally substituted C₆₋₁₀ aryl, optionally substituted (5- to 10-membered)-C₁₋₉ heteroaryl, and (5- to 10-membered)-C₂₋₉ heterocyclyl; and wherein said cycloalkyl, alkylcycloalkyl, aryl, alkylaryl, heteroaryl, alkylheteroaryl, heterocyclyl and alkylheterocyclyl is optionally fused to a further (second) ring; and
each Rb^{b} is independently hydrogen, —C(═O)Ra^{b}, —S(═O)₂Ra^{b}, —C₁₋₄ alkyl, —C₃₋₁₀ cycloalkyl, -(5- to 10-membered)-C₂₋₉ heterocyclyl, or optionally substituted —C₆₋₁₀ aryl, wherein said alkyl, cycloalkyl or heterocyclyl group is optionally substituted by 1, 2 or 3 fluorine atoms.

43. The method of any one of claims 37-42, comprising administering to a patient in need thereof an effective amount of a compound of formula (IB) where G is —C(═O)—NH—, which is a compound of formula (IIB): 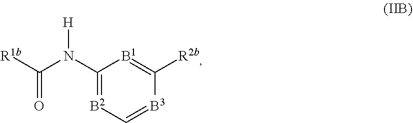
or a pharmaceutically acceptable salt or solvate thereof, wherein B¹, B², B³, R^{1b}, and R^{2b} are as defined in claim 37.

44. The method of any one of claims 37-42, comprising administering to a patient in need thereof an effective amount of a compound of formula (IB) where G is —NH—C(═O)—, which is a compound of formula (IIIB): 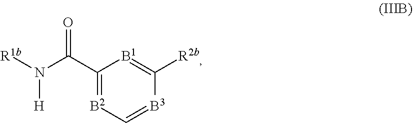
or a pharmaceutically acceptable salt or solvate thereof, wherein B¹, B², B³, R^{1b}, and R^{2b} are as defined in claim 37.

45. The method of any one of claims 37-44, wherein B¹, B², and B³ are CH.

46. The method of any one of claims 37-44, wherein one of B¹, B², and B³ is C(R^{3b}) and the ones not C(R^{3b}) are CH.

47. The method of any one of claims 37-44, wherein two of B¹, B², and B³ is C(R^{3b}) and the one not C(R^{3b}) is CH.

48. The method of any one of claims 37-44, wherein one of B¹, B² and B³ is N.

49. The method of any one of claims 37-44, wherein two of B¹, B² and B³ are N.

50. The method of any one of claims 37-44, wherein B¹, B² and B³ are N.

51. The method of any one of claims 37-44, wherein B¹ is N and B² and B³ are each independently selected from the group consisting of CH and C(R^{3b}).

52. The method of any one of claims 37-44, wherein B² is N and B¹ and B³ are each independently selected from the group consisting of CH and C(R^{3b}).

53. The method of any one of claims 37-44, wherein B³ is N and B¹ and B² are each independently selected from the group consisting of CH and C(R^{3b}).

54. The method of any one of claims 37-44, wherein B¹ and B² are both N and B³ is CH or C(R^{3b}).

55. The method of any one of claims 37-44, wherein B¹ and B³ are both N and B² is CH or C(R^{3b}).

56. The method of any one of claims 37-44, wherein B² and B³ are both N and B¹ is CH or C(R^{3b}).

57. The method of any one of claims 37-56, wherein R^{1b} is —C₆₋₁₀ aryl or —C₁₋₄ alkyl-C₆₋₁₀ aryl, wherein said aryl or alkylaryl is optionally substituted with 1, 2 or 3 substituents each independently selected from the group consisting of halogen, hydroxy, —CN, —ORb^{b}, —SRb^{b}, —N(Rb^{b})₂, —C₁₋₄ alkyl optionally substituted with 1, 2, or 3 halogen atoms, optionally substituted —C₆₋₁₀ aryl, optionally substituted -(5- to 10-membered)-C₁₋₉ heteroaryl, and -(5- to 10-membered)-C₂₋₉ heterocyclyl, wherein Rb^{b} is as defined in claim 37.

58. The method of any one of claims 37-56, wherein R^{1b} is unsubstituted —C₁₋₄ alkyl-C₆₋₁₀ aryl or —C₁₋₄ alkyl-C₆₋₁₀ aryl optionally substituted with 1, 2 or 3 substituents each independently selected from the group consisting of halogen, hydroxy, —CN, —ORb^{b}, —SRb^{b}, —N(Rb^{b})₂, —C₁₋₄alkyl optionally substituted with 1, 2, or 3 halogen atoms, optionally substituted —C₆₋₁₀ aryl, optionally substituted -(5- to 10-membered)-C₁₋₉ heteroaryl and -(5- to 10-membered)-C₂₋₉ heterocyclyl, wherein Rb^{b} is as defined in claim 37.

59. The method of any one of claims 37-56 or 58, wherein R^{1b} is —C₁₋₄ alkyl-C₆₋₁₀ aryl substituted with 1 or 2 substituents each independently selected from the group consisting of halogen, hydroxy, —CN, —O(C₁₋₄)alkyl, —S(C₁₋₄)alkyl, —N(C₁₋₄ alkyl)₂, —NH(C₁₋₄ alkyl), and —C₁₋₄ alkyl optionally substituted with 1, 2, or 3 halogen atoms.

60. The method of any one of claims 37-56, wherein R^{1b} is —C₃₋₁₀ cycloalkyl or —C₁₋₄ alkyl-C₃₋₁₀ cycloalkyl, wherein said cycloalkyl or alkylcycloalkyl is optionally substituted with 1, 2 or 3 substituents each independently selected from the group consisting of halogen, hydroxy, —CN, —ORb^{b}, —SRb^{b}, —N(Rb^{b})₂, —C₁₋₄ alkyl optionally substituted with 1, 2, or 3 halogen atoms, optionally substituted —C₆₋₁₀ aryl, optionally substituted -(5- to 10-membered)-C₁₋₉ heteroaryl, and -(5- to 10-membered)-C₂₋₉ heterocyclyl, wherein Rb^{b} is as defined in claim 35; and wherein said cycloalkyl is optionally fused to a further (second) ring.

61. The method of any one of claims 37-56, wherein R^{1b} is -(5- to 10-membered)-C₁₋₉ heteroaryl, —C₁₋₄ alkyl-(5- to 10-membered)-C₁₋₉ heteroaryl, or —C₂₋₄ alkenyl-(5- to 10-membered)-C₁₋₉ heteroaryl, wherein said heteroaryl, alkylheteroaryl, or alkenylheteroaryl is optionally substituted with 1, 2 or 3 substituents each independently selected from the group consisting of halogen, hydroxy, —CN, —ORb^{b}, —SRb^{b}, —N(Rb^{b})₂, —C₁₋₄ alkyl optionally substituted with 1, 2, or 3 halogen atoms, optionally substituted —C₆₋₁₀ aryl, optionally substituted -(5- to 10-membered)-C₁₋₉ heteroaryl, and -(5- to 10-membered)-C₂₋₉ heterocyclyl, wherein Rb^{b} is as defined in claim 37.

62. The method of any one of claims 37-57 or 61, wherein R^{1b} is unsubstituted -(5- to 10-membered)-C₁₋₉ heteroaryl or -(5- to 10-membered)-C₁₋₉ heteroaryl substituted with 1 or 2 substituents each independently selected from the group consisting of halogen, hydroxy, —CN, —O(C₁₋₄)alkyl, —S(C₁₋₄)alkyl, —N(C₁₋₄alkyl)₂, —NH(C₁₋₄ alkyl), and —C₁₋₄ alkyl optionally substituted with 1, 2, or 3 halogen atoms.

63. The method of any one of claims 37-56 or 61, wherein R^{1b} is unsubstituted —C₁₋₄ alkyl-(5- to 10-membered)-C₁₋₉ heteroaryl or —C₁₋₄ alkyl-(5- to 10-membered)-C₁₋₉ heteroaryl optionally substituted with 1, 2 or 3 substituents each independently selected from the group consisting of halogen, hydroxy, —CN, —ORb^{a}, —SRb^{a}, —N(Rb^{a})₂, —C₁₋₄alkyl optionally substituted with 1, 2, or 3 halogen atoms, optionally substituted —C₆₋₁₀ aryl, optionally substituted -(5- to 10-membered)-C₁₋₉ heteroaryl and -(5- to 10-membered)-C₂₋₉ heterocyclyl, wherein Rb^{b} is as defined in claim 37.

64. The method of any one of claims 37-56 or 61, wherein R^{1b} is unsubstituted —C₂₋₄ alkenyl-(5- to 10-membered)-C₁₋₉ heteroaryl or —C₂₋₄ alkenyl-(5- to 10-membered)-C₁₋₉ heteroaryl optionally substituted with 1, 2 or 3 substituents each independently selected from the group consisting of halogen, hydroxy, —CN, —ORb^{a}, —SRb^{a}, —N(Rb^{a})₂, —C₁₋₄alkyl optionally substituted with 1, 2, or 3 halogen atoms, optionally substituted —C₆₋₁₀ aryl, optionally substituted -(5- to 10-membered)-C₁₋₉ heteroaryl and -(5- to 10-membered)-C₂₋₉ heterocyclyl, wherein Rb^{b} is as defined in claim 37.

65. The method of any one of claims 37-56, 61, or 64, wherein R^{1b} is unsubstituted furan-2-yl-ethenyl.

66. The method of any one of claims 37-56, wherein R^{1b} is —C₁₋₄ alkyl optionally substituted with 1, 2 or 3 substituents each independently selected from the group consisting of halogen, hydroxy, —CN, —ORb^{b}, —SRb^{b}, —N(Rb^{b})₂, —C₁₋₄ alkyl optionally substituted with 1, 2, or 3 halogen atoms, optionally substituted C₆₋₁₀ aryl, optionally substituted (5- to 10-membered)-C₁₋₉ heteroaryl, and (5- to 10-membered)-C₂₋₉ heterocyclyl; and wherein said cycloalkyl, alkylcycloalkyl, aryl, alkylaryl, heteroaryl, alkylheteroaryl, alkenylheteroaryl, heterocyclyl and alkylheterocyclyl is optionally fused to a further (second) ring, wherein Rb^{b} is as defined in claim 37.

67. The method of any one of claims 37-56 or 66, wherein R^{1b} is unsubstituted —C₁₋₄ alkyl.

68. The method of any one of claims 37-56 or 66, wherein R^{1b} is —C₁₋₄ alkyl substituted with —ORb^{b}, —SRb^{b}, or —N(Rb^{b})₂, wherein Rb^{b} is as defined in claim 37.

69. The method of any one of claims 37-56, 66, or 68, wherein each Rb^{b} is independently hydrogen, —C(═O)Ra^{b}, —S(═O)₂Ra^{b}, —C₁₋₄ alkyl, —C₃₋₁₀ cycloalkyl, -(5- to 10-membered)-C₂₋₉ heterocyclyl, or optionally substituted —C₆₋₁₀ aryl, wherein said alkyl, cycloalkyl or heterocyclyl group is optionally substituted by 1, 2 or 3 fluorine atoms.

70. The method of any one of claims 37-69, wherein R^{2b} is —C₆₋₁₀ aryl, -(5- to 10-membered)-C₁₋₉ heteroaryl, —C(═O)Ra^{b}, —S(═O)₂Ra^{b}, —C(═O)—NH—Ra^{b}, —S(═O)₂—NH—Ra^{b}, —C₁₋₄ alkyl-C(═O)Ra^{b}, —C₁₋₄ alkyl-S(═O)₂Ra^{b}, or —N(Rb^{b})₂, wherein said aryl and heteroaryl groups are optionally substituted with 1, 2 or 3 substituents each independently selected from the group consisting of halogen, hydroxy, CN, —ORb^{b}, —SRb^{b}, —N(Rb^{b})₂, (═O), —C₁₋₄alkyl optionally substituted with 1, 2, or 3 substituents each independently selected from the group consisting of halogen, CN, —ORb^{b}, and —N(Rb^{b})₂, optionally substituted —C₆₋₁₀ aryl, optionally substituted -(5- to 10-membered)-C₁₋₉ heteroaryl, -(5- to 10-membered)-C₂₋₉ heterocyclyl, and —C₃₋₁₀ cycloalkyl; and wherein said aryl, heteroaryl, and heterocyclyl is optionally fused to a further (second) ring.

71. The method of any one of claims 37-70, wherein R^{2b} is —C₆₋₁₀ aryl or -(5- to 10-membered)-C₁₋₉ heteroaryl, wherein said aryl and heteroaryl groups are optionally substituted with 1, 2 or 3 substituents each independently selected from the group consisting of halogen, hydroxy, CN, —ORb^{b}, —SRb^{b}, —N(Rb^{b})₂, (═O), —C₁₋₄alkyl optionally substituted with 1, 2, or 3 substituents each independently selected from the group consisting of halogen, CN, —ORb^{b}, and —N(Rb^{b})₂, optionally substituted —C₆₋₁₀ aryl, optionally substituted -(5- to 10-membered)-C₁₋₉ heteroaryl, -(5- to 10-membered)-C₂₋₉ heterocyclyl, and —C₃₋₁₀ cycloalkyl; and wherein said aryl, heteroaryl, and heterocyclyl is optionally fused to a further (second) ring; wherein Rb^{b} is as defined in claim 37.

72. The method of any one of claims 37-69, wherein R^{2b} is —S(═O)₂Ra^{b}, —C(═O)—NH—Ra^{b}, —S(═O)₂—NH—Ra^{b}, —C₁₋₄ alkyl-C(═O)Ra^{b}, —C₁₋₄ alkyl-S(═O)₂Ra^{b}, or —N(Rb^{b})₂, wherein wherein Ra^{b} and Rb^{b} are as defined in claim 37.

73. The method of any one of claims 37-69 or 72, wherein R^{2b} is —C(═O)—NH—Ra^{b} or —S(═O)₂—NH—Ra^{b}, wherein Ra^{b} is —C₆₋₁₀ aryl optionally substituted with 1, 2 or 3 substituents each independently selected from the group consisting of halogen, hydroxy, —CN, —ORb^{b}, —SRb^{b}, —N(Rb^{b})₂, and —C₁₋₄ alkyl optionally substituted with 1, 2, or 3 halogen atoms.

74. The method of any one of claims 37-69, wherein R^{2b} and R^{3b} attached to an adjacent carbon atom together form a 5- or 6-membered N-containing heterocyclic ring substituted at the N-atom with —S(═O)₂Ra^{b}; wherein Ra^{b} is as defined in claim 37.

75. The method of any one of claims 37-74, wherein Rb^{b} is hydrogen or —C₁₋₄ alkyl.

76. The method of any one of claims 37-74, wherein Rb^{b} is hydrogen, —C(═O)Ra^{b}, —S(═O)₂Ra^{b}, —C₁₋₄ alkyl, —C₃₋₆ cycloalkyl, -(5- to 6-membered)-C₂₋₉ heterocyclyl, or —C₆₋₁₀ aryl optionally substituted with 1, 2 or 3 substituents each independently selected from the group consisting of halogen, hydroxy, CN, —O(C₁₋₄ alkyl), —S(C₁₋₄ alkyl), —NH(C₁₋₄ alkyl), —N(C₁₋₄ alkyl)₂, and —C₁₋₄alkyl optionally substituted by 1, 2 or 3 fluorine atoms.

77. The method of any one of claims 37-43, wherein the compound is selected from the group consisting of 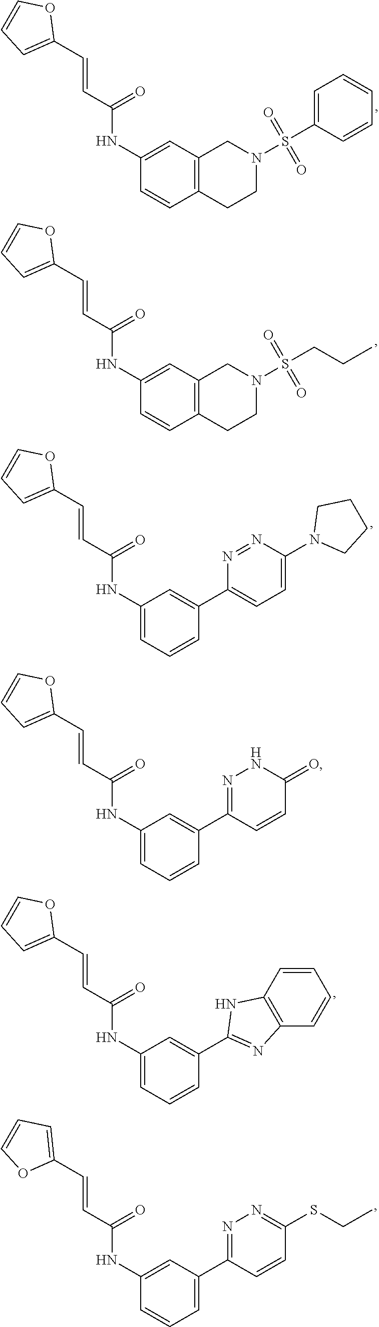 [SMILES: O=C(/C=C/c1ccco1)Nc1ccc2c(c1)CN(S(=O)(=O)c1ccccc1)CC2 ; CCCS(=O)(=O)N1CCc2ccc(NC(=O)/C=C/c3ccco3)cc2C1 ; O=C(/C=C/c1ccco1)Nc1cccc(-c2ccc(N3CCCC3)nn2)c1 ; O=C(/C=C/c1ccco1)Nc1cccc(-c2ccc(=O)[nH]n2)c1 ; O=C(/C=C/c1ccco1)Nc1cccc(-c2nc3ccccc3[nH]2)c1 ; CCSc1ccc(-c2cccc(NC(=O)/C=C/c3ccco3)c2)nn1] 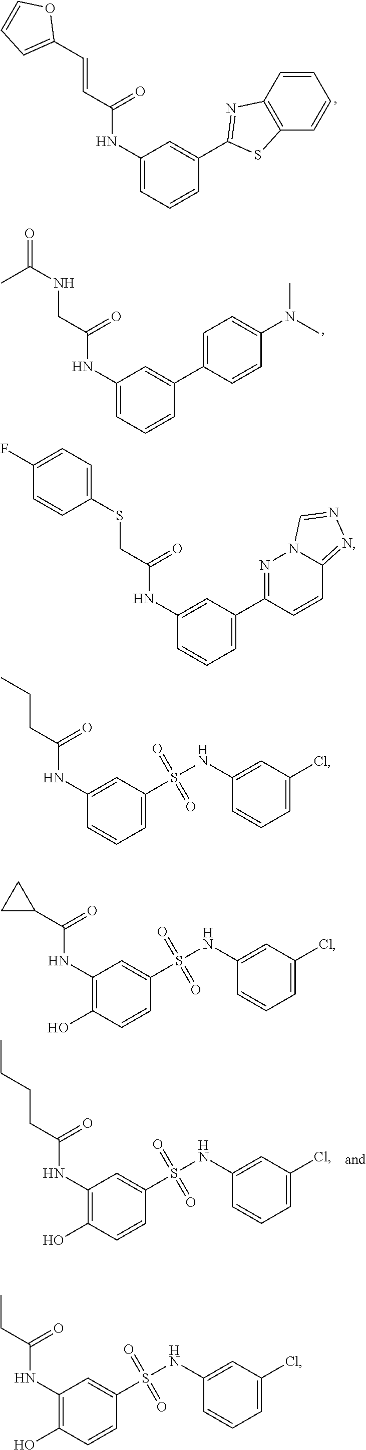
or a pharmaceutically acceptable salt or solvate thereof.

78. The method of any one of claims 37-43, wherein the compound is selected from the group consisting of 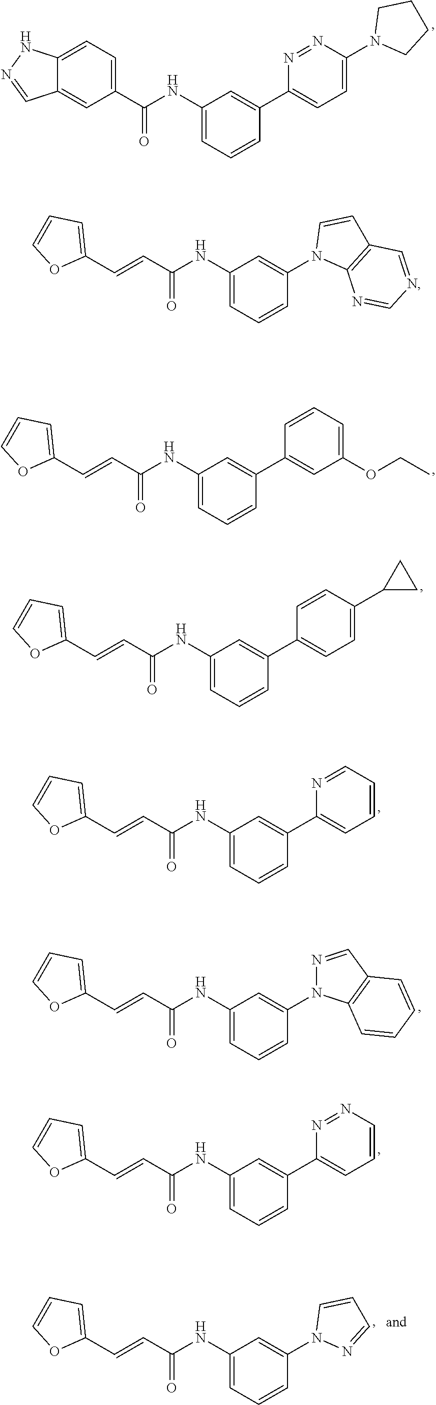 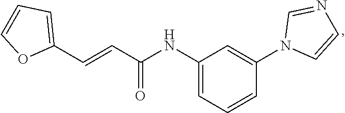 [SMILES: O=C(/C=C/c1ccco1)Nc1cccc(-n2ccnc2)c1]
or a pharmaceutically acceptable salt or solvate thereof.

79. The method of any one of claims 37-43, wherein the compound is selected from the group consisting of 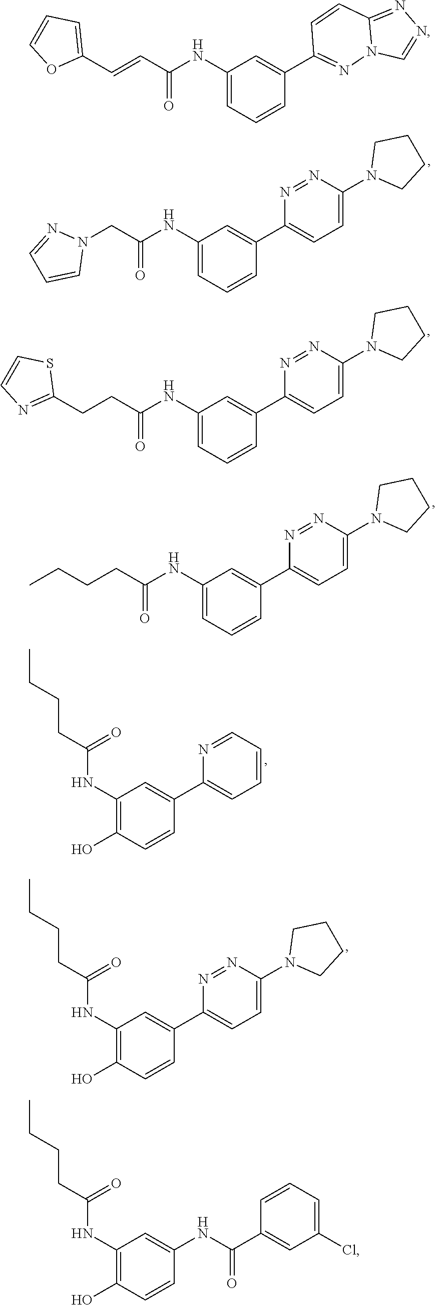 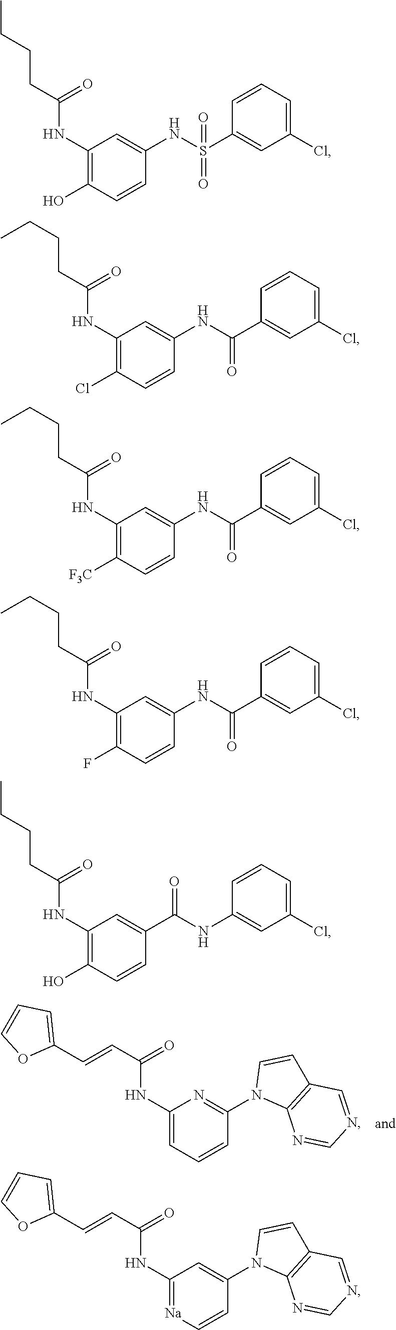
or a pharmaceutically acceptable salt or solvate thereof.

80. The method of any one of claims 37-42, or 44, wherein the compound is selected from the group consisting of 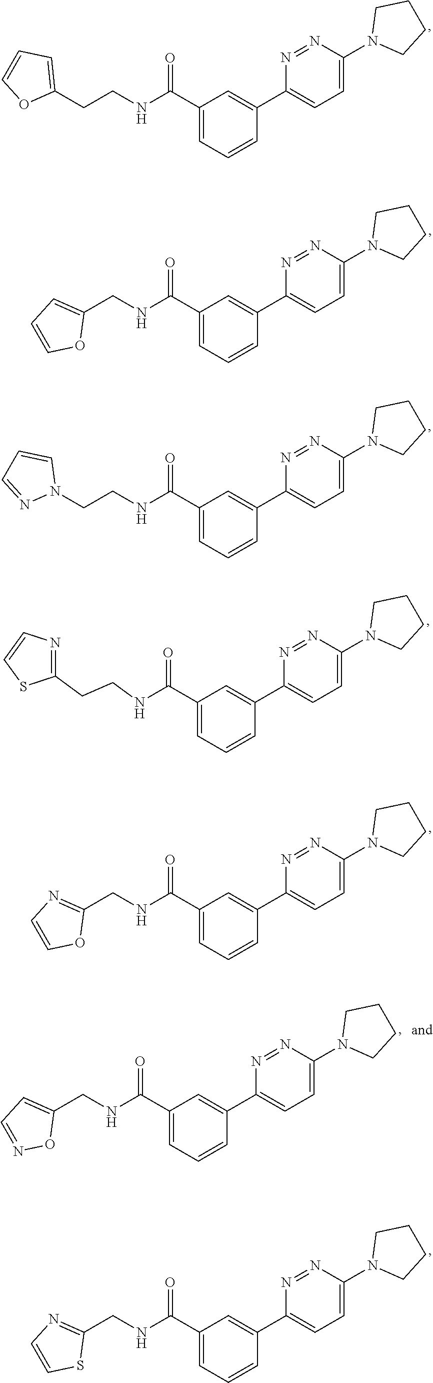
or a pharmaceutically acceptable salt or solvate thereof.

81. The method of any one of claims 1-80, further comprising administering to the patient at least one other therapeutic agent.

82. The method of claim 81, wherein the therapeutic agent is an effective amount of an enzyme for enzyme replacement therapy.

83. The method of claim 82, wherein the enzyme is galactocerebrosidase or an analog thereof.

84. The method of claim 81, wherein the therapeutic agent is an effective amount of a small molecule chaperone.

85. The method of claim 84, wherein the small molecule chaperone binds competitively to an enzyme.

86. The method of claim 84 or 85, wherein the small molecule chaperone is selected from the group consisting of iminoalditols, iminosugars, aminosugars, thiophenylglycosides, glycosidase, sulfatase, glycosyl transferase, phosphatase, and peptidase inhibitors.

87. A compound of formula (IIA): 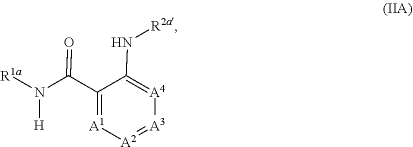
or a pharmaceutically acceptable salt or solvate thereof, wherein
A¹, A², A³, and A⁴ are each independently selected from the group consisting of N, CH and C(R^{3a}), provided that no more than one of A¹, A², A³, or A⁴ is N;
each R^{3a} is independently selected from the group consisting of halogen, —OH, C₁₋₄ alkyl, halo(C₁₋₄ alkyl), C₁₋₄ alkoxy, halo(C₁₋₄ alkoxy), and CN;
R^{1a} is selected from the group consisting of —C₁₋₄ alkyl, —C₃₋₁₀ cycloalkyl, —C₁₋₄ alkyl-C₃₋₁₀ cycloalkyl, —C₆₋₁₀ aryl, —C₁₋₄ alkyl-C₆₋₁₀ aryl, (5- to 10-membered)-C₁₋₉ heteroaryl, —C₁₋₄ alkyl-(5- to 10-membered)-C₁₋₉ heteroaryl, (5- to 10-membered)-C₂₋₉ heterocyclyl, and —C₁₋₄ alkyl-(5- to 10-membered)-C₂₋₉ heterocyclyl, wherein said alkyl, cycloalkyl, alkylcycloalkyl, aryl, alkylaryl, heteroaryl, alkylheteroaryl, heterocyclyl and alkylheterocyclyl groups are optionally substituted with 1, 2 or 3 substituents each independently selected from the group consisting of halogen, hydroxo, —CN, —ORb^{a}, —SRb^{a}, —N(Rb^{a})₂, —C₁₋₄ alkyl optionally substituted with 1, 2, or 3 halogen atoms, optionally substituted C₆₋₁₀ aryl, optionally substituted (5- to 10-membered)-C₁₋₉ heteroaryl, and (5- to 10-membered)-C₂₋₉ heterocyclyl; and wherein said cycloalkyl, alkylcycloalkyl, aryl, alkylaryl, heteroaryl, alkylheteroaryl, heterocyclyl and alkylheterocyclyl is optionally fused to a further (second) ring;
R^{2a′} is selected from the group consisting of —C(═O)Ra^{a′}, —S(═O)₂Ra^{a′}, —C₁₋₄ alkyl-C(═O)NHRa^{a′}, —C₁₋₄ alkyl-C(═O)N(Ra^{a′})₂, —C₁₋₄ alkyl-S(═O)₂—N(Ra^{a′})₂, wherein said alkyl group is optionally substituted with 1, 2 or 3 substituents each independently selected from the group consisting of halogen, hydroxy, —CN, —C₁₋₄ alkyl optionally substituted with 1, 2, or 3 halogen atoms;
Ra^{a′} is selected from the group consisting of —C₆₋₁₀ aryl, —C₁₋₄ alkyl-C₆₋₁₀ aryl, (5- to 10-membered)-C₁₋₉ heteroaryl, —C₁₋₄ alkyl-(5- to 10-membered)-C₁₋₉ heteroaryl, -(5- to 10-membered)-C₂₋₉ heterocyclyl, and —C₁₋₄ alkyl-(5- to 10-membered)-C₂₋₉ heterocyclyl, wherein said aryl, alkylaryl, heteroaryl, alkylheteroaryl, heterocyclyl and alkylheterocyclyl groups are optionally substituted with 1, 2 or 3 substituents each independently selected from the group consisting of halogen, hydroxy, —CN, —ORb^{a}, —SRb^{a}, —N(Rb^{a})₂, —C₁₋₄ alkyl optionally substituted with 1, 2, or 3 halogen atoms, optionally substituted C₆₋₁₀ aryl, optionally substituted (5- to 10-membered)-C₁₋₉ heteroaryl, and (5- to 10-membered)-C₂₋₉ heterocyclyl; and wherein said cycloalkyl, alkylcycloalkyl, aryl, alkylaryl, heteroaryl, alkylheteroaryl, heterocyclyl and alkylheterocyclyl is optionally fused to a further (second) ring; and
each Rb^{a} is independently hydrogen, —C₁₋₄ alkyl, —C₃₋₁₀ cycloalkyl, or -(5- to 10-membered)-C₂₋₉ heterocyclyl, wherein said alkyl, cycloalkyl or heterocyclyl group is optionally substituted by 1, 2 or 3 fluorine atoms.

88. The compound of claim 87, wherein 1) when A¹ is N and R^{2a′} is —C₁₋₄ alkyl-C(═O)NHRa^{a′}, then Ra^{a′} is other than -(5- to 10-membered)-C₂₋₉ heterocyclyl; or 2) when A⁴ is N, then R^{2a′} is other than —C(═O)Ra^{a′}.

89. The compound of claim 87, wherein the compound is selected from the group consisting of 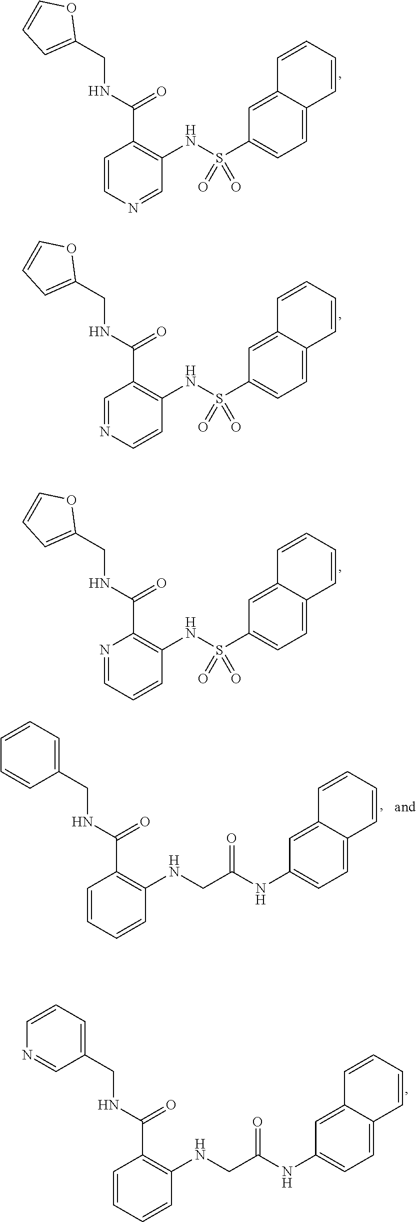 [SMILES: O=C(NCc1ccco1)c1ccncc1NS(=O)(=O)c1ccc2ccccc2c1 ; O=C(NCc1ccco1)c1cnccc1NS(=O)(=O)c1ccc2ccccc2c1 ; O=C(NCc1ccco1)c1ncccc1NS(=O)(=O)c1ccc2ccccc2c1 ; O=C(CNc1ccccc1C(=O)NCc1ccccc1)Nc1ccc2ccccc2c1 ; O=C(CNc1ccccc1C(=O)NCc1cccnc1)Nc1ccc2ccccc2c1]
or a pharmaceutically acceptable salt or solvate thereof.

90. A compound selected from the group consisting of compound is selected from the group consisting of 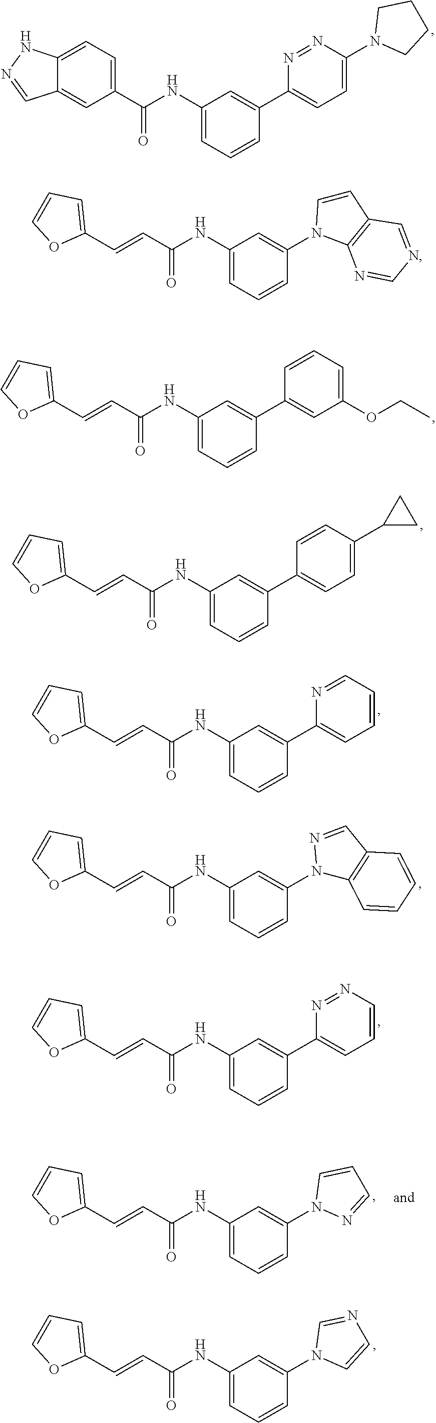
or a pharmaceutically acceptable salt or solvate thereof.

91. A compound selected from the group consisting of 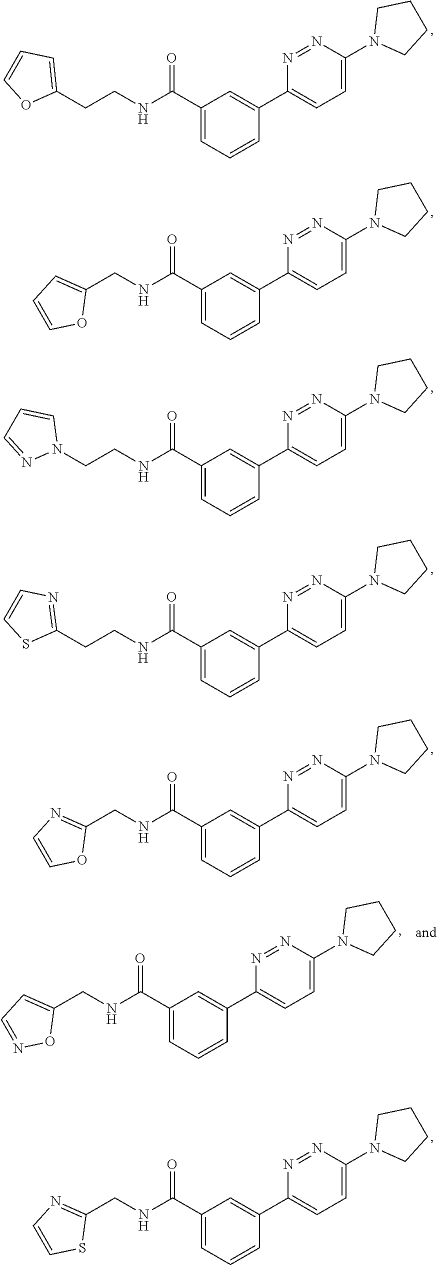
or a pharmaceutically acceptable salt or solvate thereof.

92. A compound selected from the group consisting of 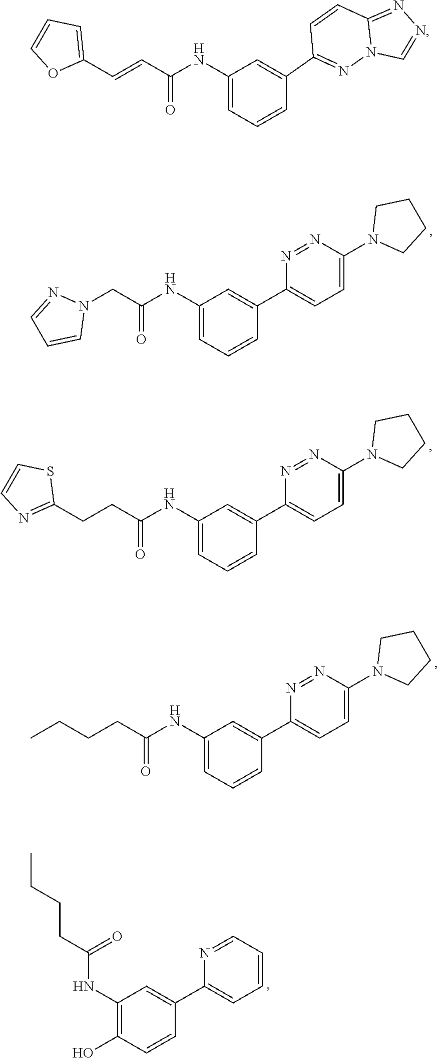 [SMILES: O=C(/C=C/c1ccco1)Nc1cccc(-c2ccc3nncn3n2)c1 ; O=C(Cn1cccn1)Nc1cccc(-c2ccc(N3CCCC3)nn2)c1 ; O=C(CCc1nccs1)Nc1cccc(-c2ccc(N3CCCC3)nn2)c1 ; CCCCC(=O)Nc1cccc(-c2ccc(N3CCCC3)nn2)c1 ; CCCCC(=O)Nc1cc(-c2ccccn2)ccc1O] 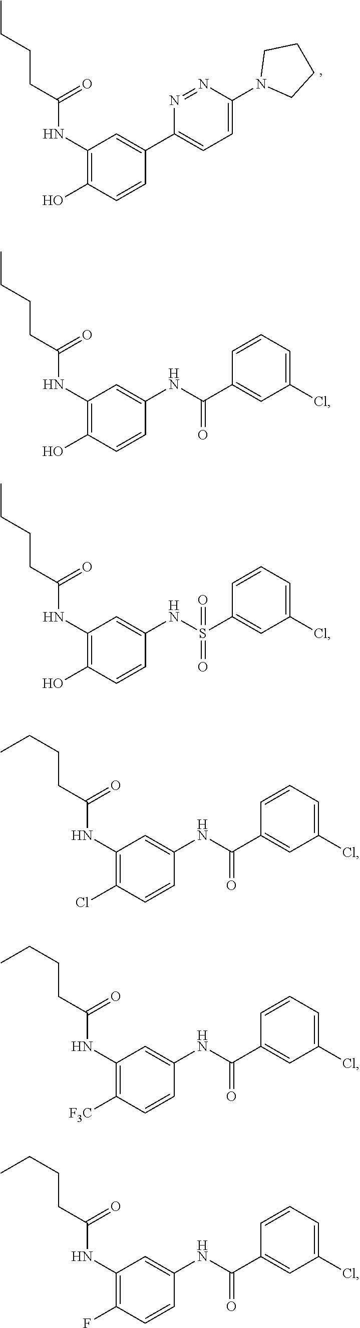 [SMILES: CCCCC(=O)Nc1cc(-c2ccc(N3CCCC3)nn2)ccc1O ; CCCCC(=O)Nc1cc(NC(=O)c2cccc(Cl)c2)ccc1O ; CCCCC(=O)Nc1cc(NS(=O)(=O)c2cccc(Cl)c2)ccc1O ; CCCCC(=O)Nc1cc(NC(=O)c2cccc(Cl)c2)ccc1Cl ; CCCCC(=O)Nc1cc(NC(=O)c2cccc(Cl)c2)ccc1C(F)(F)F ; CCCCC(=O)Nc1cc(NC(=O)c2cccc(Cl)c2)ccc1F] 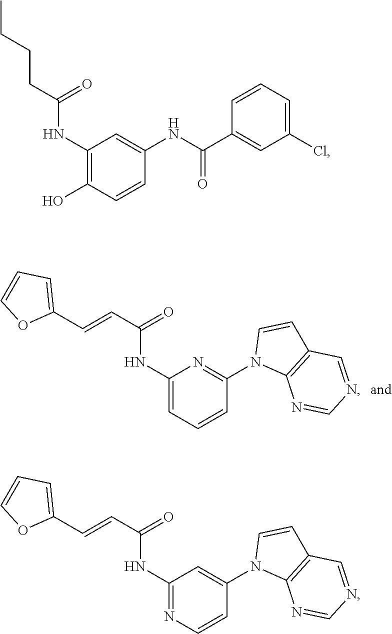 [SMILES: CCCCC(=O)Nc1cc(NC(=O)c2cccc(Cl)c2)ccc1O ; O=C(/C=C/c1ccco1)Nc1cccc(-n2ccc3cncnc32)n1 ; "and" ; O=C(/C=C/c1ccco1)Nc1cc(-n2ccc3cncnc32)ccn1]
or a pharmaceutically acceptable salt or solvate thereof.

93. A pharmaceutical composition, comprising an effective amount of a compound of formula (IA), or a pharmaceutically acceptable salt or solvate thereof, and at least one pharmaceutically acceptable excipient, wherein the compound of formula (IA) has the structure: 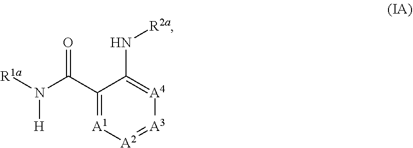
or a pharmaceutically acceptable salt or solvate thereof, wherein
A¹, A², A³, and A⁴ are each independently selected from the group consisting of N, CH and C(R^{3a});
each R^{3a} is independently selected from the group consisting of halogen, —OH, —C₁₋₄ alkyl, halo(C₁₋₄ alkyl), —C₁₋₄ alkoxy, halo(C₁₋₄ alkoxy), and —CN;
R^{1a} is selected from the group consisting of —C₁₋₄ alkyl, —C₃₋₁₀ cycloalkyl, —C₁₋₄ alkyl-C₃₋₁₀ cycloalkyl, —C₆₋₁₀ aryl, —C₁₋₄ alkyl-C₆₋₁₀ aryl, -(5- to 10-membered)-C₁₋₉ heteroaryl, —C₁₋₄ alkyl-(5- to 10-membered)-C₁₋₉ heteroaryl, -(5- to 10-membered)-C₂₋₉ heterocyclyl, and —C₁₋₄ alkyl-(5- to 10-membered)-C₂₋₉ heterocyclyl, wherein said alkyl, cycloalkyl, alkylcycloalkyl, aryl, alkylaryl, heteroaryl, alkylheteroaryl, heterocyclyl and alkylheterocyclyl groups are optionally substituted with 1, 2 or 3 substituents each independently selected from the group consisting of halogen, hydroxy, CN, —ORb^{a}, —SRb^{a}, —N(Rb^{a})₂, —C₁₋₄ alkyl optionally substituted with 1, 2, or 3 halogen atoms, optionally substituted —C₆₋₁₀ aryl, optionally substituted -(5- to 10-membered)-C₁₋₉ heteroaryl, and -(5- to 10-membered)-C₂₋₉ heterocyclyl; and wherein said cycloalkyl, alkylcycloalkyl, aryl, alkylaryl, heteroaryl, alkylheteroaryl, heterocyclyl and alkylheterocyclyl is optionally fused to a further (second) ring; and
R^{2a} is selected from the group consisting of —C₁₋₄ alkyl, —C(═O)Ra^{a}, —C(═O)NHRa^{a}, —S(═O)₂Ra^{a}, —C₁₋₄ alkyl-C(═O)Ra^{a}, —C₁₋₄ alkyl-C(═O)NHRa^{a}, —C₁₋₄ alkyl-C(═O)N(Ra^{a})₂, —C₁₋₄ alkyl-S(═O)₂Ra^{a}, —C₁₋₄ alkyl-S(═O)₂—N(Ra^{a})₂, —C₁₋₄ alkyl-C₃₋₁₀ cycloalkyl, —C₁₋₄ alkyl-C₆₋₁₀ aryl, -(5- to 10-membered)-C₁₋₉ heteroaryl, —C₁₋₄ alkyl-(5- to 10-membered)-C₁₋₉ heteroaryl, (5- to 10-membered)-C₂₋₉ heterocyclyl, and —C₁₋₄ alkyl-(5- to 10-membered)-C₂₋₉ heterocyclyl, wherein said alkyl, cycloalkyl, alkylcycloalkyl, aryl, alkylaryl, heteroaryl, alkylheteroaryl, heterocyclyl and alkylheterocyclyl groups are optionally substituted with 1, 2 or 3 substituents each independently selected from the group consisting of halogen, hydroxy, —CN, —C(═O)Ra^{a}, —ORb^{a}, —SRb^{a}, —N(Rb^{a})₂, (═O), —C₁₋₄ alkyl optionally substituted with 1, 2, or 3 halogen atoms, optionally substituted C₆₋₁₀ aryl, optionally substituted -(5- to 10-membered)-C₁₋₉ heteroaryl, and -(5- to 10-membered)-C₂₋₉ heterocyclyl; and wherein said cycloalkyl, alkylcycloalkyl, aryl, alkylaryl, heteroaryl, alkylheteroaryl, heterocyclyl and alkylheterocyclyl is optionally fused to a further (second) ring;
Ra^{a} is selected from the group consisting of —C₁₋₄ alkyl, —C₃₋₁₀ cycloalkyl, —C₁₋₄ alkyl-C₃₋₁₀ cycloalkyl, —C₆₋₁₀ aryl, —C₁₋₄ alkyl-C₆₋₁₀ aryl, -(5- to 10-membered)-C₁₋₉ heteroaryl, —C₁₋₄ alkyl-(5- to 10-membered)-C₁₋₉ heteroaryl, -(5- to 10-membered)-C₂₋₉ heterocyclyl, and —C₁₋₄ alkyl-(5- to 10-membered)-C₂₋₉ heterocyclyl, wherein said alkyl, cycloalkyl, alkylcycloalkyl, aryl, alkylaryl, heteroaryl, alkylheteroaryl, heterocyclyl and alkylheterocyclyl groups are optionally substituted with 1, 2 or 3 substituents each independently selected from the group consisting of halogen, hydroxy, —CN, —ORb^{a}, —SRb^{a}, —N(Rb^{a})₂, —C₁₋₄ alkyl optionally substituted with 1, 2, or 3 halogen atoms, optionally substituted —C₆₋₁₀ aryl, optionally substituted -(5- to 10-membered)-C₁₋₉ heteroaryl, and -(5- to 10-membered)-C₂₋₉ heterocyclyl; and wherein said cycloalkyl, alkylcycloalkyl, aryl, alkylaryl, heteroaryl, alkylheteroaryl, heterocyclyl and alkylheterocyclyl is optionally fused to a further (second) ring; and
each Rb^{a} is independently hydrogen, —C₁₋₄ alkyl, —C₃₋₁₀ cycloalkyl, or -(5- to 10-membered)-C₂₋₉ heterocyclyl, wherein said alkyl, cycloalkyl or heterocyclyl group is optionally substituted by 1, 2 or 3 fluorine atoms.

94. The pharmaceutical composition of claim 93, wherein the compound of formula (IA) is a compound of formula (IIA) having the structure: 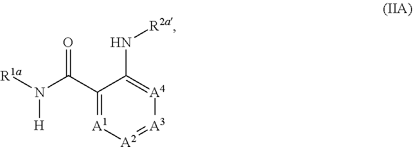
or a pharmaceutically acceptable salt or solvate thereof, wherein
A¹, A², A³, and A⁴ are each independently selected from the group consisting of N, CH and C(R^{3a}), provided that no more than one of A¹, A², A³, or A⁴ is N;
each R^{3a} is independently selected from the group consisting of halogen, —OH, C₁₋₄ alkyl, halo(C₁₋₄ alkyl), C₁₋₄ alkoxy, halo(C₁₋₄ alkoxy), and CN;
R^{1a} is selected from the group consisting of —C₁₋₄ alkyl, —C₃₋₁₀ cycloalkyl, —C₁₋₄ alkyl-C₃₋₁₀ cycloalkyl, —C₆₋₁₀ aryl, —C₁₋₄ alkyl-C₆₋₁₀ aryl, (5- to 10-membered)-C₁₋₉ heteroaryl, —C₁₋₄ alkyl-(5- to 10-membered)-C₁₋₉ heteroaryl, (5- to 10-membered)-C₂₋₉ heterocyclyl, and —C₁₋₄ alkyl-(5- to 10-membered)-C₂₋₉ heterocyclyl, wherein said alkyl, cycloalkyl, alkylcycloalkyl, aryl, alkylaryl, heteroaryl, alkylheteroaryl, heterocyclyl and alkylheterocyclyl groups are optionally substituted with 1, 2 or 3 substituents each independently selected from the group consisting of halogen, hydroxo, —CN, —ORb^{a}, —SRb^{a}, —N(Rb^{a})₂, —C₁₋₄ alkyl optionally substituted with 1, 2, or 3 halogen atoms, optionally substituted C₆₋₁₀ aryl, optionally substituted (5- to 10-membered)-C₁₋₉ heteroaryl, and (5- to 10-membered)-C₂₋₉ heterocyclyl; and wherein said cycloalkyl, alkylcycloalkyl, aryl, alkylaryl, heteroaryl, alkylheteroaryl, heterocyclyl and alkylheterocyclyl is optionally fused to a further (second) ring;
R^{2a′} is selected from the group consisting of —C(═O)Ra^{a′}, —S(═O)₂Ra^{a′}, —C₁₋₄ alkyl-C(═O)NHRa^{a′}, —C₁₋₄ alkyl-C(═O)N(Ra^{a′})₂, —C₁₋₄ alkyl-S(═O)₂—N(Ra^{a′})₂, wherein said alkyl group is optionally substituted with 1, 2 or 3 substituents each independently selected from the group consisting of halogen, hydroxy, —CN, —C₁₋₄ alkyl optionally substituted with 1, 2, or 3 halogen atoms;
Ra^{a′} is selected from the group consisting of —C₆₋₁₀ aryl, —C₁₋₄ alkyl-C₆₋₁₀ aryl, (5- to 10-membered)-C₁₋₉ heteroaryl, —C₁₋₄ alkyl-(5- to 10-membered)-C₁₋₉ heteroaryl, -(5- to 10-membered)-C₂₋₉ heterocyclyl, and —C₁₋₄ alkyl-(5- to 10-membered)-C₂₋₉ heterocyclyl, wherein said aryl, alkylaryl, heteroaryl, alkylheteroaryl, heterocyclyl and alkylheterocyclyl groups are optionally substituted with 1, 2 or 3 substituents each independently selected from the group consisting of halogen, hydroxy, —CN, —ORb^{a}, —SRb^{a}, —N(Rb^{a})₂, —C₁₋₄ alkyl optionally substituted with 1, 2, or 3 halogen atoms, optionally substituted C₆₋₁₀ aryl, optionally substituted (5- to 10-membered)-C₁₋₉ heteroaryl, and (5- to 10-membered)-C₂₋₉ heterocyclyl; and wherein said cycloalkyl, alkylcycloalkyl, aryl, alkylaryl, heteroaryl, alkylheteroaryl, heterocyclyl and alkylheterocyclyl is optionally fused to a further (second) ring; and
each Rb^{a} is independently hydrogen, —C₁₋₄ alkyl, —C₃₋₁₀ cycloalkyl, or -(5- to 10-membered)-C₂₋₉ heterocyclyl, wherein said alkyl, cycloalkyl or heterocyclyl group is optionally substituted by 1, 2 or 3 fluorine atoms.

95. The pharmaceutical composition of claim 93, wherein the compound of formula (IA) is selected from the group consisting of 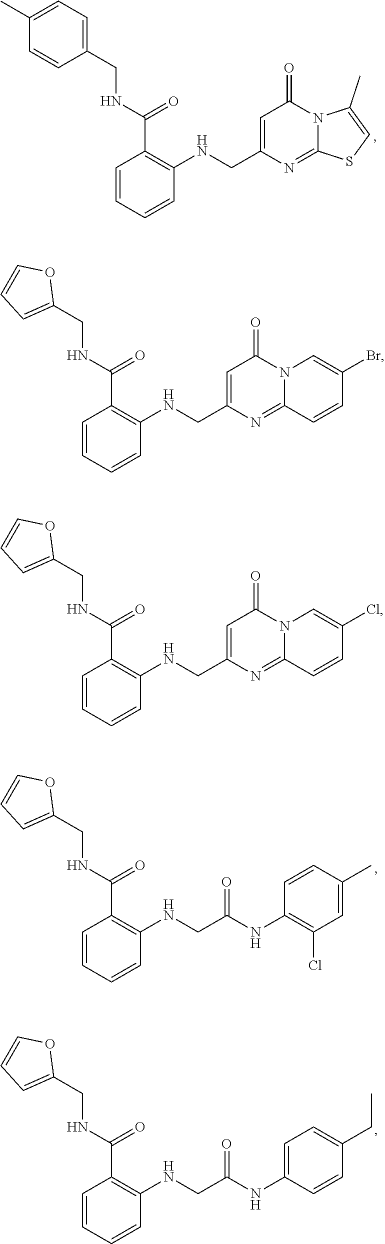 [SMILES: Cc1ccc(CNC(=O)c2ccccc2NCc2cc(=O)n3c(C)csc3n2)cc1 ; O=C(NCc1ccco1)c1ccccc1NCc1cc(=O)n2cc(Br)ccc2n1 ; O=C(NCc1ccco1)c1ccccc1NCc1cc(=O)n2cc(Cl)ccc2n1 ; Cc1ccc(NC(=O)CNc2ccccc2C(=O)NCc2ccco2)c(Cl)c1 ; CCc1ccc(NC(=O)CNc2ccccc2C(=O)NCc2ccco2)cc1] 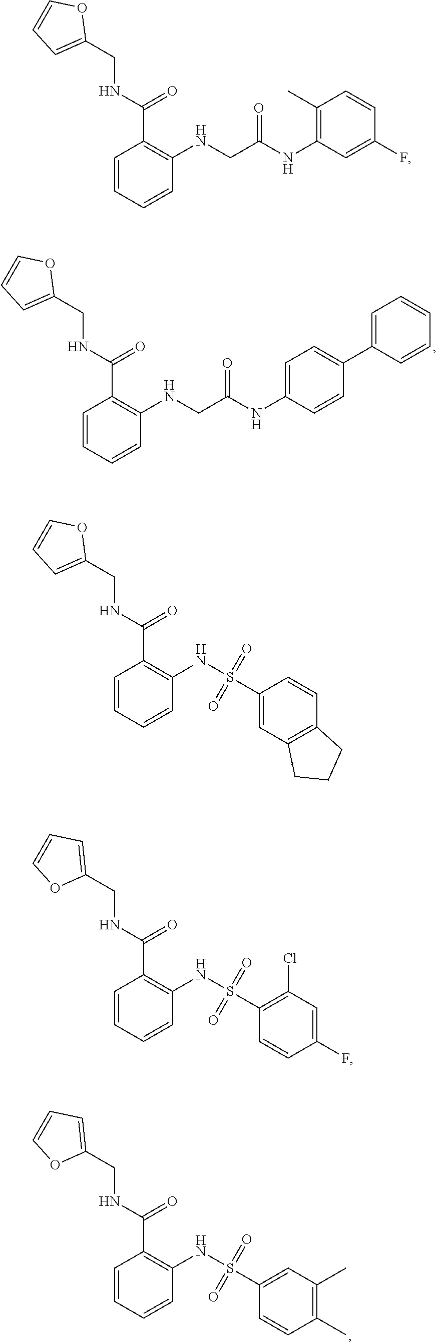 [SMILES: Cc1ccc(F)cc1NC(=O)CNc1ccccc1C(=O)NCc1ccco1 ; O=C(CNc1ccccc1C(=O)NCc1ccco1)Nc1ccc(-c2ccccc2)cc1 ; O=C(NCc1ccco1)c1ccccc1NS(=O)(=O)c1ccc2c(c1)CCC2 ; O=C(NCc1ccco1)c1ccccc1NS(=O)(=O)c1ccc(F)cc1Cl ; Cc1ccc(S(=O)(=O)Nc2ccccc2C(=O)NCc2ccco2)cc1C] 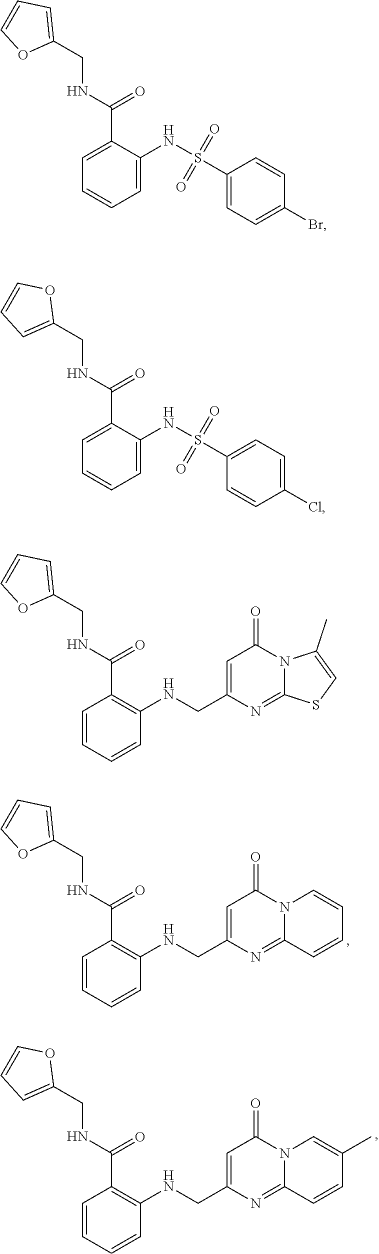 [SMILES: O=C(NCc1ccco1)c1ccccc1NS(=O)(=O)c1ccc(Br)cc1 ; O=C(NCc1ccco1)c1ccccc1NS(=O)(=O)c1ccc(Cl)cc1 ; Cc1csc2nc(CNc3ccccc3C(=O)NCc3ccco3)cc(=O)n12 ; O=C(NCc1ccco1)c1ccccc1NCc1cc(=O)n2ccccc2n1 ; Cc1ccc2nc(CNc3ccccc3C(=O)NCc3ccco3)cc(=O)n2c1] 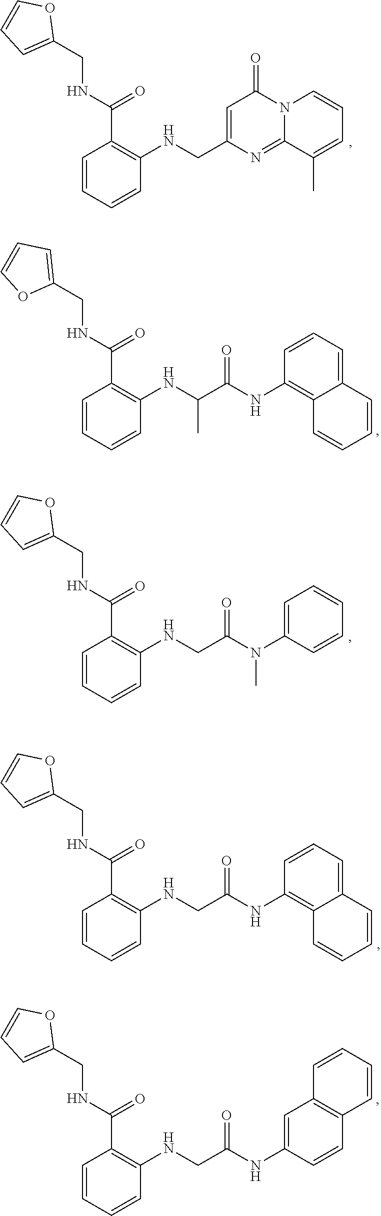 [SMILES: Cc1cccn2c(=O)cc(CNc3ccccc3C(=O)NCc3ccco3)nc12 ; CC(Nc1ccccc1C(=O)NCc1ccco1)C(=O)Nc1cccc2ccccc12 ; CN(C(=O)CNc1ccccc1C(=O)NCc1ccco1)c1ccccc1 ; O=C(CNc1ccccc1C(=O)NCc1ccco1)Nc1cccc2ccccc12 ; O=C(CNc1ccccc1C(=O)NCc1ccco1)Nc1ccc2ccccc2c1] 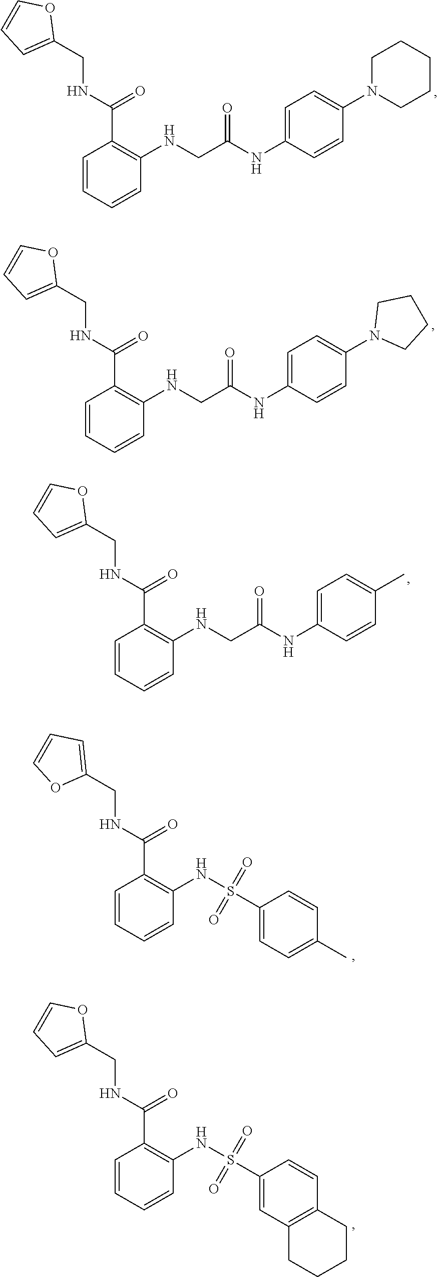 [SMILES: O=C(CNc1ccccc1C(=O)NCc1ccco1)Nc1ccc(N2CCCCC2)cc1 ; O=C(CNc1ccccc1C(=O)NCc1ccco1)Nc1ccc(N2CCCC2)cc1 ; Cc1ccc(NC(=O)CNc2ccccc2C(=O)NCc2ccco2)cc1 ; Cc1ccc(S(=O)(=O)Nc2ccccc2C(=O)NCc2ccco2)cc1 ; O=C(NCc1ccco1)c1ccccc1NS(=O)(=O)c1ccc2c(c1)CCCC2] 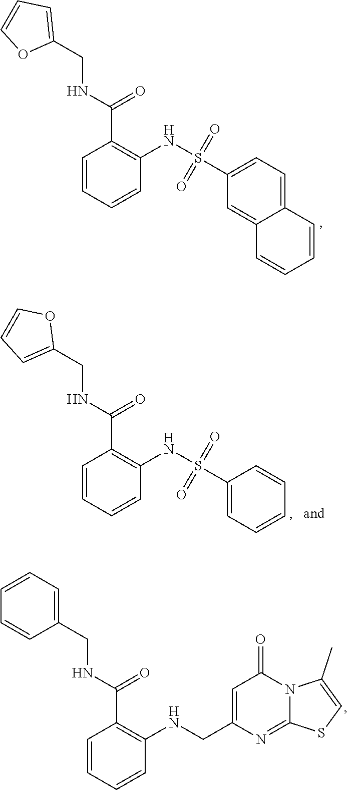 [SMILES: O=C(NCc1ccco1)c1ccccc1NS(=O)(=O)c1ccc2ccccc2c1 ; O=C(NCc1ccco1)c1ccccc1NS(=O)(=O)c1ccccc1 ; Cc1csc2nc(CNc3ccccc3C(=O)NCc3ccccc3)cc(=O)n12]
or a pharmaceutically acceptable salt or solvate thereof.

96. The pharmaceutical composition of claim 93, wherein the compound is selected form the group consisting of 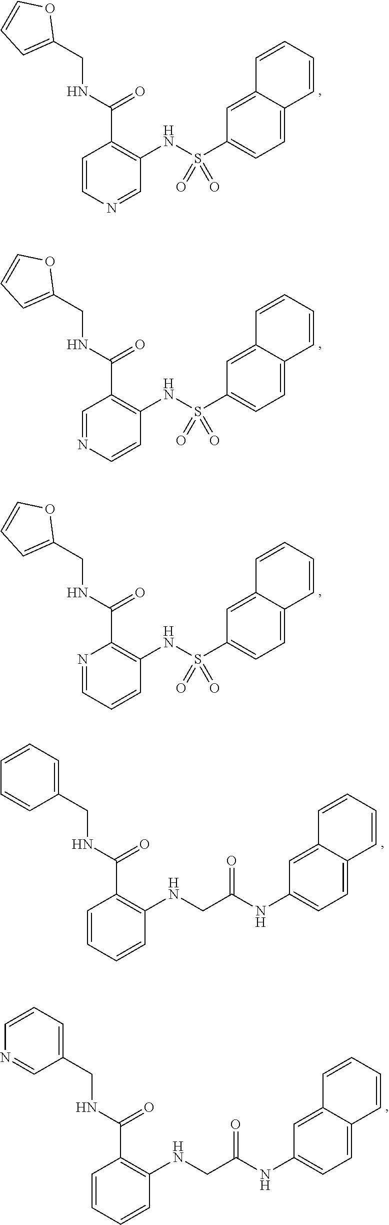 [SMILES: O=C(NCc1ccco1)c1ccncc1NS(=O)(=O)c1ccc2ccccc2c1 ; O=C(NCc1ccco1)c1cnccc1NS(=O)(=O)c1ccc2ccccc2c1 ; O=C(NCc1ccco1)c1ncccc1NS(=O)(=O)c1ccc2ccccc2c1 ; O=C(CNc1ccccc1C(=O)NCc1ccccc1)Nc1ccc2ccccc2c1 ; O=C(CNc1ccccc1C(=O)NCc1cccnc1)Nc1ccc2ccccc2c1] 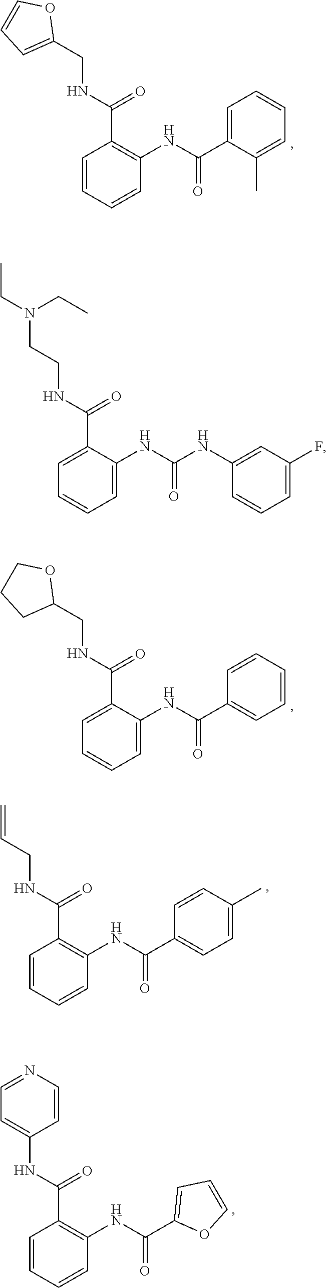 [SMILES: Cc1ccccc1C(=O)Nc1ccccc1C(=O)NCc1ccco1 ; CCN(CC)CCNC(=O)c1ccccc1NC(=O)Nc1cccc(F)c1 ; O=C(Nc1ccccc1C(=O)NCC1CCCO1)c1ccccc1 ; C=CCNC(=O)c1ccccc1NC(=O)c1ccc(C)cc1 ; O=C(Nc1ccccc1C(=O)Nc1ccncc1)c1ccco1] 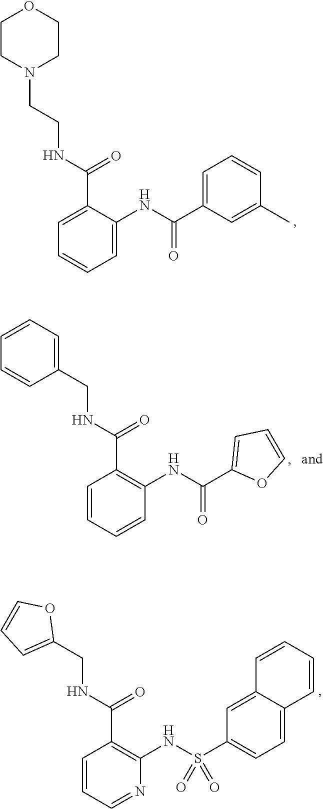 [SMILES: Cc1cccc(C(=O)Nc2ccccc2C(=O)NCCN2CCOCC2)c1 ; O=C(Nc1ccccc1C(=O)NCc1ccccc1)c1ccco1 ; O=C(NCc1ccco1)c1cccnc1NS(=O)(=O)c1ccc2ccccc2c1]
or a pharmaceutically acceptable salt or solvate thereof.

97. A pharmaceutical composition, comprising an effective amount of a compound of formula (IB), or a pharmaceutically acceptable salt or solvate thereof, and at least one pharmaceutically acceptable excipient, wherein the compound of formula (IB) has the structure: 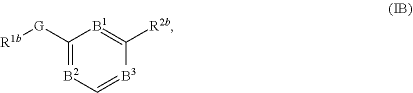
or a pharmaceutically acceptable salt or solvate thereof, wherein
G is —C(═O)—NH— or —NH—C(═O)—;
B¹, B², and B³ are each independently selected from the group consisting of N, CH and C(R^{3b});
each R^{3b} is independently selected from the group consisting of halogen, C₁₋₄ alkyl, halo(C₁₋₄ alkyl), —OH, C₁₋₄ alkoxy, halo(C₁₋₄)alkoxy, and CN;
R^{1b} is selected from the group consisting of —C₁₋₄ alkyl, —C₃₋₁₀ cycloalkyl, —C₁₋₄ alkyl-C₃₋₁₀ cycloalkyl, —C₆₋₁₀ aryl, —C₁₋₄ alkyl-C₆₋₁₀ aryl, —C₂₋₄ alkylene-C₆₋₁₀ aryl, (5- to 10-membered)-C₁₋₉ heteroaryl, —C₁₋₄ alkyl-(5- to 10-membered)-C₁₋₉ heteroaryl, —C₂₋₄ alkylene-(5- to 10-membered)-C₁₋₉ heteroaryl, (5- to 10-membered)-C₂₋₉ heterocyclyl, —C₁₋₄ alkyl-(5- to 10-membered)-C₂₋₉ heterocyclyl, and —C₂₋₄ alkenyl-(5- to 10-membered)-C₂₋₉ heterocyclyl, wherein said alkyl, cycloalkyl, alkylcycloalkyl, aryl, alkylaryl, heteroaryl, alkylheteroaryl, alkenylheteroaryl, heterocyclyl and alkylheterocyclyl groups are optionally substituted with 1, 2 or 3 substituents each independently selected from the group consisting of halogen, hydroxy, —CN, —ORb^{b}, —SRb^{b}, —N(Rb^{b})₂, —C₁₋₄ alkyl optionally substituted with 1, 2, or 3 halogen atoms, optionally substituted C₆₋₁₀ aryl, optionally substituted (5- to 10-membered)-C₁₋₉ heteroaryl, and (5- to 10-membered)-C₂₋₉ heterocyclyl; and wherein said cycloalkyl, alkylcycloalkyl, aryl, alkylaryl, heteroaryl, alkylheteroaryl, alkenylheteroaryl, heterocyclyl and alkylheterocyclyl is optionally fused to a further (second) ring;
R^{2b} is —C₆₋₁₀ aryl, -(5- to 10-membered)-C₁₋₉ heteroaryl, —C(═O)Ra^{b}, —S(═O)₂Ra^{b}, —C(═O)—NH—Ra^{b}, —S(═O)₂—NH—Ra^{b}, —C₁₋₄ alkyl-C(═O)Ra^{b}, —C₁₋₄ alkyl-S(═O)₂Ra^{b}, or —N(Rb^{b})₂, wherein said aryl and heteroaryl groups are optionally substituted with 1, 2 or 3 substituents each independently selected from the group consisting of halogen, hydroxy, CN, —ORb^{b}, —SRb^{b}, —N(Rb^{b})₂, (═O), —C₁₋₄alkyl optionally substituted with 1, 2, or 3 substituents each independently selected from the group consisting of halogen, CN, —ORb^{b}, and —N(Rb^{b})₂, optionally substituted —C₆₋₁₀ aryl, optionally substituted -(5- to 10-membered)-C₁₋₉ heteroaryl, -(5- to 10-membered)-C₂₋₉ heterocyclyl, and —C₃₋₁₀ cycloalkyl; and wherein said aryl, heteroaryl, and heterocyclyl are optionally fused to a further (second) ring; or
R^{2b} and R^{3b} attached to an adjacent carbon atom together form a 5- or 6-membered heterocyclic ring containing one N-atom substituted with —S(═O)₂Ra^{b};
Ra^{b} is selected from the group consisting of —C₁₋₄ alkyl, —C₃₋₁₀ cycloalkyl, —C₁₋₄ alkyl-C₃₋₁₀ cycloalkyl, —C₆₋₁₀ aryl, —C₁₋₄ alkyl-C₆₋₁₀ aryl, (5- to 10-membered)-C₁₋₉ heteroaryl, —C₁₋₄ alkyl-(5- to 10-membered)-C₁₋₉ heteroaryl, (5- to 10-membered)-C₂₋₉ heterocyclyl, and —C₁₋₄ alkyl-(5- to 10-membered)-C₂₋₉ heterocyclyl, wherein said alkyl, cycloalkyl, alkylcycloalkyl, aryl, alkylaryl, heteroaryl, alkylheteroaryl, heterocyclyl and alkylheterocyclyl groups are optionally substituted with 1, 2 or 3 substituents each independently selected from the group consisting of halogen, hydroxy, —CN, —ORb^{b}, —SRb^{b}, —N(Rb^{b})₂, —C₁₋₄ alkyl optionally substituted with 1, 2, or 3 halogen atoms, optionally substituted C₆₋₁₀ aryl, optionally substituted (5- to 10-membered)-C₁₋₉ heteroaryl, and (5- to 10-membered)-C₂₋₉ heterocyclyl; and wherein said cycloalkyl, alkylcycloalkyl, aryl, alkylaryl, heteroaryl, alkylheteroaryl, heterocyclyl and alkylheterocyclyl is optionally fused to a further (second) ring; and
each Rb^{b} is independently hydrogen, —C(═O)Ra^{b}, —S(═O)₂Ra^{b}, —C₁₋₄ alkyl, —C₃₋₁₀ cycloalkyl, -(5- to 10-membered)-C₂₋₉ heterocyclyl, or optionally substituted —C₆₋₁₀ aryl, wherein said alkyl, cycloalkyl or heterocyclyl group is optionally substituted by 1, 2 or 3 fluorine atoms.

98. The pharmaceutical composition of claim 97, comprising an effective amount of a compound of formula (IB) where G is —C(═O)—NH—, which is a compound of formula (IIB): 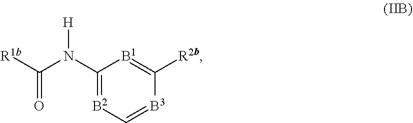
or a pharmaceutically acceptable salt or solvate thereof, wherein B¹, B², B³, R^{1b}, and R^{2b} are as defined in claim 97.

99. The pharmaceutical composition of claim 97, comprising an effective amount of a compound of formula (IB) where G is —NH—C(═O)—, which is a compound of formula (IIIB): 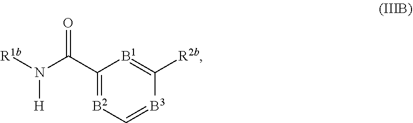
or a pharmaceutically acceptable salt or solvate thereof, wherein B¹, B², B³, R^{1b}, and R^{2b} are as defined in claim 97.

100. The pharmaceutical composition of claim 97 or 98, wherein the compound is selected from the group consisting of 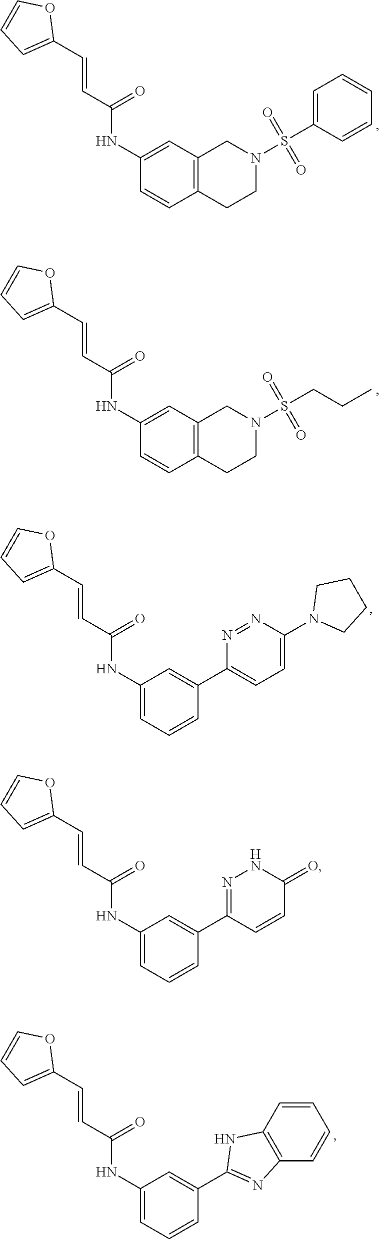 [SMILES: O=C(/C=C/c1ccco1)Nc1ccc2c(c1)CN(S(=O)(=O)c1ccccc1)CC2 ; CCCS(=O)(=O)N1CCc2ccc(NC(=O)/C=C/c3ccco3)cc2C1 ; O=C(/C=C/c1ccco1)Nc1cccc(-c2ccc(N3CCCC3)nn2)c1 ; O=C(/C=C/c1ccco1)Nc1cccc(-c2ccc(=O)[nH]n2)c1 ; O=C(/C=C/c1ccco1)Nc1cccc(-c2nc3ccccc3[nH]2)c1] 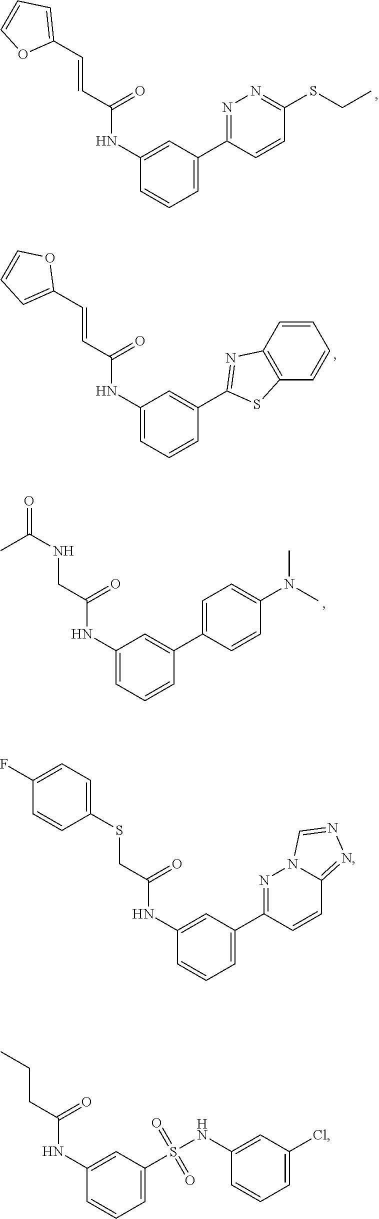 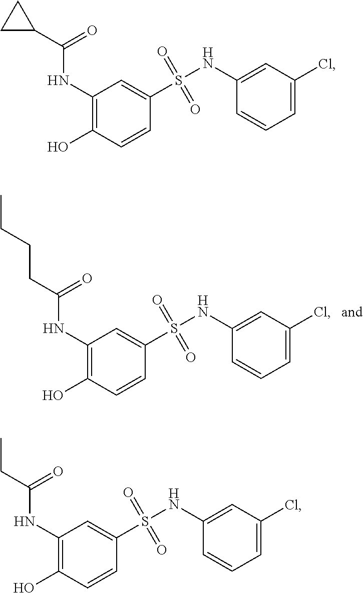 [SMILES: O=C(Nc1cc(S(=O)(=O)Nc2cccc(Cl)c2)ccc1O)C1CC1 ; CCCCC(=O)Nc1cc(S(=O)(=O)Nc2cccc(Cl)c2)ccc1O ; "and" ; CCC(=O)Nc1cc(S(=O)(=O)Nc2cccc(Cl)c2)ccc1O]
or a pharmaceutically acceptable salt or solvate thereof.

101. The pharmaceutical composition of claim 97 or 98, wherein the compound is selected from the group consisting of 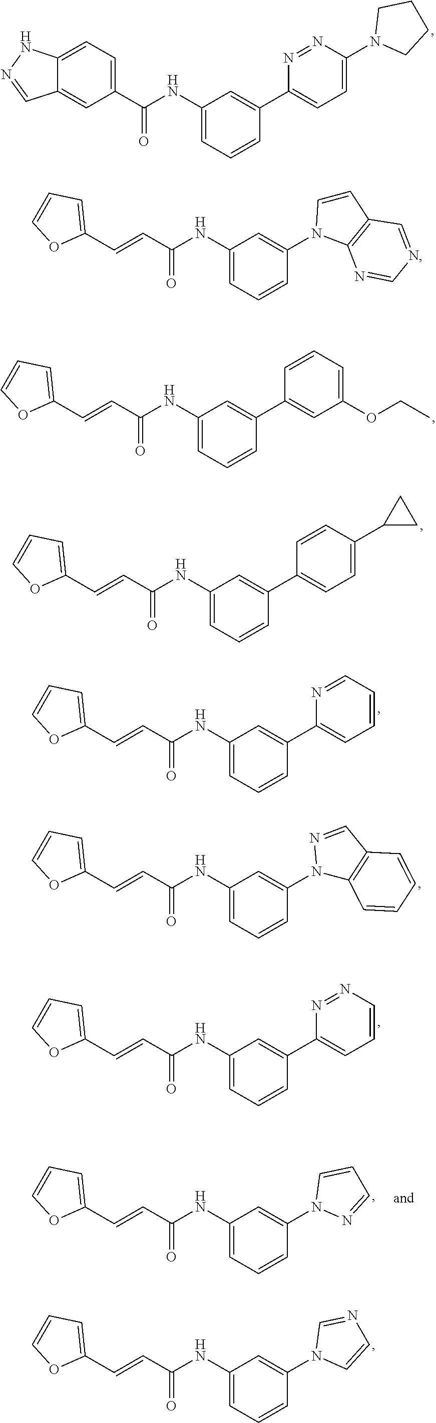
or a pharmaceutically acceptable salt or solvate thereof.

102. The pharmaceutical composition of claim 97 or 98, wherein the compound is selected form the group consisting of 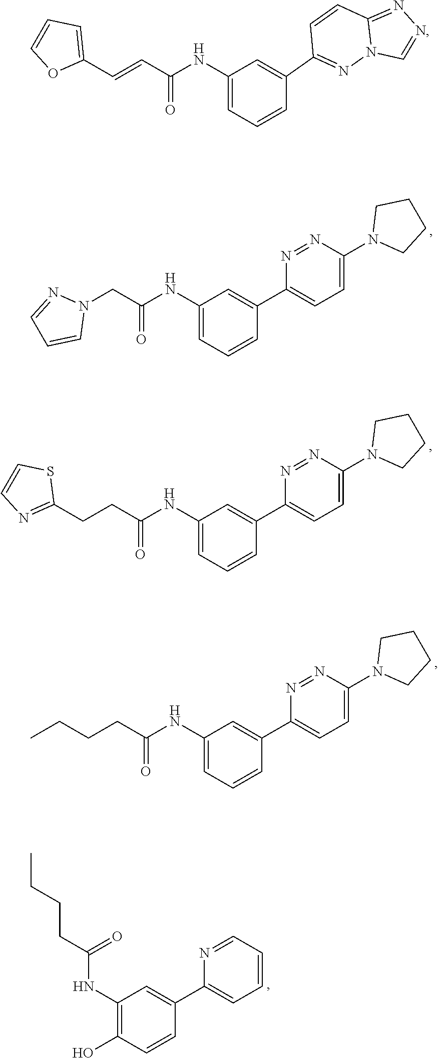 [SMILES: O=C(/C=C/c1ccco1)Nc1cccc(-c2ccc3nncn3n2)c1 ; O=C(Cn1cccn1)Nc1cccc(-c2ccc(N3CCCC3)nn2)c1 ; O=C(CCc1nccs1)Nc1cccc(-c2ccc(N3CCCC3)nn2)c1 ; CCCCC(=O)Nc1cccc(-c2ccc(N3CCCC3)nn2)c1 ; CCCCC(=O)Nc1cc(-c2ccccn2)ccc1O] 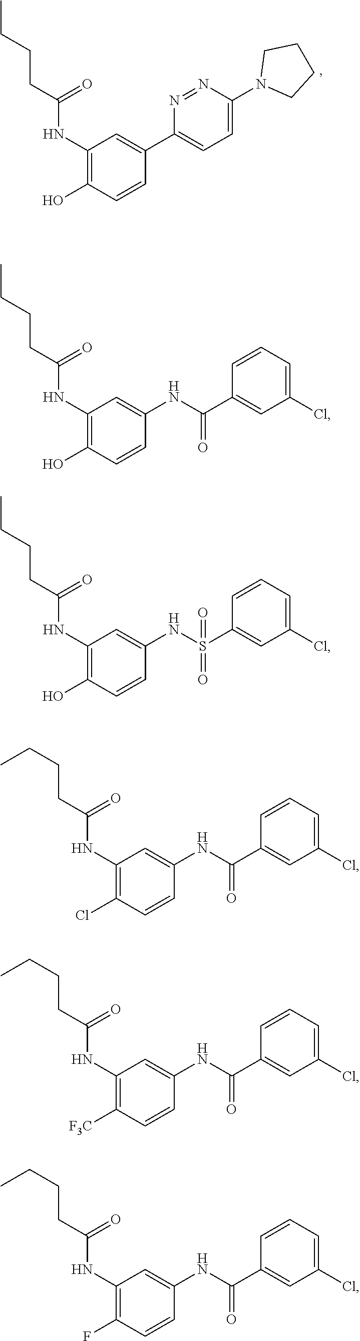 [SMILES: CCCCC(=O)Nc1cc(-c2ccc(N3CCCC3)nn2)ccc1O ; CCCCC(=O)Nc1cc(NC(=O)c2cccc(Cl)c2)ccc1O ; CCCCC(=O)Nc1cc(NS(=O)(=O)c2cccc(Cl)c2)ccc1O ; CCCCC(=O)Nc1cc(NC(=O)c2cccc(Cl)c2)ccc1Cl ; CCCCC(=O)Nc1cc(NC(=O)c2cccc(Cl)c2)ccc1C(F)(F)F ; CCCCC(=O)Nc1cc(NC(=O)c2cccc(Cl)c2)ccc1F] 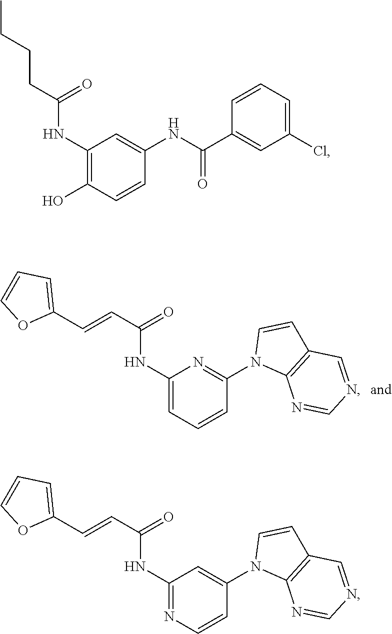 [SMILES: CCCCC(=O)Nc1cc(NC(=O)c2cccc(Cl)c2)ccc1O ; O=C(/C=C/c1ccco1)Nc1cccc(-n2ccc3cncnc32)n1 ; "and" ; O=C(/C=C/c1ccco1)Nc1cc(-n2ccc3cncnc32)ccn1]
or a pharmaceutically acceptable salt or solvate thereof.

103. The pharmaceutical composition of claim 97 or 99, wherein the compound is selected from the group consisting of 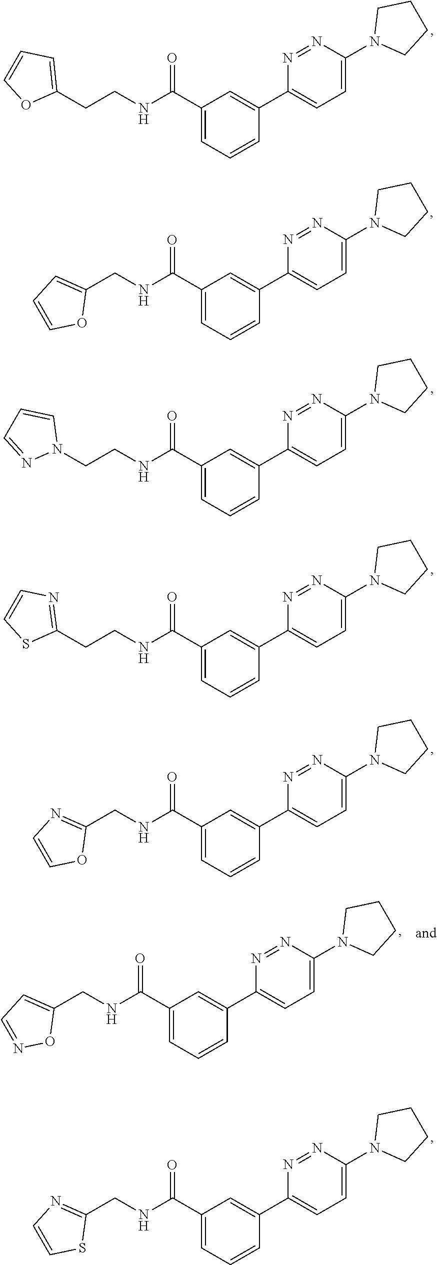
or a pharmaceutically acceptable salt or solvate thereof.

104. A compound of formula (IA): 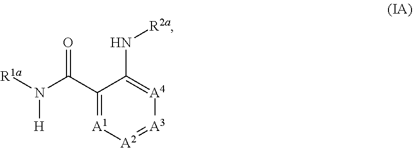
or a pharmaceutically acceptable salt or solvate thereof, for use as a medicament, wherein
A¹, A², A³, and A⁴ are each independently selected from the group consisting of N, CH and C(R^{3a});
each R^{3a} is independently selected from the group consisting of halogen, —OH, —C₁₋₄ alkyl, halo(C₁₋₄ alkyl), —C₁₋₄ alkoxy, halo(C₁₋₄ alkoxy), and —CN;
R^{1a} is selected from the group consisting of —C₁₋₄ alkyl, —C₃₋₁₀ cycloalkyl, —C₁₋₄ alkyl-C₃₋₁₀ cycloalkyl, —C₆₋₁₀ aryl, —C₁₋₄ alkyl-C₆₋₁₀ aryl, -(5- to 10-membered)-C₁₋₉ heteroaryl, —C₁₋₄ alkyl-(5- to 10-membered)-C₁₋₉ heteroaryl, -(5- to 10-membered)-C₂₋₉ heterocyclyl, and —C₁₋₄ alkyl-(5- to 10-membered)-C₂₋₉ heterocyclyl, wherein said alkyl, cycloalkyl, alkylcycloalkyl, aryl, alkylaryl, heteroaryl, alkylheteroaryl, heterocyclyl and alkylheterocyclyl groups are optionally substituted with 1, 2 or 3 substituents each independently selected from the group consisting of halogen, hydroxy, CN, —ORb^{a}, —SRb^{a}, —N(Rb^{a})₂, —C₁₋₄ alkyl optionally substituted with 1, 2, or 3 halogen atoms, optionally substituted —C₆₋₁₀ aryl, optionally substituted -(5- to 10-membered)-C₁₋₉ heteroaryl, and -(5- to 10-membered)-C₂₋₉ heterocyclyl; and wherein said cycloalkyl, alkylcycloalkyl, aryl, alkylaryl, heteroaryl, alkylheteroaryl, heterocyclyl and alkylheterocyclyl is optionally fused to a further (second) ring; and
R^{2a} is selected from the group consisting of —C₁₋₄ alkyl, —C(═O)Ra^{a}, —C(═O)NHRa^{a}, —S(═O)₂Ra^{a}, —C₁₋₄ alkyl-C(═O)Ra^{a}, —C₁₋₄ alkyl-C(═O)NHRa^{a}, —C₁₋₄ alkyl-C(═O)N(Ra^{a})₂, —C₁₋₄ alkyl-S(═O)₂Ra^{a}, —C₁₋₄ alkyl-S(═O)₂—N(Ra^{a})₂, —C₁₋₄ alkyl-C₃₋₁₀ cycloalkyl, —C₁₋₄ alkyl-C₆₋₁₀ aryl, -(5- to 10-membered)-C₁₋₉ heteroaryl, —C₁₋₄ alkyl-(5- to 10-membered)-C₁₋₉ heteroaryl, (5- to 10-membered)-C₂₋₉ heterocyclyl, and —C₁₋₄ alkyl-(5- to 10-membered)-C₂₋₉ heterocyclyl, wherein said alkyl, cycloalkyl, alkylcycloalkyl, aryl, alkylaryl, heteroaryl, alkylheteroaryl, heterocyclyl and alkylheterocyclyl groups are optionally substituted with 1, 2 or 3 substituents each independently selected from the group consisting of halogen, hydroxy, —CN, —C(═O)Ra^{a}, —ORb^{a}, —SRb^{a}, —N(Rb^{a})₂, (═O), —C₁₋₄ alkyl optionally substituted with 1, 2, or 3 halogen atoms, optionally substituted C₆₋₁₀ aryl, optionally substituted -(5- to 10-membered)-C₁₋₉ heteroaryl, and -(5- to 10-membered)-C₂₋₉ heterocyclyl; and wherein said cycloalkyl, alkylcycloalkyl, aryl, alkylaryl, heteroaryl, alkylheteroaryl, heterocyclyl and alkylheterocyclyl is optionally fused to a further (second) ring;
Ra^{a} is selected from the group consisting of —C₁₋₄ alkyl, —C₃₋₁₀ cycloalkyl, —C₁₋₄ alkyl-C₃₋₁₀ cycloalkyl, —C₆₋₁₀ aryl, —C₁₋₄ alkyl-C₆₋₁₀ aryl, -(5- to 10-membered)-C₁₋₉ heteroaryl, —C₁₋₄ alkyl-(5- to 10-membered)-C₁₋₉ heteroaryl, -(5- to 10-membered)-C₂₋₉ heterocyclyl, and —C₁₋₄ alkyl-(5- to 10-membered)-C₂₋₉ heterocyclyl, wherein said alkyl, cycloalkyl, alkylcycloalkyl, aryl, alkylaryl, heteroaryl, alkylheteroaryl, heterocyclyl and alkylheterocyclyl groups are optionally substituted with 1, 2 or 3 substituents each independently selected from the group consisting of halogen, hydroxy, —CN, —ORb^{a}, —SRb^{a}, —N(Rb^{a})₂, —C₁₋₄ alkyl optionally substituted with 1, 2, or 3 halogen atoms, optionally substituted —C₆₋₁₀ aryl, optionally substituted -(5- to 10-membered)-C₁₋₉ heteroaryl, and -(5- to 10-membered)-C₂₋₉ heterocyclyl; and wherein said cycloalkyl, alkylcycloalkyl, aryl, alkylaryl, heteroaryl, alkylheteroaryl, heterocyclyl and alkylheterocyclyl is optionally fused to a further (second) ring; and
each Rb^{a} is independently hydrogen, —C₁₋₄ alkyl, —C₃₋₁₀ cycloalkyl, or -(5- to 10-membered)-C₂₋₉ heterocyclyl, wherein said alkyl, cycloalkyl or heterocyclyl group is optionally substituted by 1, 2 or 3 fluorine atoms.

105. The compound for use according to claim 104, wherein the compound is selected from the group consisting of 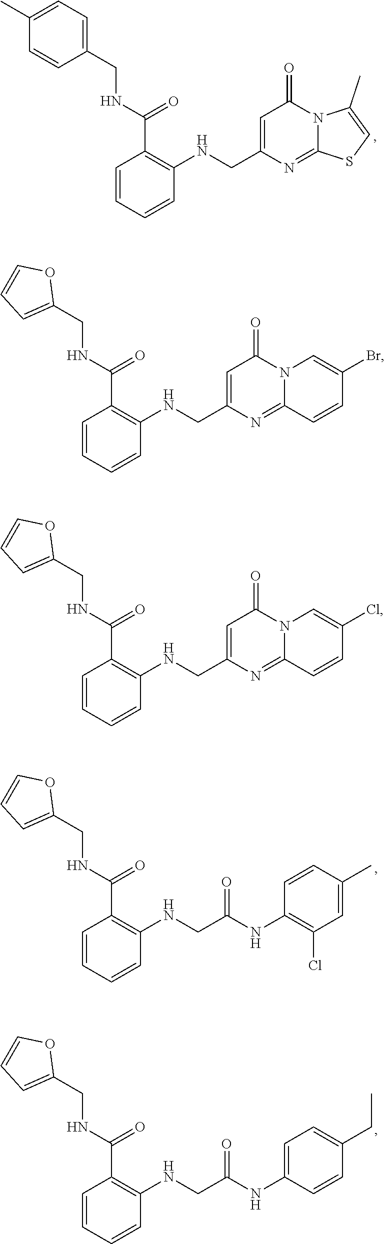 [SMILES: Cc1ccc(CNC(=O)c2ccccc2NCc2cc(=O)n3c(C)csc3n2)cc1 ; O=C(NCc1ccco1)c1ccccc1NCc1cc(=O)n2cc(Br)ccc2n1 ; O=C(NCc1ccco1)c1ccccc1NCc1cc(=O)n2cc(Cl)ccc2n1 ; Cc1ccc(NC(=O)CNc2ccccc2C(=O)NCc2ccco2)c(Cl)c1 ; CCc1ccc(NC(=O)CNc2ccccc2C(=O)NCc2ccco2)cc1] 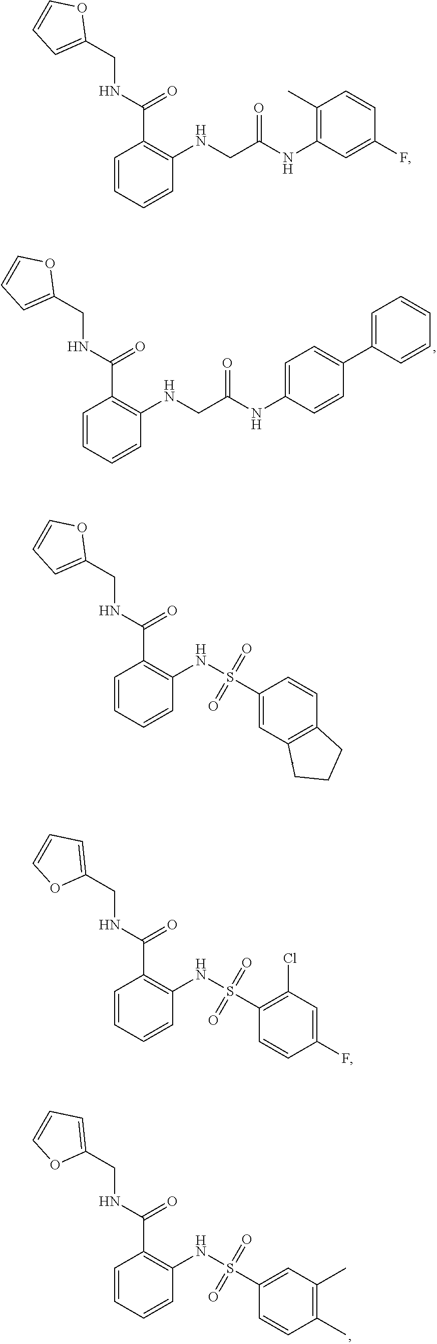 [SMILES: Cc1ccc(F)cc1NC(=O)CNc1ccccc1C(=O)NCc1ccco1 ; O=C(CNc1ccccc1C(=O)NCc1ccco1)Nc1ccc(-c2ccccc2)cc1 ; O=C(NCc1ccco1)c1ccccc1NS(=O)(=O)c1ccc2c(c1)CCC2 ; O=C(NCc1ccco1)c1ccccc1NS(=O)(=O)c1ccc(F)cc1Cl ; Cc1ccc(S(=O)(=O)Nc2ccccc2C(=O)NCc2ccco2)cc1C] 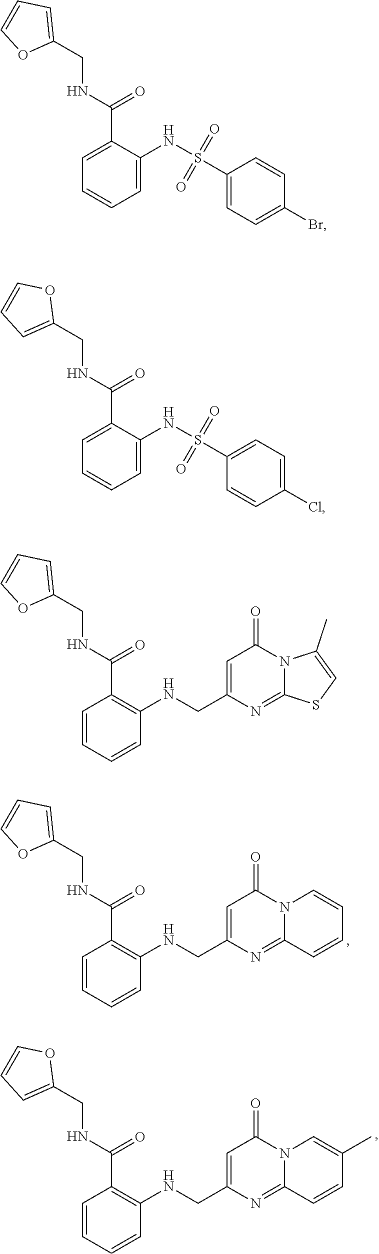 [SMILES: O=C(NCc1ccco1)c1ccccc1NS(=O)(=O)c1ccc(Br)cc1 ; O=C(NCc1ccco1)c1ccccc1NS(=O)(=O)c1ccc(Cl)cc1 ; Cc1csc2nc(CNc3ccccc3C(=O)NCc3ccco3)cc(=O)n12 ; O=C(NCc1ccco1)c1ccccc1NCc1cc(=O)n2ccccc2n1 ; Cc1ccc2nc(CNc3ccccc3C(=O)NCc3ccco3)cc(=O)n2c1] 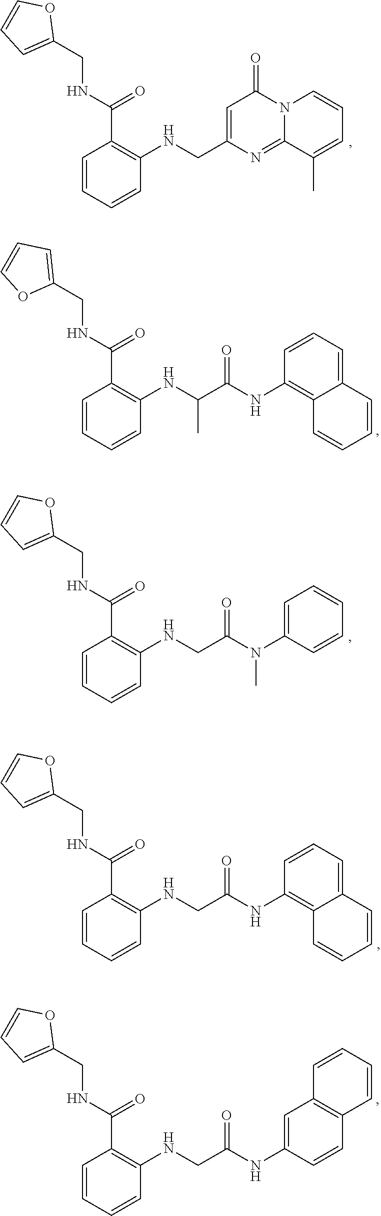 [SMILES: Cc1cccn2c(=O)cc(CNc3ccccc3C(=O)NCc3ccco3)nc12 ; CC(Nc1ccccc1C(=O)NCc1ccco1)C(=O)Nc1cccc2ccccc12 ; CN(C(=O)CNc1ccccc1C(=O)NCc1ccco1)c1ccccc1 ; O=C(CNc1ccccc1C(=O)NCc1ccco1)Nc1cccc2ccccc12 ; O=C(CNc1ccccc1C(=O)NCc1ccco1)Nc1ccc2ccccc2c1] 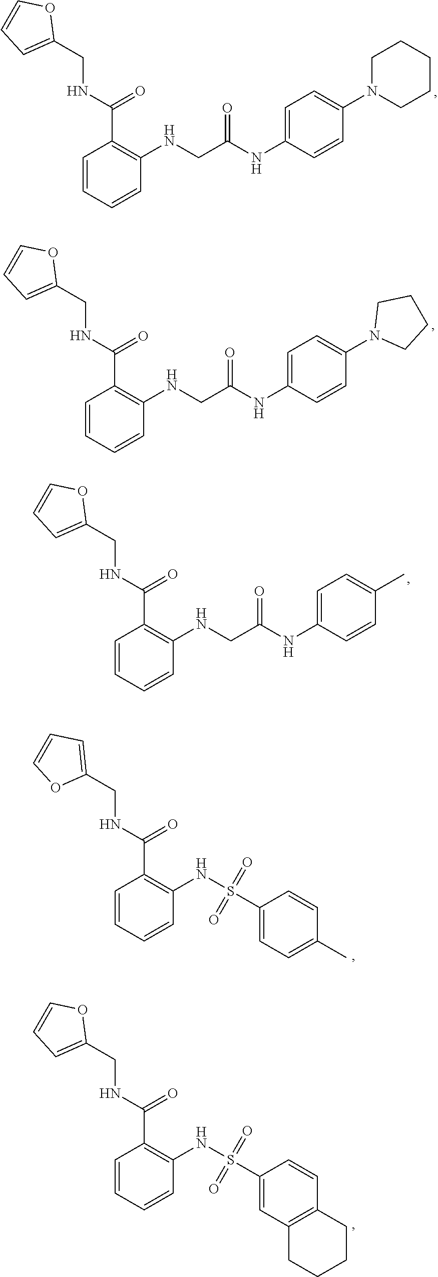 [SMILES: O=C(CNc1ccccc1C(=O)NCc1ccco1)Nc1ccc(N2CCCCC2)cc1 ; O=C(CNc1ccccc1C(=O)NCc1ccco1)Nc1ccc(N2CCCC2)cc1 ; Cc1ccc(NC(=O)CNc2ccccc2C(=O)NCc2ccco2)cc1 ; Cc1ccc(S(=O)(=O)Nc2ccccc2C(=O)NCc2ccco2)cc1 ; O=C(NCc1ccco1)c1ccccc1NS(=O)(=O)c1ccc2c(c1)CCCC2] 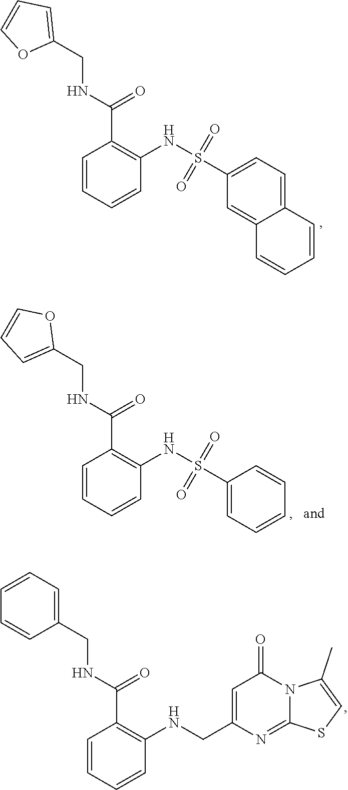 [SMILES: O=C(NCc1ccco1)c1ccccc1NS(=O)(=O)c1ccc2ccccc2c1 ; O=C(NCc1ccco1)c1ccccc1NS(=O)(=O)c1ccccc1 ; Cc1csc2nc(CNc3ccccc3C(=O)NCc3ccccc3)cc(=O)n12]
or a pharmaceutically acceptable salt or solvate thereof.

106. The compound for use according to claim 104, wherein the compound is selected from the group consisting of 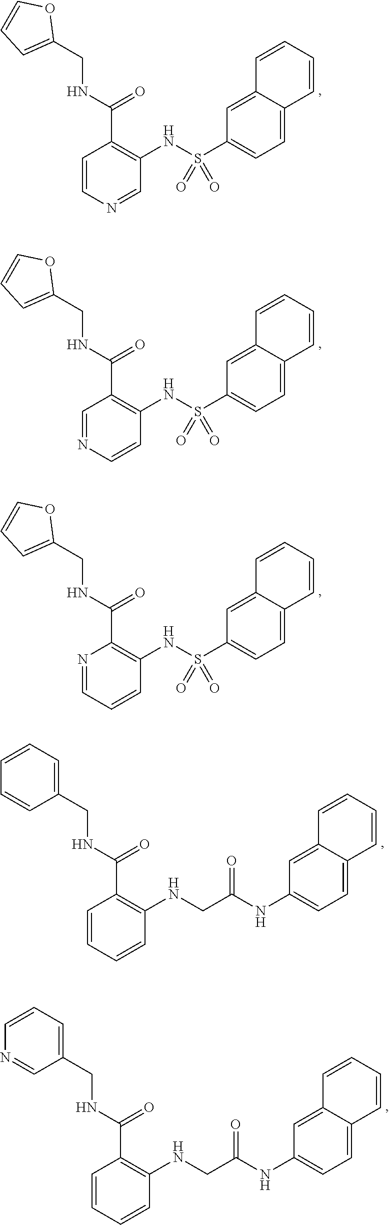 [SMILES: O=C(NCc1ccco1)c1ccncc1NS(=O)(=O)c1ccc2ccccc2c1 ; O=C(NCc1ccco1)c1cnccc1NS(=O)(=O)c1ccc2ccccc2c1 ; O=C(NCc1ccco1)c1ncccc1NS(=O)(=O)c1ccc2ccccc2c1 ; O=C(CNc1ccccc1C(=O)NCc1ccccc1)Nc1ccc2ccccc2c1 ; O=C(CNc1ccccc1C(=O)NCc1cccnc1)Nc1ccc2ccccc2c1] 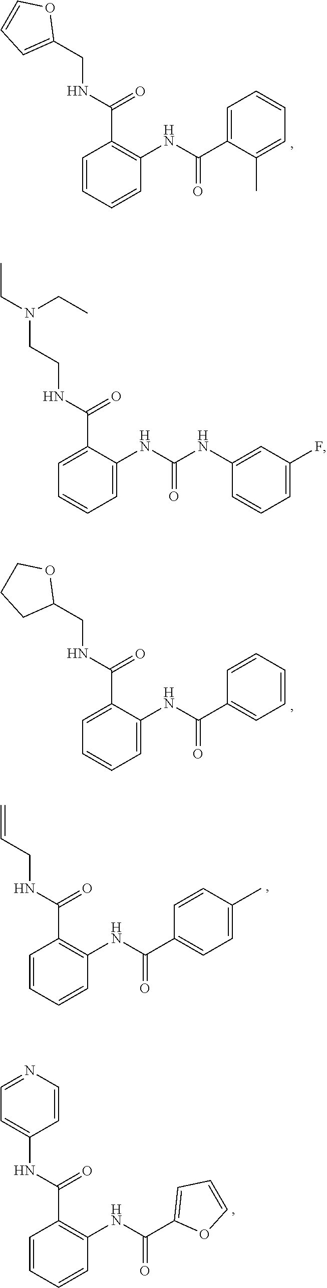 [SMILES: Cc1ccccc1C(=O)Nc1ccccc1C(=O)NCc1ccco1 ; CCN(CC)CCNC(=O)c1ccccc1NC(=O)Nc1cccc(F)c1 ; O=C(Nc1ccccc1C(=O)NCC1CCCO1)c1ccccc1 ; C=CCNC(=O)c1ccccc1NC(=O)c1ccc(C)cc1 ; O=C(Nc1ccccc1C(=O)Nc1ccncc1)c1ccco1] 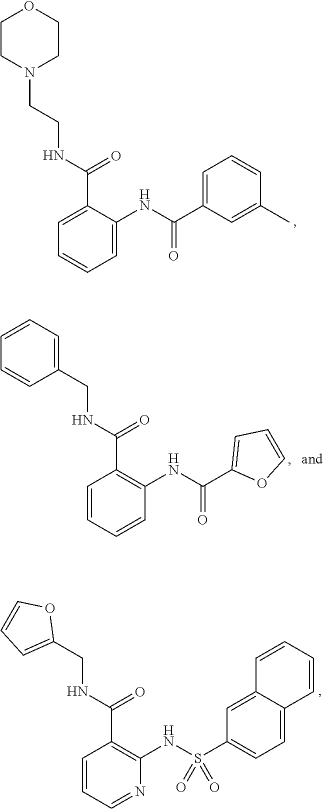 [SMILES: Cc1cccc(C(=O)Nc2ccccc2C(=O)NCCN2CCOCC2)c1 ; O=C(Nc1ccccc1C(=O)NCc1ccccc1)c1ccco1 ; O=C(NCc1ccco1)c1cccnc1NS(=O)(=O)c1ccc2ccccc2c1]
or a pharmaceutically acceptable salt of solvate thereof.

107. A compound of formula (IB) having the structure: 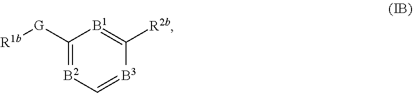
or a pharmaceutically acceptable salt or solvate thereof, for use as a medicament, wherein
G is —C(═O)—NH— or —NH—C(═O)—;
B¹, B², and B³ are each independently selected from the group consisting of N, CH and C(R^{3b});
each R^{3b} is independently selected from the group consisting of halogen, C₁₋₄ alkyl, halo(C₁₋₄ alkyl), —OH, C₁₋₄ alkoxy, halo(C₁₋₄ alkoxy), and CN;
R^{1b} is selected from the group consisting of —C₁₋₄ alkyl, —C₃₋₁₀ cycloalkyl, —C₁₋₄ alkyl-C₃₋₁₀ cycloalkyl, —C₆₋₁₀ aryl, —C₁₋₄ alkyl-C₆₋₁₀ aryl, —C₂₋₄ alkylene-C₆₋₁₀ aryl, (5- to 10-membered)-C₁₋₉ heteroaryl, —C₁₋₄ alkyl-(5- to 10-membered)-C₁₋₉ heteroaryl, —C₂₋₄ alkylene-(5- to 10-membered)-C₁₋₉ heteroaryl, (5- to 10-membered)-C₂₋₉ heterocyclyl, —C₁₋₄ alkyl-(5- to 10-membered)-C₂₋₉ heterocyclyl, and —C₂₋₄ alkenyl-(5- to 10-membered)-C₂₋₉ heterocyclyl, wherein said alkyl, cycloalkyl, alkylcycloalkyl, aryl, alkylaryl, heteroaryl, alkylheteroaryl, alkenylheteroaryl, heterocyclyl and alkylheterocyclyl groups are optionally substituted with 1, 2 or 3 substituents each independently selected from the group consisting of halogen, hydroxy, —CN, —ORb^{b}, —SRb^{b}, —N(Rb^{b})₂, —C₁₋₄ alkyl optionally substituted with 1, 2, or 3 halogen atoms, optionally substituted C₆₋₁₀ aryl, optionally substituted (5- to 10-membered)-C₁₋₉ heteroaryl, and (5- to 10-membered)-C₂₋₉ heterocyclyl; and wherein said cycloalkyl, alkylcycloalkyl, aryl, alkylaryl, heteroaryl, alkylheteroaryl, alkenylheteroaryl, heterocyclyl and alkylheterocyclyl is optionally fused to a further (second) ring;
R^{2b} is —C₆₋₁₀ aryl, -(5- to 10-membered)-C₁₋₉ heteroaryl, —C(═O)Ra^{b}, —S(═O)₂Ra^{b}, —C(═O)—NH—Ra^{b}, —S(═O)₂—NH—Ra^{b}, —C₁₋₄ alkyl-C(═O)Ra^{b}, —C₁₋₄ alkyl-S(═O)₂Ra^{b}, or —N(Rb^{b})₂, wherein said aryl and heteroaryl groups are optionally substituted with 1, 2 or 3 substituents each independently selected from the group consisting of halogen, hydroxy, CN, —ORb^{b}, —SRb^{b}, —N(Rb^{b})₂, (═O), —C₁₋₄alkyl optionally substituted with 1, 2, or 3 substituents each independently selected from the group consisting of halogen, CN, —ORb^{b}, and —N(Rb^{b})₂, optionally substituted —C₆₋₁₀ aryl, optionally substituted -(5- to 10-membered)-C₁₋₉ heteroaryl, -(5- to 10-membered)-C₂₋₉ heterocyclyl, and —C₃₋₁₀ cycloalkyl; and wherein said aryl, heteroaryl, and heterocyclyl are optionally fused to a further (second) ring; or
R^{2b} and R^{3b} attached to an adjacent carbon atom together form a 5- or 6-membered heterocyclic ring containing one N-atom substituted with —S(═O)₂Ra^{b};
Ra^{b} is selected from the group consisting of —C₁₋₄ alkyl, —C₃₋₁₀ cycloalkyl, —C₁₋₄ alkyl-C₃₋₁₀ cycloalkyl, —C₆₋₁₀ aryl, —C₁₋₄ alkyl-C₆₋₁₀ aryl, (5- to 10-membered)-C₁₋₉ heteroaryl, —C₁₋₄ alkyl-(5- to 10-membered)-C₁₋₉ heteroaryl, (5- to 10-membered)-C₂₋₉ heterocyclyl, and —C₁₋₄ alkyl-(5- to 10-membered)-C₂₋₉ heterocyclyl, wherein said alkyl, cycloalkyl, alkylcycloalkyl, aryl, alkylaryl, heteroaryl, alkylheteroaryl, heterocyclyl and alkylheterocyclyl groups are optionally substituted with 1, 2 or 3 substituents each independently selected from the group consisting of halogen, hydroxy, —CN, —ORb^{b}, —SRb^{b}, —N(Rb^{b})₂, —C₁₋₄ alkyl optionally substituted with 1, 2, or 3 halogen atoms, optionally substituted C₆₋₁₀ aryl, optionally substituted (5- to 10-membered)-C₁₋₉ heteroaryl, and (5- to 10-membered)-C₂₋₉ heterocyclyl; and wherein said cycloalkyl, alkylcycloalkyl, aryl, alkylaryl, heteroaryl, alkylheteroaryl, heterocyclyl and alkylheterocyclyl is optionally fused to a further (second) ring; and
each Rb^{b} is independently hydrogen, —C(═O)Ra^{b}, —S(═O)₂Ra^{b}, —C₁₋₄ alkyl, —C₃₋₁₀ cycloalkyl, -(5- to 10-membered)-C₂₋₉ heterocyclyl, or optionally substituted —C₆₋₁₀ aryl, wherein said alkyl, cycloalkyl or heterocyclyl group is optionally substituted by 1, 2 or 3 fluorine atoms.

108. The compound for use according to claim 107, which is compound of formula (IIB): 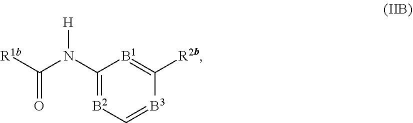
or a pharmaceutically acceptable salt or solvate thereof, wherein B¹, B², B³, R^{1b}, and R^{2b} are as defined in claim 107.

109. The compound for use according to claim 107, which is a compound of formula (IIIB): 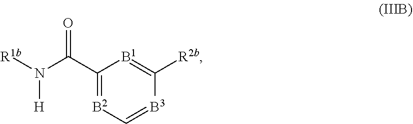
or a pharmaceutically acceptable salt or solvate thereof, wherein B, B², B³, R^{1b}, and R^{2b} are as defined in claim 107.

110. The compound for use according to claim 107 or 108, wherein the compound is selected from the group consisting of 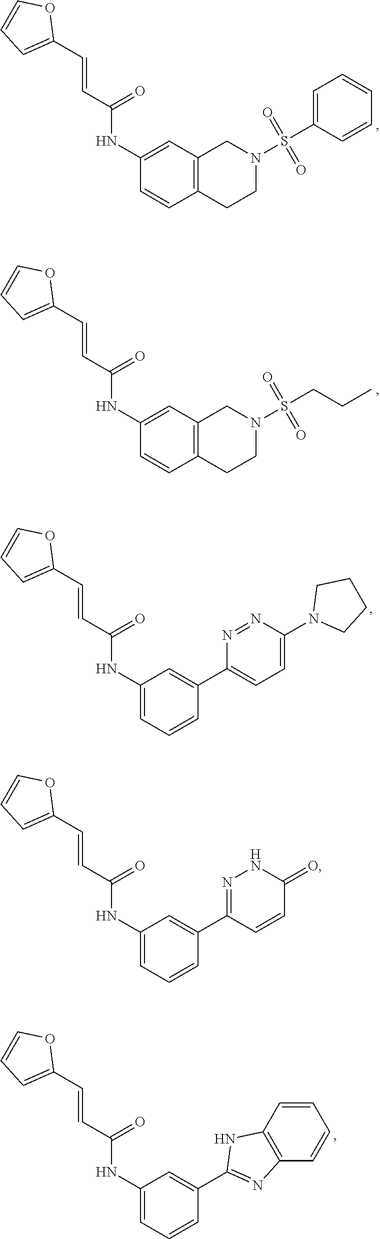 [SMILES: O=C(/C=C/c1ccco1)Nc1ccc2c(c1)CN(S(=O)(=O)c1ccccc1)CC2 ; CCCS(=O)(=O)N1CCc2ccc(NC(=O)/C=C/c3ccco3)cc2C1 ; O=C(/C=C/c1ccco1)Nc1cccc(-c2ccc(N3CCCC3)nn2)c1 ; O=C(/C=C/c1ccco1)Nc1cccc(-c2ccc(=O)[nH]n2)c1 ; O=C(/C=C/c1ccco1)Nc1cccc(-c2nc3ccccc3[nH]2)c1] 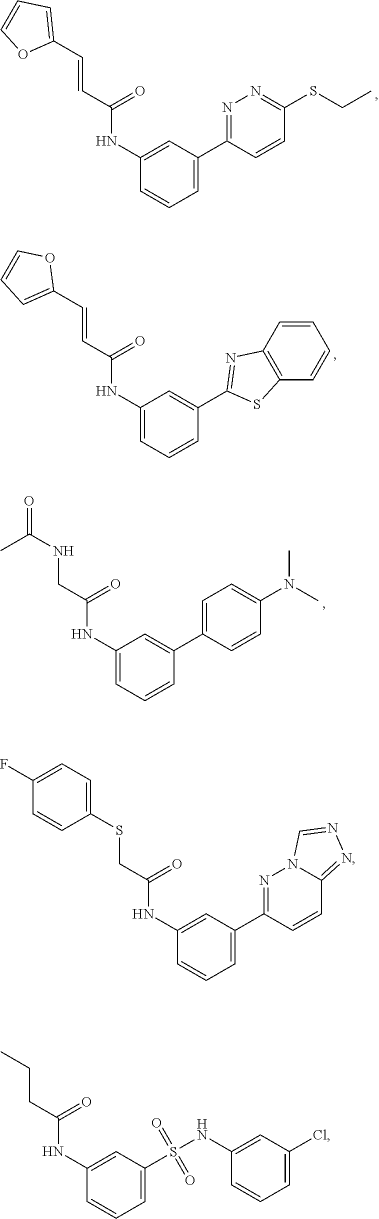 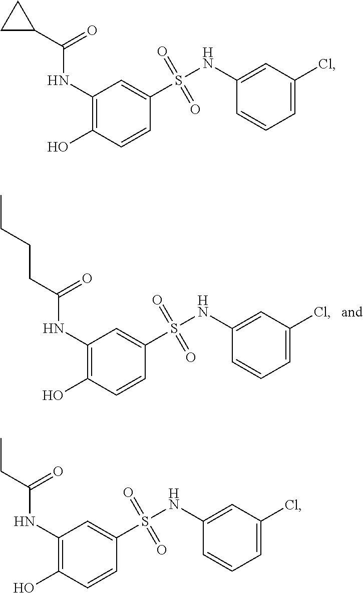 [SMILES: O=C(Nc1cc(S(=O)(=O)Nc2cccc(Cl)c2)ccc1O)C1CC1 ; CCCCC(=O)Nc1cc(S(=O)(=O)Nc2cccc(Cl)c2)ccc1O ; "and" ; CCC(=O)Nc1cc(S(=O)(=O)Nc2cccc(Cl)c2)ccc1O]
or a pharmaceutically acceptable salt or solvate thereof.

111. The compound for use according to claim 107 or 108, wherein the compound is selected from the group consisting of 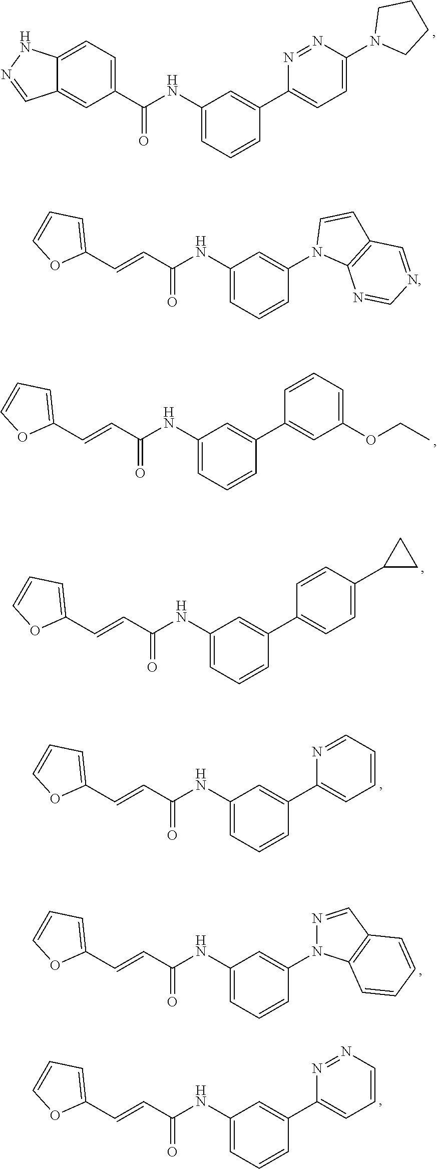 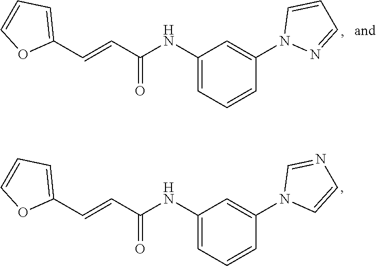 [SMILES: O=C(/C=C/c1ccco1)Nc1cccc(-n2cccn2)c1 ; O=C(/C=C/c1ccco1)Nc1cccc(-n2ccnc2)c1]
or a pharmaceutically acceptable salt or solvate thereof.

112. The compound for use according to claim 107 or 108, wherein the compound is selected from the group consisting of 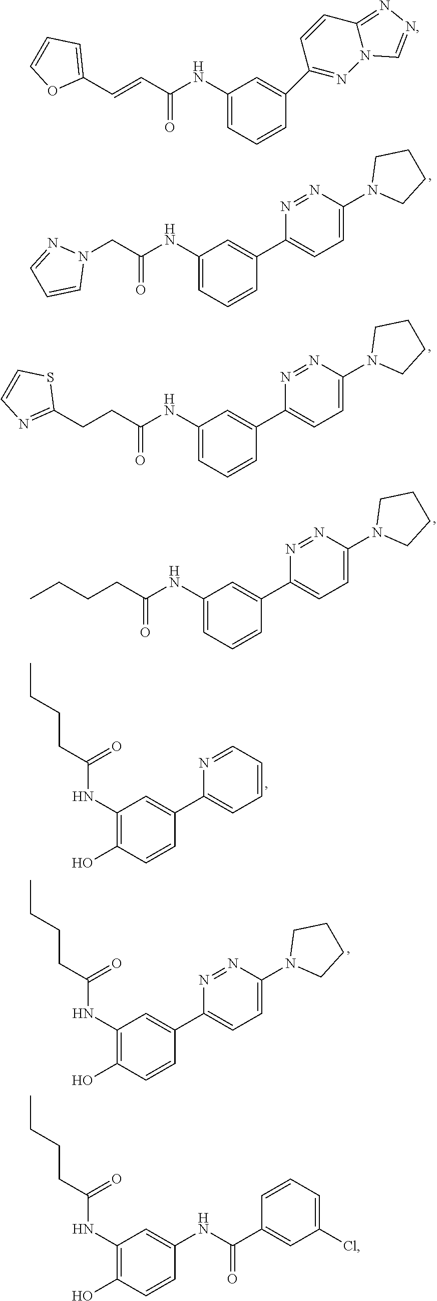 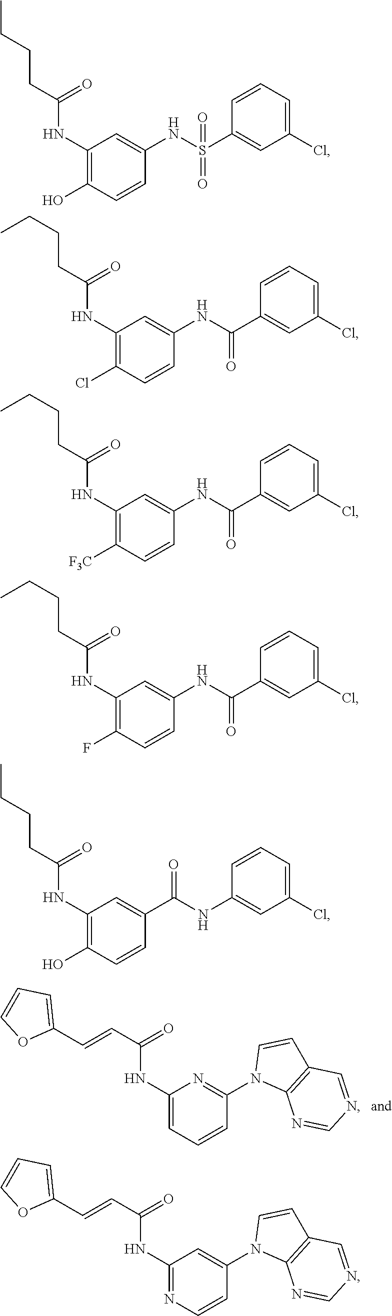
or a pharmaceutically acceptable salt or solvate thereof.

113. The compound for use according to claim 107 or 109, wherein the compound is selected from the group consisting of 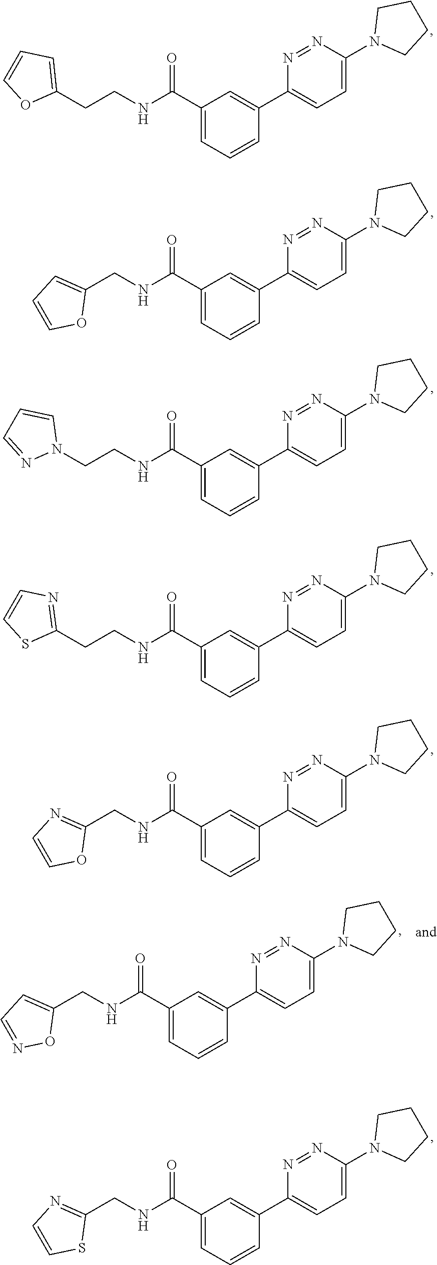
or a pharmaceutically acceptable salt or solvate thereof.

114. The compound for use according to any one of claims 104-113, wherein the medicament is for use in the treatment or prevention of a lysosomal storage disease.

115. The compound ofr use according to claim 114, wherein the lysosomal storage disease is Krabbe's disease.

116. The compound for use according to any one of claims 104-113, wherein the medicament is for use in the treatment or prevention of an α-synucleinopathy.

117. The compound for use according to any one of claims 104-113, wherein the medicament is for use in the treatment or prevention of a disease or disorder selected from the group consisting of Krabbe's disease, demyelinating disorders, galactosylsphingosine related disorders, globoid cell leukodystrophy, multiple sclerosis (MS), Parkinson's disease, peripheral neuropathy, progressive multiple sclerosis, pulmonary artery enlargement in COPD, open angle glaucoma, Lewy body dementia, and multiple system atrophy (MSA).
